(19) United States
(12) Patent Application Publication
(10) Pub. No.: US 2022/0384732 A1
(43) Pub. Date: Dec. 1, 2022

(54) MATERIALS FOR ELECTRONIC DEVICES

(71) Applicant: Merck Patent GmbH, Darmstadt (DE)

(72) Inventors: Amir PARHAM, Frankfurt am Main (DE); Jonas KROEBER, Frankfurt am Main (DE); Jens ENGELHART, Darmstadt (DE); Anja JATSCH, Frankfurt am Main (DE); Christian EICKHOFF, Mannheim (DE); Christian EHRENREICH, Darmstadt (DE)

(21) Appl. No.: 17/258,778

(22) PCT Filed: Jul. 5, 2019
(86) PCT No.: PCT/EP2019/068066

§ 371 Date: Jan. 8, 2021

(30) Foreign Application Priority Data
Jul. 9, 2018 (EP) 18182505.0

(51) Int. Cl. H01L 51/00; C09K 11/02; C09K 11/06; C07D 409/10; C07D 405/10; C07D 405/14; C07D 409/14; C07D 405/04; C07D 471/04; C07D 409/04

(52) U.S. Cl. CPC H01L 51/0072; H01L 51/5016; C09K 11/06; C07D 409/10; H01L 51/0074; H01L 51/0052; C07D 405/10; H01L 51/0073; H01L 51/0056; C07D 405/14; C07D 409/14; C07D 405/04; C07D 471/04; C07D 409/04; C09K 2211/185; C09K 2211/1029; C09K 2211/1007; C09K 11/02

(57) ABSTRACT

The invention relates to heteroaromatic, polycyclic condensed compounds according to the formulae (i) and (ii) defined below. Said compounds are suitable for use in electronic devices.

The present application relates to heteroaromatic, polycyclically fused compounds of the formulae (I) and (II) defined below. These compounds are suitable for use in electronic devices.

Electronic devices in the context of this application are understood to mean what are called organic electronic devices, which contain organic semiconductor materials as functional materials. More particularly, these are understood to mean OLEDs (organic electroluminescent devices). The term OLEDs is understood to mean electronic devices which have one or more layers comprising organic compounds and emit light on application of electrical voltage. The construction and general principle of function of OLEDs are known to those skilled in the art.

In electronic devices, especially OLEDs, there is great interest in an improvement in the performance data, especially lifetime, efficiency and operating voltage. In these aspects, it has not yet been possible to find any entirely satisfactory solution.

There is an ongoing search for materials having high oxidation stability, especially in solution, high thermal stability, such that they can be evaporated under high vacuum without decomposition, and high glass transition temperature T_{G}.

A major influence on the performance data of electronic devices is possessed by phosphorescent emission layers. These typically contain at least two different matrix materials and at least one phosphorescent emitter. For use in these layers, there is an ongoing search for new matrix materials, especially those that have a large energy gap between HOMO and LUMO (wide bandgap materials). More particularly, there is a search for new matrix materials that can be used in combination with a further matrix material in emitting layers containing a triplet emitter. The further matrix material is preferably selected from hole-conducting compounds, from electron-conducting compounds, and from compounds having both hole-conducting and electron-conducting properties. The latter compounds are referred to as bipolar matrix materials.

The prior art discloses a multitude of heteroaromatic compounds for use in OLEDs.

However, there is still a need for alternative compounds suitable for the purpose. There is also a need for improvement with regard to the performance data in the case of use in electronic devices, especially with regard to lifetime, operating voltage and efficiency, and with respect to the abovementioned properties of oxidation stability, thermal stability and high glass transition temperature T_{G}.

It has now been found that particular compounds from the abovementioned structure class are of excellent suitability for use in electronic devices, especially for use in OLEDs, more particularly for use therein as matrix materials for phosphorescent emitters, more particularly for use as wide-bandgap matrix materials in combination with at least one further matrix material and at least one phosphorescent emitter. The compounds preferably lead to an improvement in the abovementioned material properties and to an improvement in the abovementioned properties of the OLEDs.

These compounds are provided by the present application. They conform to a formula (I) or (II)

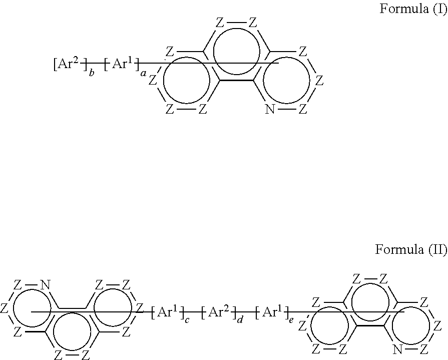

where the variables that occur are as follows:
Z, when no Ar¹ unit binds thereto, is the same or different at each instance and is selected from CR¹ and N; and Z, when an Ar¹ unit binds thereto, is C;
Ar¹ is the same of different at each instance and is selected from a single bond, aromatic ring system which has 6 to 40 aromatic ring atoms and may be substituted by one or more R² radicals, dibenzothiophene which may be substituted by one or more R² radicals, and dibenzofuran which may be substituted by one or more R¹ radicals;
Ar² is the same or different at each instance and is a group of formula (Ar²)

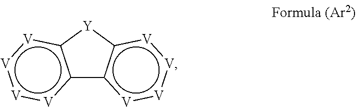

Y is the same or different at each instance and is selected from O, S, C(R³)₂, Si(R³)₂,

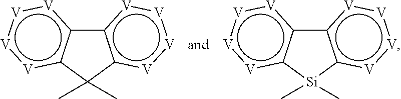

where, in the formulae

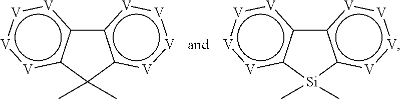

the free bonds are the bonds proceeding from the Y group to the rest of the group of the formula (Ar²);
V is the same or different at each instance and is CR³ or N if the bond to the rest of the formula is not at the position in question, and V is C if the bond to the rest of the formula is at the position in question;
where one or more pairs V-V may each be replaced by a unit selected from the following units:

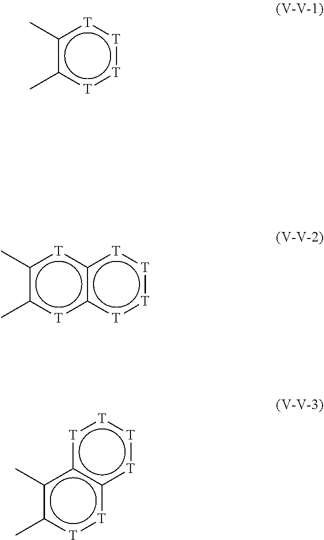

where the free bonds indicate the bonds to the rest of the formula, and where T is the same or different at each instance and is CR³ or N if the bond to the rest of the formula is not at the position in question, and where T is C if the bond to the rest of the formula is at the position in question;
R¹, R² are the same or different at each instance and are selected from H, D, F, C(═O)R⁴, CN, Si(R⁴)₃, P(═O)(R⁴)₂, OR⁴, S(═O)R⁴, S(═O)₂R⁴, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R¹ or R² radicals may be joined to one another and may form a ring; where the alkyl, alkoxy, alkenyl and alkynyl groups mentioned and the aromatic ring systems and heteroaromatic ring systems mentioned may each be substituted by one or more R⁴ radicals; and where one or more CH₂ groups in the alkyl, alkoxy, alkenyl and alkynyl groups mentioned may be replaced by —R⁴C═CR⁴—, —C≡C—, Si(R⁴)₂, C═O, C═NR⁴, —C(═O)O—, —C(═O)NR⁴—, P(═O)(R⁴), —O—, —S—, SO or SO₂;
R³ is the same or different at each instance and is selected from H, D, F, C(═O)R⁴, CN, Si(R⁴)₃, P(═O)(R⁴)₂, OR⁴, S(═O)R⁴, S(═O)₂R⁴, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, heteroaromatic N-free ring systems having 5 to 40 aromatic ring atoms; and electron-deficient heteroaryl groups having 6 to 40 aromatic ring atoms, where two or more R³ radicals may be joined to one another and may form a ring; where the alkyl, alkoxy, alkenyl and alkynyl groups mentioned and the aromatic ring systems and heteroaromatic N-free ring systems and electron-deficient heteroaryl groups mentioned may each be substituted by one or more R⁴ radicals; and where one or more CH₂ groups in the alkyl, alkoxy, alkenyl and alkynyl groups mentioned may be replaced by —R⁴C═CR⁴—, —C≡C—, Si(R⁴)₂, C═O, C═NR⁴, —C(═O)O—, —C(═O)NR⁴—, P(═O)(R⁴), —O—, —S—, SO or SO₂;
R⁴ is the same or different at each instance and is selected from H, D, F, C(═O)R⁵, CN, Si(R⁵)₃, P(═O)(R⁵)₂, OR⁵, S(═O)R⁵, S(═O)₂R⁵, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R⁴ radicals may be joined to one another and may form a ring; where the alkyl, alkoxy, alkenyl and alkynyl groups mentioned and the aromatic ring systems and heteroaromatic ring systems mentioned may each be substituted by one or more R⁵ radicals; and where one or more CH₂ groups in the alkyl, alkoxy, alkenyl and alkynyl groups mentioned may be replaced by —R⁵C═CR⁵—, —C≡C—, Si(R⁵)₂, C═O, C═NR⁵, —C(═O)O—, —C(═O)NR⁵—, P(═O)(R⁵), —O—, —S—, SO or SO₂;
R⁵ is the same or different at each instance and is selected from H, D, F, CN, alkyl or alkoxy groups having 1 to 20 carbon atoms, alkenyl or alkynyl groups having 2 to 20 carbon atoms, aromatic ring systems having 6 to 40 aromatic ring atoms and heteroaromatic ring systems having 5 to 40 aromatic ring atoms; where the alkyl, alkoxy, alkenyl and alkynyl groups, aromatic ring systems and heteroaromatic ring systems mentioned may be substituted by one or more radicals selected from F and CN;
a, b, c, d, e are the same or different and are selected from 1, 2, 3 and 4.

The circles drawn in the six-membered rings mean that the six-membered rings in question are aromatic or heteroaromatic.

If any index selected from indices a, b, c, d and e is greater than 1, this means that the group in question that has been given the index occurs more than once in succession in a chain. What is meant, for example, by —[Ar¹]ₐ— when a=2 is that an —Ar¹-Ar¹— unit is present. When a=3, it correspondingly means that an —Ar¹-Ar¹-Ar¹— unit is present.

The bond drawn through the three six-membered rings fused to one another in the skeleton means that the bond may be at any position in the skeleton.

The definitions which follow are applicable to the chemical groups that are used in the present applications. They are applicable unless any more specific definitions are given.

An aryl group in the context of this invention is understood to mean either a single aromatic cycle, i.e. benzene, or a fused aromatic polycycle, for example naphthalene, phenanthrene or anthracene. A fused aromatic polycycle in the context of the present application consists of two or more single aromatic cycles fused to one another. Fusion between cycles is understood here to mean that the cycles share at least one edge with one another. An aryl group in the context of this invention contains 6 to 40 aromatic ring atoms of which none is a heteroatom.

A heteroaryl group in the context of this invention is understood to mean either a single heteroaromatic cycle, for example pyridine, pyrimidine or thiophene, or a fused heteroaromatic polycycle, for example quinoline or carbazole. A fused heteroaromatic polycycle in the context of the present application consists of two or more single aromatic or heteroaromatic cycles that are fused to one another, where at least one of the aromatic and heteroaromatic cycles is a heteroaromatic cycle. Fusion between cycles is understood here to mean that the cycles share at least one edge with one another. A heteroaryl group in the context of this invention contains 5 to 40 aromatic ring atoms of which at least one is a heteroatom. The heteroatoms of the heteroaryl group are preferably selected from N, O and S.

The term “electron-deficient heteroaryl group” is understood in the usual way by the person skilled in the art in the field of organic chemistry. This term is especially understood to mean a heteroaryl group having at least one unit selected from the following units: i) a heteroaromatic six-membered ring containing at least one nitrogen atom; ii) a heteroaromatic five-membered ring containing at least two nitrogen atoms.

An aryl or heteroaryl group, each of which may be substituted by the abovementioned radicals, is especially understood to mean groups derived from benzene, naphthalene, anthracene, phenanthrene, pyrene, dihydropyrene, chrysene, perylene, triphenylene, fluoranthene, benzanthracene, benzophenanthrene, tetracene, pentacene, benzopyrene, furan, benzofuran, isobenzofuran, dibenzofuran, thiophene, benzothiophene, isobenzothiophene, dibenzothiophene, pyrrole, indole, isoindole, carbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, benzimidazolo[1,2-a]benzimidazole, naphthimidazole, phenanthrimidazole, pyridimidazole, pyrazinimidazole, quinoxalinimidazole, oxazole, benzoxazole, naphthoxazole, anthroxazole, phenanthroxazole, isoxazole, 1,2-thiazole, 1,3-thiazole, benzothiazole, pyridazine, benzopyridazine, pyrimidine, benzopyrimidine, quinoxaline, pyrazine, phenazine, naphthyridine, azacarbazole, benzocarboline, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole, 1,2,4,5-tetrazine, 1,2,3,4-tetrazine, 1,2,3,5-tetrazine, purine, pteridine, indolizine and benzothiadiazole.

An aromatic ring system in the context of this invention is a system which does not necessarily contain solely aryl groups, but which may additionally contain one or more non-aromatic rings fused to at least one aryl group. These non-aromatic rings contain exclusively carbon atoms as ring atoms. Examples of groups covered by this definition are tetrahydronaphthalene, fluorene and spirobifluorene. In addition, the term “aromatic ring system” includes systems that consist of two or more aromatic ring systems joined to one another via single bonds, for example biphenyl, terphenyl, 7-phenyl-2-fluorenyl, quaterphenyl and 3,5-diphenyl-1-phenyl. An aromatic ring system in the context of this invention contains 6 to 40 carbon atoms and no heteroatoms in the ring system. The definition of “aromatic ring system” does not include heteroaryl groups.

A heteroaromatic ring system conforms to the abovementioned definition of an aromatic ring system, except that it must contain at least one heteroatom as ring atom. As is the case for the aromatic ring system, the heteroaromatic ring system need not contain exclusively aryl groups and heteroaryl groups, but may additionally contain one or more non-aromatic rings fused to at least one aryl or heteroaryl group. The nonaromatic rings may contain exclusively carbon atoms as ring atoms, or they may additionally contain one or more heteroatoms, where the heteroatoms are preferably selected from N, O and S. One example of such a heteroaromatic ring system is benzopyranyl. In addition, the term “heteroaromatic ring system” is understood to mean systems that consist of two or more aromatic or heteroaromatic ring systems that are bonded to one another via single bonds, for example 4,6-diphenyl-2-triazinyl. A heteroaromatic ring system in the context of this invention contains 5 to 40 ring atoms selected from carbon and heteroatoms, where at least one of the ring atoms is a heteroatom. The heteroatoms of the heteroaromatic ring system are preferably selected from N, O and S.

The terms “heteroaromatic ring system” and “aromatic ring system” as defined in the present application thus differ from one another in that an aromatic ring system cannot have a heteroatom as ring atom, whereas a heteroaromatic ring system must have at least one heteroatom as ring atom. This heteroatom may be present as a ring atom of a non-aromatic heterocyclic ring or as a ring atom of an aromatic heterocyclic ring.

In accordance with the above definitions, any aryl group is covered by the term “aromatic ring system”, and any heteroaryl group is covered by the term “heteroaromatic ring system”.

The term “heteroaromatic N-free ring system” is understood to mean any heteroaromatic ring system as defined above that does not have any nitrogen atoms as constituents of the ring system. Bridging or bonding nonaromatic groups are likewise regarded here as constituents of the ring system. More particularly, the term “heteroaromatic N-free ring system” excludes carbazole, dihydroacridine and derivatives thereof.

An aromatic ring system having 6 to 40 aromatic ring atoms or a heteroaromatic ring system having 5 to 40 aromatic ring atoms is especially understood to mean groups derived from the groups mentioned above under aryl groups and heteroaryl groups, and from biphenyl, terphenyl, quaterphenyl, fluorene, spirobifluorene, dihydrophenanthrene, dihydropyrene, tetrahydropyrene, indenofluorene, truxene, isotruxene, spirotruxene, spiroisotruxene, indenocarbazole, or from combinations of these groups.

In the context of the present invention, a straight-chain alkyl group having 1 to 20 carbon atoms and a branched or cyclic alkyl group having 3 to 20 carbon atoms and an alkenyl or alkynyl group having 2 to 40 carbon atoms in which individual hydrogen atoms or CH₂ groups may also be substituted by the groups mentioned above in the definition of the radicals are preferably understood to mean the methyl, ethyl, n-propyl, i-propyl, n-butyl, i-butyl, s-butyl, t-butyl, 2-methylbutyl, n-pentyl, s-pentyl, cyclopentyl, neopentyl, n-hexyl, cyclohexyl, neohexyl, n-heptyl, cycloheptyl, n-octyl, cyclooctyl, 2-ethylhexyl, trifluoromethyl, pentafluoroethyl, 2,2,2-trifluoroethyl, ethenyl, propenyl, butenyl, pentenyl, cyclopentenyl, hexenyl, cyclohexenyl, heptenyl, cycloheptenyl, octenyl, cyclooctenyl, ethynyl, propynyl, butynyl, pentynyl, hexynyl or octynyl radicals.

An alkoxy or thioalkyl group having 1 to 20 carbon atoms in which individual hydrogen atoms or CH₂ groups may also be replaced by the groups mentioned above in the definition of the radicals is preferably understood to mean methoxy, trifluoromethoxy, ethoxy, n-propoxy, i-propoxy, n-butoxy, i-butoxy, s-butoxy, t-butoxy, n-pentoxy, s-pentoxy, 2-methylbutoxy, n-hexoxy, cyclohexyloxy, n-heptoxy, cycloheptyloxy, n-octyloxy, cyclooctyloxy, 2-ethylhexyloxy, pentafluoroethoxy, 2,2,2-trifluoroethoxy, methylthio, ethylthio, n-propylthio, i-propylthio, n-butylthio, i-butylthio, s-butylthio, t-butylthio, n-pentylthio, s-pentylthio, n-hexylthio, cyclohexylthio, n-heptylthio, cycloheptylthio, n-octylthio, cyclooctylthio, 2-ethylhexylthio, trifluoromethylthio, pentafluoroethylthio, 2,2,2-trifluoroethylthio, ethenylthio, propenylthio, butenylthio, pentenylthio, cyclopentenylthio, hexenylthio, cyclohexenylthio, heptenylthio, cycloheptenylthio, octenylthio, cyclooctenylthio, ethynylthio, propynylthio, butynylthio, pentynylthio, hexynylthio, heptynylthio or octynylthio.

The wording that two or more radicals together may form a ring, in the context of the present application, shall be understood to mean, inter alia, that the two radicals are joined to one another by a chemical bond. In addition, however, the abovementioned wording shall also be understood to mean that, if one of the two radicals is hydrogen, the second radical binds to the position to which the hydrogen atom was bonded, forming a ring.

The compound of the abovementioned formulae preferably does not contain any anthracene group. More preferably, the compound of the abovementioned formulae does not contain any fused aryl group having more than 10 aromatic ring atoms.

Preferably not more than 2 Z groups for each abovementioned formula are N; more preferably, not more than 1 Z group is N. Most preferably, none of the Z groups in the abovementioned formulae is N.

It is further preferable that, for each six-membered ring in a unit

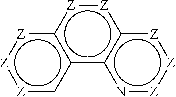

in the abovementioned formulae, not more than one Z group is N.

Preferably, Ar¹ is the same or different at each instance and is selected from a single bond, dibenzofuran, dibenzothiophene, benzene, naphthalene, biphenyl, terphenyl, fluorene, and spirobifluorene, each of which may be substituted by one or more R² radicals.

More preferably, Ar¹ is the same or different at each instance and is selected from a single bond and the formulae shown below:

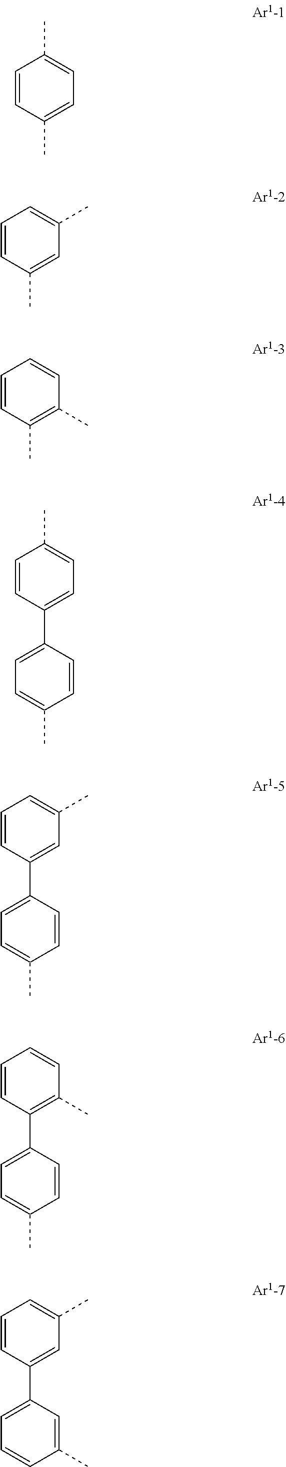

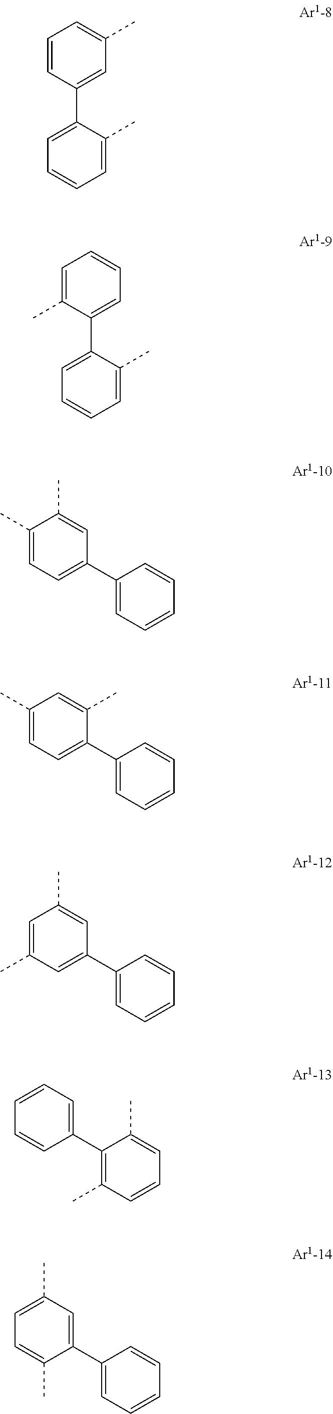

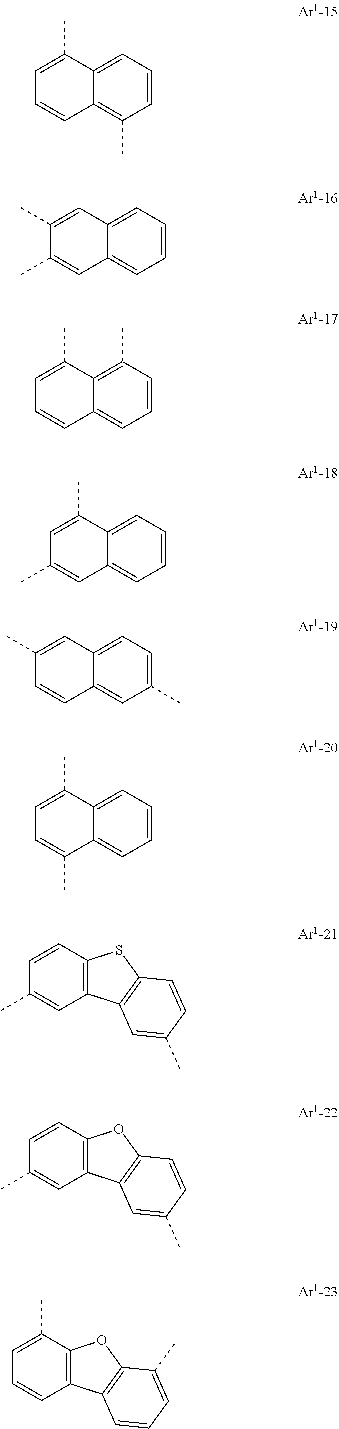

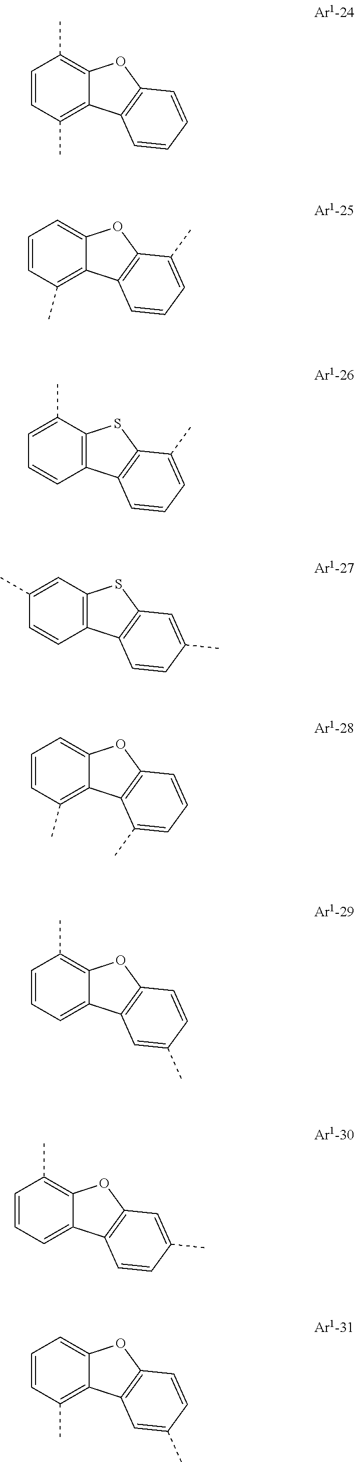

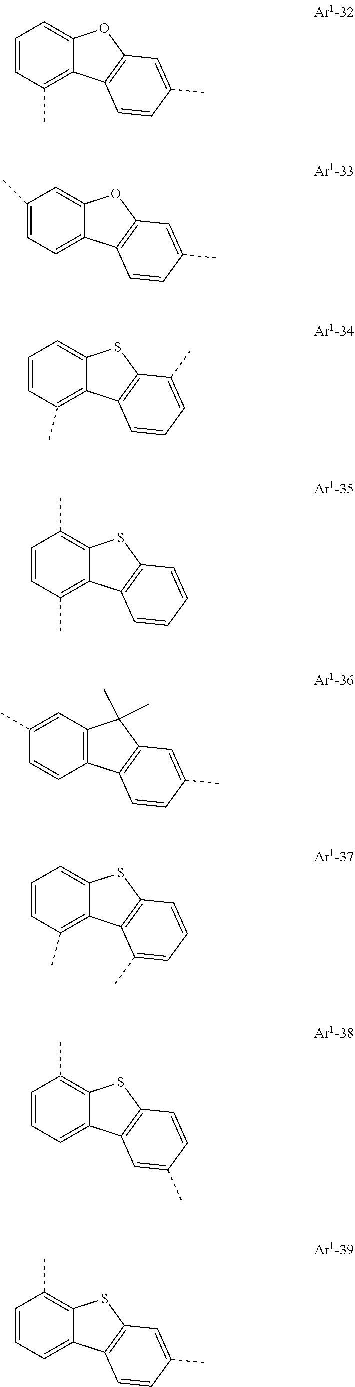

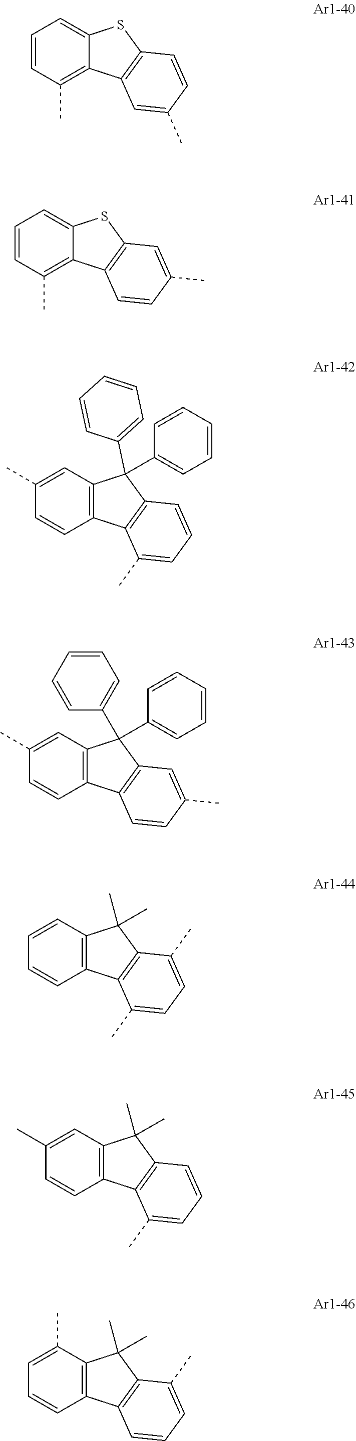

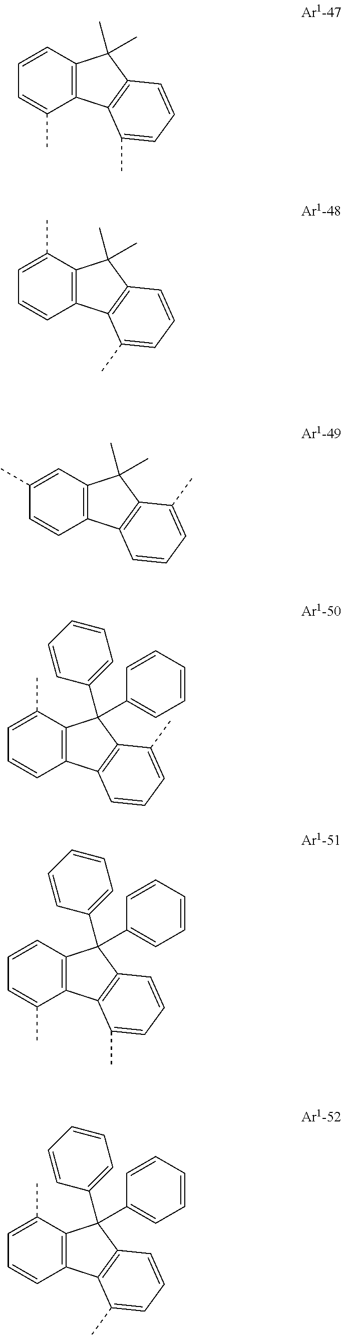

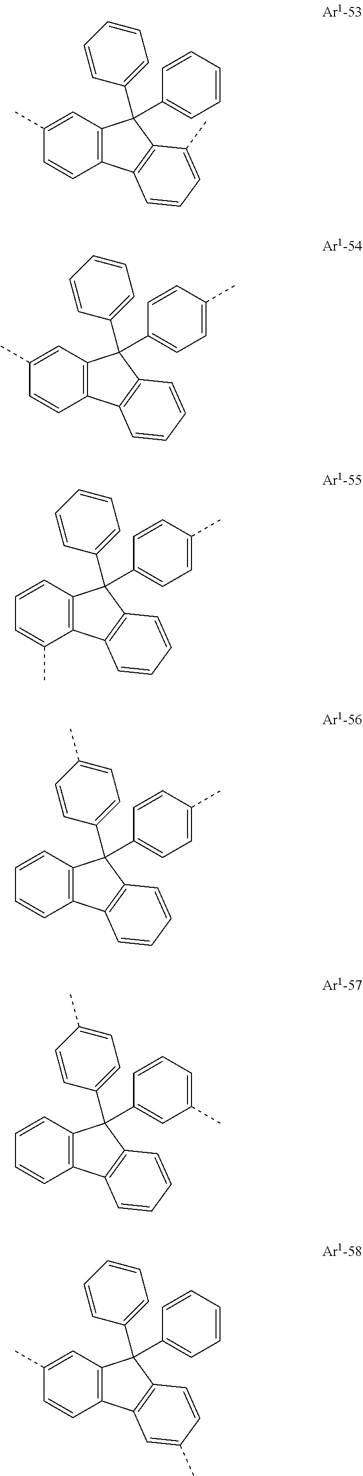

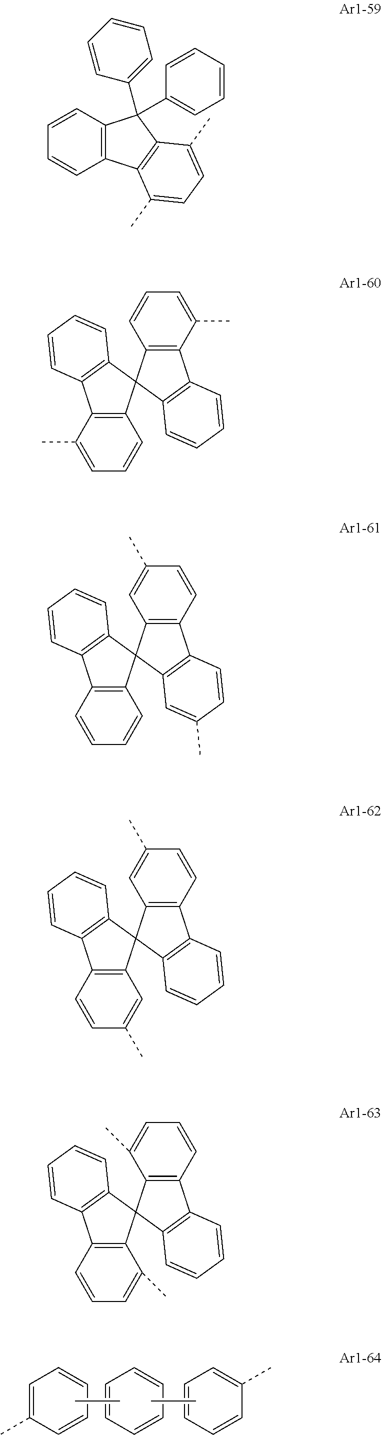

where the formulae may each be substituted by one or more R².

Y is preferably the same or different at each instance and is selected from O and S.

Preferably, Ar² is the same or different at each instance and is selected from the formulae

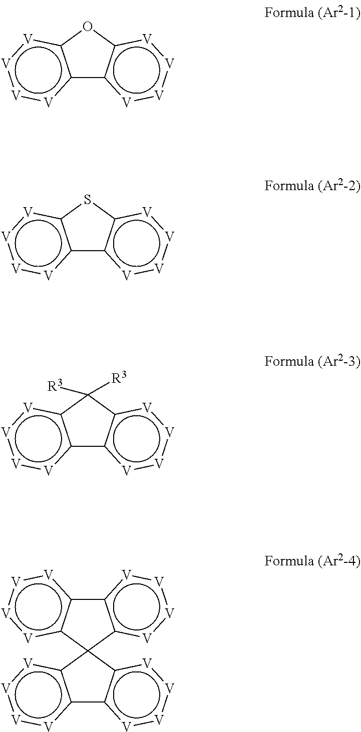

where V is the same or different at each instance and is CR³ or N if the bond to the rest of the formula is not at the position in question, and where V is C if the bond to the rest of the formula is at the position in question;
where one or more pairs V-V may each be replaced by a unit selected from the following units:

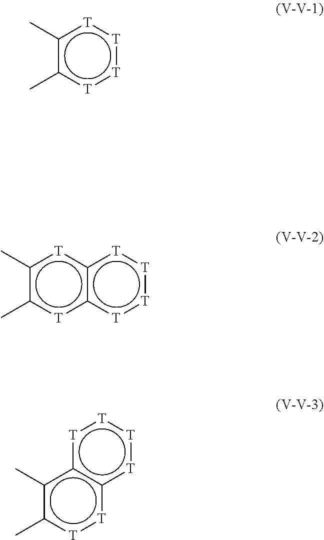

where the free bonds indicate the bonds to the rest of the formula, and where T is the same or different at each instance and is CR³ or N if the bond to the rest of the formula is not at the position in question, and where T is C if the bond to the rest of the formula is at the position in question. Preferably not more than 2 V groups for each abovementioned formula are N; more preferably, not more than 1 V group is N. Most preferably, V is CR³ if the bond to the rest of the formula is not at the position in question, and is C if the bond to the rest of the formula is at the position in question.

Preferably not more than 2 T groups for each abovementioned formula are N; more preferably, not more than 1 T group is N. Most preferably, T is CR³ if the bond to the rest of the formula is not at the position in question, and is C if the bond to the rest of the formula is at the position in question.

Among the abovementioned formulae, particular preference is given to the formula (V-V-1), most preferably with all T groups ═CR³.

Preferred embodiments of the formula (Ar²-1) and (Ar²-2) are thus the following formulae:

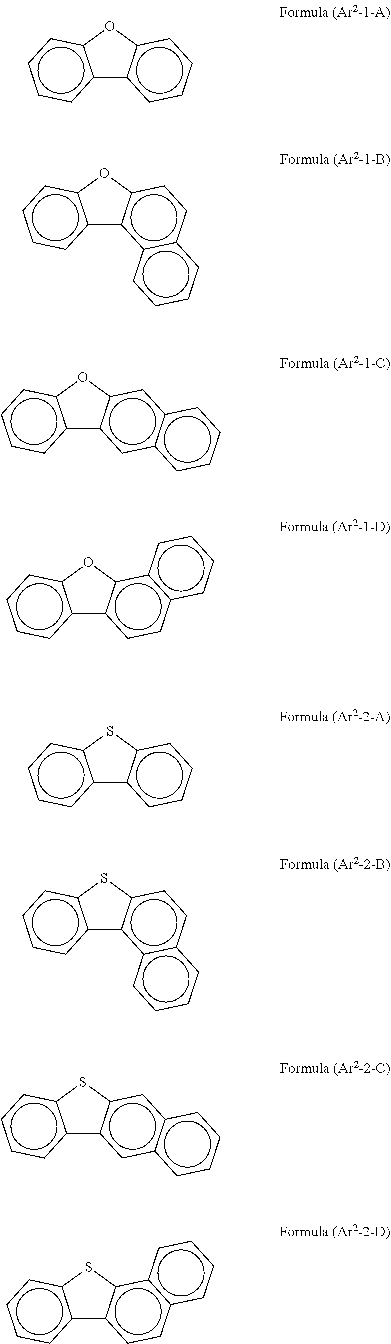

where the groups at each of the unoccupied positions may each be substituted by an R³ radical, and where the groups at any unoccupied position may be bonded to the rest of the compound.

Particularly preferred embodiments of the abovementioned formulae are the following formulae:

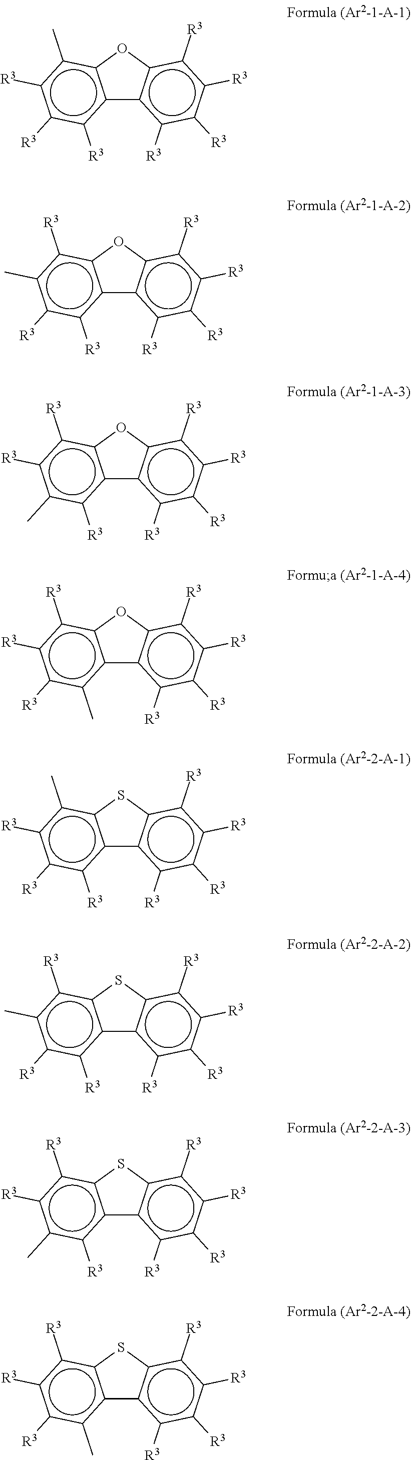

where the free bond denotes the bond to the rest of the formula.

Preferred units —[Ar¹]ₐ—[Ar²]_{b} in formula (I) are selected from units of the following formulae —Ar¹-Ar² in which Ar¹ and Ar² are selected as follows:

| Formula | Ar¹ | Ar² |
|---|---|---|
| (Ar1-Ar2-1) | Single bond | (Ar²-1-A-1) |
| (Ar1-Ar2-2) | Ar¹-1 | (Ar²-1-A-1) |
| (Ar1-Ar2-3) | Ar¹-2 | (Ar²-1-A-1) |
| (Ar1-Ar2-4) | Ar¹-3 | (Ar²-1-A-1) |
| (Ar1-Ar2-5) | Ar¹-4 | (Ar²-1-A-1) |
| (Ar1-Ar2-6) | Ar¹-5 | (Ar²-1-A-1) |
| (Ar1-Ar2-7) | Ar¹-6 | (Ar²-1-A-1) |
| (Ar1-Ar2-8) | Ar¹-7 | (Ar²-1-A-1) |
| (Ar1-Ar2-9) | Ar¹-8 | (Ar²-1-A-1) |
| (Ar1-Ar2-10) | Ar¹-9 | (Ar²-1-A-1) |
| (Ar1-Ar2-11) | Ar¹-15 | (Ar²-1-A-1) |
| (Ar1-Ar2-12) | Ar¹-16 | (Ar²-1-A-1) |
| (Ar1-Ar2-13) | Ar¹-17 | (Ar²-1-A-1) |
| (Ar1-Ar2-14) | Ar¹-18 | (Ar²-1-A-1) |
| (Ar1-Ar2-15) | Ar¹-19 | (Ar²-1-A-1) |
| (Ar1-Ar2-16) | Ar¹-20 | (Ar²-1-A-1) |
| (Ar1-Ar2-17) | Ar¹-36 | (Ar²-1-A-1) |
| (Ar1-Ar2-18) | Ar¹-42 | (Ar²-1-A-1) |
| (Ar1-Ar2-19) | Ar¹-43 | (Ar²-1-A-1) |
| (Ar1-Ar2-20) | Ar¹-44 | (Ar²-1-A-1) |
| (Ar1-Ar2-21) | Ar¹-45 | (Ar²-1-A-1) |
| (Ar1-Ar2-22) | Ar¹-46 | (Ar²-1-A-1) |
| (Ar1-Ar2-23) | Ar¹-47 | (Ar²-1-A-1) |
| (Ar1-Ar2-24) | Ar¹-48 | (Ar²-1-A-1) |
| (Ar1-Ar2-25) | Ar¹-49 | (Ar²-1-A-1) |
| (Ar1-Ar2-26) | Ar¹-50 | (Ar²-1-A-1) |
| (Ar1-Ar2-27) | Ar¹-51 | (Ar²-1-A-1) |
| (Ar1-Ar2-28) | Ar¹-52 | (Ar²-1-A-1) |
| (Ar1-Ar2-29) | Ar¹-53 | (Ar²-1-A-1) |
| (Ar1-Ar2-30) | Ar¹-58 | (Ar²-1-A-1) |
| (Ar1-Ar2-31) | Ar¹-59 | (Ar²-1-A-1) |
| (Ar1-Ar2-32) | Ar¹-60 | (Ar²-1-A-1) |
| (Ar1-Ar2-33) | Ar¹-61 | (Ar²-1-A-1) |
| (Ar1-Ar2-34) | Ar¹-62 | (Ar²-1-A-1) |
| (Ar1-Ar2-35) | Ar¹-63 | (Ar²-1-A-1) |
| (Ar1-Ar2-36) | Ar¹-64 | (Ar²-1-A-1) |
| (Ar1-Ar2-37) | Single bond | (Ar²-1-A-2) |
| (Ar1-Ar2-38) | Ar¹-1 | (Ar²-1-A-2) |
| (Ar1-Ar2-39) | Ar¹-2 | (Ar²-1-A-2) |
| (Ar1-Ar2-40) | Ar¹-3 | (Ar²-1-A-2) |
| (Ar1-Ar2-41) | Ar¹-4 | (Ar²-1-A-2) |
| (Ar1-Ar2-42) | Ar¹-5 | (Ar²-1-A-2) |
| (Ar1-Ar2-43) | Ar¹-6 | (Ar²-1-A-2) |
| (Ar1-Ar2-44) | Ar¹-7 | (Ar²-1-A-2) |
| (Ar1-Ar2-45) | Ar¹-8 | (Ar²-1-A-2) |
| (Ar1-Ar2-46) | Ar¹-9 | (Ar²-1-A-2) |
| (Ar1-Ar2-47) | Ar¹-15 | (Ar²-1-A-2) |
| (Ar1-Ar2-48) | Ar¹-16 | (Ar²-1-A-2) |
| (Ar1-Ar2-49) | Ar¹-17 | (Ar²-1-A-2) |
| (Ar1-Ar2-50) | Ar¹-18 | (Ar²-1-A-2) |
| (Ar1-Ar2-51) | Ar¹-19 | (Ar²-1-A-2) |
| (Ar1-Ar2-52) | Ar¹-20 | (Ar²-1-A-2) |
| (Ar1-Ar2-53) | Ar¹-36 | (Ar²-1-A-2) |
| (Ar1-Ar2-54) | Ar¹-42 | (Ar²-1-A-2) |
| (Ar1-Ar2-55) | Ar¹-43 | (Ar²-1-A-2) |
| (Ar1-Ar2-56) | Ar¹-44 | (Ar²-1-A-2) |
| (Ar1-Ar2-57) | Ar¹-45 | (Ar²-1-A-2) |
| (Ar1-Ar2-58) | Ar¹-46 | (Ar²-1-A-2) |
| (Ar1-Ar2-59) | Ar¹-47 | (Ar²-1-A-2) |
| (Ar1-Ar2-60) | Ar¹-48 | (Ar²-1-A-2) |
| (Ar1-Ar2-61) | Ar¹-49 | (Ar²-1-A-2) |
| (Ar1-Ar2-62) | Ar¹-50 | (Ar²-1-A-2) |
| (Ar1-Ar2-63) | Ar¹-51 | (Ar²-1-A-2) |
| (Ar1-Ar2-64) | Ar¹-52 | (Ar²-1-A-2) |
| (Ar1-Ar2-65) | Ar¹-53 | (Ar²-1-A-2) |
| (Ar1-Ar2-66) | Ar¹-58 | (Ar²-1-A-2) |
| (Ar1-Ar2-67) | Ar¹-59 | (Ar²-1-A-2) |
| (Ar1-Ar2-68) | Ar¹-60 | (Ar²-1-A-2) |
| (Ar1-Ar2-69) | Ar¹-61 | (Ar²-1-A-2) |
| (Ar1-Ar2-70) | Ar¹-62 | (Ar²-1-A-2) |
| (Ar1-Ar2-71) | Ar¹-63 | (Ar²-1-A-2) |
| (Ar1-Ar2-72) | Ar¹-64 | (Ar²-1-A-2) |
| (Ar1-Ar2-73) | Single bond | (Ar²-1-A-3) |
| (Ar1-Ar2-74) | Ar¹-1 | (Ar²-1-A-3) |
| (Ar1-Ar2-75) | Ar¹-2 | (Ar²-1-A-3) |
| (Ar1-Ar2-76) | Ar¹-3 | (Ar²-1-A-3) |
| (Ar1-Ar2-77) | Ar¹-4 | (Ar²-1-A-3) |
| (Ar1-Ar2-78) | Ar¹-5 | (Ar²-1-A-3) |
| (Ar1-Ar2-79) | Ar¹-6 | (Ar²-1-A-3) |
| (Ar1-Ar2-80) | Ar¹-7 | (Ar²-1-A-3) |
| (Ar1-Ar2-81) | Ar¹-8 | (Ar²-1-A-3) |
| (Ar1-Ar2-82) | Ar¹-9 | (Ar²-1-A-3) |
| (Ar1-Ar2-83) | Ar¹-15 | (Ar²-1-A-3) |
| (Ar1-Ar2-84) | Ar¹-16 | (Ar²-1-A-3) |
| (Ar1-Ar2-85) | Ar¹-17 | (Ar²-1-A-3) |
| (Ar1-Ar2-86) | Ar¹-18 | (Ar²-1-A-3) |
| (Ar1-Ar2-87) | Ar¹-19 | (Ar²-1-A-3) |
| (Ar1-Ar2-88) | Ar¹-20 | (Ar²-1-A-3) |
| (Ar1-Ar2-89) | Ar¹-36 | (Ar²-1-A-3) |
| (Ar1-Ar2-90) | Ar¹-42 | (Ar²-1-A-3) |
| (Ar1-Ar2-91) | Ar¹-43 | (Ar²-1-A-3) |
| (Ar1-Ar2-92) | Ar¹-44 | (Ar²-1-A-3) |
| (Ar1-Ar2-93) | Ar¹-45 | (Ar²-1-A-3) |
| (Ar1-Ar2-94) | Ar¹-46 | (Ar²-1-A-3) |
| (Ar1-Ar2-95) | Ar¹-47 | (Ar²-1-A-3) |
| (Ar1-Ar2-96) | Ar¹-48 | (Ar²-1-A-3) |
| (Ar1-Ar2-97) | Ar¹-49 | (Ar²-1-A-3) |
| (Ar1-Ar2-98) | Ar¹-50 | (Ar²-1-A-3) |
| (Ar1-Ar2-99) | Ar¹-51 | (Ar²-1-A-3) |
| (Ar1-Ar2-100) | Ar¹-52 | (Ar²-1-A-3) |
| (Ar1-Ar2-101) | Ar¹-53 | (Ar²-1-A-3) |
| (Ar1-Ar2-102) | Ar¹-58 | (Ar²-1-A-3) |
| (Ar1-Ar2-103) | Ar¹-59 | (Ar²-1-A-3) |
| (Ar1-Ar2-104) | Ar¹-60 | (Ar²-1-A-3) |
| (Ar1-Ar2-105) | Ar¹-61 | (Ar²-1-A-3) |
| (Ar1-Ar2-106) | Ar¹-62 | (Ar²-1-A-3) |
| (Ar1-Ar2-107) | Ar¹-63 | (Ar²-1-A-3) |
| (Ar1-Ar2-108) | Ar¹-64 | (Ar²-1-A-3) |
| (Ar1-Ar2-109) | Single bond | (Ar²-1-A-4) |
| (Ar1-Ar2-110) | Ar¹-1 | (Ar²-1-A-4) |
| (Ar1-Ar2-111) | Ar¹-2 | (Ar²-1-A-4) |
| (Ar1-Ar2-112) | Ar¹-3 | (Ar²-1-A-4) |
| (Ar1-Ar2-113) | Ar¹-4 | (Ar²-1-A-4) |
| (Ar1-Ar2-114) | Ar¹-5 | (Ar²-1-A-4) |
| (Ar1-Ar2-115) | Ar¹-6 | (Ar²-1-A-4) |
| (Ar1-Ar2-116) | Ar¹-7 | (Ar²-1-A-4) |
| (Ar1-Ar2-117) | Ar¹-8 | (Ar²-1-A-4) |
| (Ar1-Ar2-118) | Ar¹-9 | (Ar²-1-A-4) |
| (Ar1-Ar2-119) | Ar¹-15 | (Ar²-1-A-4) |
| (Ar1-Ar2-120) | Ar¹-16 | (Ar²-1-A-4) |
| (Ar1-Ar2-121) | Ar¹-17 | (Ar²-1-A-4) |
| (Ar1-Ar2-122) | Ar¹-18 | (Ar²-1-A-4) |
| (Ar1-Ar2-123) | Ar¹-19 | (Ar²-1-A-4) |
| (Ar1-Ar2-124) | Ar¹-20 | (Ar²-1-A-4) |
| (Ar1-Ar2-125) | Ar¹-36 | (Ar²-1-A-4) |
| (Ar1-Ar2-126) | Ar¹-42 | (Ar²-1-A-4) |
| (Ar1-Ar2-127) | Ar¹-43 | (Ar²-1-A-4) |
| (Ar1-Ar2-128) | Ar¹-44 | (Ar²-1-A-4) |
| (Ar1-Ar2-129) | Ar¹-45 | (Ar²-1-A-4) |
| (Ar1-Ar2-130) | Ar¹-46 | (Ar²-1-A-4) |
| (Ar1-Ar2-131) | Ar¹-47 | (Ar²-1-A-4) |
| (Ar1-Ar2-132) | Ar¹-48 | (Ar²-1-A-4) |
| (Ar1-Ar2-133) | Ar¹-49 | (Ar²-1-A-4) |
| (Ar1-Ar2-134) | Ar¹-50 | (Ar²-1-A-4) |
| (Ar1-Ar2-135) | Ar¹-51 | (Ar²-1-A-4) |
| (Ar1-Ar2-136) | Ar¹-52 | (Ar²-1-A-4) |
| (Ar1-Ar2-137) | Ar¹-53 | (Ar²-1-A-4) |
| (Ar1-Ar2-138) | Ar¹-58 | (Ar²-1-A-4) |
| (Ar1-Ar2-139) | Ar¹-59 | (Ar²-1-A-4) |
| (Ar1-Ar2-140) | Ar¹-60 | (Ar²-1-A-4) |
| (Ar1-Ar2-141) | Ar¹-61 | (Ar²-1-A-4) |
| (Ar1-Ar2-142) | Ar¹-62 | (Ar²-1-A-4) |
| (Ar1-Ar2-143) | Ar¹-63 | (Ar²-1-A-4) |
| (Ar1-Ar2-144) | Ar¹-64 | (Ar²-1-A-4) |
| (Ar1-Ar2-145) | Single bond | (Ar²-2-A-1) |
| (Ar1-Ar2-146) | Ar¹-1 | (Ar²-2-A-1) |
| (Ar1-Ar2-147) | Ar¹-2 | (Ar²-2-A-1) |
| (Ar1-Ar2-148) | Ar¹-3 | (Ar²-2-A-1) |
| (Ar1-Ar2-149) | Ar¹-4 | (Ar²-2-A-1) |
| (Ar1-Ar2-150) | Ar¹-5 | (Ar²-2-A-1) |
| (Ar1-Ar2-151) | Ar¹-6 | (Ar²-2-A-1) |
| (Ar1-Ar2-152) | Ar¹-7 | (Ar²-2-A-1) |
| (Ar1-Ar2-153) | Ar¹-8 | (Ar²-2-A-1) |
| (Ar1-Ar2-154) | Ar¹-9 | (Ar²-2-A-1) |
| (Ar1-Ar2-155) | Ar¹-15 | (Ar²-2-A-1) |
| (Ar1-Ar2-156) | Ar¹-16 | (Ar²-2-A-1) |
| (Ar1-Ar2-157) | Ar¹-17 | (Ar²-2-A-1) |
| (Ar1-Ar2-158) | Ar¹-18 | (Ar²-2-A-1) |
| (Ar1-Ar2-159) | Ar¹-19 | (Ar²-2-A-1) |
| (Ar1-Ar2-160) | Ar¹-20 | (Ar²-2-A-1) |
| (Ar1-Ar2-161) | Ar¹-36 | (Ar²-2-A-1) |
| (Ar1-Ar2-162) | Ar¹-42 | (Ar²-2-A-1) |
| (Ar1-Ar2-163) | Ar¹-43 | (Ar²-2-A-1) |
| (Ar1-Ar2-164) | Ar¹-44 | (Ar²-2-A-1) |
| (Ar1-Ar2-165) | Ar¹-45 | (Ar²-2-A-1) |
| (Ar1-Ar2-166) | Ar¹-46 | (Ar²-2-A-1) |
| (Ar1-Ar2-167) | Ar¹-47 | (Ar²-2-A-1) |
| (Ar1-Ar2-168) | Ar¹-48 | (Ar²-2-A-1) |
| (Ar1-Ar2-169) | Ar¹-49 | (Ar²-2-A-1) |
| (Ar1-Ar2-170) | Ar¹-50 | (Ar²-2-A-1) |
| (Ar1-Ar2-171) | Ar¹-51 | (Ar²-2-A-1) |
| (Ar1-Ar2-172) | Ar¹-52 | (Ar²-2-A-1) |
| (Ar1-Ar2-173) | Ar¹-53 | (Ar²-2-A-1) |
| (Ar1-Ar2-174) | Ar¹-58 | (Ar²-2-A-1) |
| (Ar1-Ar2-175) | Ar¹-59 | (Ar²-2-A-1) |
| (Ar1-Ar2-176) | Ar¹-60 | (Ar²-2-A-1) |
| (Ar1-Ar2-177) | Ar¹-61 | (Ar²-2-A-1) |
| (Ar1-Ar2-178) | Ar¹-62 | (Ar²-2-A-1) |
| (Ar1-Ar2-179) | Ar¹-63 | (Ar²-2-A-1) |
| (Ar1-Ar2-180) | Ar¹-64 | (Ar²-2-A-1) |
| (Ar1-Ar2-181) | Single bond | (Ar²-2-A-2) |
| (Ar1-Ar2-182) | Ar¹-1 | (Ar²-2-A-2) |
| (Ar1-Ar2-183) | Ar¹-2 | (Ar²-2-A-2) |
| (Ar1-Ar2-184) | Ar¹-3 | (Ar²-2-A-2) |
| (Ar1-Ar2-185) | Ar¹-4 | (Ar²-2-A-2) |
| (Ar1-Ar2-186) | Ar¹-5 | (Ar²-2-A-2) |
| (Ar1-Ar2-187) | Ar¹-6 | (Ar²-2-A-2) |
| (Ar1-Ar2-188) | Ar¹-7 | (Ar²-2-A-2) |
| (Ar1-Ar2-189) | Ar¹-8 | (Ar²-2-A-2) |
| (Ar1-Ar2-190) | Ar¹-9 | (Ar²-2-A-2) |
| (Ar1-Ar2-191) | Ar¹-15 | (Ar²-2-A-2) |
| (Ar1-Ar2-192) | Ar¹-16 | (Ar²-2-A-2) |
| (Ar1-Ar2-193) | Ar¹-17 | (Ar²-2-A-2) |
| (Ar1-Ar2-194) | Ar¹-18 | (Ar²-2-A-2) |
| (Ar1-Ar2-195) | Ar¹-19 | (Ar²-2-A-2) |
| (Ar1-Ar2-196) | Ar¹-20 | (Ar²-2-A-2) |
| (Ar1-Ar2-197) | Ar¹-36 | (Ar²-2-A-2) |
| (Ar1-Ar2-198) | Ar¹-42 | (Ar²-2-A-2) |
| (Ar1-Ar2-199) | Ar¹-43 | (Ar²-2-A-2) |
| (Ar1-Ar2-200) | Ar¹-44 | (Ar²-2-A-2) |
| (Ar1-Ar2-201) | Ar¹-45 | (Ar²-2-A-2) |
| (Ar1-Ar2-202) | Ar¹-46 | (Ar²-2-A-2) |
| (Ar1-Ar2-203) | Ar¹-47 | (Ar²-2-A-2) |
| (Ar1-Ar2-204) | Ar¹-48 | (Ar²-2-A-2) |
| (Ar1-Ar2-205) | Ar¹-49 | (Ar²-2-A-2) |
| (Ar1-Ar2-206) | Ar¹-50 | (Ar²-2-A-2) |
| (Ar1-Ar2-207) | Ar¹-51 | (Ar²-2-A-2) |
| (Ar1-Ar2-208) | Ar¹-52 | (Ar²-2-A-2) |
| (Ar1-Ar2-209) | Ar¹-53 | (Ar²-2-A-2) |
| (Ar1-Ar2-210) | Ar¹-58 | (Ar²-2-A-2) |
| (Ar1-Ar2-211) | Ar¹-59 | (Ar²-2-A-2) |
| (Ar1-Ar2-212) | Ar¹-60 | (Ar²-2-A-2) |
| (Ar1-Ar2-213) | Ar¹-61 | (Ar²-2-A-2) |
| (Ar1-Ar2-214) | Ar¹-62 | (Ar²-2-A-2) |
| (Ar1-Ar2-215) | Ar¹-63 | (Ar²-2-A-2) |
| (Ar1-Ar2-216) | Ar¹-64 | (Ar²-2-A-2) |
| (Ar1-Ar2-217) | Single bond | (Ar²-2-A-3) |
| (Ar1-Ar2-218) | Ar¹-1 | (Ar²-2-A-3) |
| (Ar1-Ar2-219) | Ar¹-2 | (Ar²-2-A-3) |
| (Ar1-Ar2-220) | Ar¹-3 | (Ar²-2-A-3) |
| (Ar1-Ar2-221) | Ar¹-4 | (Ar²-2-A-3) |
| (Ar1-Ar2-222) | Ar¹-5 | (Ar²-2-A-3) |
| (Ar1-Ar2-223) | Ar¹-6 | (Ar²-2-A-3) |
| (Ar1-Ar2-224) | Ar¹-7 | (Ar²-2-A-3) |
| (Ar1-Ar2-225) | Ar¹-8 | (Ar²-2-A-3) |
| (Ar1-Ar2-226) | Ar¹-9 | (Ar²-2-A-3) |
| (Ar1-Ar2-227) | Ar¹-15 | (Ar²-2-A-3) |
| (Ar1-Ar2-228) | Ar¹-16 | (Ar²-2-A-3) |
| (Ar1-Ar2-229) | Ar¹-17 | (Ar²-2-A-3) |
| (Ar1-Ar2-230) | Ar¹-18 | (Ar²-2-A-3) |
| (Ar1-Ar2-231) | Ar¹-19 | (Ar²-2-A-3) |
| (Ar1-Ar2-232) | Ar¹-20 | (Ar²-2-A-3) |
| (Ar1-Ar2-233) | Ar¹-36 | (Ar²-2-A-3) |
| (Ar1-Ar2-234) | Ar¹-42 | (Ar²-2-A-3) |
| (Ar1-Ar2-235) | Ar¹-43 | (Ar²-2-A-3) |
| (Ar1-Ar2-236) | Ar¹-44 | (Ar²-2-A-3) |
| (Ar1-Ar2-237) | Ar¹-45 | (Ar²-2-A-3) |
| (Ar1-Ar2-238) | Ar¹-46 | (Ar²-2-A-3) |
| (Ar1-Ar2-239) | Ar¹-47 | (Ar²-2-A-3) |
| (Ar1-Ar2-240) | Ar¹-48 | (Ar²-2-A-3) |
| (Ar1-Ar2-241) | Ar¹-49 | (Ar²-2-A-3) |
| (Ar1-Ar2-242) | Ar¹-50 | (Ar²-2-A-3) |
| (Ar1-Ar2-243) | Ar¹-51 | (Ar²-2-A-3) |
| (Ar1-Ar2-244) | Ar¹-52 | (Ar²-2-A-3) |
| (Ar1-Ar2-245) | Ar¹-53 | (Ar²-2-A-3) |
| (Ar1-Ar2-246) | Ar¹-58 | (Ar²-2-A-3) |
| (Ar1-Ar2-247) | Ar¹-59 | (Ar²-2-A-3) |
| (Ar1-Ar2-248) | Ar¹-60 | (Ar²-2-A-3) |
| (Ar1-Ar2-249) | Ar¹-61 | (Ar²-2-A-3) |
| (Ar1-Ar2-250) | Ar¹-62 | (Ar²-2-A-3) |
| (Ar1-Ar2-251) | Ar¹-63 | (Ar²-2-A-3) |
| (Ar1-Ar2-252) | Ar¹-64 | (Ar²-2-A-3) |
| (Ar1-Ar2-253) | Single bond | (Ar²-2-A-4) |
| (Ar1-Ar2-254) | Ar¹-1 | (Ar²-2-A-4) |
| (Ar1-Ar2-255) | Ar¹-2 | (Ar²-2-A-4) |
| (Ar1-Ar2-256) | Ar¹-3 | (Ar²-2-A-4) |
| (Ar1-Ar2-257) | Ar¹-4 | (Ar²-2-A-4) |
| (Ar1-Ar2-258) | Ar¹-5 | (Ar²-2-A-4) |
| (Ar1-Ar2-259) | Ar¹-6 | (Ar²-2-A-4) |
| (Ar1-Ar2-260) | Ar¹-7 | (Ar²-2-A-4) |
| (Ar1-Ar2-261) | Ar¹-8 | (Ar²-2-A-4) |
| (Ar1-Ar2-262) | Ar¹-9 | (Ar²-2-A-4) |
| (Ar1-Ar2-263) | Ar¹-15 | (Ar²-2-A-4) |
| (Ar1-Ar2-264) | Ar¹-16 | (Ar²-2-A-4) |
| (Ar1-Ar2-265) | Ar¹-17 | (Ar²-2-A-4) |
| (Ar1-Ar2-266) | Ar¹-18 | (Ar²-2-A-4) |
| (Ar1-Ar2-267) | Ar¹-19 | (Ar²-2-A-4) |
| (Ar1-Ar2-268) | Ar¹-20 | (Ar²-2-A-4) |
| (Ar1-Ar2-269) | Ar¹-36 | (Ar²-2-A-4) |
| (Ar1-Ar2-270) | Ar¹-42 | (Ar²-2-A-4) |
| (Ar1-Ar2-271) | Ar¹-43 | (Ar²-2-A-4) |
| (Ar1-Ar2-272) | Ar¹-44 | (Ar²-2-A-4) |
| (Ar1-Ar2-273) | Ar¹-45 | (Ar²-2-A-4) |
| (Ar1-Ar2-274) | Ar¹-46 | (Ar²-2-A-4) |
| (Ar1-Ar2-275) | Ar¹-47 | (Ar²-2-A-4) |
| (Ar1-Ar2-276) | Ar¹-48 | (Ar²-2-A-4) |
| (Ar1-Ar2-277) | Ar¹-49 | (Ar²-2-A-4) |
| (Ar1-Ar2-278) | Ar¹-50 | (Ar²-2-A-4) |
| (Ar1-Ar2-279) | Ar¹-51 | (Ar²-2-A-4) |
| (Ar1-Ar2-280) | Ar¹-52 | (Ar²-2-A-4) |
| (Ar1-Ar2-281) | Ar¹-53 | (Ar²-2-A-4) |
| (Ar1-Ar2-282) | Ar¹-58 | (Ar²-2-A-4) |
| (Ar1-Ar2-283) | Ar¹-59 | (Ar²-2-A-4) |
| (Ar1-Ar2-284) | Ar¹-60 | (Ar²-2-A-4) |
| (Ar1-Ar2-285) | Ar¹-61 | (Ar²-2-A-4) |
| (Ar1-Ar2-286) | Ar¹-62 | (Ar²-2-A-4) |
| (Ar1-Ar2-287) | Ar¹-63 | (Ar²-2-A-4) |
| (Ar1-Ar2-288) | Ar¹-64 | (Ar²-2-A-4) |

or from units of the following formulae —Ar¹-Ar²—Ar² where Ar¹ is a single bond

| Formula | -Ar²- | -Ar² |
|---|---|---|
| (Ar1-Ar2-Ar2-1) | 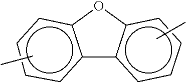 | (Ar²-1-A-1) |
| (Ar1-Ar2-Ar2-2) | 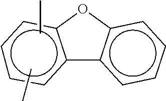 | (Ar²-1-A-1) |
| (Ar1-Ar2-Ar2-3) | 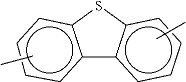 | (Ar²-1-A-1) |
| (Ar1-Ar2-Ar2-4) | 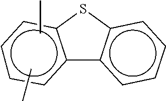 | (Ar²-1-A-1) |
| (Ar1-Ar2-Ar2-5) | 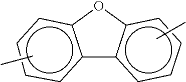 | (Ar²-1-A-2) |
| (Ar1-Ar2-Ar2-6) | 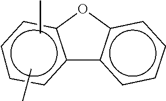 | (Ar²-1-A-2) |
| (Ar1-Ar2-Ar2-7) | 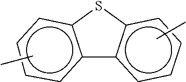 | (Ar²-1-A-2) |
| (Ar1-Ar2-Ar2-8) | 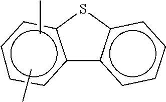 | (Ar²-1-A-2) |
| (Ar1-Ar2-Ar2-9) | 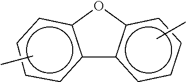 | (Ar²-1-A-3) |
| (Ar1-Ar2-Ar2-10) | 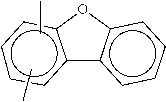 | (Ar²-1-A-3) |
| (Ar1-Ar2-Ar2-11) | 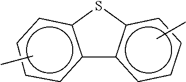 | (Ar²-1-A-3) |
| (Ar1-Ar2-Ar2-12) | 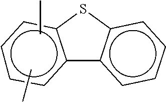 | (Ar²-1-A-3) |
| (Ar1-Ar2-Ar2-13) | 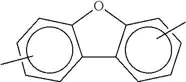 | (Ar²-1-A-4) |
| (Ar1-Ar2-Ar2-14) | 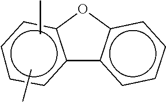 | (Ar²-1-A-4) |
| (Ar1-Ar2-Ar2-15) | 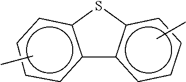 | (Ar²-1-A-4) |
| (Ar1-Ar2-Ar2-16) | 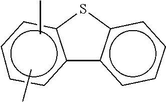 | (Ar²-1-A-4) |
| (Ar1-Ar2-Ar2-17) | 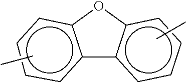 | (Ar²-2-A-1) |
| (Ar1-Ar2-Ar2-18) | 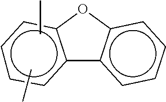 | (Ar²-2-A-1) |
| (Ar1-Ar2-Ar2-19) | 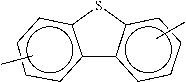 | (Ar²-2-A-1) |
| (Ar1-Ar2-Ar2-20) | 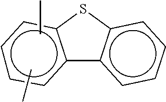 | (Ar²-2-A-1) |
| (Ar1-Ar2-Ar2-21) | 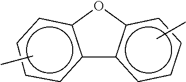 | (Ar²-2-A-2) |
| (Ar1-Ar2-Ar2-22) | 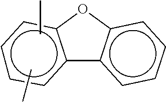 | (Ar²-2-A-2) |
| (Ar1-Ar2-Ar2-23) | 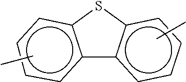 | (Ar²-2-A-2) |
| (Ar1-Ar2-Ar2-24) | 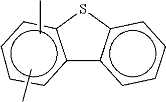 | (Ar²-2-A-2) |
| (Ar1-Ar2-Ar2-25) | 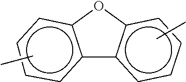 | (Ar²-2-A-3) |
| (Ar1-Ar2-Ar2-26) | 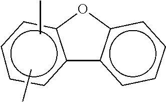 | (Ar²-2-A-3) |
| (Ar1-Ar2-Ar2-27) | 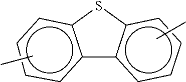 | (Ar²-2-A-3) |
| (Ar1-Ar2-Ar2-28) | 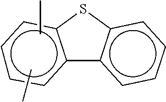 | (Ar²-2-A-3) |
| (Ar1-Ar2-Ar2-29) | 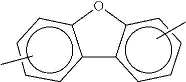 | (Ar²-2-A-4) |
| (Ar1-Ar2-Ar2-30) | 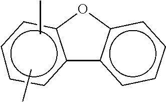 | (Ar²-2-A-4) |
| (Ar1-Ar2-Ar2-31) | 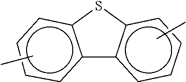 | (Ar²-2-A-4) |
| (Ar1-Ar2-Ar2-32) | 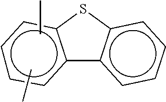 | (Ar²-2-A-4) |

and where the units —Ar²— may each be substituted at their unoccupied positions by an R³ radical.

Preferred units —[Ar¹]_{c}—[Ar²]_{d}—[Ar¹]ₑ— in formula (II) are selected from units —Ar²— of the following formulae:

| Formula | -Ar²- |
|---|---|
| (Ar²-1-5) | 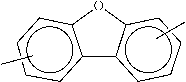 |
| (Ar²-1-6) | 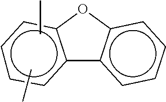 |
| (Ar²-2-5) | 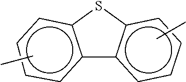 |
| (Ar²-2-6) | 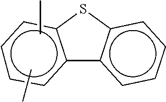 |

where the units —Ar²— may each be substituted at their unoccupied positions by an R³ radical, or from units —Ar²—Ar²— of the following formulae:

| Formula | -Ar²- | -Ar²- |
|---|---|---|
| (Ar2-Ar2-1) | 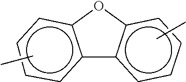 | 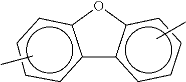 |
| (Ar2-Ar2-2) | 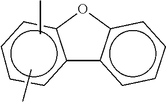 | 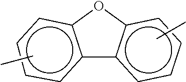 |
| (Ar2-Ar2-3) | 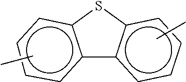 | 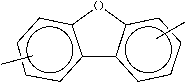 |
| (Ar2-Ar2-4) | 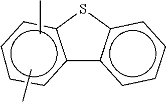 | 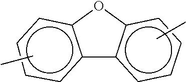 |
| (Ar2-Ar2-5) | 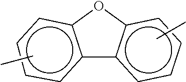 | 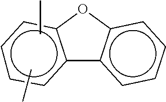 |
| (Ar2-Ar2-6) | 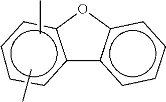 | 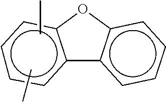 |
| (Ar2-Ar2-7) | 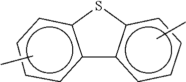 | 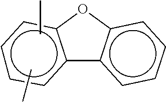 |
| (Ar2-Ar2-8) | 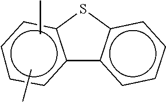 | 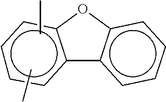 |
| (Ar2-Ar2-9) | 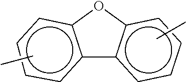 | 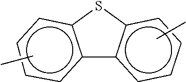 |
| (Ar2-Ar2-10) | 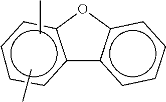 | 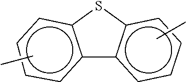 |
| (Ar2-Ar2-11) | 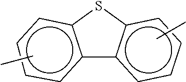 | 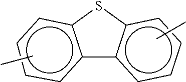 |
| (Ar2-Ar2-12) | 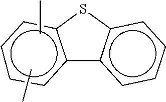 | 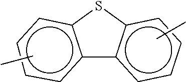 |
| (Ar2-Ar2-13) | 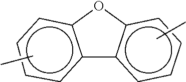 | 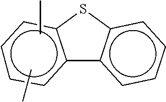 |
| (Ar2-Ar2-14) | 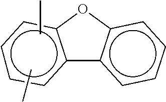 | 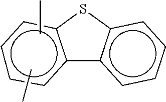 |
| (Ar2-Ar2-15) | 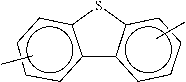 | 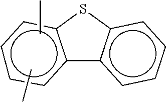 |
| (Ar2-Ar2-16) | 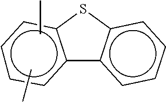 | 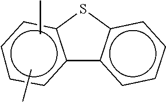 |

where the units —Ar²— may each be substituted at their unoccupied positions by an R³ radical.

R¹ is preferably the same or different at each instance and is selected from H, D, F, CN, Si(R⁴)₃, straight-chain alkyl or alkoxy groups having 1 to 20 carbon atoms, branched or cyclic alkyl or alkoxy groups having 3 to 20 carbon atoms, aromatic ring systems having 6 to 40 aromatic ring atoms, and heteroaromatic ring systems having 5 to 40 aromatic ring atoms, where the alkyl and alkoxy groups mentioned, the aromatic ring systems mentioned and the heteroaromatic ring systems mentioned may each be substituted by one or more R⁴ radicals; and where one or more CH₂ groups in the alkyl or alkoxy groups mentioned may be replaced by —C≡C—, —R⁴C═CR⁴—, Si(R⁴)₂, C═O, C═NR⁴, —O—, —S—, —C(═O)O— or —C(═O)NR⁴—. More preferably, R¹ is the same or different at each instance and is selected from H, D, F, CN, aromatic ring systems which have 6 to 40 ring atoms and may each be substituted by one or more R⁴ radicals, especially phenyl, biphenyl, fluorenyl, spirobifluorenyl and naphthyl, each of which may be substituted by one or more R⁴ radicals, and heteroaromatic ring systems which have 5 to 40 aromatic ring atoms and may each be substituted by one or more R⁴ radicals, especially dibenzofuranyl, dibenzothiophenyl, pyridyl, pyrimidyl, benzoquinoline and triazinyl, each of which may be substituted by one or more R⁴ radicals. Even more preferably, R¹ is H.

R² is preferably the same or different at each instance and is selected from H, D, F, CN, Si(R⁴)₃, straight-chain alkyl or alkoxy groups having 1 to 20 carbon atoms, branched or cyclic alkyl or alkoxy groups having 3 to 20 carbon atoms, aromatic ring systems having 6 to 40 aromatic ring atoms, and heteroaromatic ring systems having 5 to 40 aromatic ring atoms, where the alkyl and alkoxy groups mentioned, the aromatic ring systems mentioned and the heteroaromatic ring systems mentioned may each be substituted by one or more R⁴ radicals; and where one or more CH₂ groups in the alkyl or alkoxy groups mentioned may be replaced by —C≡C—, —R⁴C═CR⁴—, Si(R⁴)₂, C═O, C═NR⁴, —O—, —S—, —C(═O)O— or —C(═O)NR⁴—. More preferably, R² is the same or different at each instance and is selected from H, D, F, CN, aromatic ring systems which have 6 to 40 aromatic ring atoms and may each be substituted by one or more R⁴ radicals, especially phenyl, biphenyl, fluorenyl, spirobifluorenyl and naphthyl, each of which may be substituted by one or more R⁴ radicals, and heteroaromatic ring systems which have 5 to 40 aromatic ring atoms and may each be substituted by one or more R⁴ radicals, especially dibenzofuranyl, dibenzothiophenyl, pyridyl, pyrimidyl, benzoquinolinyl and triazinyl, each of which may be substituted by one or more R⁴ radicals. Even more preferably, R² is H.

R³ is preferably the same or different at each instance and is selected from H, D, F, CN, Si(R⁴)₃, straight-chain alkyl or alkoxy groups having 1 to 20 carbon atoms, branched or cyclic alkyl or alkoxy groups having 3 to 20 carbon atoms, aromatic ring systems having 6 to 40 aromatic ring atoms, and electron-deficient heteroaryl groups having 6 to 40 aromatic ring atoms; where the alkyl and alkoxy groups mentioned, the aromatic ring systems mentioned and the electron-deficient heteroaryl groups mentioned may each be substituted by one or more R⁴ radicals; and where one or more CH₂ groups in the alkyl or alkoxy groups mentioned may be replaced by —C≡C—, —R⁴C═CR⁴—, Si(R⁴)₂, C═O, C═NR⁴, —O—, —S—, —C(═O)O— or —C(═O)NR⁴—. More preferably, R³ is the same or different at each instance and is selected from H, D, F, CN, aromatic ring systems which have 6 to 40 aromatic ring atoms and may each be substituted by one or more R⁴ radicals, especially phenyl, biphenyl, fluorenyl, spirobifluorenyl and naphthyl, each of which may be substituted by one or more R⁴ radicals, and electron-deficient heteroaryl groups which have 6 to 40 aromatic ring atoms and may each be substituted by one or more R⁴ radicals, especially pyridyl, pyrimidyl, benzoquinolinyl and triazinyl, each of which may be substituted by one or more R⁴ radicals.

R⁴ is preferably the same or different at each instance and is selected from H, D, F, CN, Si(R⁵)₃, straight-chain alkyl or alkoxy groups having 1 to 20 carbon atoms, branched or cyclic alkyl or alkoxy groups having 3 to 20 carbon atoms, aromatic ring systems having 6 to 40 aromatic ring atoms, and heteroaromatic ring systems having 5 to 40 aromatic ring atoms, where the alkyl and alkoxy groups mentioned, the aromatic ring systems mentioned and the heteroaromatic ring systems mentioned may each be substituted by one or more R⁵ radicals; and where one or more CH₂ groups in the alkyl or alkoxy groups mentioned may be replaced by —C≡C—, —R⁵C═CR⁵—, Si(R⁵)₂, C═O, C═NR⁵, —O—, —S—, —C(═O)O— or —C(═O)NR⁵—. More preferably, R⁴ is H.

Indices a, b, c, d and e are preferably the same or different at each instance and are selected from 1 and 2.

In a preferred embodiment, in formula (I), a=1 and b=1. In an alternative preferred embodiment, in formula (I), a=1 and b=2, where the two Ar² groups selected are the same or different.

In a preferred embodiment, in formula (II), c=1, e=1 and d=1. In an alternative preferred embodiment, in formula (II), c=1, e=1 and d=2, where the two Ar² groups selected are the same or different.

Preferred embodiments of the formula (I) conform to the following formulae:

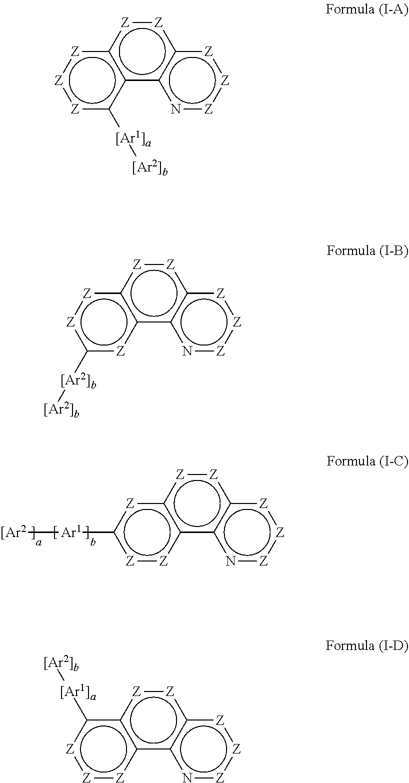

where the groups that occur are as defined above. Preferably, Z is CR¹ in the abovementioned formulae. Among the formulae, particular preference is given to the formula (I-A).

A preferred embodiment of the formula (II) is the following formula:

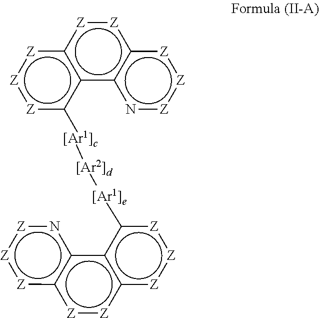

where the groups that occur are as defined above. Preferably, Z in the formula is CR¹.

Further preferred embodiments of the formula (I) conform to the following formulae:

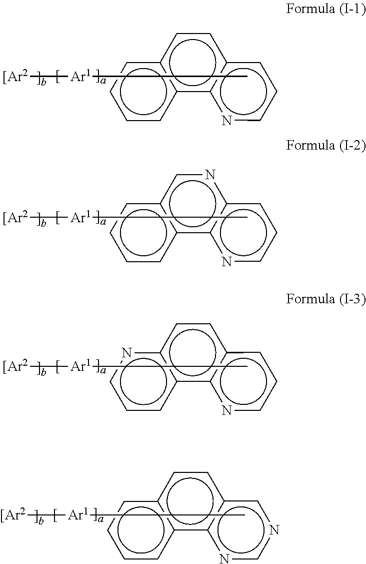

where the groups that occur are as defined above and the unoccupied positions on the azaphenanthrene base skeleton may each be substituted by an R¹ radical.

Preferred embodiments of the formula (I-A) conform to the following formulae:

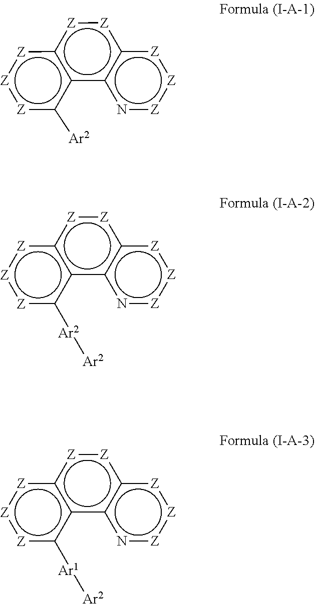

where the groups that occur are as defined above, except that Ar¹ is not a single bond. Preferably, Z in the formulae is CR¹. In particular, the Ar² groups in formula (I-A-2) may each be the same or different.

Preferred embodiments of the formula (II-A) conform to the following formulae:

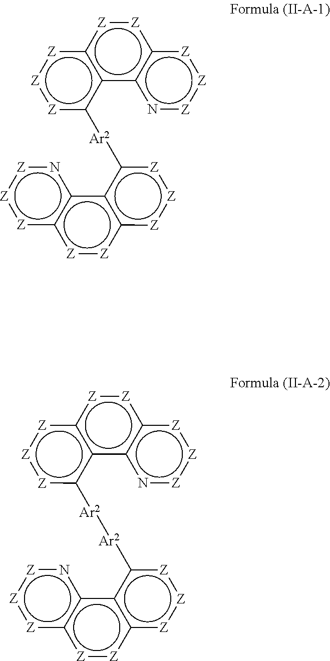

where the groups that occur are as defined above. Preferably, Z in the formulae is CR¹. In particular, the Ar² groups in formula (II-A-2) may each be the same or different.

Preferred embodiments of the compounds are listed below:

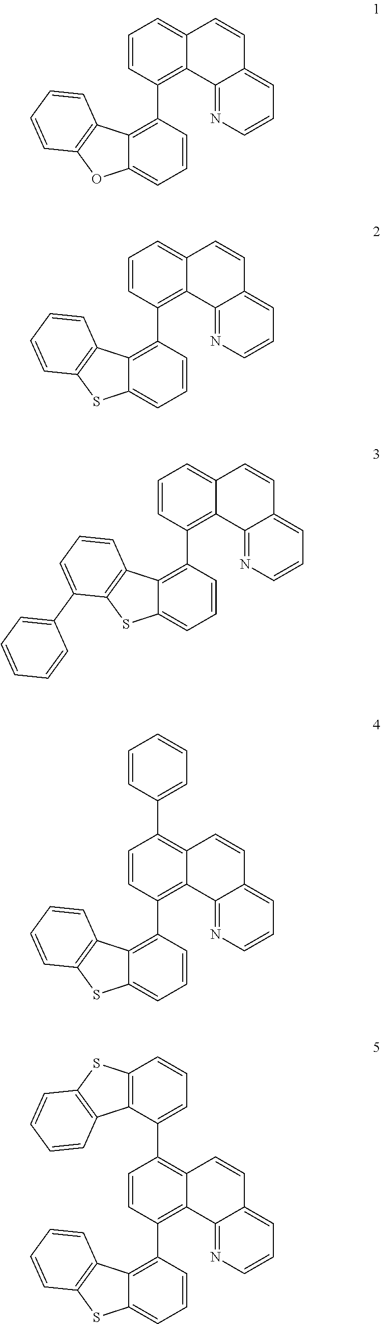 [SMILES: c1cnc2c(c1)ccc1cccc(-c3cccc4oc5ccccc5c34)c12 ; c1cnc2c(c1)ccc1cccc(-c3cccc4sc5ccccc5c34)c12 ; c1ccc(-c2cccc3c2sc2cccc(-c4cccc5ccc6cccnc6c45)c23)cc1 ; c1ccc(-c2ccc(-c3cccc4sc5ccccc5c34)c3c2ccc2cccnc23)cc1 ; c1cnc2c(c1)ccc1c(-c3cccc4sc5ccccc5c34)ccc(-c3cccc4sc5ccccc5c34)c12]

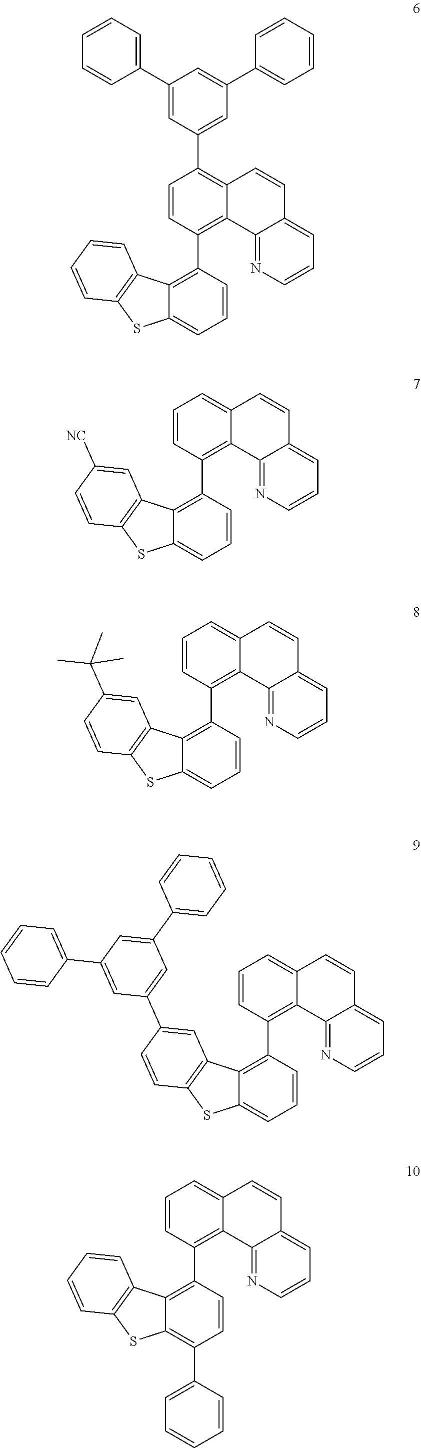 [SMILES: c1ccc(-c2cc(-c3ccccc3)cc(-c3ccc(-c4cccc5sc6ccccc6c45)c4c3ccc3cccnc34)c2)cc1 ; N#Cc1ccc2sc3cccc(-c4cccc5ccc6cccnc6c45)c3c2c1 ; CC(C)(C)c1ccc2sc3cccc(-c4cccc5ccc6cccnc6c45)c3c2c1 ; c1ccc(-c2cc(-c3ccccc3)cc(-c3ccc4sc5cccc(-c6cccc7ccc8cccnc8c67)c5c4c3)c2)cc1 ; c1ccc(-c2ccc(-c3cccc4ccc5cccnc5c34)c3c2sc2ccccc23)cc1]

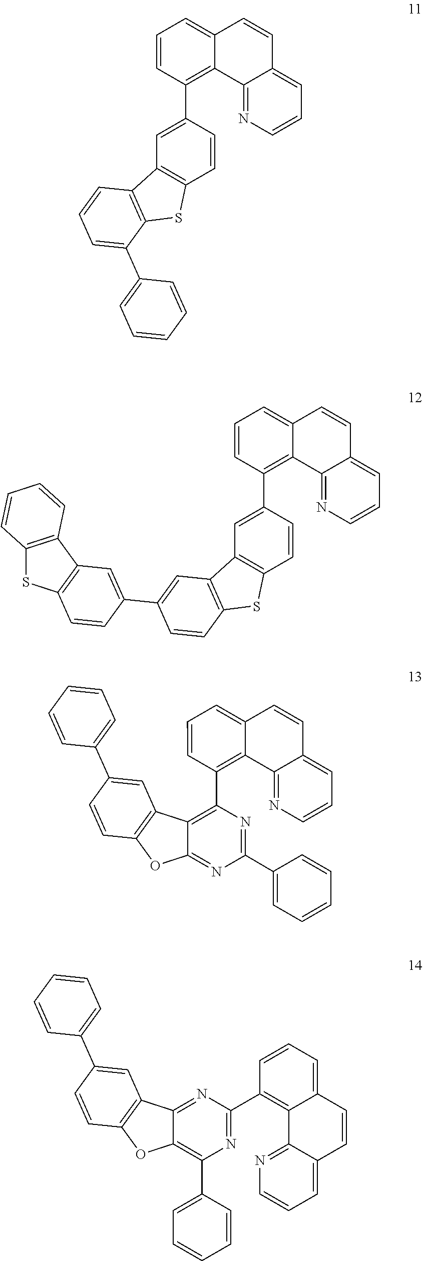 [SMILES: c1ccc(-c2cccc3c2sc2ccc(-c4cccc5ccc6cccnc6c45)cc23)cc1 ; c1cnc2c(c1)ccc1cccc(-c3ccc4sc5ccc(-c6ccc7sc8ccccc8c7c6)cc5c4c3)c12 ; c1ccc(-c2ccc3oc4nc(-c5ccccc5)nc(-c5cccc6ccc7cccnc7c56)c4c3c2)cc1 ; c1ccc(-c2ccc3oc4c(-c5ccccc5)nc(-c5cccc6ccc7cccnc7c56)nc4c3c2)cc1]

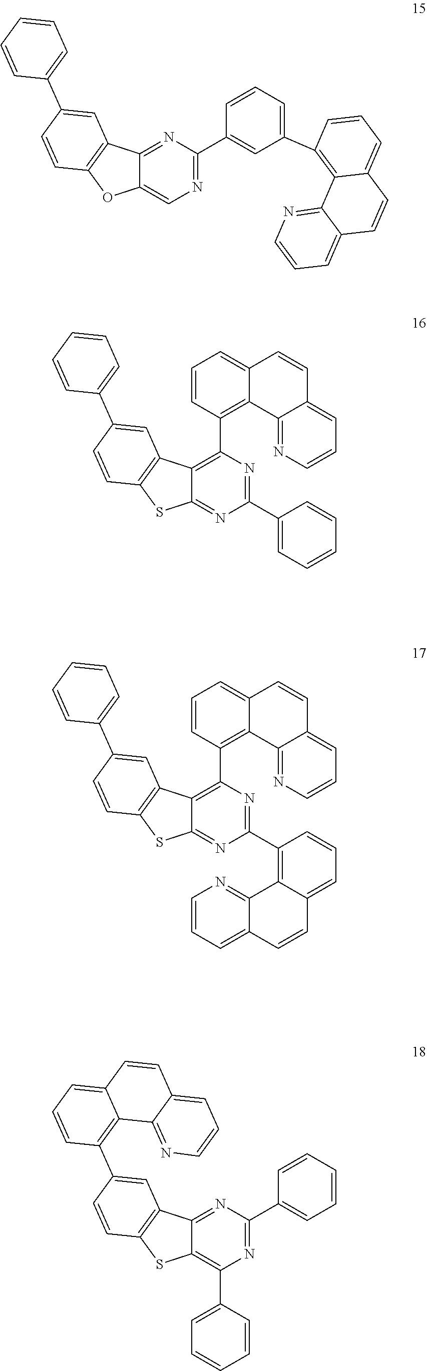 [SMILES: c1ccc(-c2ccc3oc4cnc(-c5cccc(-c6cccc7ccc8cccnc8c67)c5)nc4c3c2)cc1 ; c1ccc(-c2ccc3sc4nc(-c5ccccc5)nc(-c5cccc6ccc7cccnc7c56)c4c3c2)cc1 ; c1ccc(-c2ccc3sc4nc(-c5cccc6ccc7cccnc7c56)nc(-c5cccc6ccc7cccnc7c56)c4c3c2)cc1 ; c1ccc(-c2nc(-c3ccccc3)c3sc4ccc(-c5cccc6ccc7cccnc7c56)cc4c3n2)cc1]

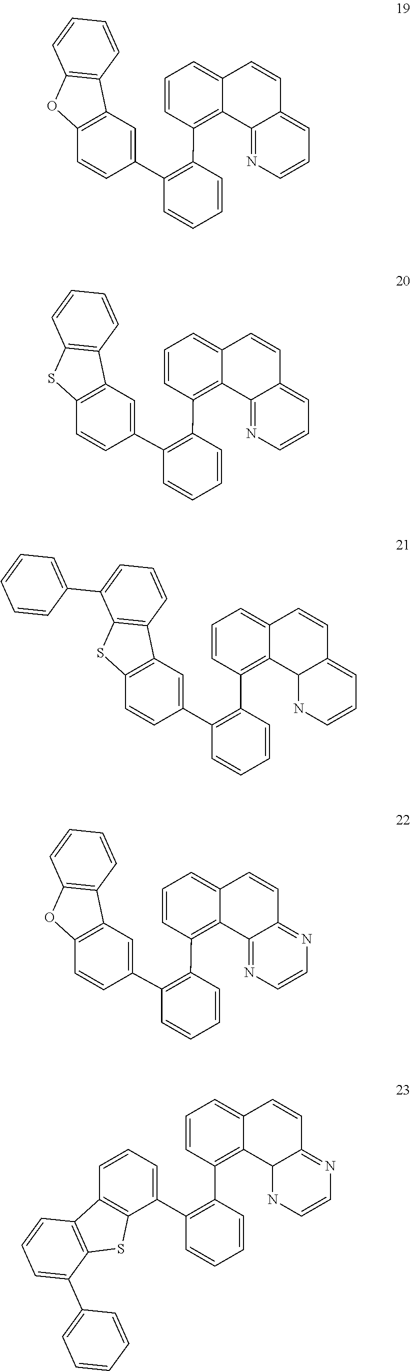

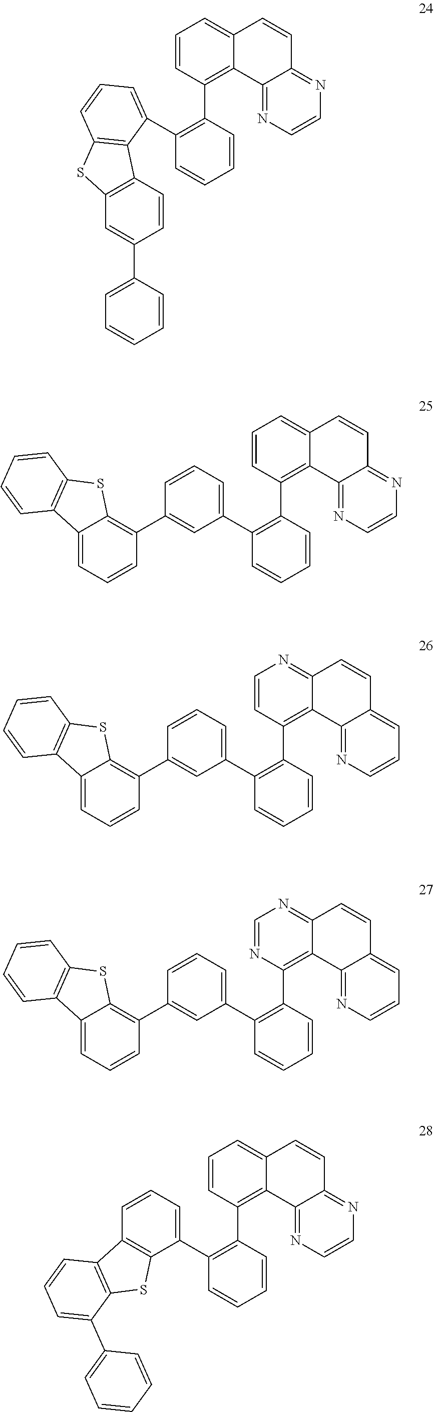 [SMILES: c1ccc(-c2ccc3c(c2)sc2cccc(-c4ccccc4-c4cccc5ccc6nccnc6c45)c23)cc1 ; c1cc(-c2ccccc2-c2cccc3ccc4nccnc4c23)cc(-c2cccc3c2sc2ccccc23)c1 ; c1cc(-c2ccccc2-c2ccnc3ccc4cccnc4c23)cc(-c2cccc3c2sc2ccccc23)c1 ; c1cc(-c2ccccc2-c2ncnc3ccc4cccnc4c23)cc(-c2cccc3c2sc2ccccc23)c1 ; c1ccc(-c2cccc3c2sc2c(-c4ccccc4-c4cccc5ccc6nccnc6c45)cccc23)cc1]

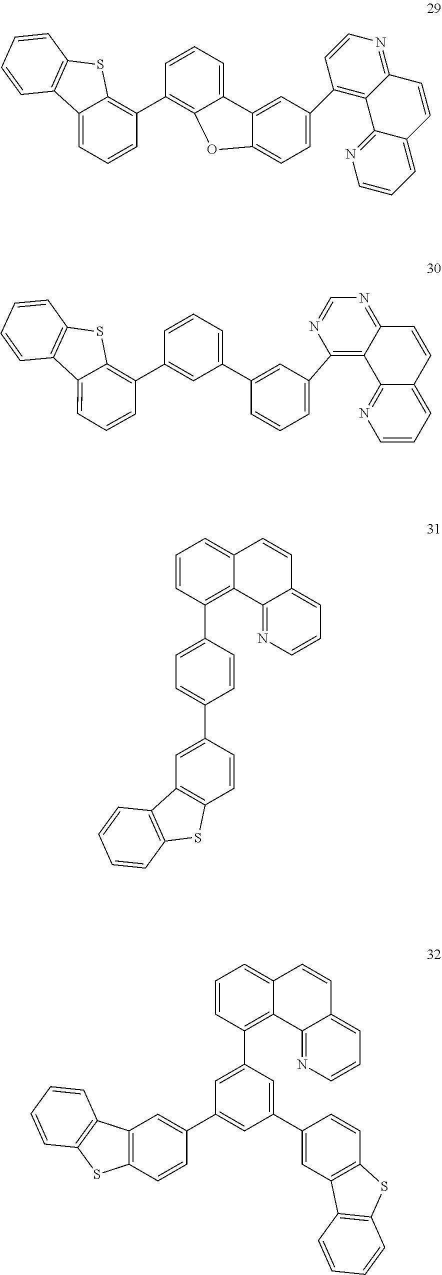 [SMILES: c1cnc2c(c1)ccc1nccc(-c3ccc4oc5c(-c6cccc7c6sc6ccccc67)cccc5c4c3)c12 ; c1cc(-c2cccc(-c3ncnc4ccc5cccnc5c34)c2)cc(-c2cccc3c2sc2ccccc23)c1 ; c1cnc2c(c1)ccc1cccc(-c3ccc(-c4ccc5sc6ccccc6c5c4)cc3)c12 ; c1cnc2c(c1)ccc1cccc(-c3cc(-c4ccc5sc6ccccc6c5c4)cc(-c4ccc5sc6ccccc6c5c4)c3)c12]

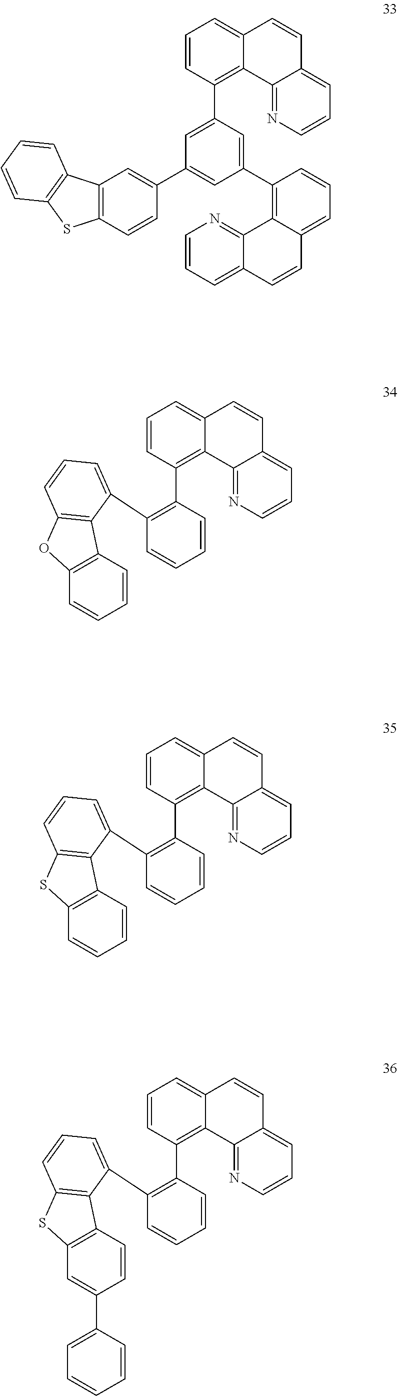 [SMILES: c1cnc2c(c1)ccc1cccc(-c3cc(-c4ccc5sc6ccccc6c5c4)cc(-c4cccc5ccc6cccnc6c45)c3)c12 ; c1ccc(-c2cccc3ccc4cccnc4c23)c(-c2cccc3oc4ccccc4c23)c1 ; c1ccc(-c2cccc3ccc4cccnc4c23)c(-c2cccc3sc4ccccc4c23)c1 ; c1ccc(-c2ccc3c(c2)sc2cccc(-c4ccccc4-c4cccc5ccc6cccnc6c45)c23)cc1]

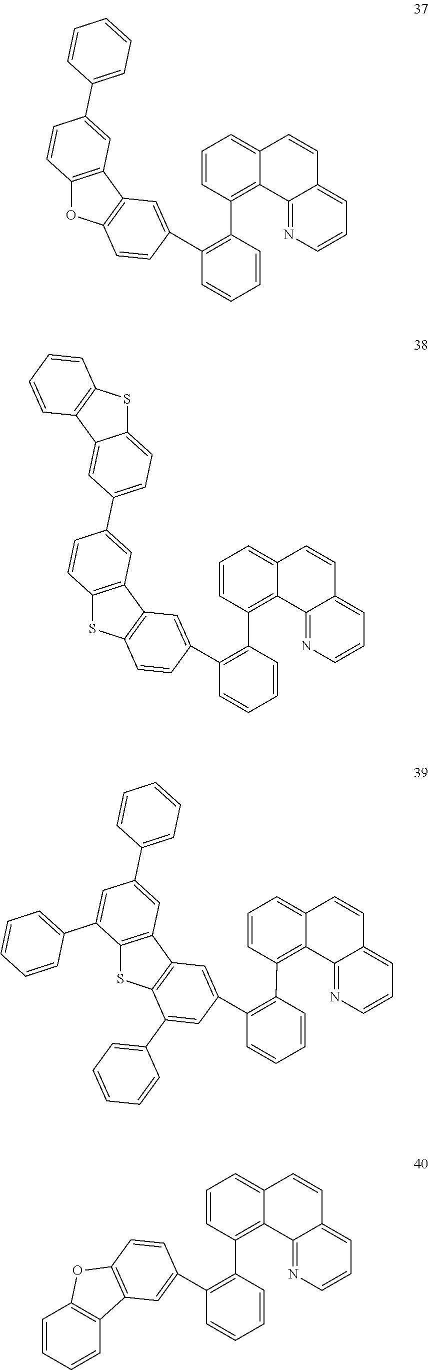 [SMILES: c1ccc(-c2ccc3oc4ccc(-c5ccccc5-c5cccc6ccc7cccnc7c56)cc4c3c2)cc1 ; c1ccc(-c2cccc3ccc4cccnc4c23)c(-c2ccc3sc4ccc(-c5ccc6sc7ccccc7c6c5)cc4c3c2)c1 ; c1ccc(-c2cc(-c3ccccc3)c3sc4c(-c5ccccc5)cc(-c5ccccc5-c5cccc6ccc7cccnc7c56)cc4c3c2)cc1 ; c1ccc(-c2cccc3ccc4cccnc4c23)c(-c2ccc3oc4ccccc4c3c2)c1]

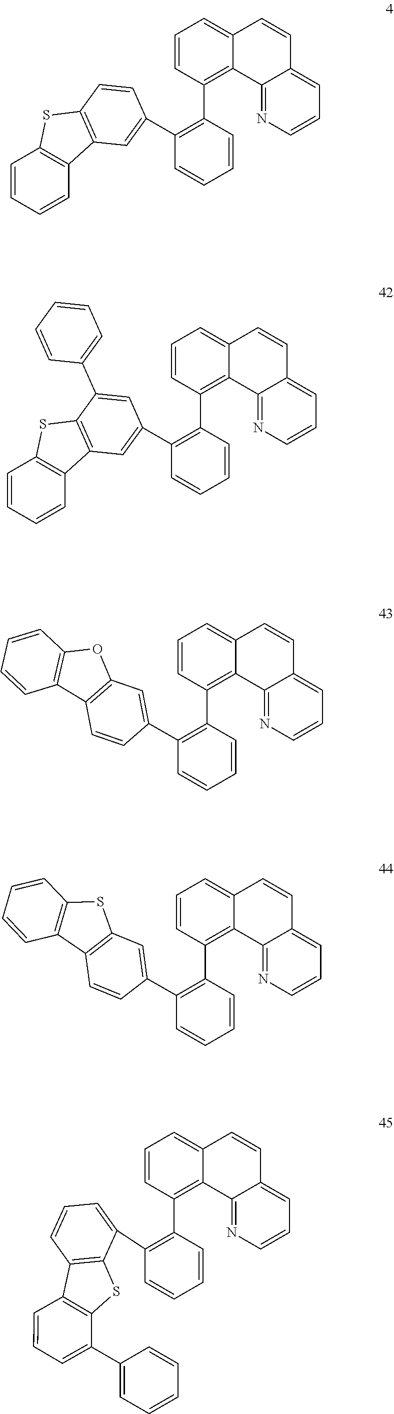 [SMILES: c1ccc(-c2cccc3ccc4cccnc4c23)c(-c2ccc3sc4ccccc4c3c2)c1 ; c1ccc(-c2cc(-c3ccccc3-c3cccc4ccc5cccnc5c34)cc3c2sc2ccccc23)cc1 ; c1ccc(-c2cccc3ccc4cccnc4c23)c(-c2ccc3c(c2)oc2ccccc23)c1 ; c1ccc(-c2cccc3ccc4cccnc4c23)c(-c2ccc3c(c2)sc2ccccc23)c1 ; c1ccc(-c2cccc3c2sc2c(-c4ccccc4-c4cccc5ccc6cccnc6c45)cccc23)cc1]

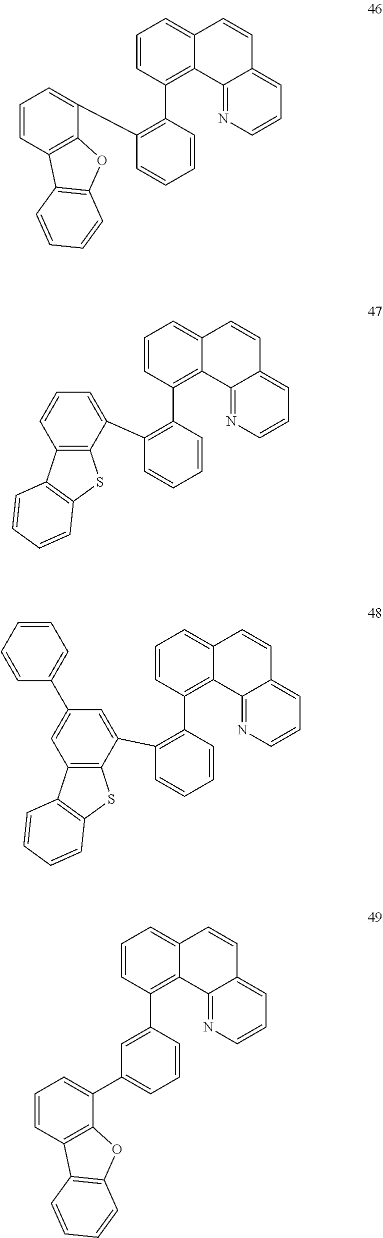 [SMILES: c1ccc(-c2cccc3ccc4cccnc4c23)c(-c2cccc3c2oc2ccccc23)c1 ; c1ccc(-c2cccc3ccc4cccnc4c23)c(-c2cccc3c2sc2ccccc23)c1 ; c1ccc(-c2cc(-c3ccccc3-c3cccc4ccc5cccnc5c34)c3sc4ccccc4c3c2)cc1 ; c1cc(-c2cccc3c2oc2ccccc23)cc(-c2cccc3ccc4cccnc4c23)c1]

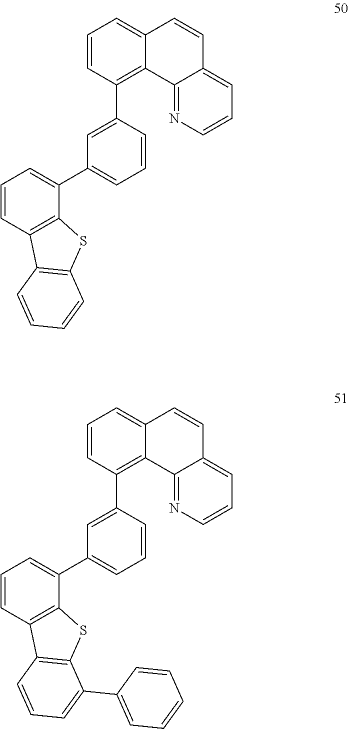 [SMILES: c1cc(-c2cccc3c2sc2ccccc23)cc(-c2cccc3ccc4cccnc4c23)c1 ; c1ccc(-c2cccc3c2sc2c(-c4cccc(-c5cccc6ccc7cccnc7c56)c4)cccc23)cc1]

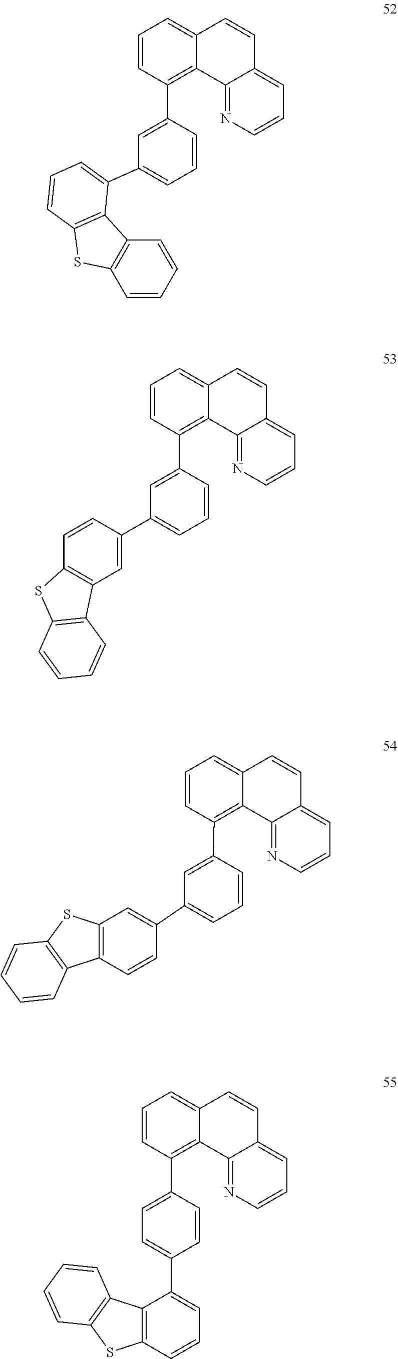 [SMILES: c1cc(-c2cccc3sc4ccccc4c23)cc(-c2cccc3ccc4cccnc4c23)c1 ; c1cc(-c2ccc3sc4ccccc4c3c2)cc(-c2cccc3ccc4cccnc4c23)c1 ; c1cc(-c2ccc3c(c2)sc2ccccc23)cc(-c2cccc3ccc4cccnc4c23)c1 ; c1cnc2c(c1)ccc1cccc(-c3ccc(-c4cccc5sc6ccccc6c45)cc3)c12]

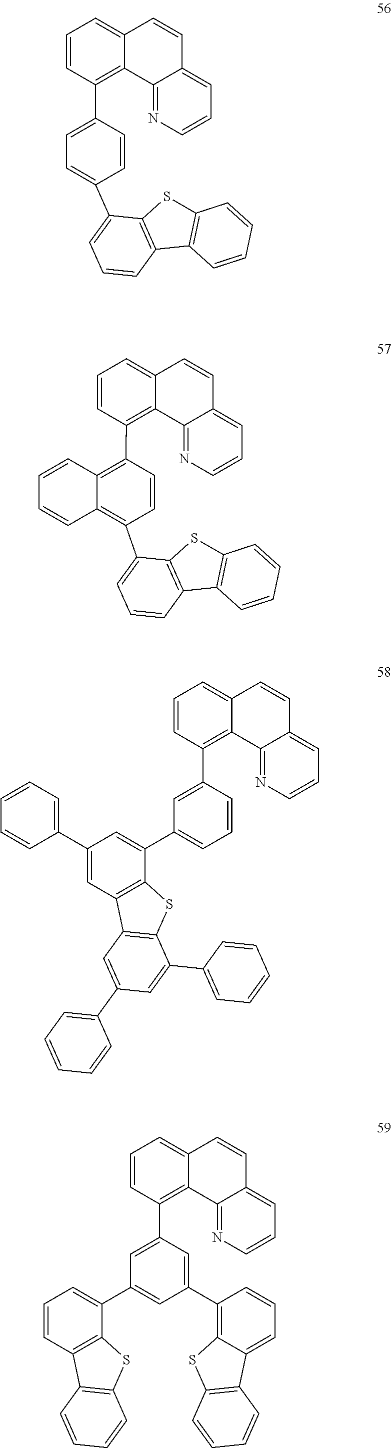 [SMILES: c1cnc2c(c1)ccc1cccc(-c3ccc(-c4cccc5c4sc4ccccc45)cc3)c12 ; c1cnc2c(c1)ccc1cccc(-c3ccc(-c4cccc5c4sc4ccccc45)c4ccccc34)c12 ; c1ccc(-c2cc(-c3ccccc3)c3sc4c(-c5cccc(-c6cccc7ccc8cccnc8c67)c5)cc(-c5ccccc5)cc4c3c2)cc1 ; c1cnc2c(c1)ccc1cccc(-c3cc(-c4cccc5c4sc4ccccc45)cc(-c4cccc5c4sc4ccccc45)c3)c12]

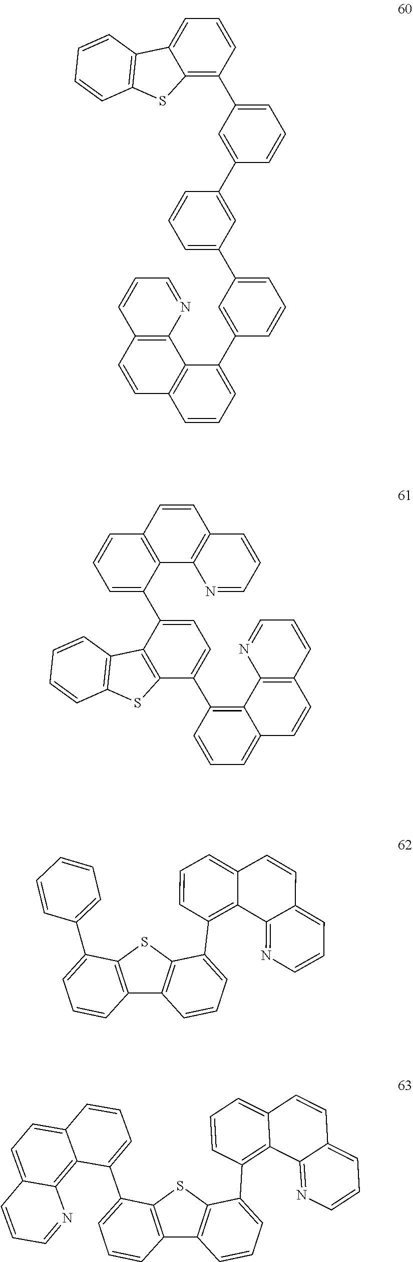 [SMILES: c1cc(-c2cccc(-c3cccc4c3sc3ccccc34)c2)cc(-c2cccc(-c3cccc4ccc5cccnc5c34)c2)c1 ; c1cnc2c(c1)ccc1cccc(-c3ccc(-c4cccc5ccc6cccnc6c45)c4c3sc3ccccc34)c12 ; c1ccc(-c2cccc3c2sc2c(-c4cccc5ccc6cccnc6c45)cccc23)cc1 ; c1cnc2c(c1)ccc1cccc(-c3cccc4c3sc3c(-c5cccc6ccc7cccnc7c56)cccc34)c12]

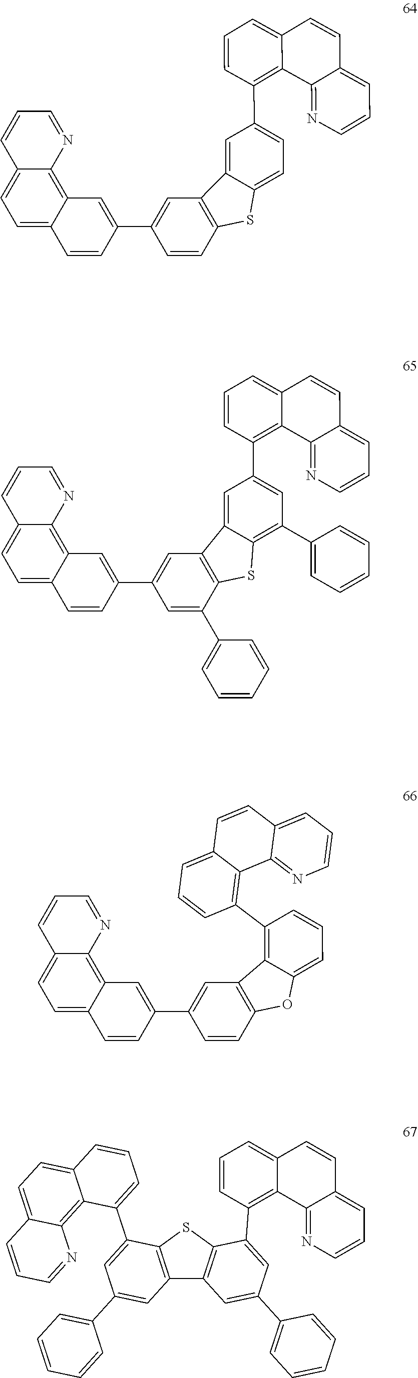 [SMILES: c1cnc2c(c1)ccc1ccc(-c3ccc4sc5ccc(-c6cccc7ccc8cccnc8c67)cc5c4c3)cc12 ; c1ccc(-c2cc(-c3ccc4ccc5cccnc5c4c3)cc3c2sc2c(-c4ccccc4)cc(-c4cccc5ccc6cccnc6c45)cc23)cc1 ; c1cnc2c(c1)ccc1ccc(-c3ccc4oc5cccc(-c6cccc7ccc8cccnc8c67)c5c4c3)cc12 ; c1ccc(-c2cc(-c3cccc4ccc5cccnc5c34)c3sc4c(-c5cccc6ccc7cccnc7c56)cc(-c5ccccc5)cc4c3c2)cc1]

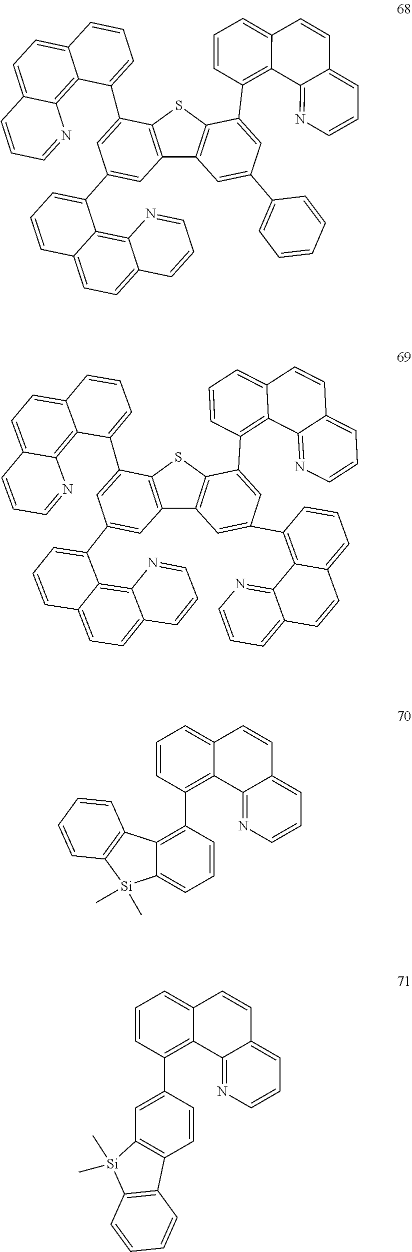 [SMILES: c1ccc(-c2cc(-c3cccc4ccc5cccnc5c34)c3sc4c(-c5cccc6ccc7cccnc7c56)cc(-c5cccc6ccc7cccnc7c56)cc4c3c2)cc1 ; c1cnc2c(c1)ccc1cccc(-c3cc(-c4cccc5ccc6cccnc6c45)c4sc5c(-c6cccc7ccc8cccnc8c67)cc(-c6cccc7ccc8cccnc8c67)cc5c4c3)c12 ; C[Si]1(C)c2ccccc2-c2c(-c3cccc4ccc5cccnc5c34)cccc21 ; C[Si]1(C)c2ccccc2-c2ccc(-c3cccc4ccc5cccnc5c34)cc21]

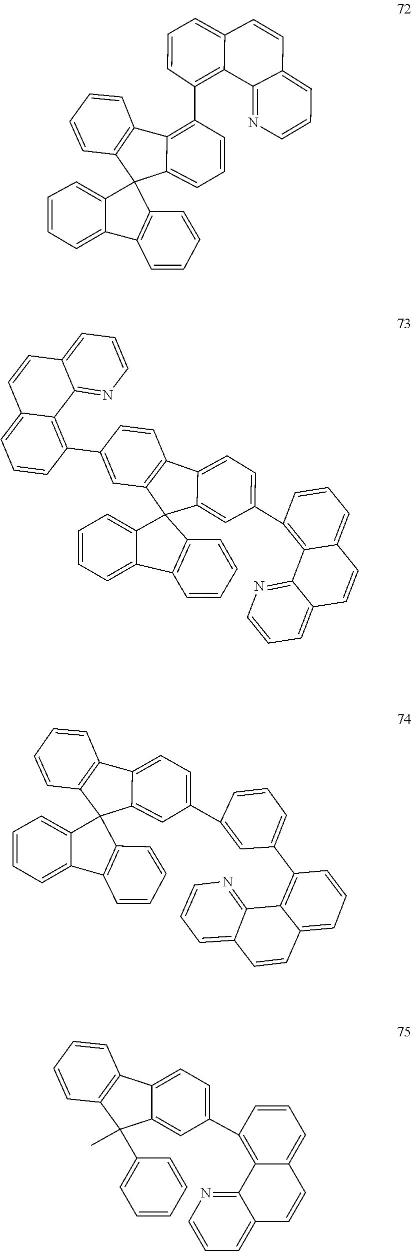 [SMILES: c1ccc2c(c1)-c1ccccc1C21c2ccccc2-c2c(-c3cccc4ccc5cccnc5c34)cccc21 ; c1ccc2c(c1)-c1ccccc1C21c2cc(-c3cccc4ccc5cccnc5c34)ccc2-c2ccc(-c3cccc4ccc5cccnc5c34)cc21 ; c1cc(-c2ccc3c(c2)C2(c4ccccc4-c4ccccc42)c2ccccc2-3)cc(-c2cccc3ccc4cccnc4c23)c1 ; CC1(c2ccccc2)c2ccccc2-c2ccc(-c3cccc4ccc5cccnc5c34)cc21]

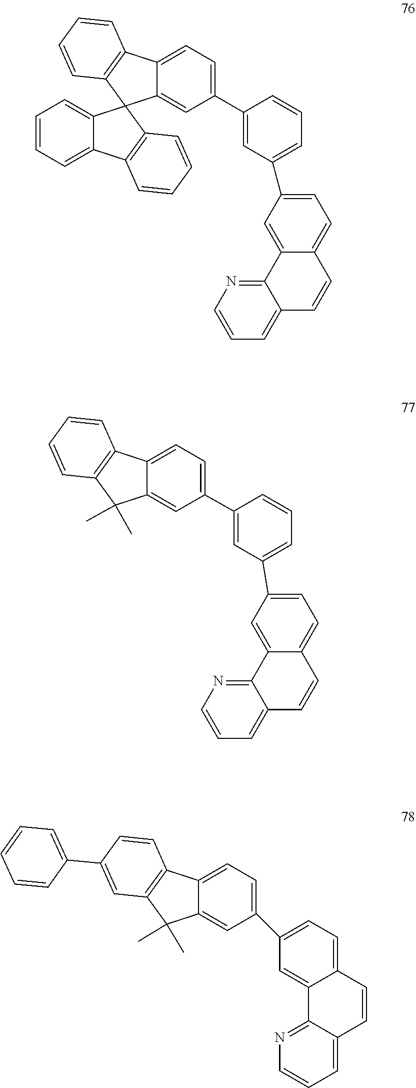 [SMILES: c1cc(-c2ccc3c(c2)C2(c4ccccc4-c4ccccc42)c2ccccc2-3)cc(-c2ccc3ccc4cccnc4c3c2)c1 ; CC1(C)c2ccccc2-c2ccc(-c3cccc(-c4ccc5ccc6cccnc6c5c4)c3)cc21 ; CC1(C)c2cc(-c3ccccc3)ccc2-c2ccc(-c3ccc4ccc5cccnc5c4c3)cc21]

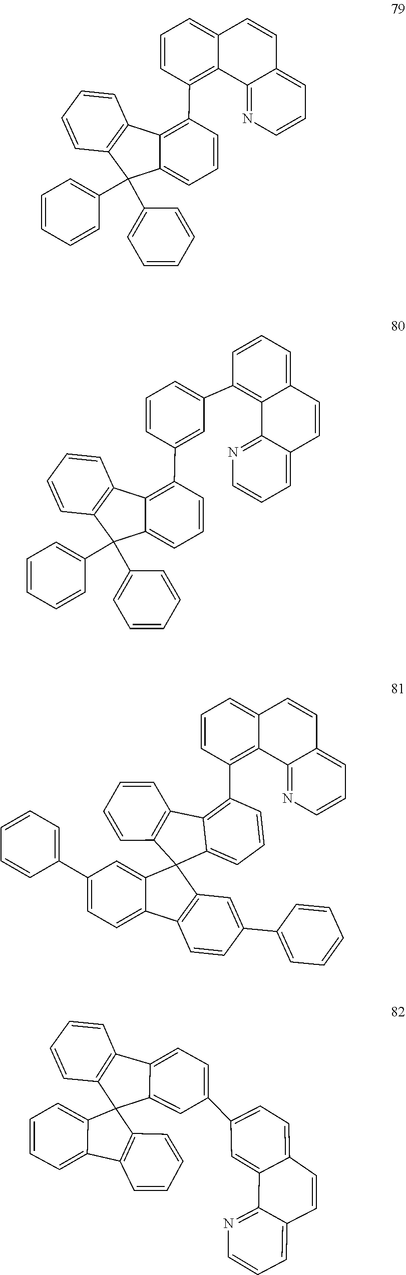 [SMILES: c1ccc(C2(c3ccccc3)c3ccccc3-c3c(-c4cccc5ccc6cccnc6c45)cccc32)cc1 ; c1ccc(C2(c3ccccc3)c3ccccc3-c3c(-c4cccc(-c5cccc6ccc7cccnc7c56)c4)cccc32)cc1 ; c1ccc(-c2ccc3c(c2)C2(c4cc(-c5ccccc5)ccc4-3)c3ccccc3-c3c(-c4cccc5ccc6cccnc6c45)cccc32)cc1 ; c1ccc2c(c1)-c1ccccc1C21c2ccccc2-c2ccc(-c3ccc4ccc5cccnc5c4c3)cc21]

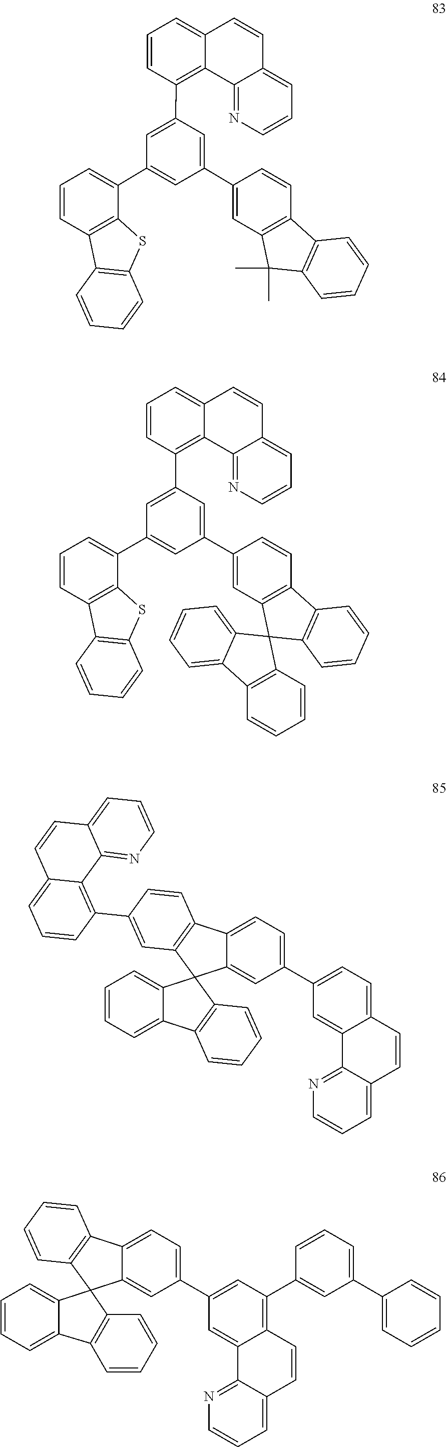 [SMILES: CC1(C)c2ccccc2-c2ccc(-c3cc(-c4cccc5c4sc4ccccc45)cc(-c4cccc5ccc6cccnc6c45)c3)cc21 ; c1ccc2c(c1)-c1ccccc1C21c2ccccc2-c2ccc(-c3cc(-c4cccc5c4sc4ccccc45)cc(-c4cccc5ccc6cccnc6c45)c3)cc21 ; c1ccc2c(c1)-c1ccccc1C21c2cc(-c3ccc4ccc5cccnc5c4c3)ccc2-c2ccc(-c3cccc4ccc5cccnc5c34)cc21 ; c1ccc(-c2cccc(-c3cc(-c4ccc5c(c4)C4(c6ccccc6-c6ccccc64)c4ccccc4-5)cc4c3ccc3cccnc34)c2)cc1]

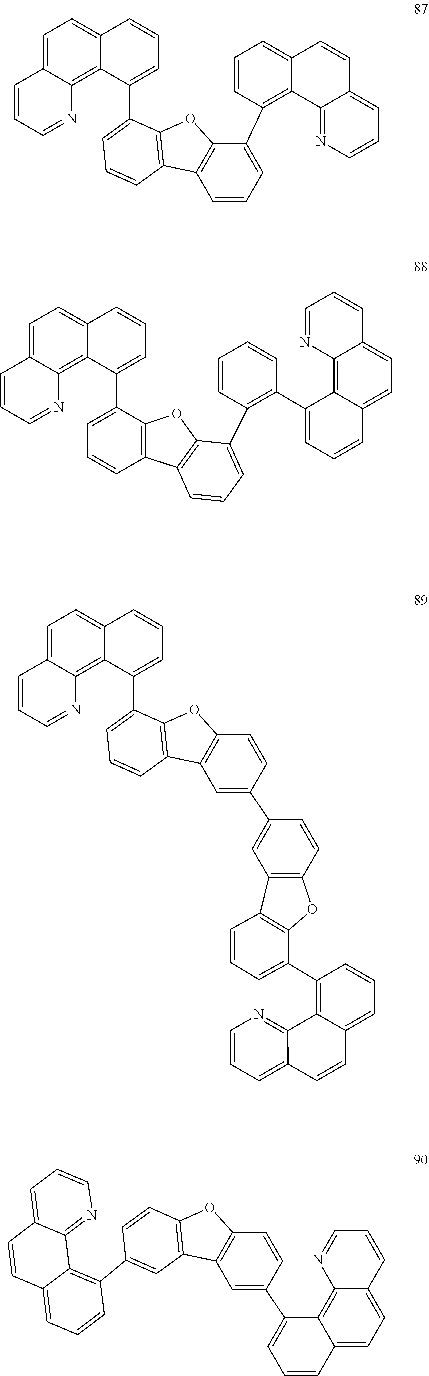 [SMILES: c1cnc2c(c1)ccc1cccc(-c3cccc4c3oc3c(-c5cccc6ccc7cccnc7c56)cccc34)c12 ; c1ccc(-c2cccc3ccc4cccnc4c23)c(-c2cccc3c2oc2c(-c4cccc5ccc6cccnc6c45)cccc23)c1 ; c1cnc2c(c1)ccc1cccc(-c3cccc4c3oc3ccc(-c5ccc6oc7c(-c8cccc9ccc%10cccnc%10c89)cccc7c6c5)cc34)c12 ; c1cnc2c(c1)ccc1cccc(-c3ccc4oc5ccc(-c6cccc7ccc8cccnc8c67)cc5c4c3)c12]

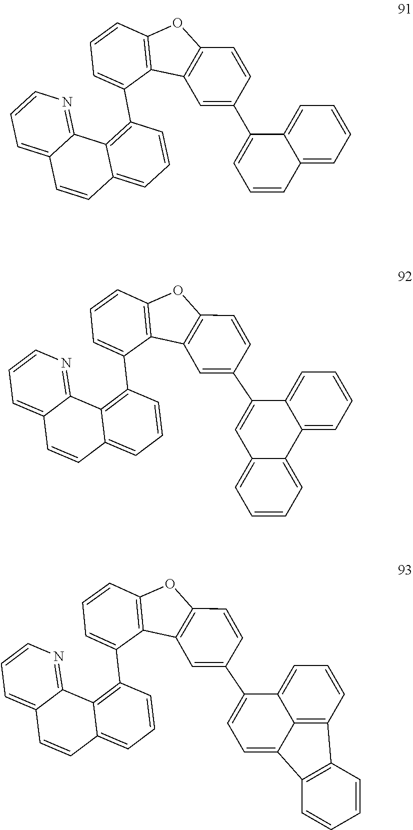 [SMILES: c1ccc2c(-c3ccc4oc5cccc(-c6cccc7ccc8cccnc8c67)c5c4c3)cccc2c1 ; c1ccc2c(c1)cc(-c1ccc3oc4cccc(-c5cccc6ccc7cccnc7c56)c4c3c1)c1ccccc12 ; c1ccc2c(c1)-c1cccc3c(-c4ccc5oc6cccc(-c7cccc8ccc9cccnc9c78)c6c5c4)ccc-2c13]

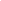

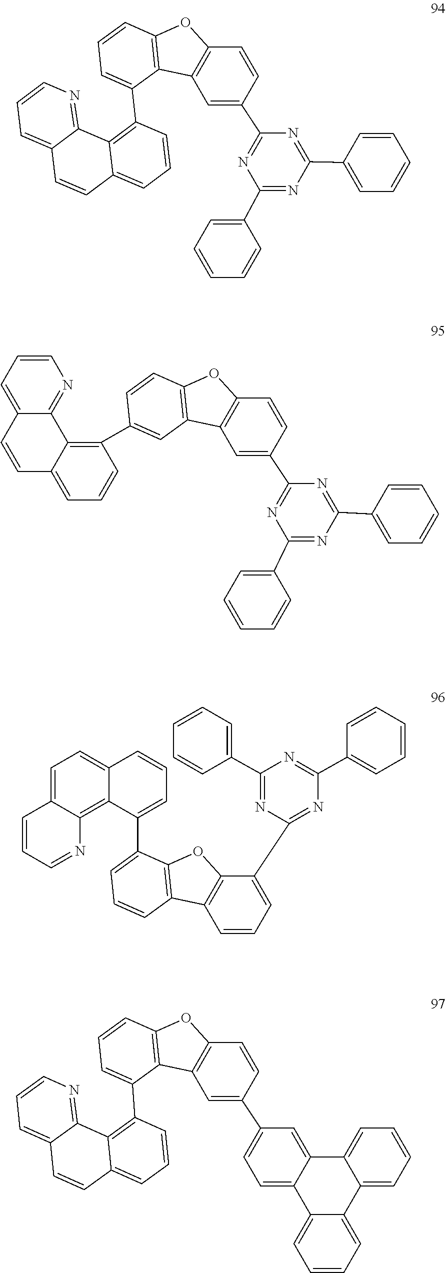 [SMILES: c1ccc(-c2nc(-c3ccccc3)nc(-c3ccc4oc5cccc(-c6cccc7ccc8cccnc8c67)c5c4c3)n2)cc1 ; c1ccc(-c2nc(-c3ccccc3)nc(-c3ccc4oc5ccc(-c6cccc7ccc8cccnc8c67)cc5c4c3)n2)cc1 ; c1ccc(-c2nc(-c3ccccc3)nc(-c3cccc4c3oc3c(-c5cccc6ccc7cccnc7c56)cccc34)n2)cc1 ; c1cnc2c(c1)ccc1cccc(-c3cccc4oc5ccc(-c6ccc7c8ccccc8c8ccccc8c7c6)cc5c34)c12]

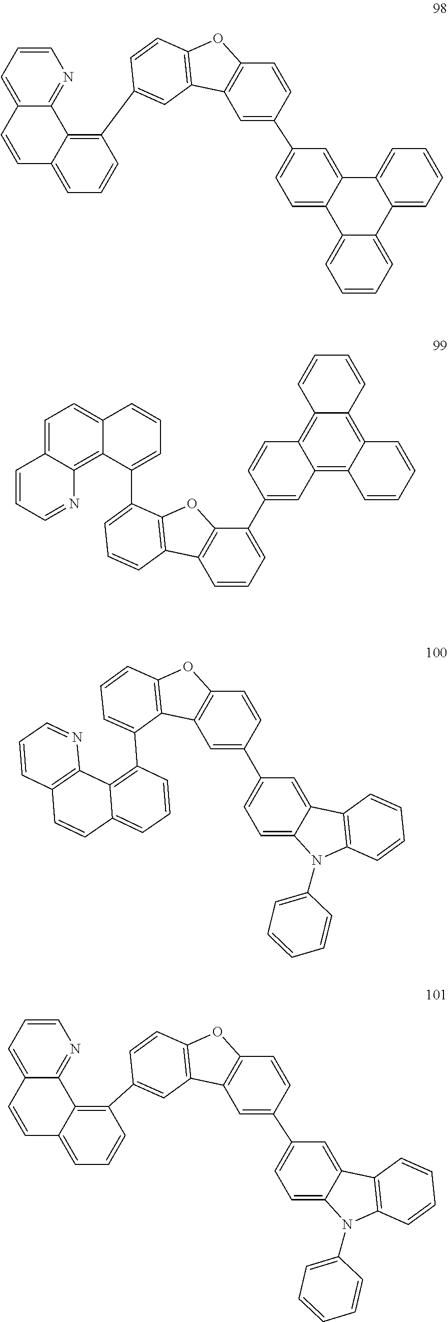 [SMILES: c1cnc2c(c1)ccc1cccc(-c3ccc4oc5ccc(-c6ccc7c8ccccc8c8ccccc8c7c6)cc5c4c3)c12 ; c1cnc2c(c1)ccc1cccc(-c3cccc4c3oc3c(-c5ccc6c7ccccc7c7ccccc7c6c5)cccc34)c12 ; c1ccc(-n2c3ccccc3c3cc(-c4ccc5oc6cccc(-c7cccc8ccc9cccnc9c78)c6c5c4)ccc32)cc1 ; c1ccc(-n2c3ccccc3c3cc(-c4ccc5oc6ccc(-c7cccc8ccc9cccnc9c78)cc6c5c4)ccc32)cc1]

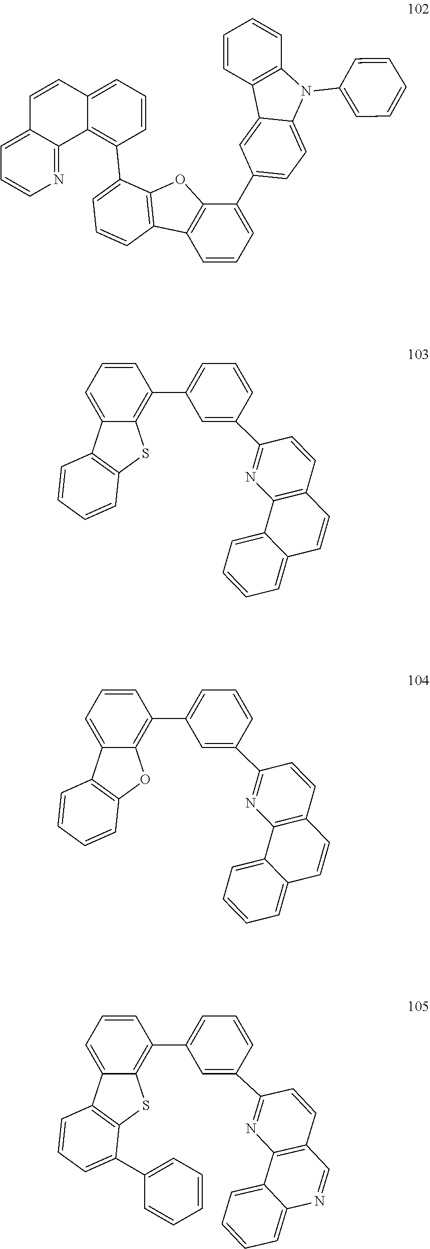 [SMILES: c1ccc(-n2c3ccccc3c3cc(-c4cccc5c4oc4c(-c6cccc7ccc8cccnc8c67)cccc45)ccc32)cc1 ; c1cc(-c2ccc3ccc4ccccc4c3n2)cc(-c2cccc3c2sc2ccccc23)c1 ; c1cc(-c2ccc3ccc4ccccc4c3n2)cc(-c2cccc3c2oc2ccccc23)c1 ; c1ccc(-c2cccc3c2sc2c(-c4cccc(-c5ccc6cnc7ccccc7c6n5)c4)cccc23)cc1]

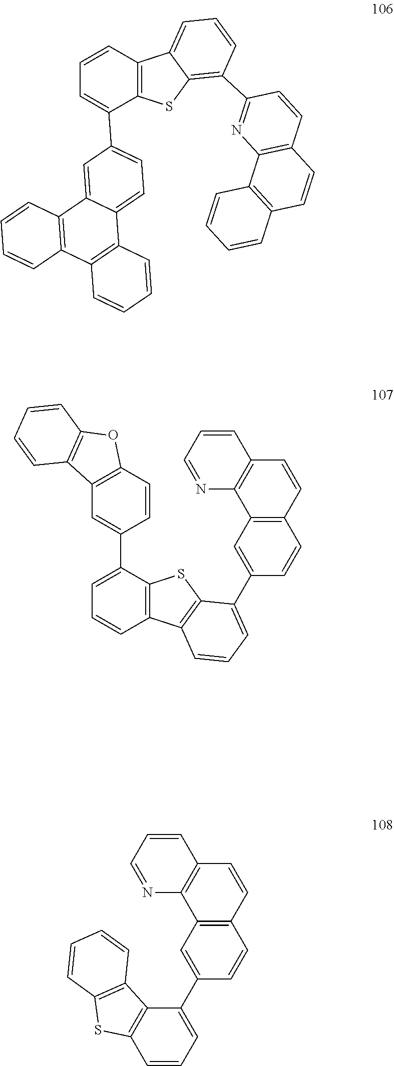 [SMILES: c1ccc2c(c1)ccc1ccc(-c3cccc4c3sc3c(-c5ccc6c7ccccc7c7ccccc7c6c5)cccc34)nc12 ; c1cnc2c(c1)ccc1ccc(-c3cccc4c3sc3c(-c5ccc6oc7ccccc7c6c5)cccc34)cc12 ; c1cnc2c(c1)ccc1ccc(-c3cccc4sc5ccccc5c34)cc12]

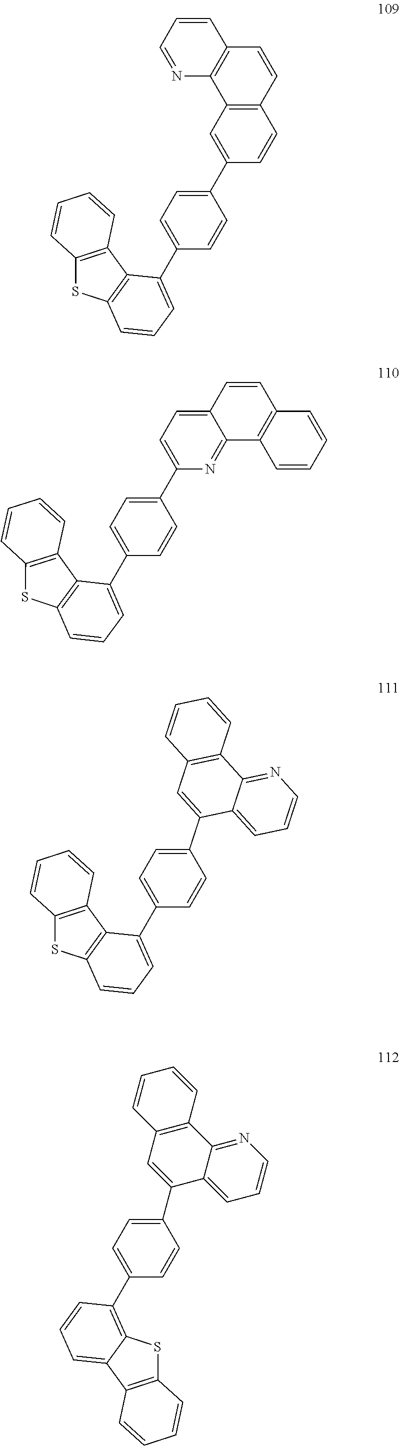 [SMILES: c1cnc2c(c1)ccc1ccc(-c3ccc(-c4cccc5sc6ccccc6c45)cc3)cc12 ; c1ccc2c(c1)ccc1ccc(-c3ccc(-c4cccc5sc6ccccc6c45)cc3)nc12 ; c1ccc2c(c1)cc(-c1ccc(-c3cccc4sc5ccccc5c34)cc1)c1cccnc12 ; c1ccc2c(c1)cc(-c1ccc(-c3cccc4c3sc3ccccc34)cc1)c1cccnc12]

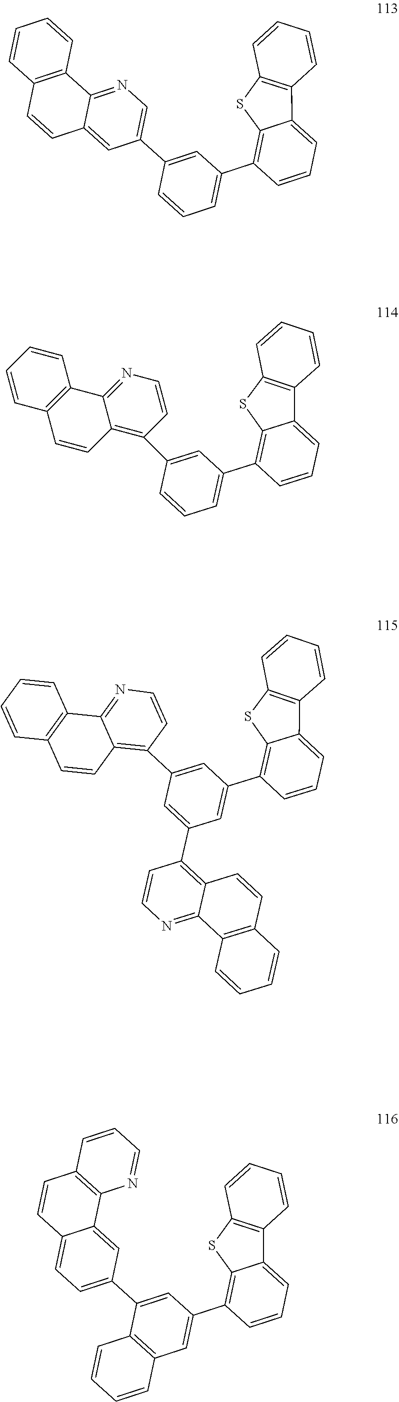 [SMILES: c1cc(-c2cnc3c(ccc4ccccc43)c2)cc(-c2cccc3c2sc2ccccc23)c1 ; c1cc(-c2ccnc3c2ccc2ccccc23)cc(-c2cccc3c2sc2ccccc23)c1 ; c1ccc2c(c1)ccc1c(-c3cc(-c4ccnc5c4ccc4ccccc45)cc(-c4cccc5c4sc4ccccc45)c3)ccnc12 ; c1ccc2c(-c3ccc4ccc5cccnc5c4c3)cc(-c3cccc4c3sc3ccccc34)cc2c1]

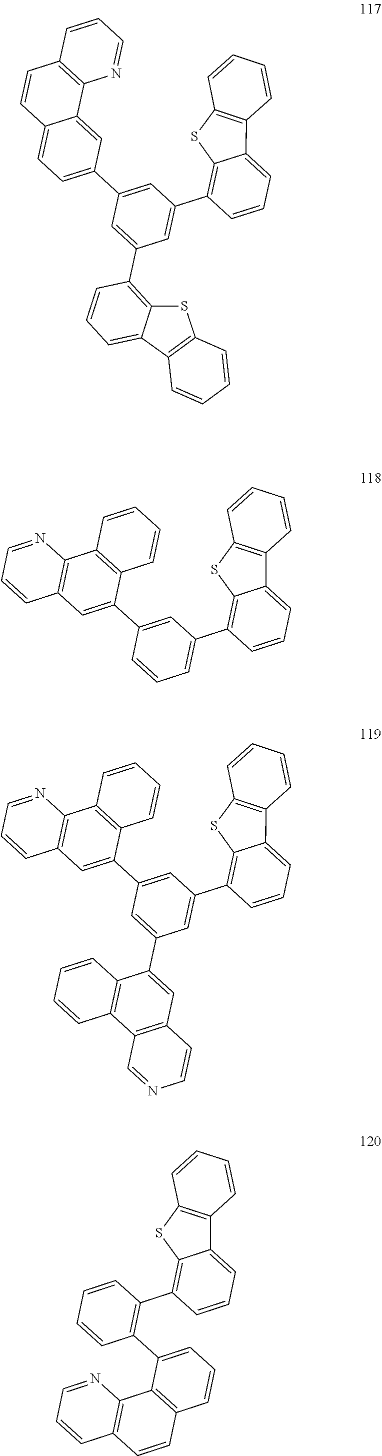 [SMILES: c1cnc2c(c1)ccc1ccc(-c3cc(-c4cccc5c4sc4ccccc45)cc(-c4cccc5c4sc4ccccc45)c3)cc12 ; c1cc(-c2cc3cccnc3c3ccccc23)cc(-c2cccc3c2sc2ccccc23)c1 ; c1cnc2c(c1)cc(-c1cc(-c3cc4ccncc4c4ccccc34)cc(-c3cccc4c3sc3ccccc34)c1)c1ccccc12 ; c1ccc(-c2cccc3ccc4cccnc4c23)c(-c2cccc3c2sc2ccccc23)c1]

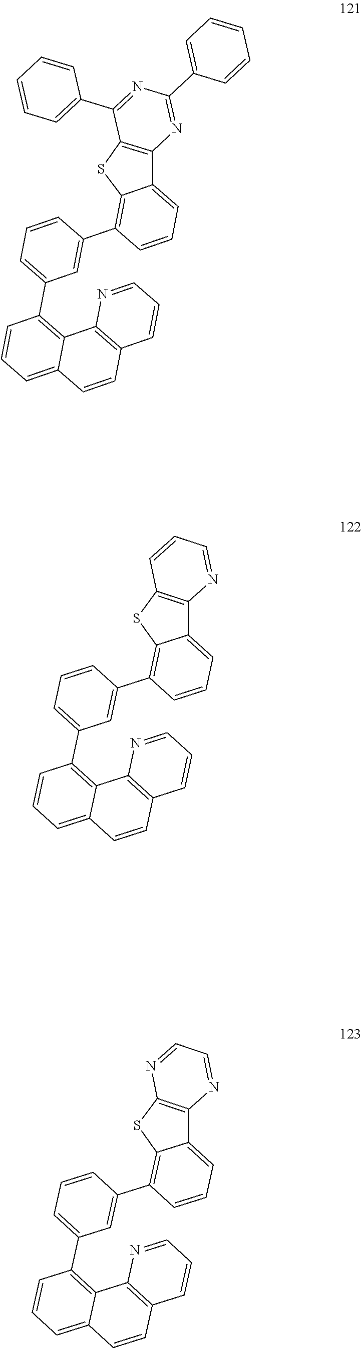 [SMILES: c1ccc(-c2nc(-c3ccccc3)c3sc4c(-c5cccc(-c6cccc7ccc8cccnc8c67)c5)cccc4c3n2)cc1 ; c1cc(-c2cccc3c2sc2cccnc23)cc(-c2cccc3ccc4cccnc4c23)c1 ; c1cc(-c2cccc3c2sc2nccnc23)cc(-c2cccc3ccc4cccnc4c23)c1]

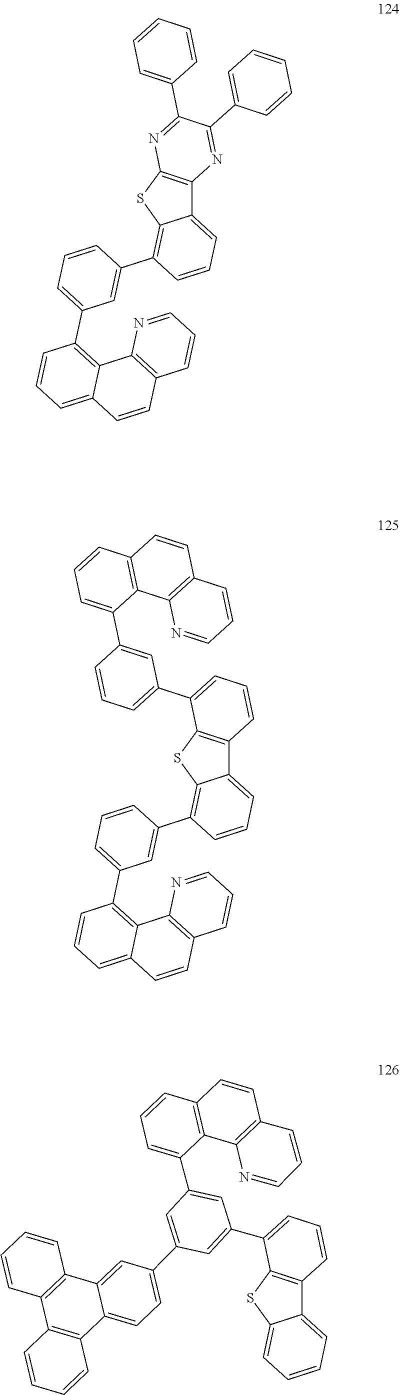 [SMILES: c1ccc(-c2nc3sc4c(-c5cccc(-c6cccc7ccc8cccnc8c67)c5)cccc4c3nc2-c2ccccc2)cc1 ; c1cc(-c2cccc3c2sc2c(-c4cccc(-c5cccc6ccc7cccnc7c56)c4)cccc23)cc(-c2cccc3ccc4cccnc4c23)c1 ; c1cnc2c(c1)ccc1cccc(-c3cc(-c4ccc5c6ccccc6c6ccccc6c5c4)cc(-c4cccc5c4sc4ccccc45)c3)c12]

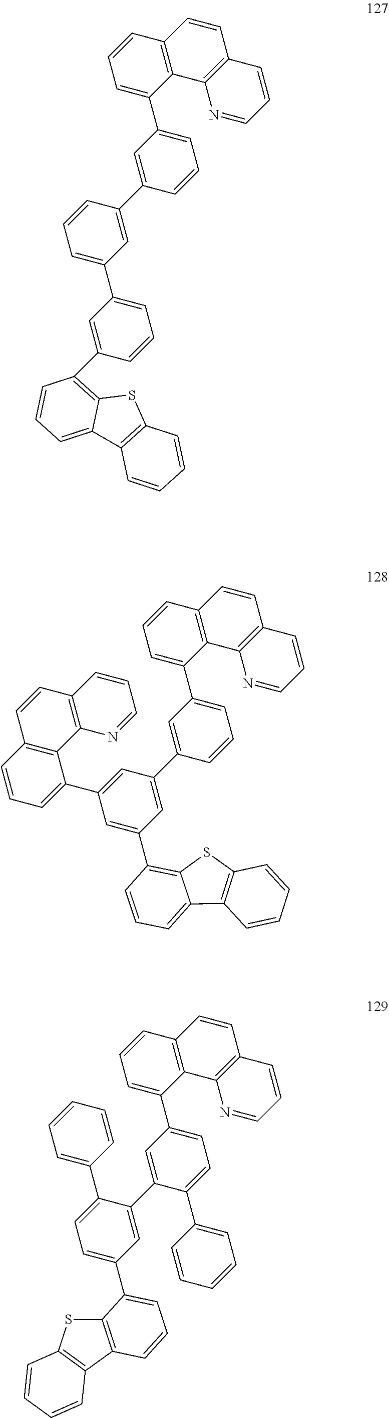 [SMILES: c1cc(-c2cccc(-c3cccc4c3sc3ccccc34)c2)cc(-c2cccc(-c3cccc4ccc5cccnc5c34)c2)c1 ; c1cc(-c2cc(-c3cccc4c3sc3ccccc34)cc(-c3cccc4ccc5cccnc5c34)c2)cc(-c2cccc3ccc4cccnc4c23)c1 ; c1ccc(-c2ccc(-c3cccc4c3sc3ccccc34)cc2-c2cc(-c3cccc4ccc5cccnc5c34)ccc2-c2ccccc2)cc1]

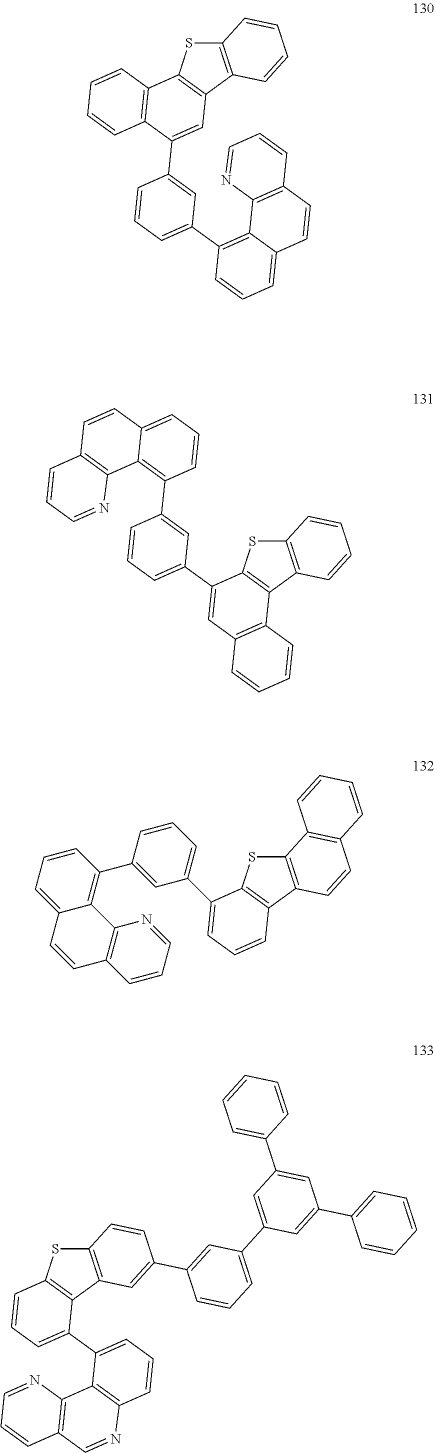 [SMILES: c1cc(-c2cc3c4ccccc4sc3c3ccccc23)cc(-c2cccc3ccc4cccnc4c23)c1 ; c1cc(-c2cc3ccccc3c3c2sc2ccccc23)cc(-c2cccc3ccc4cccnc4c23)c1 ; c1cc(-c2cccc3c2sc2c4ccccc4ccc32)cc(-c2cccc3ccc4cccnc4c23)c1 ; c1ccc(-c2cc(-c3ccccc3)cc(-c3cccc(-c4ccc5sc6cccc(-c7cccc8ncc9cccnc9c78)c6c5c4)c3)c2)cc1]

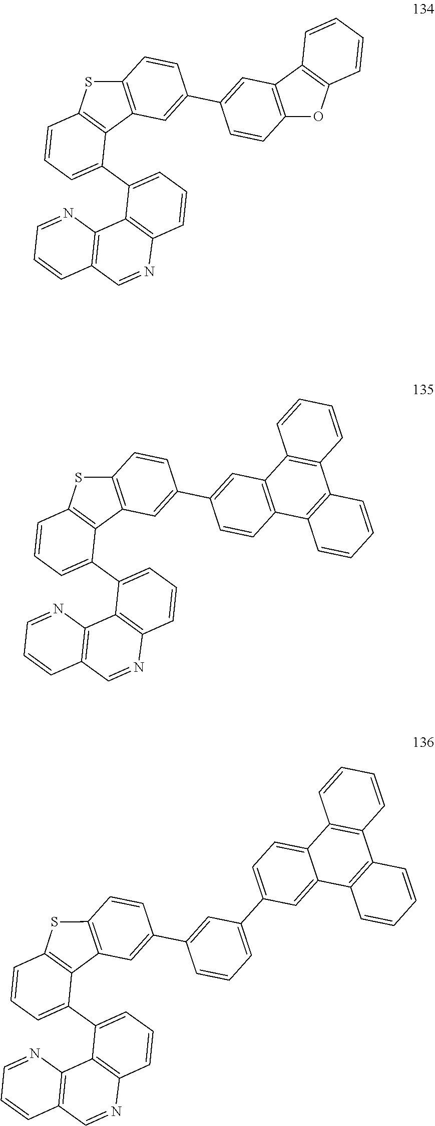 [SMILES: c1cnc2c(c1)cnc1cccc(-c3cccc4sc5ccc(-c6ccc7oc8ccccc8c7c6)cc5c34)c12 ; c1cnc2c(c1)cnc1cccc(-c3cccc4sc5ccc(-c6ccc7c8ccccc8c8ccccc8c7c6)cc5c34)c12 ; c1cc(-c2ccc3c4ccccc4c4ccccc4c3c2)cc(-c2ccc3sc4cccc(-c5cccc6ncc7cccnc7c56)c4c3c2)c1]

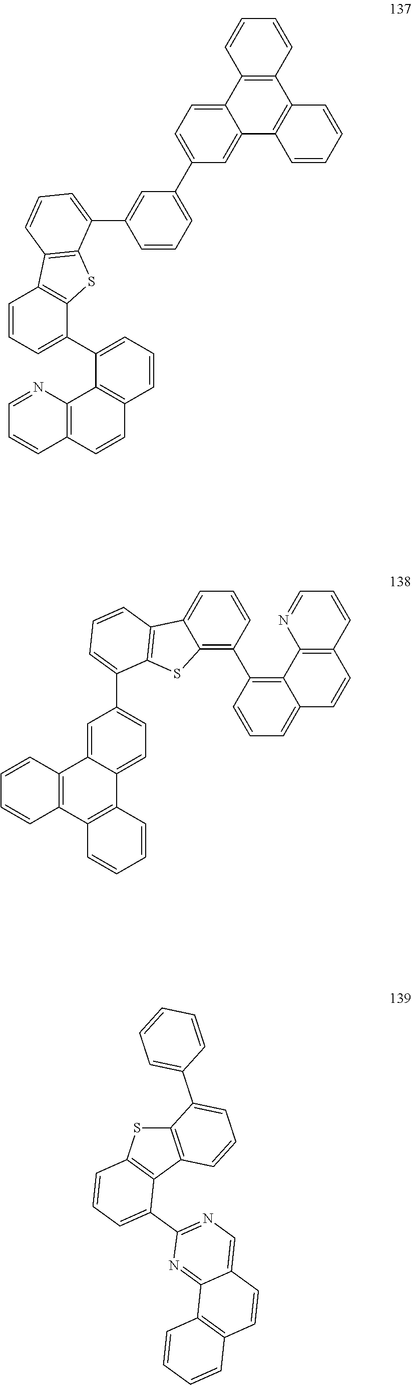 [SMILES: c1cc(-c2ccc3c4ccccc4c4ccccc4c3c2)cc(-c2cccc3c2sc2c(-c4cccc5ccc6cccnc6c45)cccc23)c1 ; c1cnc2c(c1)ccc1cccc(-c3cccc4c3sc3c(-c5ccc6c7ccccc7c7ccccc7c6c5)cccc34)c12 ; c1ccc(-c2cccc3c2sc2cccc(-c4ncc5ccc6ccccc6c5n4)c23)cc1]

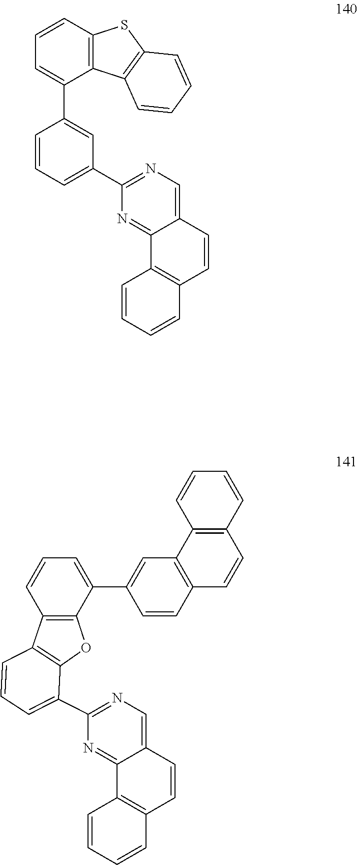 [SMILES: c1cc(-c2ncc3ccc4ccccc4c3n2)cc(-c2cccc3sc4ccccc4c23)c1 ; c1ccc2c(c1)ccc1ccc(-c3cccc4c3oc3c(-c5ncc6ccc7ccccc7c6n5)cccc34)cc12]

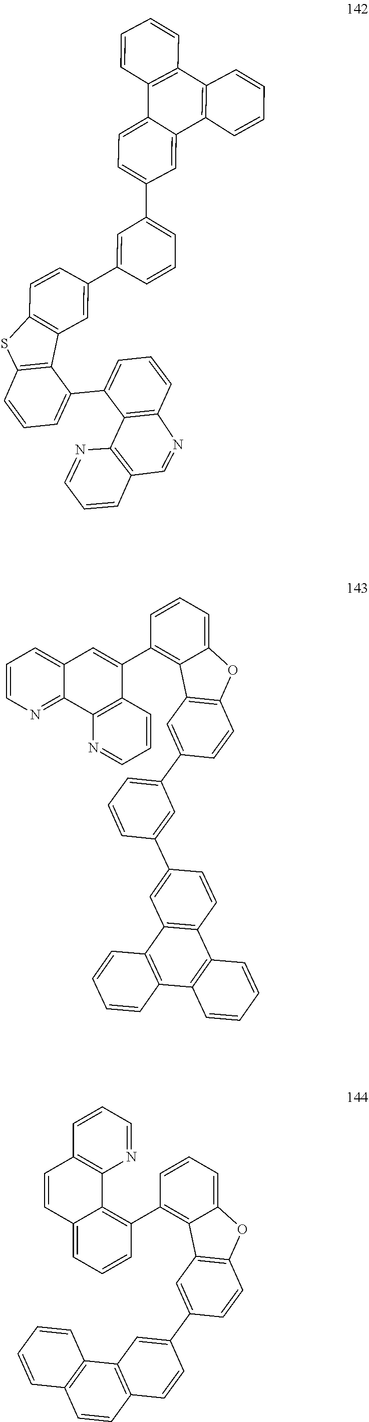 [SMILES: c1cc(-c2ccc3c4ccccc4c4ccccc4c3c2)cc(-c2ccc3sc4cccc(-c5cccc6ncc7cccnc7c56)c4c3c2)c1 ; c1cc(-c2ccc3c4ccccc4c4ccccc4c3c2)cc(-c2ccc3oc4cccc(-c5cc6cccnc6c6ncccc56)c4c3c2)c1 ; c1ccc2c(c1)ccc1ccc(-c3ccc4oc5cccc(-c6cccc7ccc8cccnc8c67)c5c4c3)cc12]

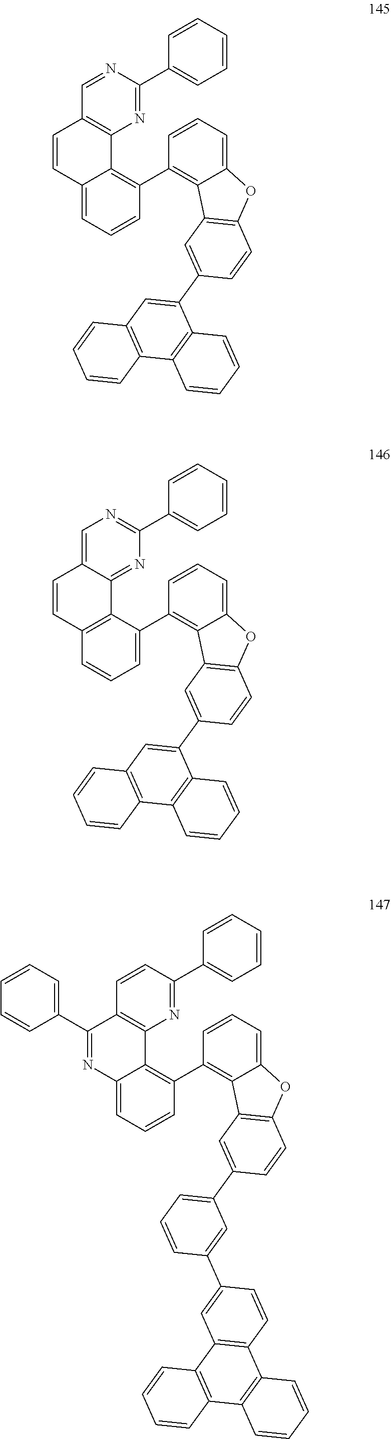 [SMILES: c1ccc(-c2ncc3ccc4cccc(-c5cccc6oc7ccc(-c8cc9ccccc9c9ccccc89)cc7c56)c4c3n2)cc1 ; c1ccc(-c2ncc3ccc4cccc(-c5cccc6oc7ccc(-c8cc9ccccc9c9ccccc89)cc7c56)c4c3n2)cc1 ; c1ccc(-c2ccc3c(-c4ccccc4)nc4cccc(-c5cccc6oc7ccc(-c8cccc(-c9ccc%10c%11ccccc%11c%11ccccc%11c%10c9)c8)cc7c56)c4c3n2)cc1]

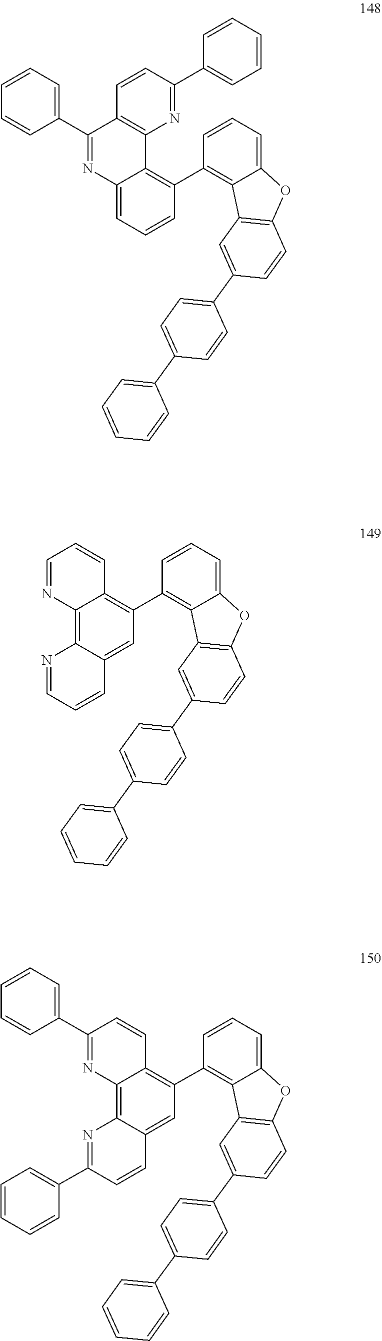 [SMILES: c1ccc(-c2ccc(-c3ccc4oc5cccc(-c6cccc7nc(-c8ccccc8)c8ccc(-c9ccccc9)nc8c67)c5c4c3)cc2)cc1 ; c1ccc(-c2ccc(-c3ccc4oc5cccc(-c6cc7cccnc7c7ncccc67)c5c4c3)cc2)cc1 ; c1ccc(-c2ccc(-c3ccc4oc5cccc(-c6cc7ccc(-c8ccccc8)nc7c7nc(-c8ccccc8)ccc67)c5c4c3)cc2)cc1]

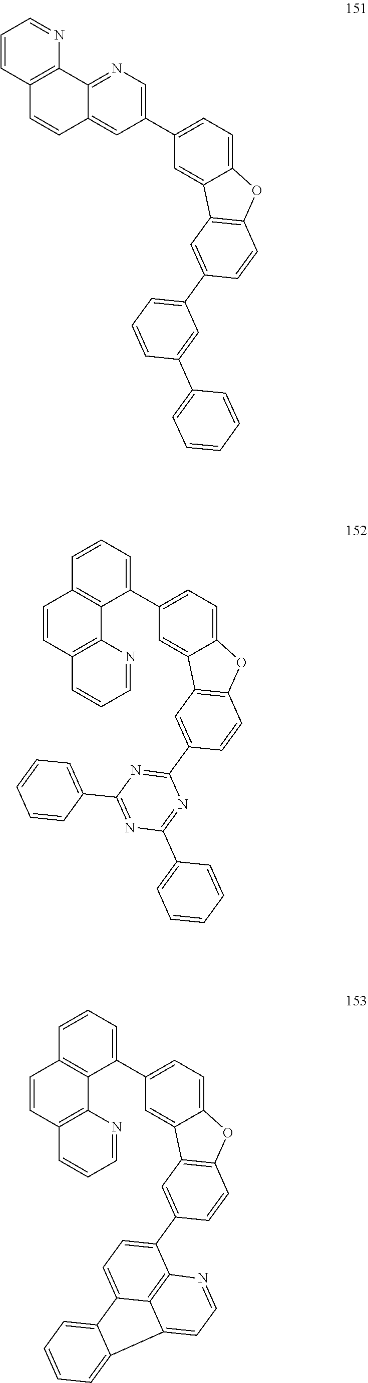 [SMILES: c1ccc(-c2cccc(-c3ccc4oc5ccc(-c6cnc7c(ccc8cccnc87)c6)cc5c4c3)c2)cc1 ; c1ccc(-c2nc(-c3ccccc3)nc(-c3ccc4oc5ccc(-c6cccc7ccc8cccnc8c67)cc5c4c3)n2)cc1 ; c1ccc2c(c1)-c1ccnc3c(-c4ccc5oc6ccc(-c7cccc8ccc9cccnc9c78)cc6c5c4)ccc-2c13]

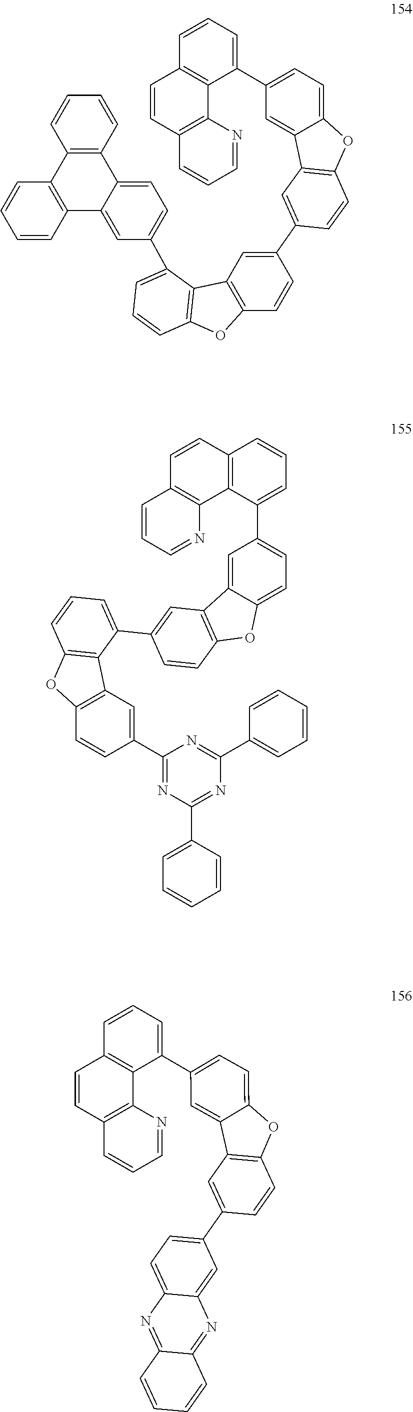 [SMILES: c1cnc2c(c1)ccc1cccc(-c3ccc4oc5ccc(-c6ccc7oc8cccc(-c9ccc%10c%11ccccc%11c%11ccccc%11c%10c9)c8c7c6)cc5c4c3)c12 ; c1ccc(-c2nc(-c3ccccc3)nc(-c3ccc4oc5cccc(-c6ccc7oc8ccc(-c9cccc%10ccc%11cccnc%11c9%10)cc8c7c6)c5c4c3)n2)cc1 ; c1cnc2c(c1)ccc1cccc(-c3ccc4oc5ccc(-c6ccc7nc8ccccc8nc7c6)cc5c4c3)c12]

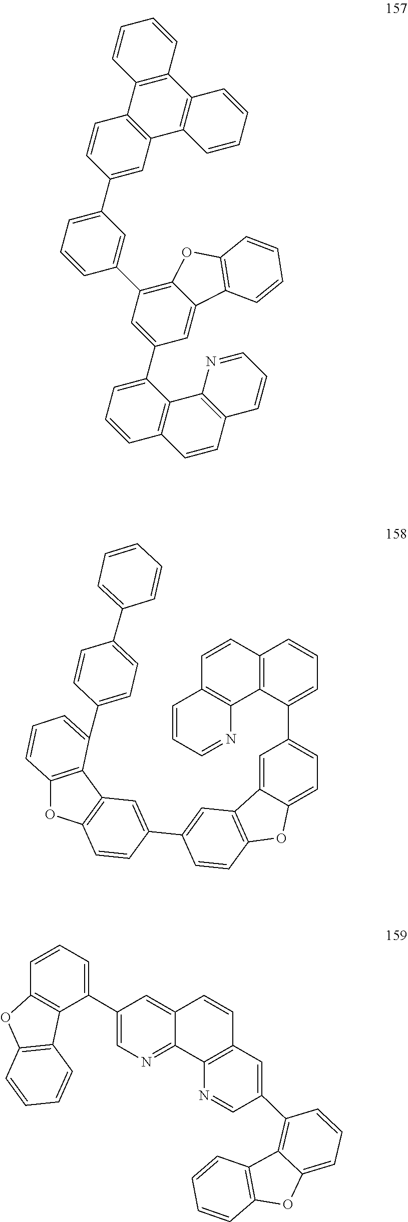 [SMILES: c1cc(-c2ccc3c4ccccc4c4ccccc4c3c2)cc(-c2cc(-c3cccc4ccc5cccnc5c34)cc3c2oc2ccccc23)c1 ; c1ccc(-c2ccc(-c3cccc4oc5ccc(-c6ccc7oc8ccc(-c9cccc%10ccc%11cccnc%11c9%10)cc8c7c6)cc5c34)cc2)cc1 ; c1ccc2c(c1)oc1cccc(-c3cnc4c(ccc5cc(-c6cccc7oc8ccccc8c67)cnc54)c3)c12]

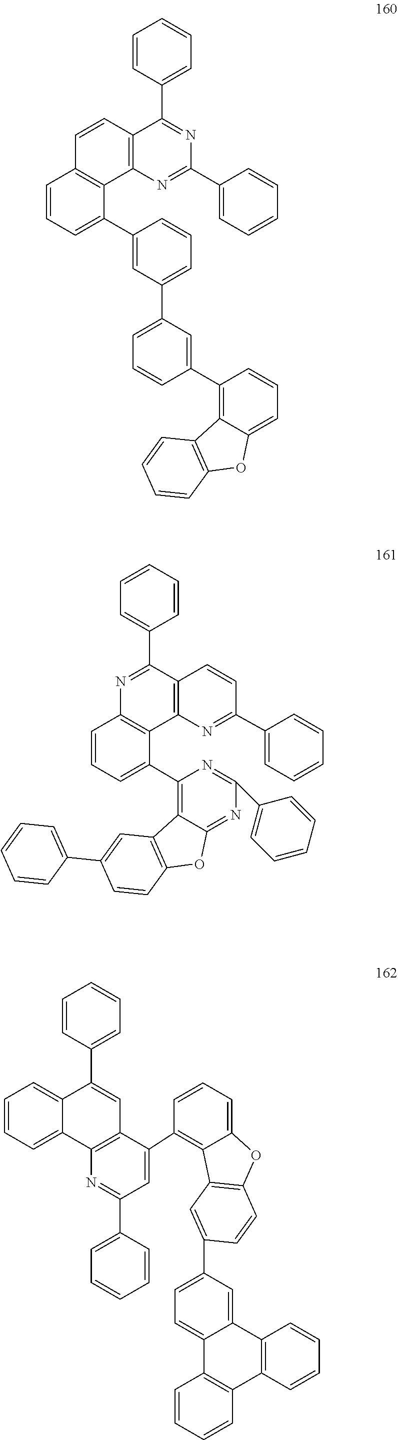 [SMILES: c1ccc(-c2nc(-c3ccccc3)c3ccc4cccc(-c5cccc(-c6cccc(-c7cccc8oc9ccccc9c78)c6)c5)c4c3n2)cc1 ; c1ccc(-c2ccc3oc4nc(-c5ccccc5)nc(-c5cccc6nc(-c7ccccc7)c7ccc(-c8ccccc8)nc7c56)c4c3c2)cc1 ; c1ccc(-c2cc(-c3cccc4oc5ccc(-c6ccc7c8ccccc8c8ccccc8c7c6)cc5c34)c3cc(-c4ccccc4)c4ccccc4c3n2)cc1]

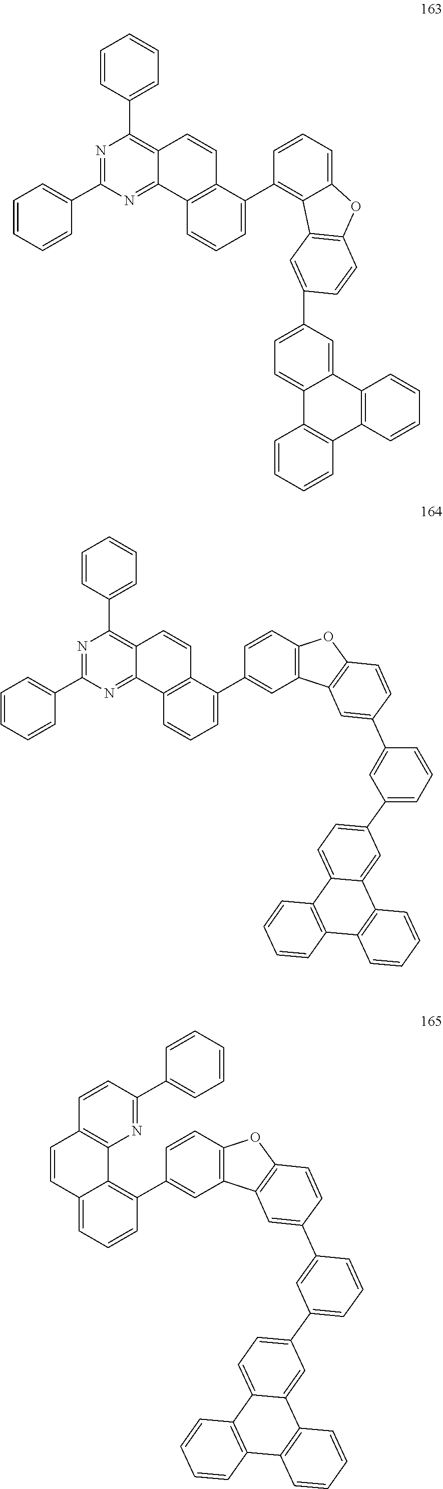 [SMILES: c1ccc(-c2nc(-c3ccccc3)c3ccc4c(-c5cccc6oc7ccc(-c8ccc9c%10ccccc%10c%10ccccc%10c9c8)cc7c56)cccc4c3n2)cc1 ; c1ccc(-c2nc(-c3ccccc3)c3ccc4c(-c5ccc6oc7ccc(-c8cccc(-c9ccc%10c%11ccccc%11c%11ccccc%11c%10c9)c8)cc7c6c5)cccc4c3n2)cc1 ; c1ccc(-c2ccc3ccc4cccc(-c5ccc6oc7ccc(-c8cccc(-c9ccc%10c%11ccccc%11c%11ccccc%11c%10c9)c8)cc7c6c5)c4c3n2)cc1]

The compound of the abovementioned formulae is preferably a compound having a large energy gap between HOMO and LUMO, preferably an energy gap of 2.5 eV or more, more preferably 3 eV or more, most preferably 3.5 eV or more. Such materials are referred to as wide bandgap materials, especially wide bandgap matrix materials.

The HOMO and LUMO energies are determined via quantum-chemical calculations. For this purpose, in the present case, the “Gaussian09, Revision D.01” software package (Gaussian Inc.) is used. For calculation of organic substances without metals (referred to as the “org.” method), a geometry optimization is first conducted by the semi-empirical method AM1 (Gaussian input line “#AM1 opt”) with charge 0 and multiplicity 1. Subsequently, on the basis of the optimized geometry, a (single-point) energy calculation is effected for the electronic ground state and the triplet level. This is done using the TDDFT (time dependent density functional theory) method B3PW91 with the 6-31G(d) basis set (Gaussian input line “#B3PW91/6-31G(d) td=(50−50,nstates=4)”) (charge 0, multiplicity 1). For organometallic compounds (referred to as the “M-org.” method), the geometry is optimized by the Hartree-Fock method and the LanL2 MB basis set (Gaussian input line “#HF/LanL2 MB opt”) (charge 0, multiplicity 1). The energy calculation is effected, as described above, analogously to that for the organic substances, except that the “LanL2DZ” basis set is used for the metal atom and the “6-31G(d)” basis set for the ligands (Gaussian input line “#B3PW91/gen pseudo=|an|2 td=(50−50,nstates=4)”). From the energy calculation, the HOMO is obtained as the last orbital occupied by two electrons (alpha occ. eigenvalues) and LUMO as the first unoccupied orbital (alpha virt. eigenvalues) in Hartree units, where HEh and LEh represent the HOMO energy in Hartree units and the LUMO energy in Hartree units respectively. This is used to determine the HOMO and LUMO value in electron volts, calibrated by cyclic voltammetry measurements, as follows:

HOMO(eV)=(HEh*27.212)*0.8308−1.118

LUMO(eV)=(LEh*27.212)*1.0658−0.5049

These values are to be regarded as HOMO and as LUMO of the materials in the context of this application. The magnitude of the difference between the two values is regarded as the band gap in the context of this application.

The compounds of the abovementioned formulae may be prepared by known methods of organic synthetic chemistry, especially by means of Suzuki coupling reactions. Preferred processes for preparing the compounds according to the application are detailed hereinafter.

According to the method shown in scheme 1, proceeding from a boronic acid-substituted benzoquinoline derivative, in a Suzuki coupling, a Ar¹-Ar² unit is introduced as halogenated reagent. Alternatively, the benzoquinoline derivative may also be halogen-substituted, and the Ar¹-Ar² unit is introduced as boronic acid-substituted reagent.

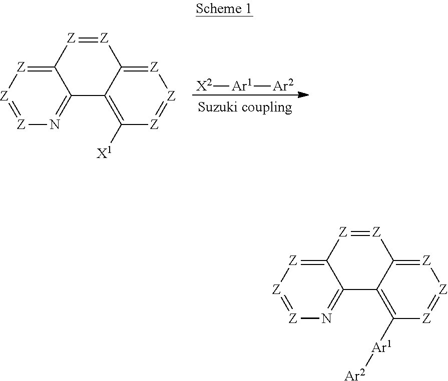

X¹, X²=halogen or boronic acid derivative

In the method shown in scheme 2, first of all, proceeding from a boronic acid-substituted benzoquinoline derivative, a spacer group Ar¹ is introduced in a Suzuki reaction. The latter bears a further reactive group that does not react in the first Suzuki reaction. Then, in a second Suzuki reaction, an Are group is introduced.

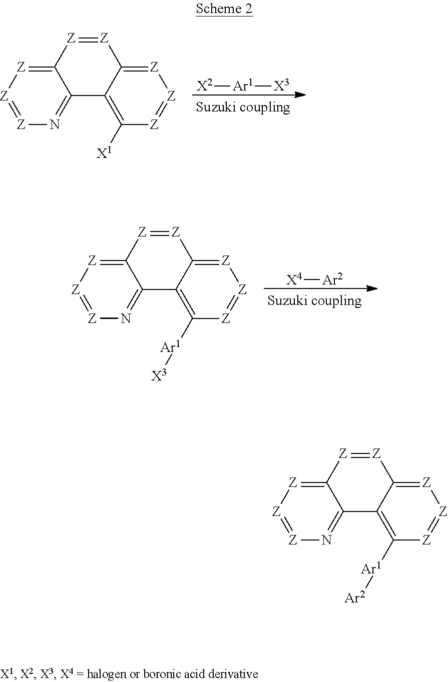

Corresponding methods can also be used to prepare compounds of the formula (II).

In a further variation of the process of the invention, rather than compounds

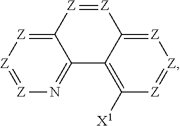

it is possible to use alternative compounds that bear the X¹ group at any other position on the base skeleton, for example compounds of the following formula:

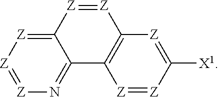

The present application thus further provides a process for preparing a compound of the application, characterized in that a compound of the formula (Z)

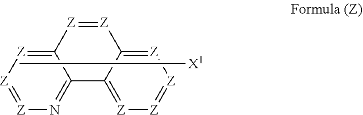

where X¹ is selected from B(OR⁴)₂, Cl, Br and I is converted in a Suzuki reaction.

The above-described compounds, especially compounds substituted by reactive leaving groups, such as bromine, iodine, chlorine, boronic acid or boronic ester, may find use as monomers for production of corresponding oligomers, dendrimers or polymers. Suitable reactive leaving groups are, for example, bromine, iodine, chlorine, boronic acids, boronic esters, amines, alkenyl or alkynyl groups having a terminal C═C double bond or C—C triple bond, oxiranes, oxetanes, groups which enter into a cycloaddition, for example a 1,3-dipolar cycloaddition, for example dienes or azides, carboxylic acid derivatives, alcohols and silanes.

The invention therefore further provides oligomers, polymers or dendrimers containing one or more compounds of the abovementioned formulae, wherein the bond(s) to the polymer, oligomer or dendrimer may be localized at any desired positions substituted by R¹, R² or R³ in the formulae. According to the linkage of the compound, the compound is part of a side chain of the oligomer or polymer or part of the main chain. An oligomer in the context of this invention is understood to mean a compound formed from at least three monomer units. A polymer in the context of the invention is understood to mean a compound formed from at least ten monomer units. The polymers, oligomers or dendrimers of the invention may be conjugated, partly conjugated or nonconjugated. The oligomers or polymers of the invention may be linear, branched or dendritic. In the structures having linear linkage, the units the abovementioned formulae may be joined directly to one another, or they may be joined to one another via a bivalent group, for example via a substituted or unsubstituted alkylene group, via a heteroatom or via a bivalent aromatic or heteroaromatic group. In branched and dendritic structures, it is possible, for example, for three or more units of the abovementioned formulae to be joined via a trivalent or higher-valency group, for example via a trivalent or higher-valency aromatic or heteroaromatic group, to give a branched or dendritic oligomer or polymer.

For the repeat units of the abovementioned formulae in oligomers, dendrimers and polymers, the same preferences apply as described above for compounds of the abovementioned formulae.

For preparation of the oligomers or polymers, the monomers of the invention are homopolymerized or copolymerized with further monomers. Suitable and preferred comonomers are selected from fluorenes, spirobifluorenes, paraphenylenes, carbazoles, thiophenes, dihydrophenanthrenes, cis- and trans-indenofluorenes, ketones, phenanthrenes or two or more of these units. The polymers, oligomers and dendrimers typically contain still further units, for example emitting (fluorescent or phosphorescent) units, for example vinyltriarylamines or phosphorescent metal complexes, and/or charge transport units, especially those based on triarylamines.

The polymers and oligomers of the invention are generally prepared by polymerization of one or more monomer types, of which at least one monomer leads to repeat units of the abovementioned formulae in the polymer. Suitable polymerization reactions are known to those skilled in the art and are described in the literature. Particularly suitable and preferred polymerization reactions which lead to formation of C—C or C—N bonds are the Suzuki polymerization, the Yamamoto polymerization, the Stille polymerization and the Hartwig-Buchwald polymerization.

For the processing of the compounds of the invention from a liquid phase, for example by spin-coating or by printing methods, formulations of the compounds of the invention are required. These formulations may, for example, be solutions, dispersions or emulsions. For this purpose, it may be preferable to use mixtures of two or more solvents. Suitable and preferred solvents are, for example, toluene, anisole, o-, m- or p-xylene, methyl benzoate, mesitylene, tetralin, veratrole, THF, methyl-THF, THP, chlorobenzene, dioxane, phenoxytoluene, especially 3-phenoxytoluene, (−)-fenchone, 1,2,3,5-tetramethylbenzene, 1,2,4,5-tetramethylbenzene, 1-methylnaphthalene, 2-methylbenzothiazole, 2-phenoxyethanol, 2-pyrrolidinone, 3-methylanisole, 4-methylanisole, 3,4-dimethylanisole, 3,5-dimethylanisole, acetophenone, α-terpineol, benzothiazole, butyl benzoate, cumene, cyclohexanol, cyclohexanone, cyclohexylbenzene, decalin, dodecylbenzene, ethyl benzoate, indane, methyl benzoate, NMP, p-cymene, phenetole, 1,4-diisopropylbenzene, dibenzyl ether, diethylene glycol butyl methyl ether, triethylene glycol butyl methyl ether, diethylene glycol dibutyl ether, triethylene glycol dimethyl ether, diethylene glycol monobutyl ether, tripropylene glycol dimethyl ether, tetraethylene glycol dimethyl ether, 2-isopropylnaphthalene, pentylbenzene, hexylbenzene, heptylbenzene, octylbenzene, 1,1-bis(3,4-dimethylphenyl)ethane or mixtures of these solvents.

The invention therefore further provides a formulation, especially a solution, dispersion or emulsion, comprising at least one compound of the abovementioned formulae and at least one solvent, preferably an organic solvent. The way in which such solutions can be prepared is known to those skilled in the art and is described, for example, in WO 2002/072714, WO 2003/019694 and the literature cited therein.

In a preferred embodiment of the invention, the formulation, apart from the compound of the application, also contains at least one further matrix material and at least one phosphorescent emitter. The at least one further matrix material and the at least one phosphorescent emitter are each selected from the embodiments specified as preferred below. Application and evaporation of the solvent out of the formulation leaves the mixture of the materials as phosphorescent emitting layer with a mixed matrix.

The compounds of the application are suitable for use in electronic devices, especially in organic electroluminescent devices (OLEDs). Depending on the substitution, the compounds are used in different functions and layers.

The invention therefore further provides for the use of the compounds of the application in electronic devices. These electronic devices are preferably selected from the group consisting of organic integrated circuits (OICs), organic field-effect transistors (OFETs), organic thin-film transistors (OTFTs), organic light-emitting transistors (OLETs), organic solar cells (OSCs), organic optical detectors, organic photoreceptors, organic field-quench devices (OFQDs), organic light-emitting electrochemical cells (OLECs), organic laser diodes (O-lasers) and more preferably organic electroluminescent devices (OLEDs).

The invention further provides, as already set out above, an electronic device comprising at least one compound as defined above. This electronic device is preferably selected from the abovementioned devices.

It is more preferably an organic electroluminescent device (OLED) comprising anode, cathode and at least one emitting layer, characterized in that the at least one organic layer, which is preferably selected from emitting layers, electron transport layers and hole blocker layers, and which is more preferably selected from emitting layers, most preferably phosphorescent emitting layers, comprises at least one compound as defined above.

Apart from the cathode, anode and at least one emitting layer, the organic electroluminescent device may also comprise further layers. These are selected, for example, from in each case one or more hole injection layers, hole transport layers, hole blocker layers, electron transport layers, electron injection layers, electron blocker layers, exciton blocker layers, interlayers, charge generation layers and/or organic or inorganic p/n junctions.

The sequence of layers in the organic electroluminescent device is preferably as follows:

anode-hole injection layer-hole transport layer-optionally further hole transport layer(s)-optionally electron blocker layer-emitting layer-optionally hole blocker layer-electron transport layer-optionally further electron transport layer(s)-electron injection layer-cathode. It is additionally possible for further layers to be present in the OLED.

It is preferable when at least one hole-transporting layer of the apparatus is p-doped, i.e. contains at least one p-dopant. p-Dopants are preferably selected from electron acceptor compounds.

Particularly preferred p-dopants are quinodimethane compounds, azaindenofluorenediones, azaphenalenes, azatriphenylenes, I₂, metal halides, preferably transition metal halides, metal oxides, preferably metal oxides containing at least one transition metal or a metal of main group 3, and transition metal complexes, preferably complexes of Cu, Co, Ni, Pd and Pt with ligands containing at least one oxygen atom as bonding site. Preference is further given to transition metal oxides as dopants, preferably oxides of rhenium, molybdenum and tungsten, more preferably Re₂O₇, MoO₃, WO₃ and ReO₃. Also preferred are bismuth complexes, especially Bi(III) complexes, especially bismuth complexes with benzoic acid derivatives as complex ligands.

The organic electroluminescent device of the invention may contain two or more emitting layers. More preferably, these emission layers have several emission maxima between 380 nm and 750 nm overall, such that the overall result is white emission; in other words, various emitting compounds which may fluoresce or phosphoresce and which emit blue, green, yellow, orange or red light are used in the emitting layers. Especially preferred are three-layer systems, i.e. systems having three emitting layers, wherein one of the three layers in each case shows blue emission, one of the three layers in each case shows green emission, and one of the three layers in each case shows orange or red emission. The compounds of the invention are preferably present in the emitting layer. For the generation of white light, rather than multiple color-emitting emitter compounds, an emitter compound used individually that emits over a broad wavelength range is also suitable.

It is preferable in accordance with the invention when the compounds are used in an electronic device comprising one or more phosphorescent emitting compounds in an emitting layer. The compounds are preferably present in the emitting layer in combination with the phosphorescent emitting compound, more preferably in a mixture with at least one further matrix material. The latter is preferably selected from hole-conducting matrix materials, electron-conducting matrix materials and matrix materials having both hole-conducting and electron-conducting properties (bipolar matrix materials).

The term “phosphorescent emitting compounds” shall preferably be considered to include those compounds where light is emitted through a spin-forbidden transition, for example a transition from an excited triplet state or a state having a higher spin quantum number, for example a quintet state.

In a preferred embodiment of the present invention, the compound of the abovementioned formulae is used in an emitting layer as matrix material in combination with one or more phosphorescent emitting compounds. The phosphorescent emitting compound is preferably a red- or green-phosphorescing emitter, more preferably a green-phosphorescing emitter.

The total proportion of all matrix materials in the phosphorescent emitting layer in this case is between 50.0% and 99.9% by volume, preferably between 80.0% and 99.5% by volume, and more preferably between 85.0% and 97.0% by volume.

Correspondingly, the proportion of the phosphorescent emitting compound is between 0.1% and 50.0% by volume, preferably between 0.5% and 20.0% by volume, and more preferably between 3.0% and 15.0% by volume.

The emitting layer of the organic electroluminescent device preferably comprises two or more matrix materials (mixed matrix systems). The mixed matrix systems preferably comprise two or three different matrix materials, more preferably two different matrix materials.

In a preferred embodiment, one of the two matrix materials fulfills the function of a hole-transporting material, and the other of the two matrix materials fulfills the function of an electron-transporting material.

In a further preferred embodiment of the invention, one of the two materials is a wide bandgap material, and one or two further matrix materials are present in the emitting layer, which fulfill an electron-transporting function and/or a hole-transporting function of the mixed matrix. In a preferred embodiment, this can be accomplished in that not only the wide bandgap material but also a further matrix material having electron-transporting properties is present in the emitting layer, and yet a further matrix material having hole-transporting properties is present in the emitting layer. Alternatively and more preferably, this can be accomplished in that not only the wide bandgap material but also a single further matrix material having both electron-transporting and hole-transporting properties is present in the emitting layer. Such matrix materials are also referred to as bipolar matrix materials.

The bipolar matrix material for use in combination with the compound of the application in a phosphorescent emitting layer is preferably selected from compounds containing at least one triazine group. Particular preference is given to the triazine compounds of the formula (1) disclosed in as yet unpublished application EP17201480.5. The disclosure of this application in that regard is hereby incorporated into the present application.

Very particular preference is given to compounds of the formulae shown below

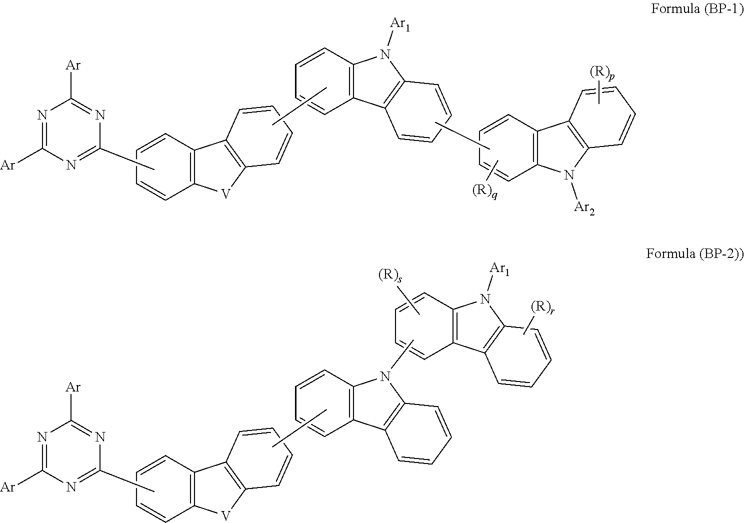

where the variables that occur are as follows:
V is O or S;
Ar, Ar₁, Ar₂ at each instance are each independently an aryl or heteroaryl group which has 5 to 40 ring atoms and may be substituted by one or more R₃ radicals or an aromatic or heteroaromatic ring system which has 6 to 40 ring atoms and may be substituted by one or more R₃ radicals;
p, q are each independently 0, 1, 2, 3 or 4;
s, r are each independently 0, 1, 2, 3 or 4;
R is the same or different at each instance and is selected from the group consisting of D, F, Cl, Br, I, CN, NO₂, N(Ar)₂, N(R₂)₂, C(═O)Ar, C(═O)R₂, P(═O)(Ar)₂, P(Ar)₂, B(Ar)₂, Si(Ar)₃, Si(R₂)₃, a straight-chain alkyl, alkoxy or thioalkyl group having 1 to 20 carbon atoms or a branched or cyclic alkyl, alkoxy or thioalkyl group having 3 to 20 carbon atoms or an alkenyl group having 2 to 20 carbon atoms, each of which may be substituted by one or more R² radicals, where one or more nonadjacent CH₂ groups may be replaced by R₂C═CR₂, Si(R₂)₂, C═O, C═S, C═NR₂, P(═O)(R₂), SO, SO₂, NR₂, O, S or CONR₂ and where one or more hydrogen atoms may be replaced by D, F, Cl, Br, I, CN or NO₂, an aromatic or heteroaromatic ring system which has 5 to 40 ring atoms and may be substituted in each case by one or more R₂ radicals, an aryloxy or heteroaryloxy group which has 5 to 40 ring atoms and may be substituted by one or more R₂ radicals, or an aralkyl or heteroaralkyl group which has 5 to 40 ring atoms and may be substituted by one or more R₂ radicals; at the same time, it is optionally possible for a maximum of one substituent R together with Ar₁ to form a monocyclic or polycyclic, aliphatic, aromatic or heteroaromatic ring system which may be substituted by one or more R₂ radicals;
R₂ is the same or different at each instance and is selected from the group consisting of H, D, F, Cl, Br, I, CN, NO₂, N(Ar)₂, NH₂, N(R₃)₂, C(═O)Ar, C(═O)H, C(═O)R₃, P(═O)(Ar)₂, a straight-chain alkyl, alkoxy or thioalkyl group having 1 to 40 carbon atoms or a branched or cyclic alkyl, alkoxy or thioalkyl group having 3 to 40 carbon atoms or an alkenyl or alkynyl group having 2 to 40 carbon atoms, each of which may be substituted by one or more R₃ radicals, where one or more nonadjacent CH₂ groups may be replaced by HC═CH, R₃C═CR₃, C≡C, Si(R₃)₂, Ge(R₃)₂, Sn(R₃)₂, C═O, C═S, C═Se, C═NR₃, P(═O)(R₃), SO, SO₂, NH, NR₃, O, S, CONH or CONR₃ and where one or more hydrogen atoms may be replaced by D, F, Cl, Br, I, CN or NO₂, an aromatic or heteroaromatic ring system which has 5 to 60 ring atoms and may be substituted in each case by one or more R₃ radicals, an aryloxy or heteroaryloxy group which has 5 to 60 ring atoms and may be substituted by one or more R₃ radicals, or a combination of these systems; where it is optionally possible for two or more adjacent substituents R₂ to form a monocyclic or polycyclic, aliphatic, aromatic or heteroaromatic ring system which may be substituted by one or more R₃ radicals;

R₃ is the same or different at each instance and is selected from the group consisting of H, D, F, CN, an aliphatic hydrocarbyl radical having 1 to 20 carbon atoms, or an aromatic or heteroaromatic ring system having 5 to 30 ring atoms in which one or more hydrogen atoms may be replaced by D, F, Cl, Br, I or CN and which may be substituted by one or more alkyl groups each having 1 to 4 carbon atoms; at the same time, it is possible for two or more adjacent substituents R₃ together to form a mono- or polycyclic, aliphatic ring system.

In another alternative embodiment, as well as the wide bandgap matrix material, only a single further matrix material having either predominantly hole-transporting properties or predominantly electron-transporting properties may be present in the emitting layer.

In the preferred case that two different matrix materials are present in the emitting layer, these may be present in a volume ratio of 1:50 to 1:1, preferably 1:20 to 1:1, more preferably 1:10 to 1:1 and most preferably 1:4 to 1:1. Preferably, the compound of the abovementioned formulae is present in the same proportion as the further matrix compound, or it is present in a higher proportion than the further matrix compound. Preferably, the ratio of compound of abovementioned formulae (M1) and further matrix compound (M2) is M1:M2 between 4:1 and 1:1.

The absolute proportion of the compound of the abovementioned formulae in the mixture of the emitting layer, in the case of use as matrix material in a phosphorescent emitting layer, is preferably 10% by volume to 85% by volume, more preferably 20% by volume to 85% by volume, even more preferably 30% by volume to 80% by volume, very especially preferably 20% by volume to 60% by volume and most preferably 30% by volume to 50% by volume. The absolute proportion of the second matrix compound in this case is preferably 15% by volume to 90% by volume, more preferably 15% by volume to 80% by volume, even more preferably 20% by volume to 70% by volume, very especially preferably 40% by volume to 80% by volume, and most preferably 50% by volume to 70% by volume.

For production of phosphorescent emitting layers of the mixed matrix type, in a preferred embodiment of the invention, a solution comprising the phosphorescent emitter and the two or more matrix materials may be produced. This can be applied by means of spin-coating, printing methods or other methods. Evaporation of the solvent in this case leaves the phosphorescent emitting layer of the mixed matrix type.

In an alternative, more preferred embodiment of the invention, the phosphorescent emitter layer of the mixed matrix type is produced by vapor phase deposition. For this purpose, there are two methods by which the layer can be applied. Firstly, each of the at least two different matrix materials may be initially charged in a material source, followed by simultaneous evaporation (“coevaporation”) from the two or more different material sources. Secondly, the at least two matrix materials may be premixed and the mixture obtained may be initially charged in a single material source from which it is ultimately evaporated. The latter method is referred to as the premix method.

The present application therefore also provide a mixture comprising a compound of the above-specified formulae and at least one further compound selected from matrix compounds, and preferably selected from the above-specified bipolar matrix compounds, especially from compounds of the formulae (BP-1) and (BP-2). In this respect, the preferred embodiments with regard to proportions of the matrix compound and their chemical structure that are specified in this application are likewise considered to be applicable.

In an alternative preferred embodiment of the invention, the compound is used as electron-transporting material. This is especially true when the compound contains at least one group selected from electron-deficient heteroaryl groups, preferably azine groups, especially triazine groups, pyrimidine groups and pyridine groups, and benzimidazole groups.

When the compound is used as electron-transporting material, it is preferably used in a hole blocker layer, an electron transport layer or an electron injection layer. In a preferred embodiment, the layer mentioned is n-doped. The compound may alternatively be in the form of a pure material in the layer in question.

In the present context, an n-dopant is understood to mean an organic or inorganic compound capable of releasing electrons (electron donor), i.e. a compound that acts as a reducing agent. The compounds used for n-doping can be used in the form of a precursor, in which case these precursor compounds release n-dopants through activation. Preferably, n-dopants are selected from electron-rich metal complexes; P═N compounds; N-heterocycles, more preferably naphthylenecarbodiimides, pyridines, acridines and phenazines; fluorenes and free-radical compounds.

Preferred embodiments of the different functional materials in the electronic device are listed hereinafter.

Preferred fluorescent emitting compounds are selected from the class of the arylamines. An arylamine or an aromatic amine in the context of this invention is understood to mean a compound containing three substituted or unsubstituted aromatic or heteroaromatic ring systems bonded directly to the nitrogen. Preferably, at least one of these aromatic or heteroaromatic ring systems is a fused ring system, more preferably having at least 14 aromatic ring atoms. Preferred examples of these are aromatic anthraceneamines, aromatic anthracenediamines, aromatic pyreneamines, aromatic pyrenediamines, aromatic chryseneamines or aromatic chrysenediamines. An aromatic anthraceneamine is understood to mean a compound in which a diarylamino group is bonded directly to an anthracene group, preferably in the 9 position. An aromatic anthracenediamine is understood to mean a compound in which two diarylamino groups are bonded directly to an anthracene group, preferably in the 9,10 positions. Aromatic pyreneamines, pyrenediamines, chryseneamines and chrysenediamines are defined analogously, where the diarylamino groups are bonded to the pyrene preferably in the 1 position or 1,6 position. Further preferred emitting compounds are indenofluoreneamines or -diamines, benzoindenofluoreneamines or -diamines, and dibenzoindenofluoreneamines or -diamines, and indenofluorene derivatives having fused aryl groups. Likewise preferred are pyrenearylamines. Likewise preferred are benzoindenofluoreneamines, benzofluoreneamines, extended benzoindenofluorenes, phenoxazines, and fluorene derivatives joined to furan units or to thiophene units.

Preferred matrix materials for fluorescent emitters are selected from the classes of the oligoarylenes (e.g. 2,2′,7,7′-tetraphenylspirobifluorene), especially the oligoarylenes containing fused aromatic groups, the oligoarylenevinylenes, the polypodal metal complexes, the hole-conducting compounds, the electron-conducting compounds, especially ketones, phosphine oxides and sulfoxides; the atropisomers, the boronic acid derivatives or the benzanthracenes. Particularly preferred matrix materials are selected from the classes of the oligoarylenes comprising naphthalene, anthracene, benzanthracene and/or pyrene or atropisomers of these compounds, the oligoarylenevinylenes, the ketones, the phosphine oxides and the sulfoxides. Very particularly preferred matrix materials are selected from the classes of the oligoarylenes comprising anthracene, benzanthracene, benzophenanthrene and/or pyrene or atropisomers of these compounds. An oligoarylene in the context of this invention shall be understood to mean a compound in which at least three aryl or arylene groups are bonded to one another.

Suitable phosphorescent emitting compounds (=triplet emitters) are especially compounds which, when suitably excited, emit light, preferably in the visible region, and also contain at least one atom of atomic number greater than 20, preferably greater than 38, and less than 84, more preferably greater than 56 and less than 80. Preference is given to using, as phosphorescent emitting compounds, compounds containing copper, molybdenum, tungsten, rhenium, ruthenium, osmium, rhodium, iridium, palladium, platinum, silver, gold or europium, especially compounds containing iridium, platinum or copper. In the context of the present invention, all luminescent iridium, platinum or copper complexes are considered to be phosphorescent emitting compounds.

In general, all phosphorescent complexes as used for phosphorescent OLEDs according to the prior art and as known to those skilled in the art in the field of organic electroluminescent devices are suitable. Explicit examples of particularly suitable complexes are shown in the following table:

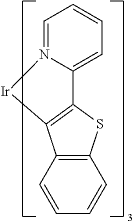

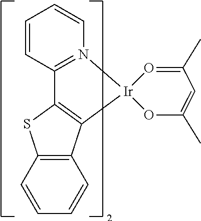

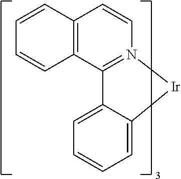

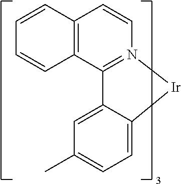

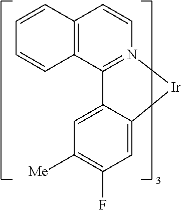

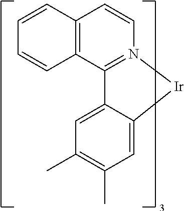

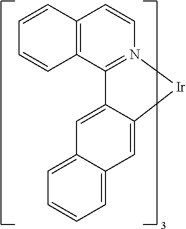

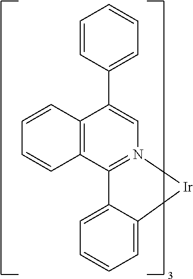

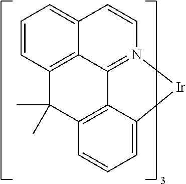

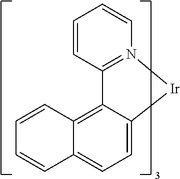

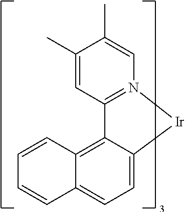

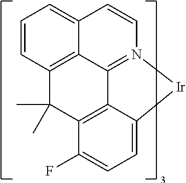

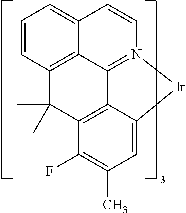

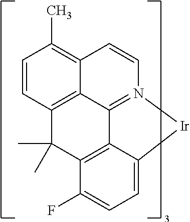

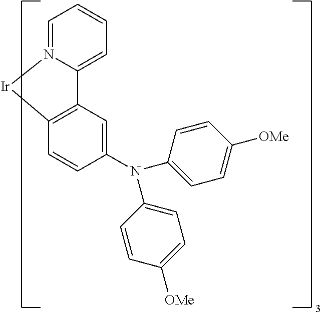

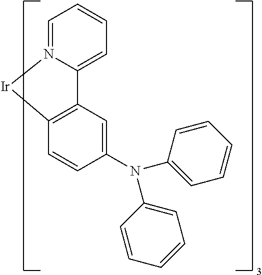

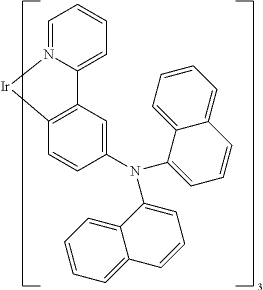

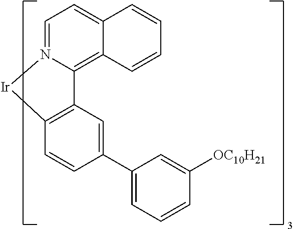

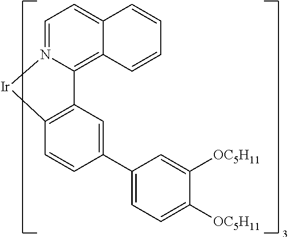

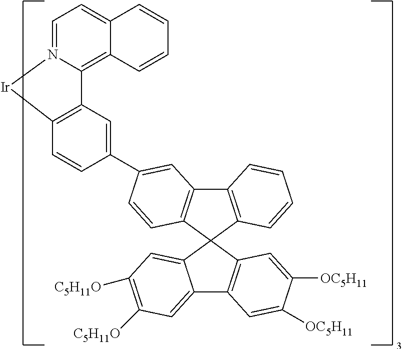

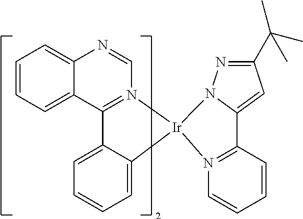

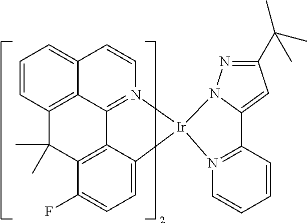

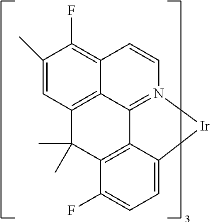

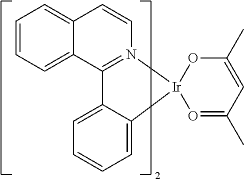

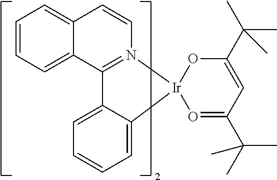

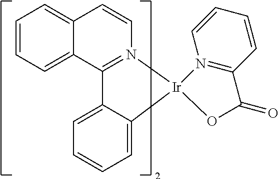

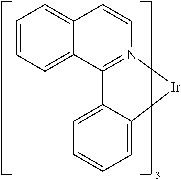

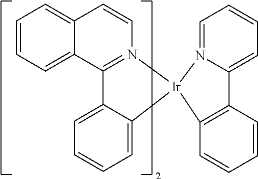

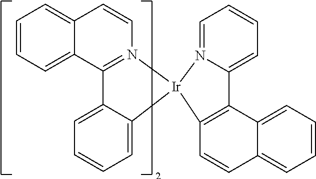

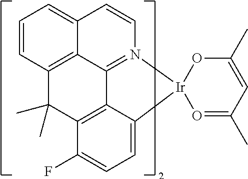

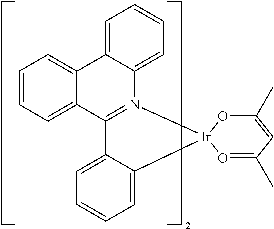

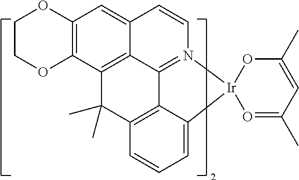

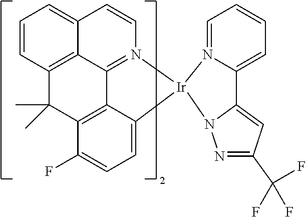

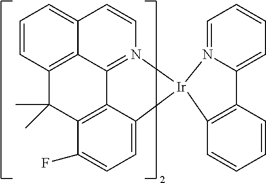

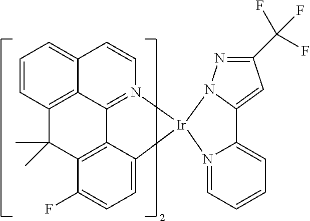

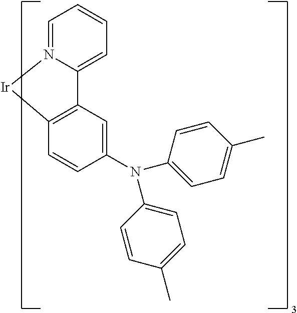

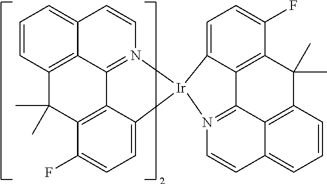

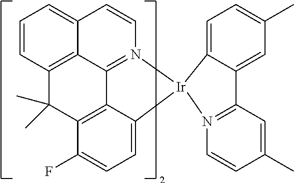

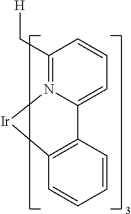

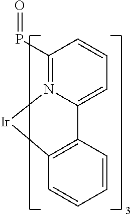

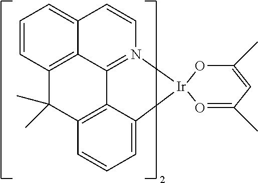

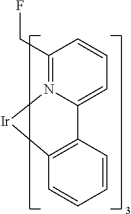

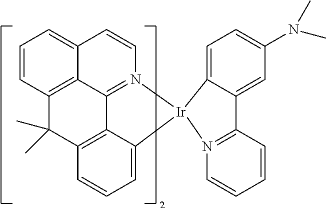

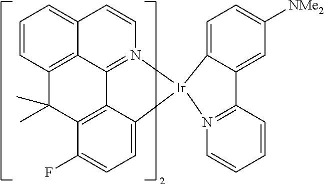

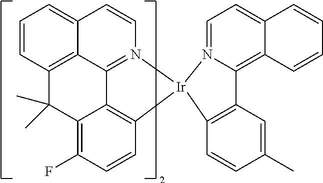

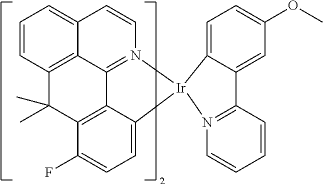

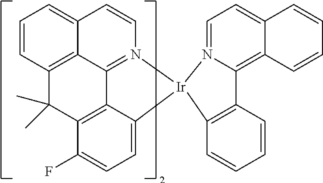

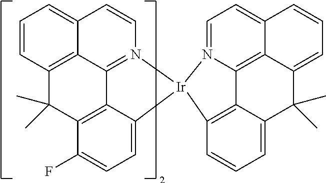

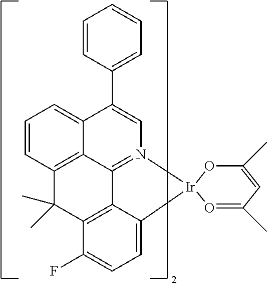

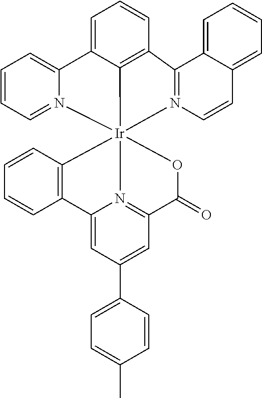

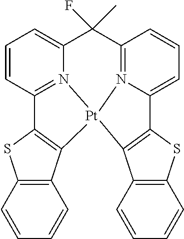

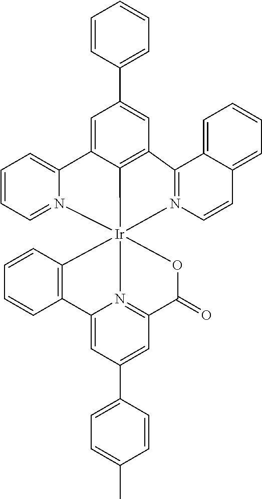

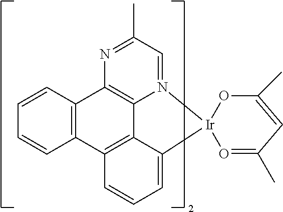

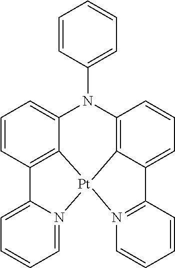

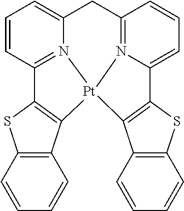

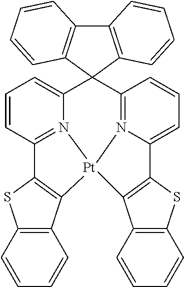

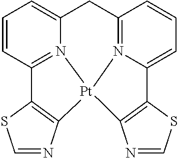

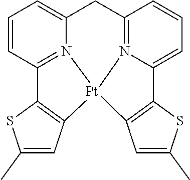

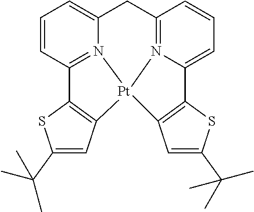

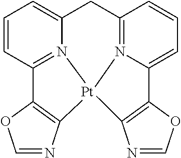

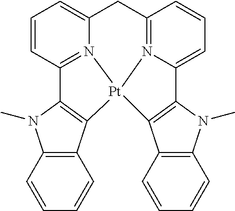

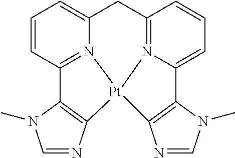

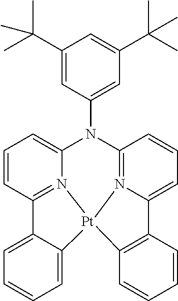

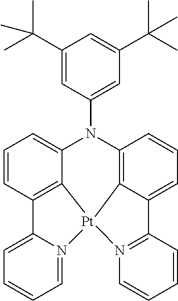

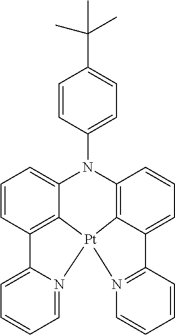

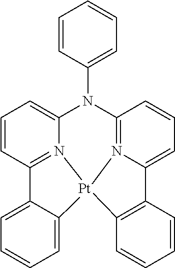

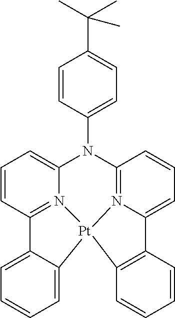

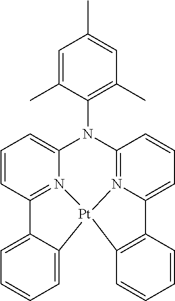

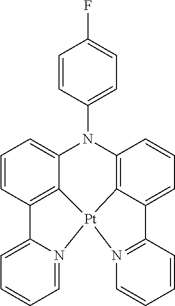

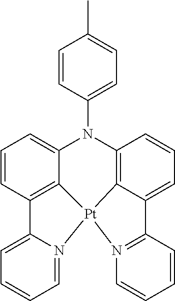

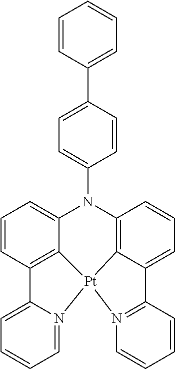

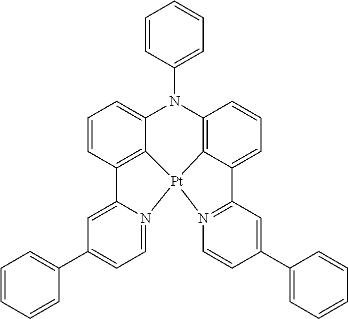

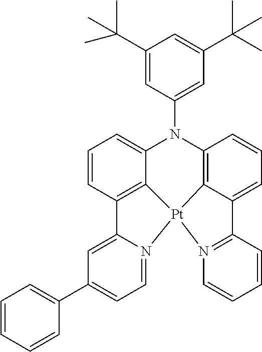

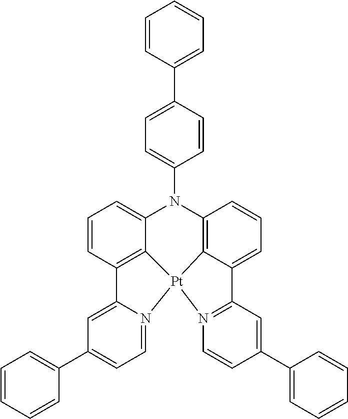

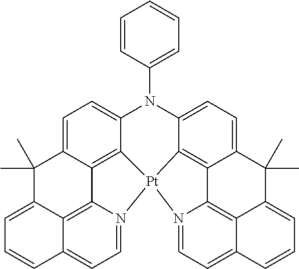

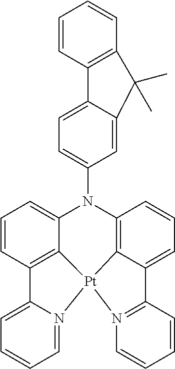

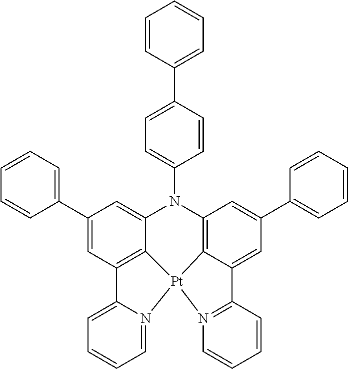

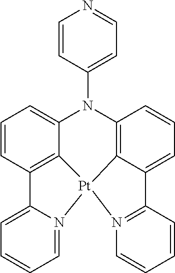

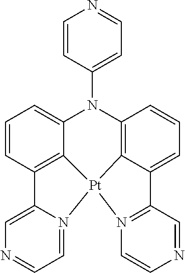

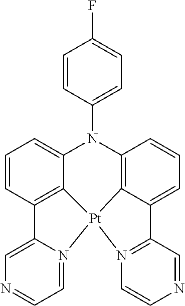

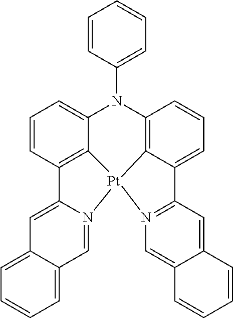

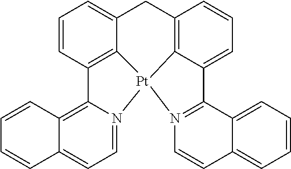

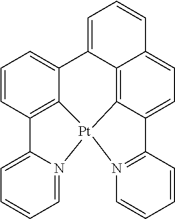

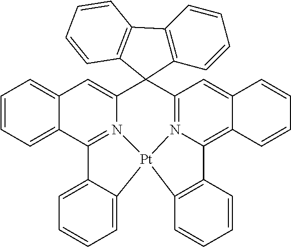

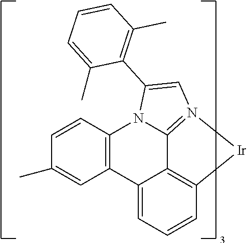

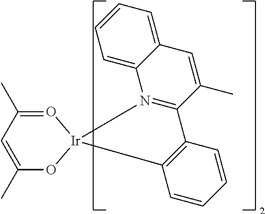

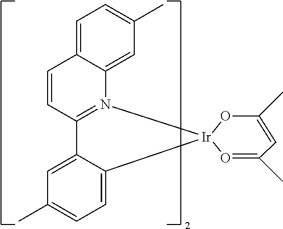

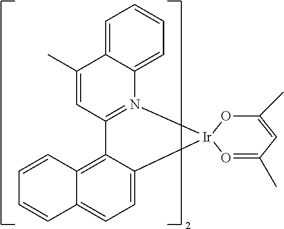

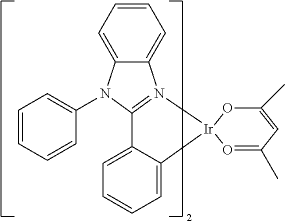

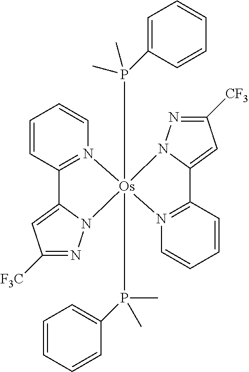

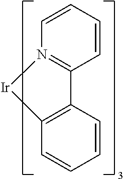

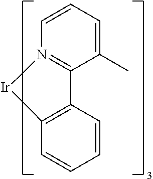

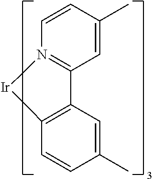

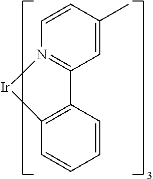

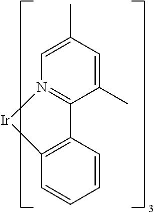

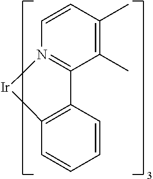

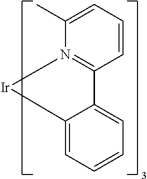

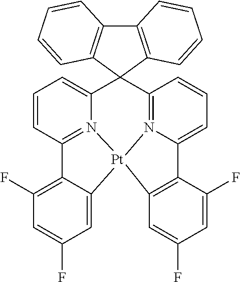

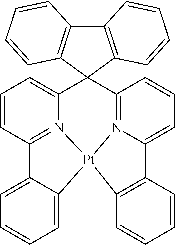

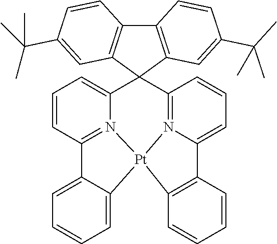

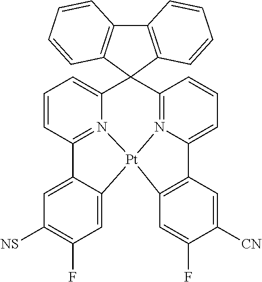

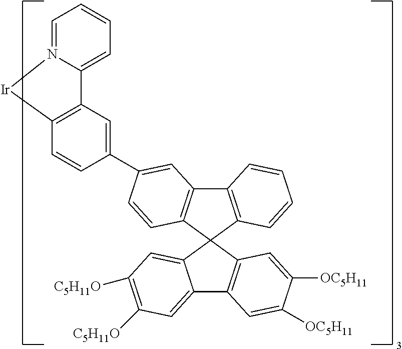

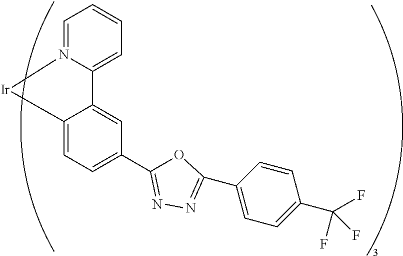

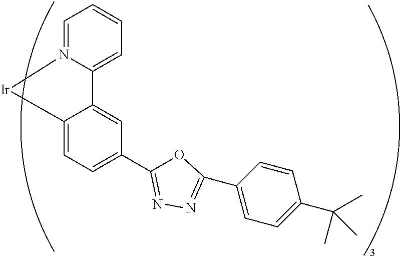

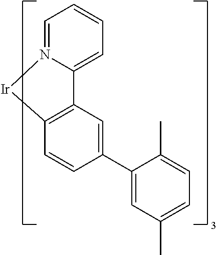

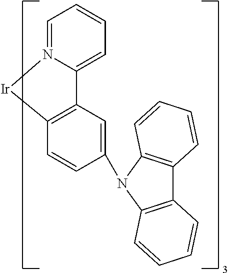

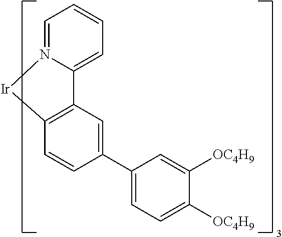

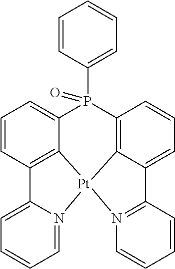

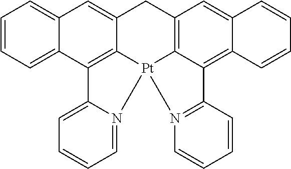

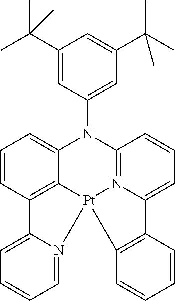

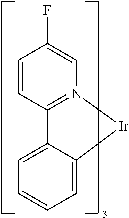

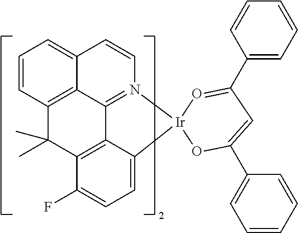

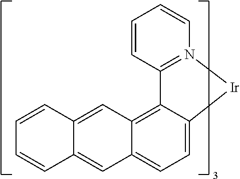

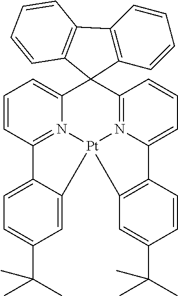

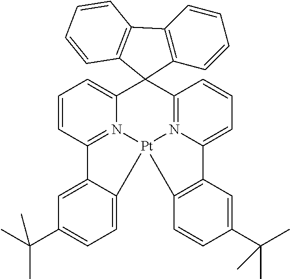

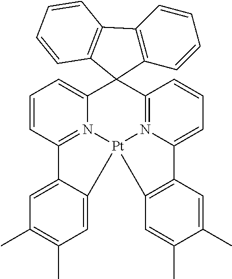

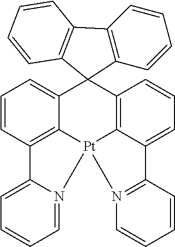

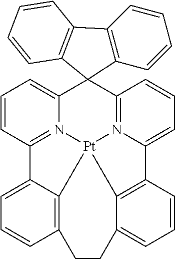

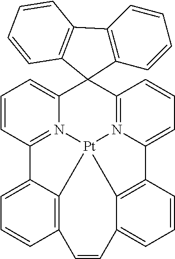

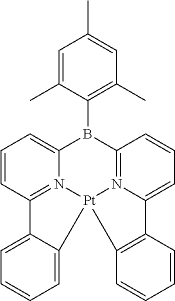

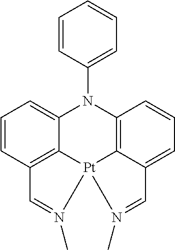

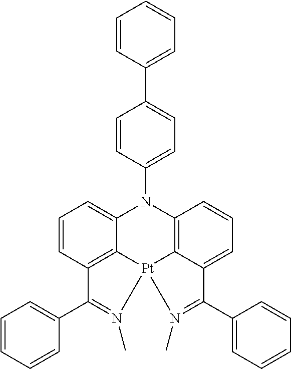

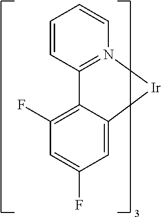

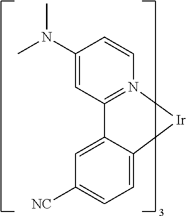

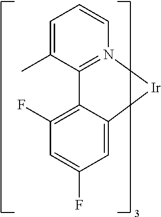

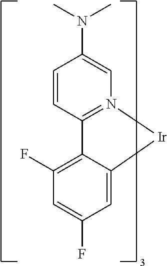

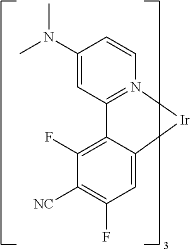

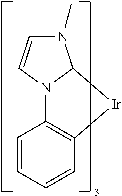

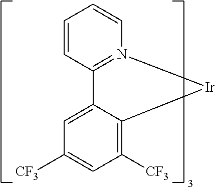

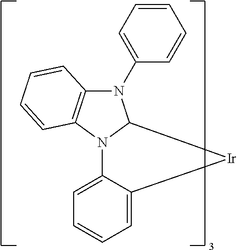

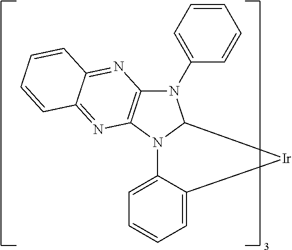

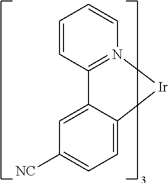

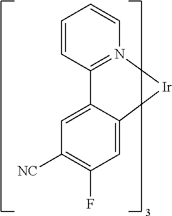

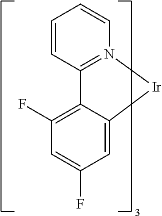

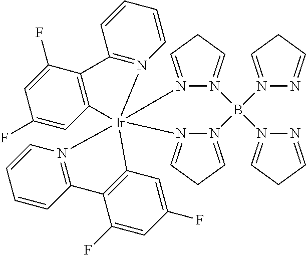

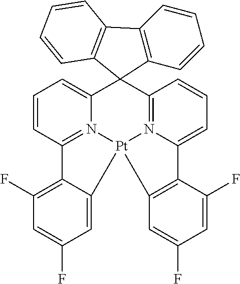

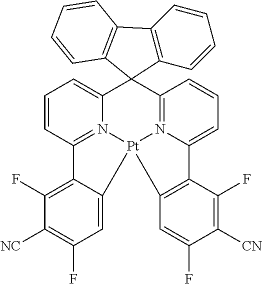

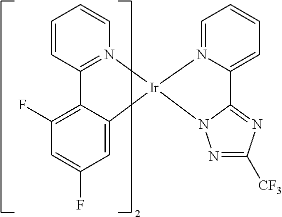

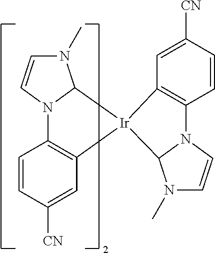

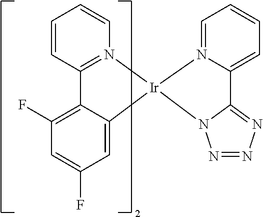

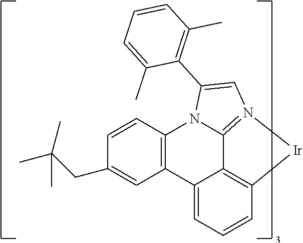

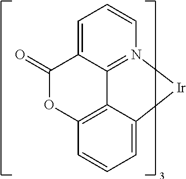

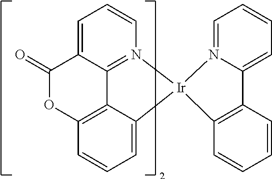

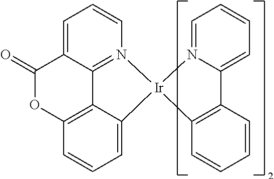

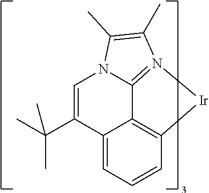

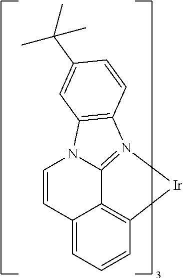

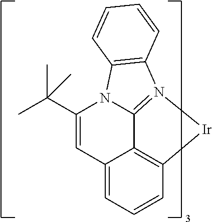

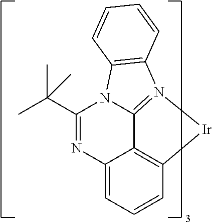

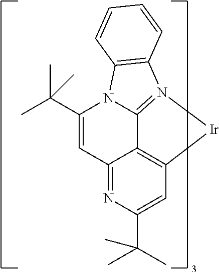

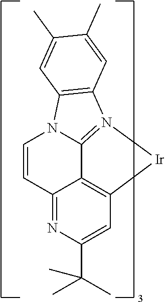

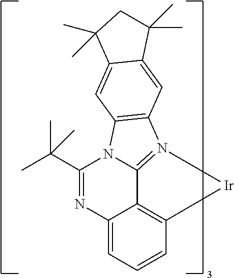

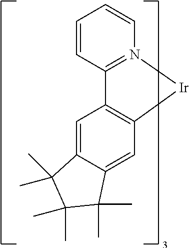

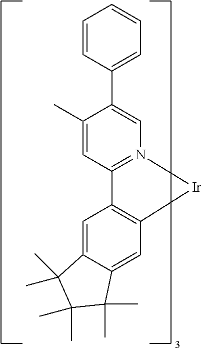

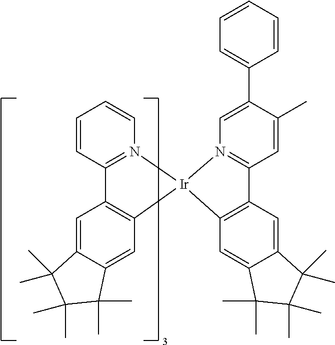

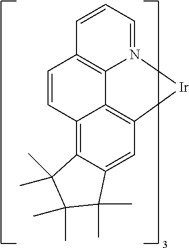

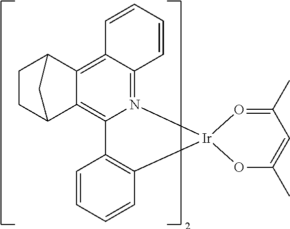

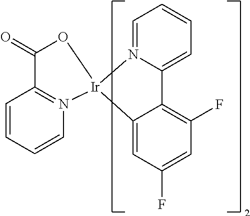

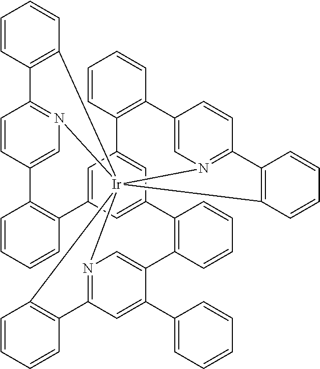

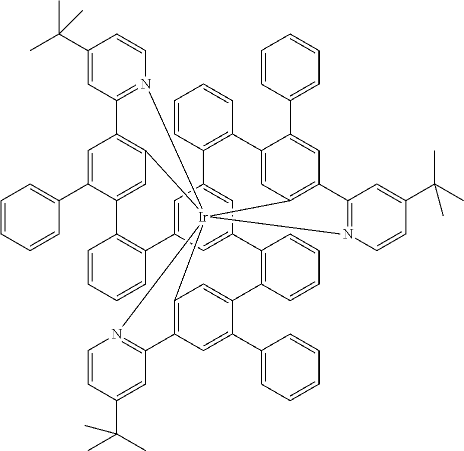

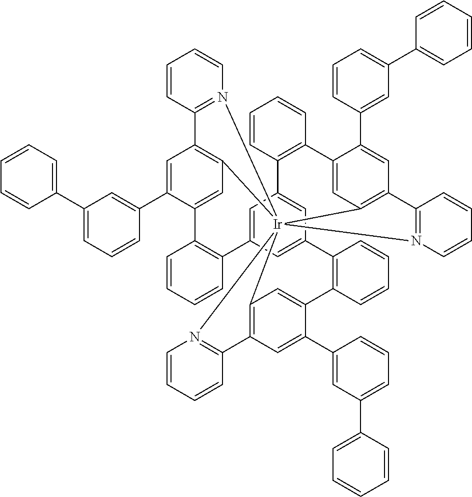

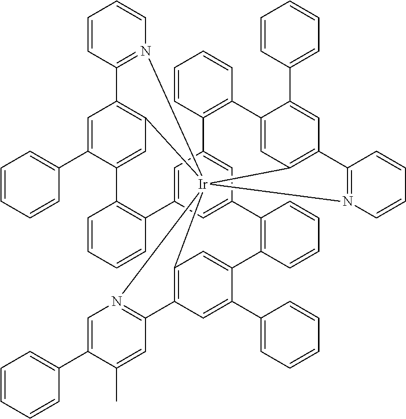

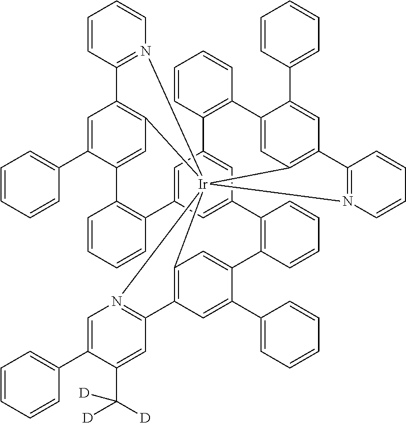

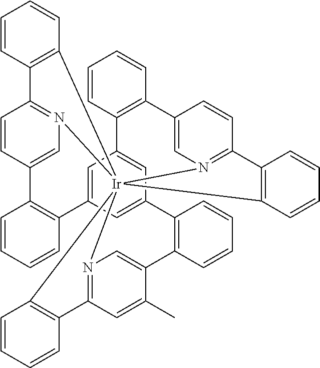

Preferred matrix materials for phosphorescent emitters, as well as the compounds of the application, are aromatic ketones, aromatic phosphine oxides or aromatic sulfoxides or sulfones, triarylamines, carbazole derivatives, e.g. CBP (N,N-biscarbazolylbiphenyl), indolocarbazole derivatives, indenocarbazole derivatives, azacarbazole derivatives, bipolar matrix materials, silanes, azaboroles or boronic esters, triazine derivatives, zinc complexes, diazasilole or tetraazasilole derivatives, diazaphosphole derivatives, bridged carbazole derivatives, triphenylene derivatives, or lactams.

Preferred matrix materials for use in a mixture with the compounds of the application in phosphorescent emitting layers are selected from the following compounds:

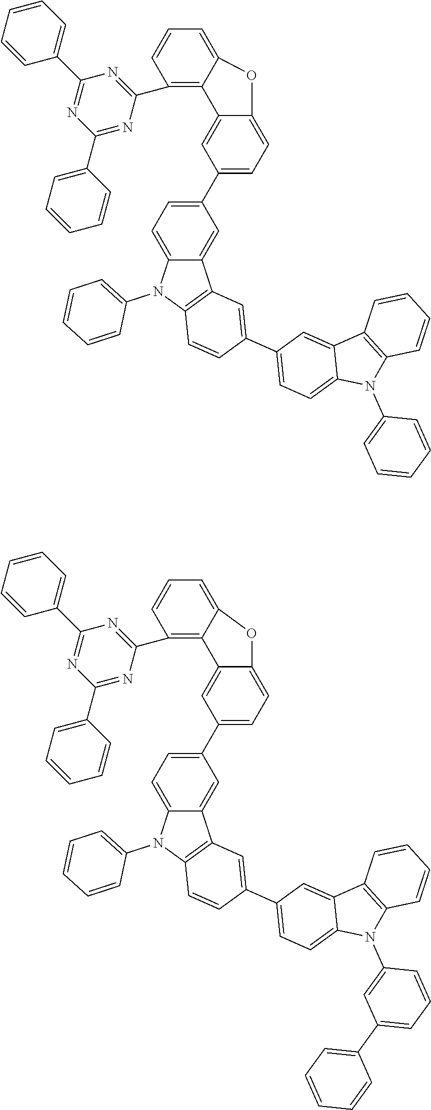 [SMILES: c1ccc(-c2nc(-c3ccccc3)nc(-c3cccc4oc5ccc(-c6ccc7c(c6)c6cc(-c8ccc9c(c8)c8ccccc8n9-c8ccccc8)ccc6n7-c6ccccc6)cc5c34)n2)cc1 ; c1ccc(-c2cccc(-n3c4ccccc4c4cc(-c5ccc6c(c5)c5cc(-c7ccc8oc9cccc(-c%10nc(-c%11ccccc%11)nc(-c%11ccccc%11)n%10)c9c8c7)ccc5n6-c5ccccc5)ccc43)c2)cc1]

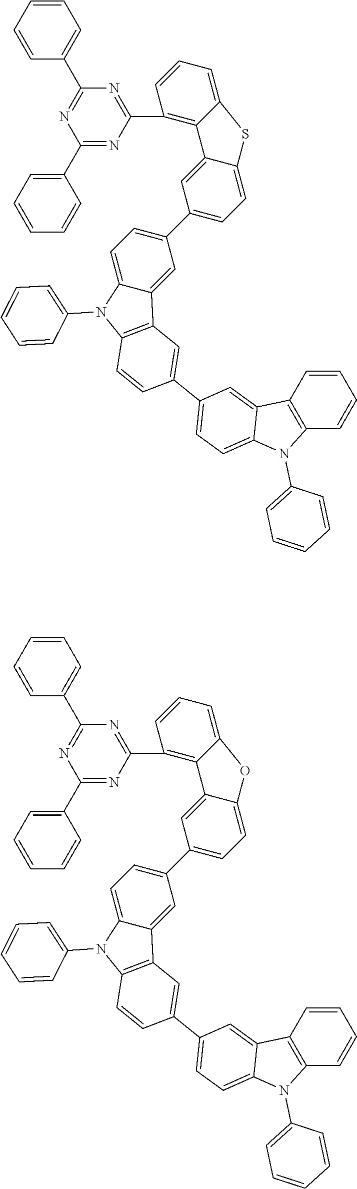 [SMILES: c1ccc(-c2nc(-c3ccccc3)nc(-c3cccc4sc5ccc(-c6ccc7c(c6)c6cc(-c8ccc9c(c8)c8ccccc8n9-c8ccccc8)ccc6n7-c6ccccc6)cc5c34)n2)cc1 ; c1ccc(-c2nc(-c3ccccc3)nc(-c3cccc4oc5ccc(-c6ccc7c(c6)c6cc(-c8ccc9c(c8)c8ccccc8n9-c8ccccc8)ccc6n7-c6ccccc6)cc5c34)n2)cc1]

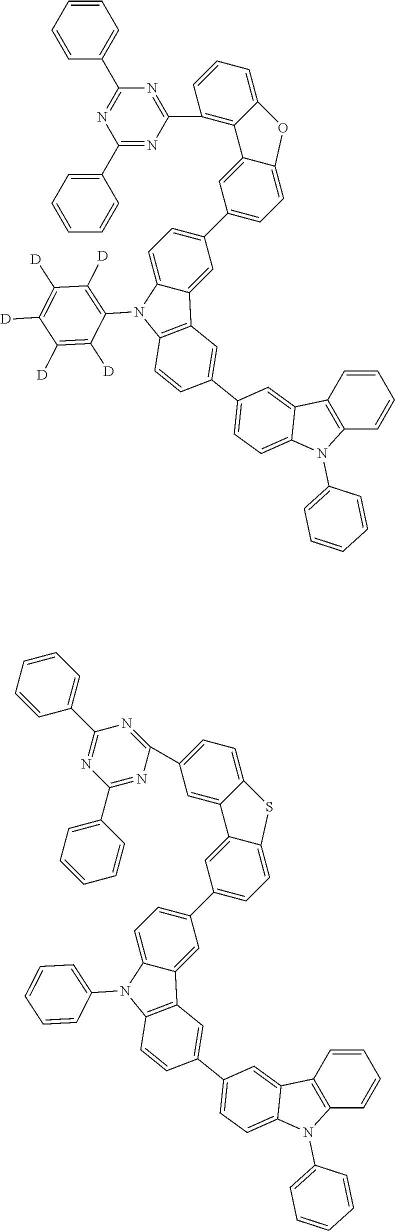 [SMILES: [2H]c1c([2H])c([2H])c(-n2c3ccc(-c4ccc5oc6cccc(-c7nc(-c8ccccc8)nc(-c8ccccc8)n7)c6c5c4)cc3c3cc(-c4ccc5c(c4)c4ccccc4n5-c4ccccc4)ccc32)c([2H])c1[2H] ; c1ccc(-c2nc(-c3ccccc3)nc(-c3ccc4sc5ccc(-c6ccc7c(c6)c6cc(-c8ccc9c(c8)c8ccccc8n9-c8ccccc8)ccc6n7-c6ccccc6)cc5c4c3)n2)cc1]

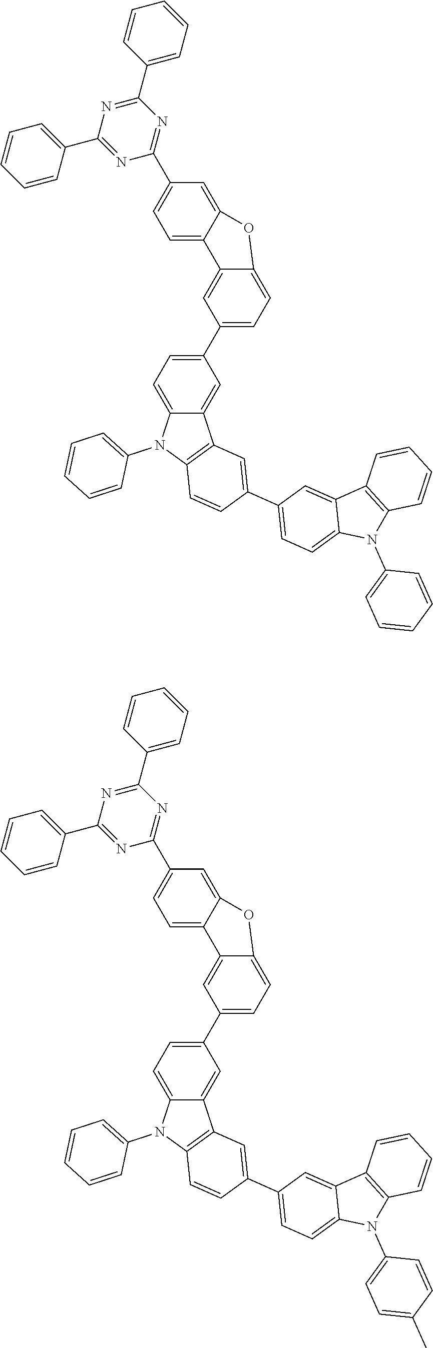 [SMILES: c1ccc(-c2nc(-c3ccccc3)nc(-c3ccc4c(c3)oc3ccc(-c5ccc6c(c5)c5cc(-c7ccc8c(c7)c7ccccc7n8-c7ccccc7)ccc5n6-c5ccccc5)cc34)n2)cc1 ; Cc1ccc(-n2c3ccccc3c3cc(-c4ccc5c(c4)c4cc(-c6ccc7oc8cc(-c9nc(-c%10ccccc%10)nc(-c%10ccccc%10)n9)ccc8c7c6)ccc4n5-c4ccccc4)ccc32)cc1]

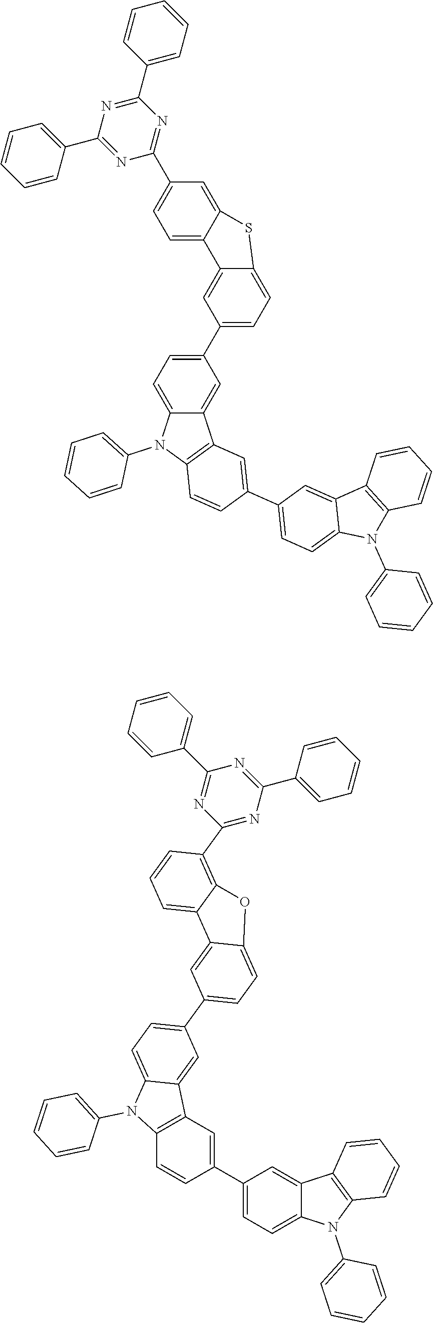 [SMILES: c1ccc(-c2nc(-c3ccccc3)nc(-c3ccc4c(c3)sc3ccc(-c5ccc6c(c5)c5cc(-c7ccc8c(c7)c7ccccc7n8-c7ccccc7)ccc5n6-c5ccccc5)cc34)n2)cc1 ; c1ccc(-c2nc(-c3ccccc3)nc(-c3cccc4c3oc3ccc(-c5ccc6c(c5)c5cc(-c7ccc8c(c7)c7ccccc7n8-c7ccccc7)ccc5n6-c5ccccc5)cc34)n2)cc1]

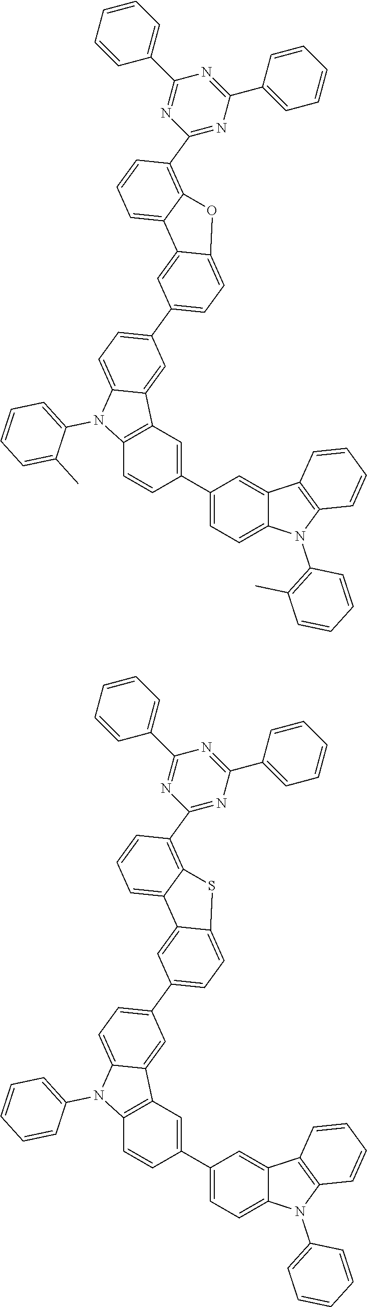 [SMILES: Cc1ccccc1-n1c2ccccc2c2cc(-c3ccc4c(c3)c3cc(-c5ccc6oc7c(-c8nc(-c9ccccc9)nc(-c9ccccc9)n8)cccc7c6c5)ccc3n4-c3ccccc3C)ccc21 ; c1ccc(-c2nc(-c3ccccc3)nc(-c3cccc4c3sc3ccc(-c5ccc6c(c5)c5cc(-c7ccc8c(c7)c7ccccc7n8-c7ccccc7)ccc5n6-c5ccccc5)cc34)n2)cc1]

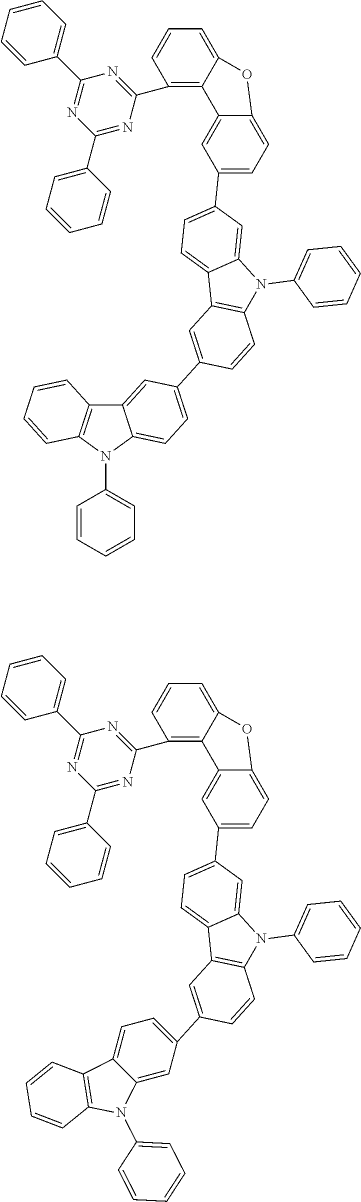 [SMILES: c1ccc(-c2nc(-c3ccccc3)nc(-c3cccc4oc5ccc(-c6ccc7c8cc(-c9ccc%10c(c9)c9ccccc9n%10-c9ccccc9)ccc8n(-c8ccccc8)c7c6)cc5c34)n2)cc1 ; c1ccc(-c2nc(-c3ccccc3)nc(-c3cccc4oc5ccc(-c6ccc7c8cc(-c9ccc%10c%11ccccc%11n(-c%11ccccc%11)c%10c9)ccc8n(-c8ccccc8)c7c6)cc5c34)n2)cc1]

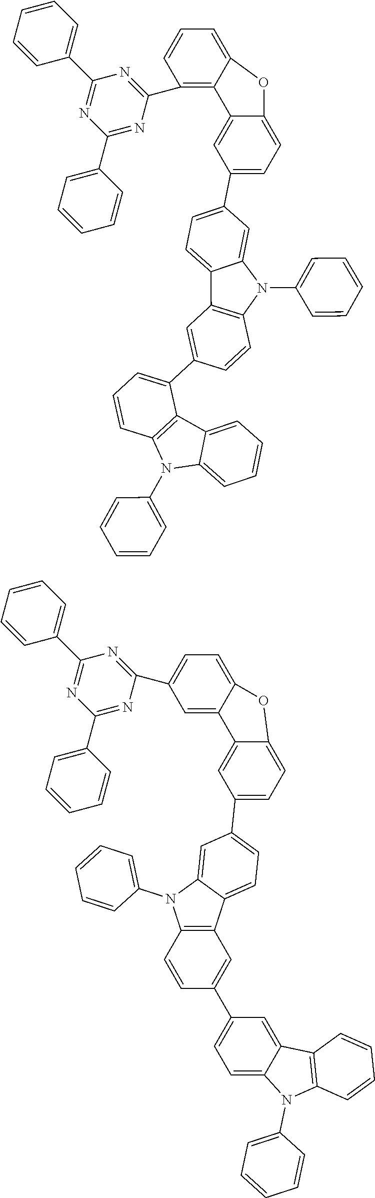 [SMILES: c1ccc(-c2nc(-c3ccccc3)nc(-c3cccc4oc5ccc(-c6ccc7c8cc(-c9cccc%10c9c9ccccc9n%10-c9ccccc9)ccc8n(-c8ccccc8)c7c6)cc5c34)n2)cc1 ; c1ccc(-c2nc(-c3ccccc3)nc(-c3ccc4oc5ccc(-c6ccc7c8cc(-c9ccc%10c(c9)c9ccccc9n%10-c9ccccc9)ccc8n(-c8ccccc8)c7c6)cc5c4c3)n2)cc1]

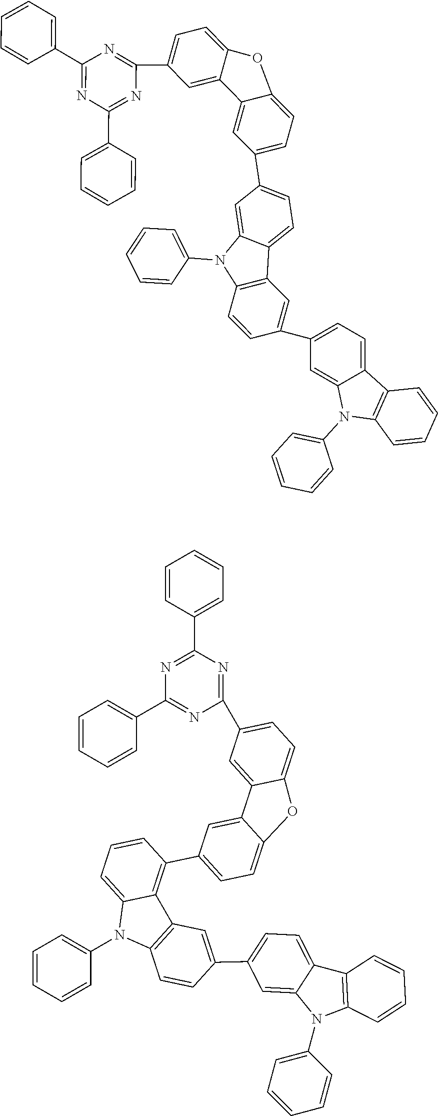 [SMILES: c1ccc(-c2nc(-c3ccccc3)nc(-c3ccc4oc5ccc(-c6ccc7c8cc(-c9ccc%10c%11ccccc%11n(-c%11ccccc%11)c%10c9)ccc8n(-c8ccccc8)c7c6)cc5c4c3)n2)cc1 ; c1ccc(-c2nc(-c3ccccc3)nc(-c3ccc4oc5ccc(-c6cccc7c6c6cc(-c8ccc9c%10ccccc%10n(-c%10ccccc%10)c9c8)ccc6n7-c6ccccc6)cc5c4c3)n2)cc1]

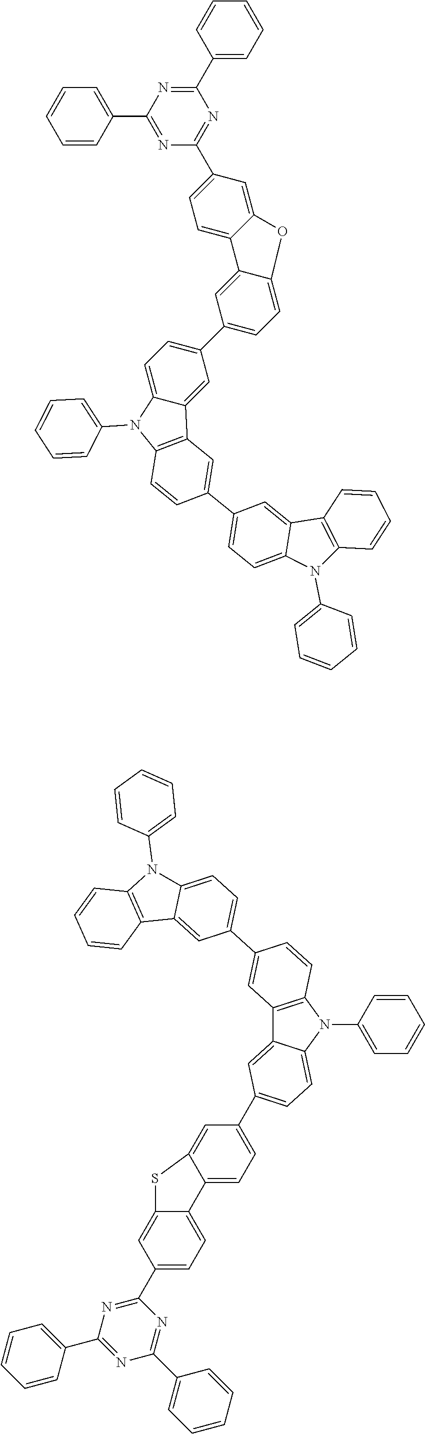 [SMILES: c1ccc(-c2nc(-c3ccccc3)nc(-c3ccc4c(c3)oc3ccc(-c5ccc6c(c5)c5cc(-c7ccc8c(c7)c7ccccc7n8-c7ccccc7)ccc5n6-c5ccccc5)cc34)n2)cc1 ; c1ccc(-c2nc(-c3ccccc3)nc(-c3ccc4c(c3)sc3cc(-c5ccc6c(c5)c5cc(-c7ccc8c(c7)c7ccccc7n8-c7ccccc7)ccc5n6-c5ccccc5)ccc34)n2)cc1]

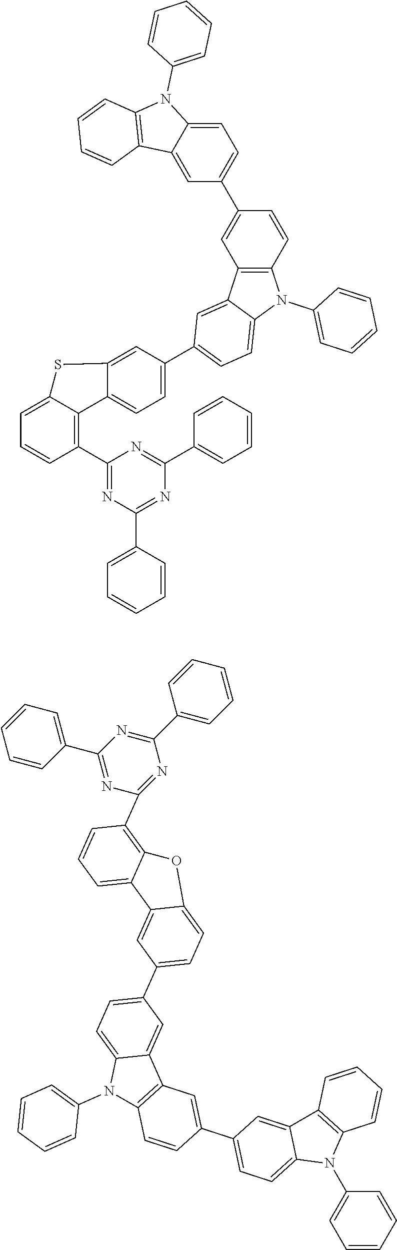 [SMILES: c1ccc(-c2nc(-c3ccccc3)nc(-c3cccc4sc5cc(-c6ccc7c(c6)c6cc(-c8ccc9c(c8)c8ccccc8n9-c8ccccc8)ccc6n7-c6ccccc6)ccc5c34)n2)cc1 ; c1ccc(-c2nc(-c3ccccc3)nc(-c3cccc4c3oc3ccc(-c5ccc6c(c5)c5cc(-c7ccc8c(c7)c7ccccc7n8-c7ccccc7)ccc5n6-c5ccccc5)cc34)n2)cc1]

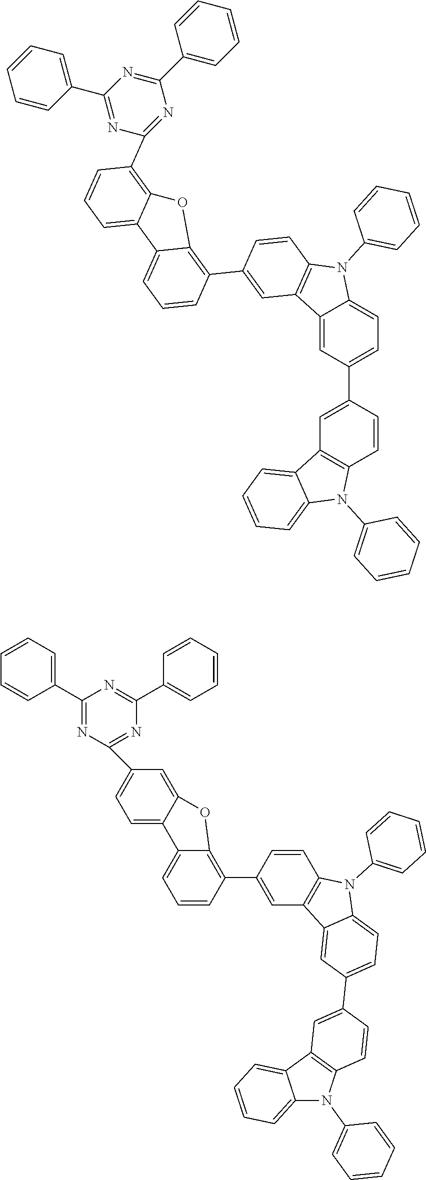 [SMILES: c1ccc(-c2nc(-c3ccccc3)nc(-c3cccc4c3oc3c(-c5ccc6c(c5)c5cc(-c7ccc8c(c7)c7ccccc7n8-c7ccccc7)ccc5n6-c5ccccc5)cccc34)n2)cc1 ; c1ccc(-c2nc(-c3ccccc3)nc(-c3ccc4c(c3)oc3c(-c5ccc6c(c5)c5cc(-c7ccc8c(c7)c7ccccc7n8-c7ccccc7)ccc5n6-c5ccccc5)cccc34)n2)cc1]

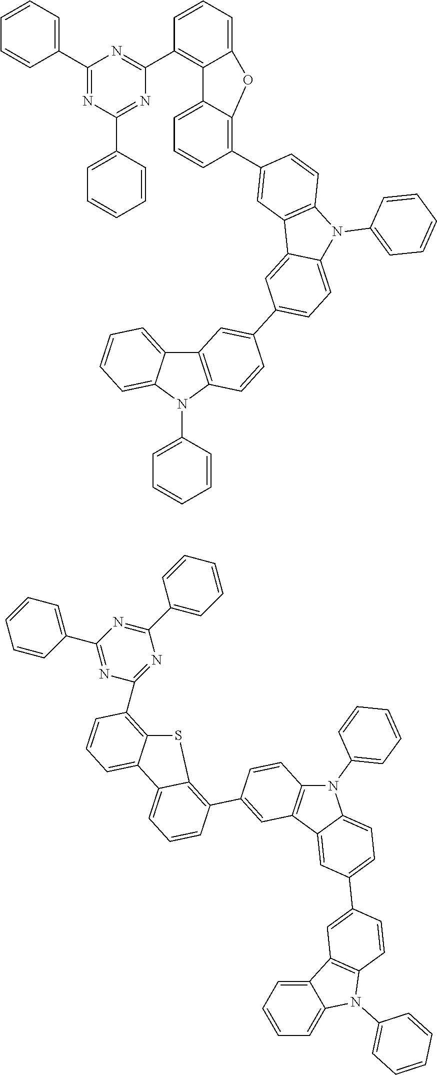 [SMILES: c1ccc(-c2nc(-c3ccccc3)nc(-c3cccc4oc5c(-c6ccc7c(c6)c6cc(-c8ccc9c(c8)c8ccccc8n9-c8ccccc8)ccc6n7-c6ccccc6)cccc5c34)n2)cc1 ; c1ccc(-c2nc(-c3ccccc3)nc(-c3cccc4c3sc3c(-c5ccc6c(c5)c5cc(-c7ccc8c(c7)c7ccccc7n8-c7ccccc7)ccc5n6-c5ccccc5)cccc34)n2)cc1]

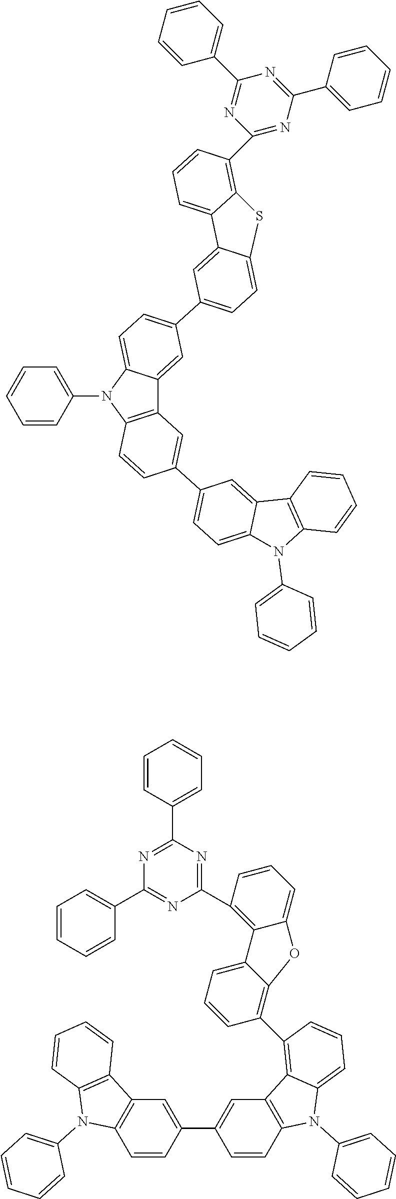 [SMILES: c1ccc(-c2nc(-c3ccccc3)nc(-c3cccc4c3sc3ccc(-c5ccc6c(c5)c5cc(-c7ccc8c(c7)c7ccccc7n8-c7ccccc7)ccc5n6-c5ccccc5)cc34)n2)cc1 ; c1ccc(-c2nc(-c3ccccc3)nc(-c3cccc4oc5c(-c6cccc7c6c6cc(-c8ccc9c(c8)c8ccccc8n9-c8ccccc8)ccc6n7-c6ccccc6)cccc5c34)n2)cc1]

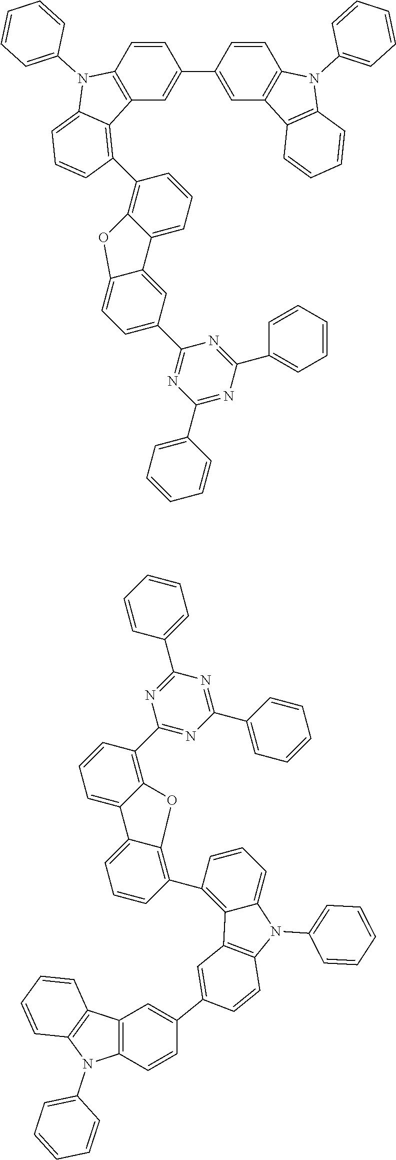 [SMILES: c1ccc(-c2nc(-c3ccccc3)nc(-c3ccc4oc5c(-c6cccc7c6c6cc(-c8ccc9c(c8)c8ccccc8n9-c8ccccc8)ccc6n7-c6ccccc6)cccc5c4c3)n2)cc1 ; c1ccc(-c2nc(-c3ccccc3)nc(-c3cccc4c3oc3c(-c5cccc6c5c5cc(-c7ccc8c(c7)c7ccccc7n8-c7ccccc7)ccc5n6-c5ccccc5)cccc34)n2)cc1]

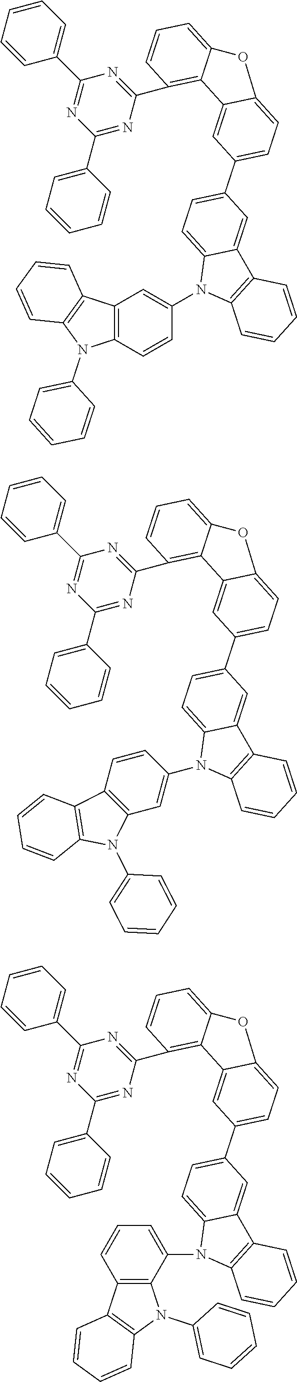

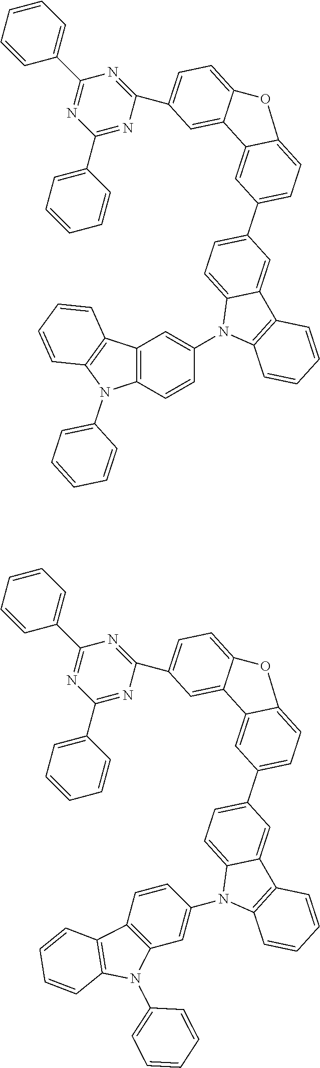 [SMILES: c1ccc(-c2nc(-c3ccccc3)nc(-c3ccc4oc5ccc(-c6ccc7c(c6)c6ccccc6n7-c6ccc7c(c6)c6ccccc6n7-c6ccccc6)cc5c4c3)n2)cc1 ; c1ccc(-c2nc(-c3ccccc3)nc(-c3ccc4oc5ccc(-c6ccc7c(c6)c6ccccc6n7-c6ccc7c8ccccc8n(-c8ccccc8)c7c6)cc5c4c3)n2)cc1]

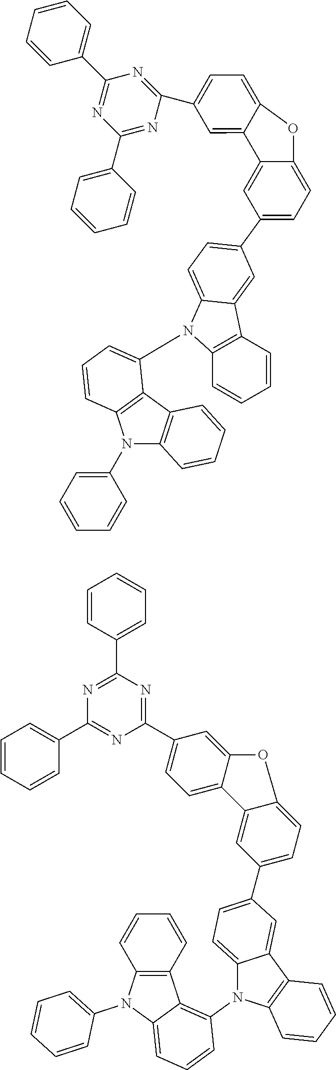

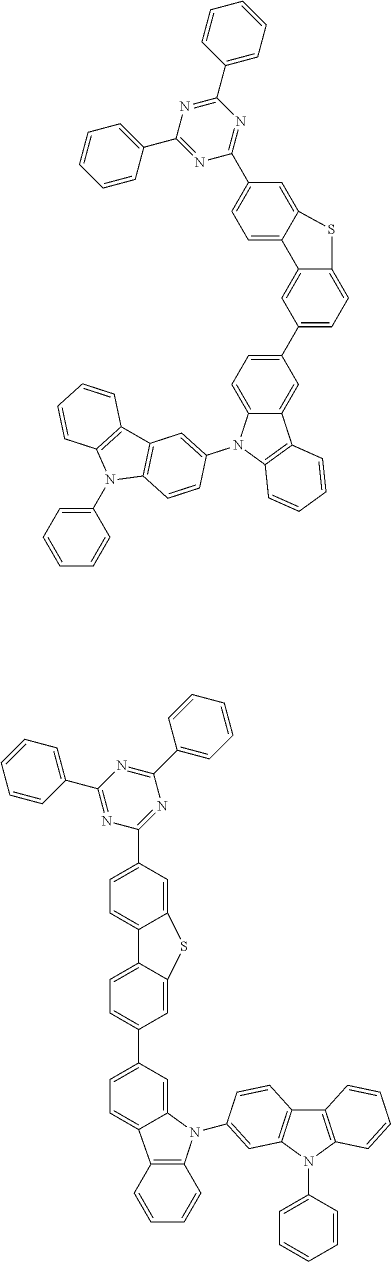 [SMILES: c1ccc(-c2nc(-c3ccccc3)nc(-c3ccc4c(c3)sc3ccc(-c5ccc6c(c5)c5ccccc5n6-c5ccc6c(c5)c5ccccc5n6-c5ccccc5)cc34)n2)cc1 ; c1ccc(-c2nc(-c3ccccc3)nc(-c3ccc4c(c3)sc3cc(-c5ccc6c7ccccc7n(-c7ccc8c9ccccc9n(-c9ccccc9)c8c7)c6c5)ccc34)n2)cc1]

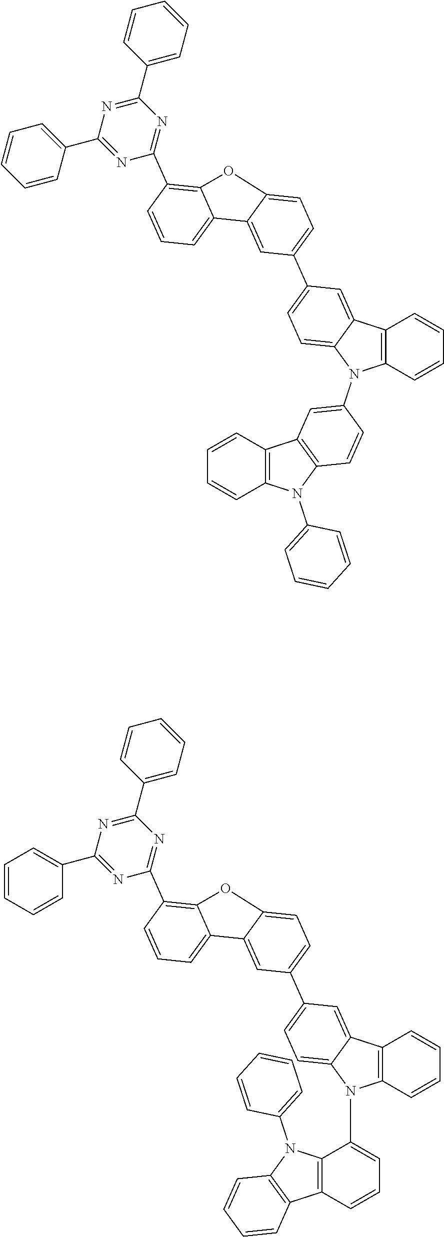 [SMILES: c1ccc(-c2nc(-c3ccccc3)nc(-c3cccc4c3oc3ccc(-c5ccc6c(c5)c5ccccc5n6-c5ccc6c(c5)c5ccccc5n6-c5ccccc5)cc34)n2)cc1 ; c1ccc(-c2nc(-c3ccccc3)nc(-c3cccc4c3oc3ccc(-c5ccc6c(c5)c5ccccc5n6-c5cccc6c7ccccc7n(-c7ccccc7)c56)cc34)n2)cc1]

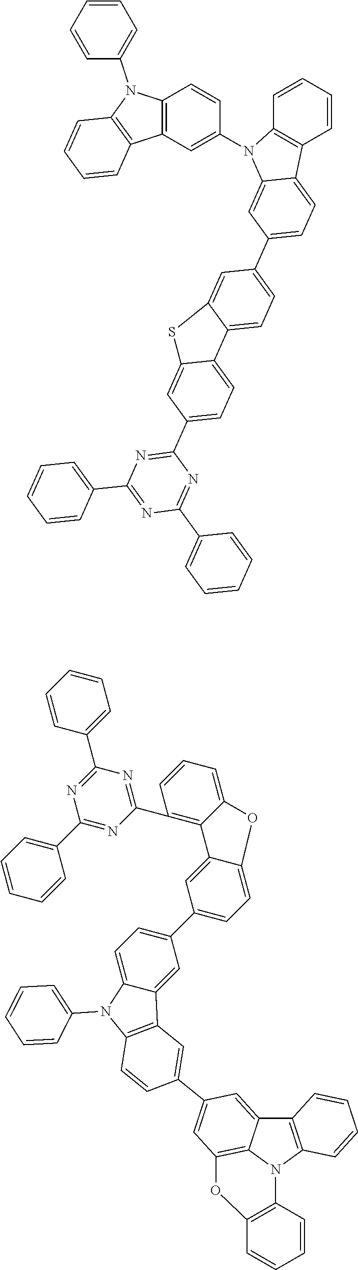 [SMILES: c1ccc(-c2nc(-c3ccccc3)nc(-c3ccc4c(c3)sc3cc(-c5ccc6c7ccccc7n(-c7ccc8c(c7)c7ccccc7n8-c7ccccc7)c6c5)ccc34)n2)cc1 ; c1ccc(-c2nc(-c3ccccc3)nc(-c3cccc4oc5ccc(-c6ccc7c(c6)c6cc(-c8cc9c%10c(c8)c8ccccc8n%10-c8ccccc8O9)ccc6n7-c6ccccc6)cc5c34)n2)cc1]

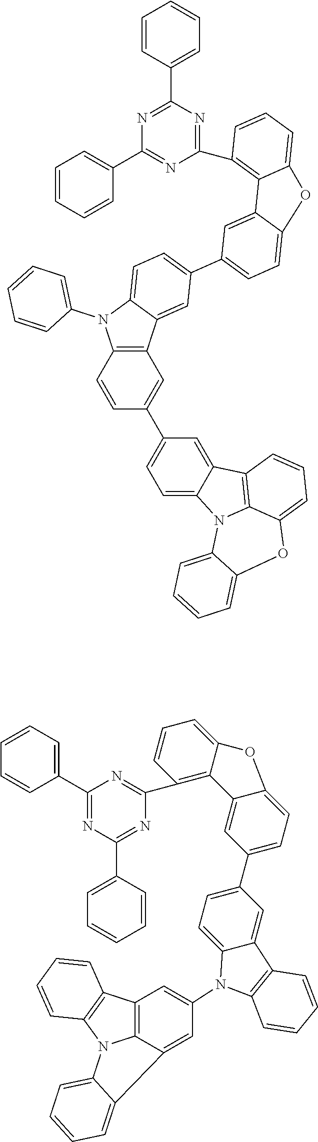 [SMILES: c1ccc(-c2nc(-c3ccccc3)nc(-c3cccc4oc5ccc(-c6ccc7c(c6)c6cc(-c8ccc9c(c8)c8cccc%10c8n9-c8ccccc8O%10)ccc6n7-c6ccccc6)cc5c34)n2)cc1 ; c1ccc(-c2nc(-c3ccccc3)nc(-c3cccc4oc5ccc(-c6ccc7c(c6)c6ccccc6n7-c6cc7c8ccccc8n8c9ccccc9c(c6)c78)cc5c34)n2)cc1]

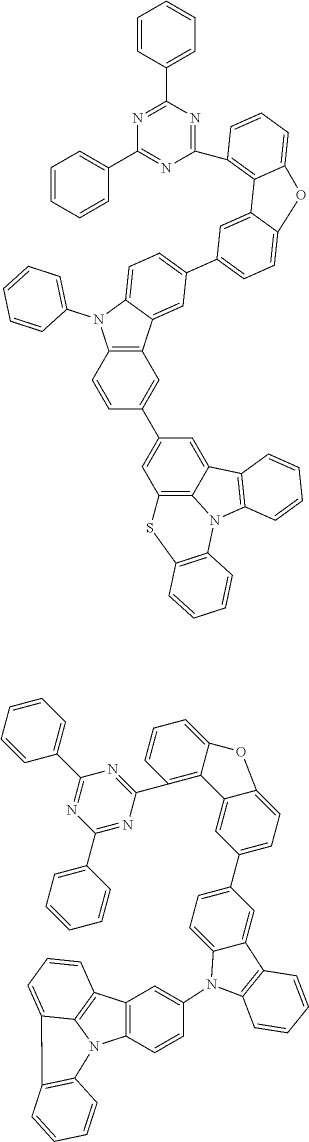 [SMILES: c1ccc(-c2nc(-c3ccccc3)nc(-c3cccc4oc5ccc(-c6ccc7c(c6)c6cc(-c8cc9c%10c(c8)c8ccccc8n%10-c8ccccc8S9)ccc6n7-c6ccccc6)cc5c34)n2)cc1 ; c1ccc(-c2nc(-c3ccccc3)nc(-c3cccc4oc5ccc(-c6ccc7c(c6)c6ccccc6n7-c6ccc7c(c6)c6cccc8c9ccccc9n7c86)cc5c34)n2)cc1]

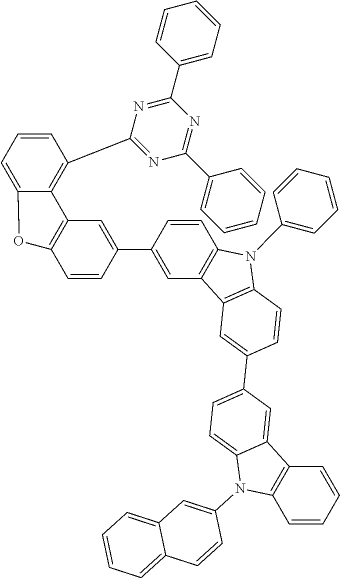 [SMILES: c1ccc(-c2nc(-c3ccccc3)nc(-c3cccc4oc5ccc(-c6ccc7c(c6)c6cc(-c8ccc9c(c8)c8ccccc8n9-c8ccc9ccccc9c8)ccc6n7-c6ccccc6)cc5c34)n2)cc1]

Suitable charge transport materials as usable in the hole injection layer or hole transport layer or electron blocker layer or in the electron transport layer of the electronic device of the invention are, for example, the compounds disclosed in Y. Shirota et al., Chem. Rev. 2007, 107(4), 953-1010, or other materials as used in these layers according to the prior art.

Suitable materials for the electron-transporting layers of the device are especially aluminum complexes, for example Alq₃, zirconium complexes, for example Zrq₄, lithium complexes, for example Liq, benzimidazole derivatives, triazine derivatives, pyrimidine derivatives, pyridine derivatives, pyrazine derivatives, quinoxaline derivatives, quinoline derivatives, oxadiazole derivatives, aromatic ketones, lactams, boranes, diazaphosphole derivatives and phosphine oxide derivatives.

Particularly preferred compounds for use in electron-transporting layers are shown in the following table:

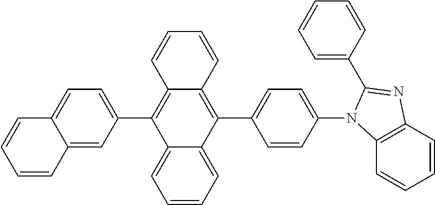 [SMILES: c1ccc(-c2nc3ccccc3n2-c2ccc(-c3c4ccccc4c(-c4ccc5ccccc5c4)c4ccccc34)cc2)cc1]

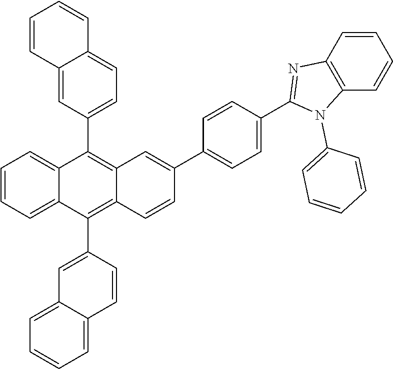 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1]

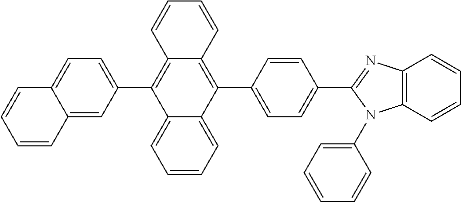 [SMILES: c1ccc(-n2c(-c3ccc(-c4c5ccccc5c(-c5ccc6ccccc6c5)c5ccccc45)cc3)nc3ccccc32)cc1]

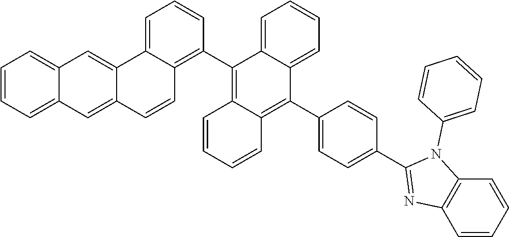 [SMILES: c1ccc(-n2c(-c3ccc(-c4c5ccccc5c(-c5cccc6c5ccc5cc7ccccc7cc56)c5ccccc45)cc3)nc3ccccc32)cc1]

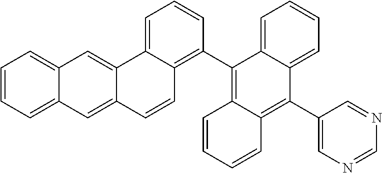 [SMILES: c1ccc2cc3c(ccc4c(-c5c6ccccc6c(-c6cncnc6)c6ccccc56)cccc43)cc2c1]

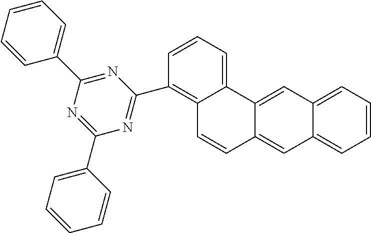 [SMILES: c1ccc(-c2nc(-c3ccccc3)nc(-c3cccc4c3ccc3cc5ccccc5cc34)n2)cc1]

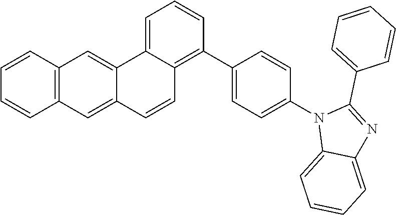 [SMILES: c1ccc(-c2nc3ccccc3n2-c2ccc(-c3cccc4c3ccc3cc5ccccc5cc34)cc2)cc1]

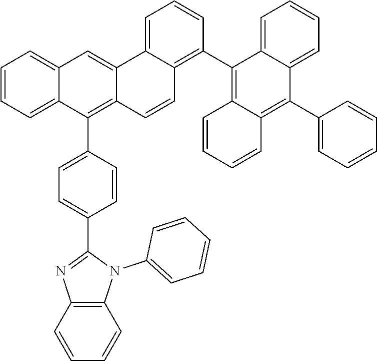 [SMILES: c1ccc(-c2c3ccccc3c(-c3cccc4c3ccc3c(-c5ccc(-c6nc7ccccc7n6-c6ccccc6)cc5)c5ccccc5cc34)c3ccccc23)cc1]

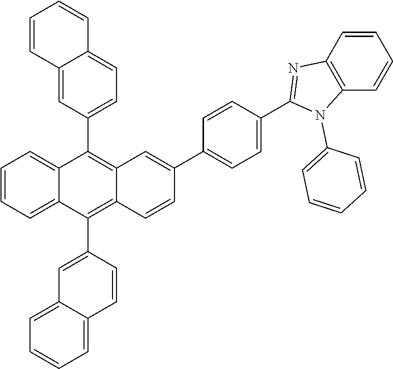 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1]

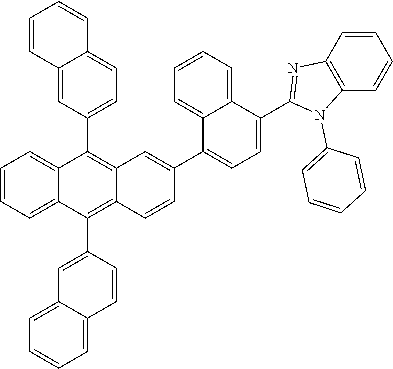 [SMILES: c1ccc(-n2c(-c3ccc(-c4ccc5c(-c6ccc7ccccc7c6)c6ccccc6c(-c6ccc7ccccc7c6)c5c4)c4ccccc34)nc3ccccc32)cc1]

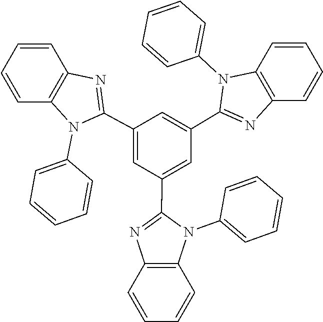 [SMILES: c1ccc(-n2c(-c3cc(-c4nc5ccccc5n4-c4ccccc4)cc(-c4nc5ccccc5n4-c4ccccc4)c3)nc3ccccc32)cc1]

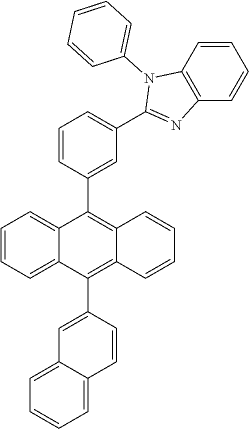 [SMILES: c1ccc(-n2c(-c3cccc(-c4c5ccccc5c(-c5ccc6ccccc6c5)c5ccccc45)c3)nc3ccccc32)cc1]

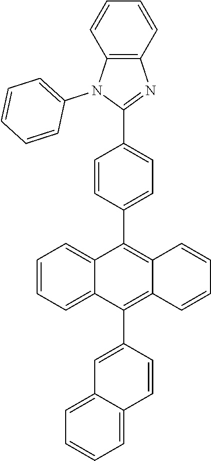 [SMILES: c1ccc(-n2c(-c3ccc(-c4c5ccccc5c(-c5ccc6ccccc6c5)c5ccccc45)cc3)nc3ccccc32)cc1]

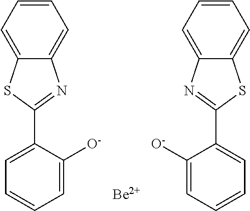

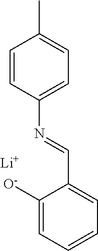 [SMILES: Cc1ccc(/N=C/c2ccccc2[O-])cc1 ; [Li+]]

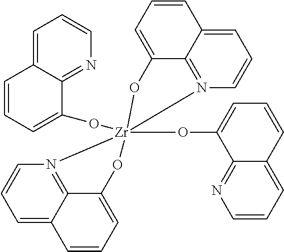

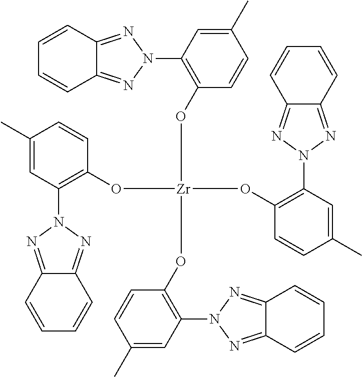

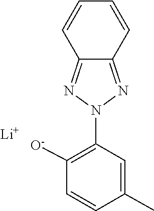 [SMILES: [Li+] ; Cc1ccc([O-])c(-n2nc3ccccc3n2)c1]

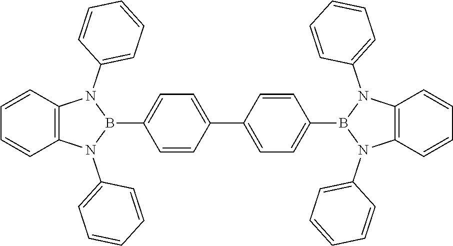 [SMILES: c1ccc(N2B(c3ccc(-c4ccc(B5N(c6ccccc6)c6ccccc6N5c5ccccc5)cc4)cc3)N(c3ccccc3)c3ccccc32)cc1]

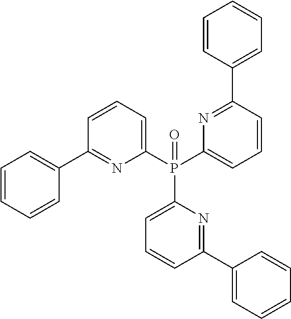 [SMILES: O=P(c1cccc(-c2ccccc2)n1)(c1cccc(-c2ccccc2)n1)c1cccc(-c2ccccc2)n1]

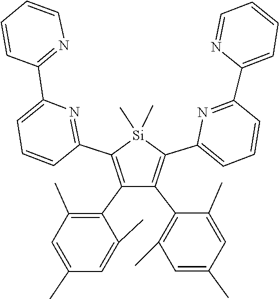 [SMILES: Cc1cc(C)c(C2=C(c3cccc(-c4ccccn4)n3)[Si](C)(C)C(c3cccc(-c4ccccn4)n3)=C2c2c(C)cc(C)cc2C)c(C)c1]

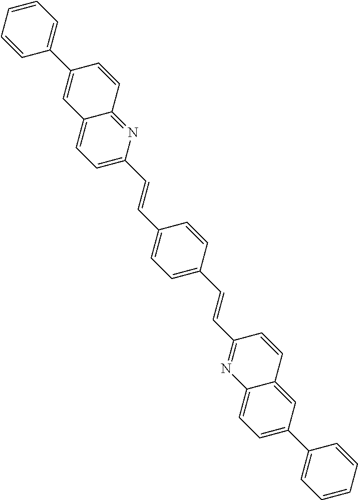 [SMILES: C(=C\c1ccc2cc(-c3ccccc3)ccc2n1)/c1ccc(/C=C/c2ccc3cc(-c4ccccc4)ccc3n2)cc1]

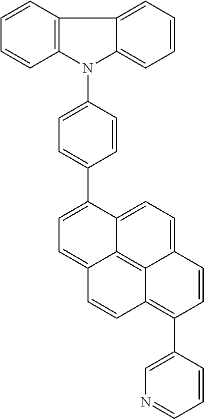 [SMILES: c1cncc(-c2ccc3ccc4c(-c5ccc(-n6c7ccccc7c7ccccc76)cc5)ccc5ccc2c3c54)c1]

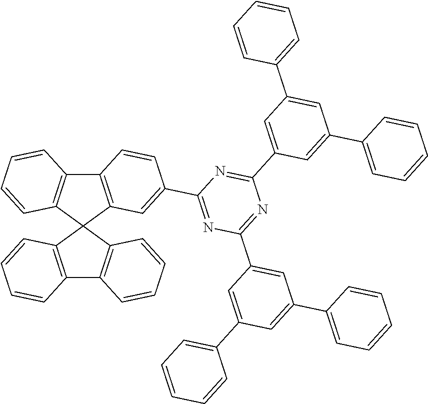 [SMILES: c1ccc(-c2cc(-c3ccccc3)cc(-c3nc(-c4cc(-c5ccccc5)cc(-c5ccccc5)c4)nc(-c4ccc5c(c4)C4(c6ccccc6-c6ccccc64)c4ccccc4-5)n3)c2)cc1]

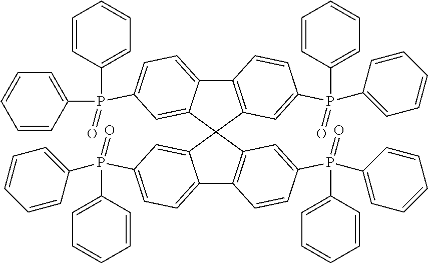 [SMILES: O=P(c1ccccc1)(c1ccccc1)c1ccc2c(c1)C1(c3cc(P(=O)(c4ccccc4)c4ccccc4)ccc3-2)c2cc(P(=O)(c3ccccc3)c3ccccc3)ccc2-c2ccc(P(=O)(c3ccccc3)c3ccccc3)cc21]

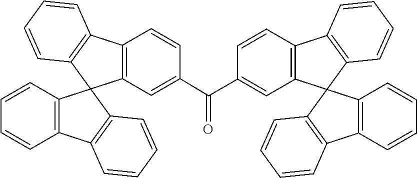 [SMILES: O=C(c1ccc2c(c1)C1(c3ccccc3-c3ccccc31)c1ccccc1-2)c1ccc2c(c1)C1(c3ccccc3-c3ccccc31)c1ccccc1-2]

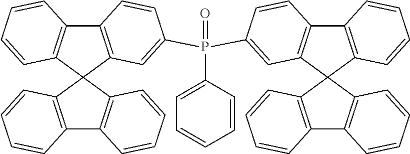 [SMILES: O=P(c1ccccc1)(c1ccc2c(c1)C1(c3ccccc3-c3ccccc31)c1ccccc1-2)c1ccc2c(c1)C1(c3ccccc3-c3ccccc31)c1ccccc1-2]

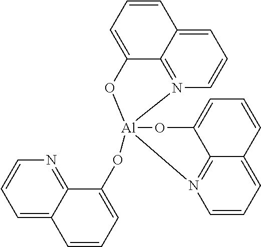

Materials used for hole-transporting layers of OLEDs may preferably be indenofluoreneamine derivatives, amine derivatives, hexaazatriphenylene derivatives, amine derivatives with fused aromatic systems, monobenzoindenofluoreneamines, dibenzoindenofluoreneamines, spirobifluoreneamines, fluoreneamines, spirodibenzopyranamines, dihydroacridine derivatives, spirodibenzofurans and spirodibenzothiophenes, phenanthrenediarylamines, spirotribenzotropolones, spirobifluorenes having meta-phenyldiamine groups, spirobisacridines, xanthenediarylamines, and 9,10-dihydroanthracene spiro compounds having diarylamino groups. More particularly, the following compounds are suitable for this purpose:

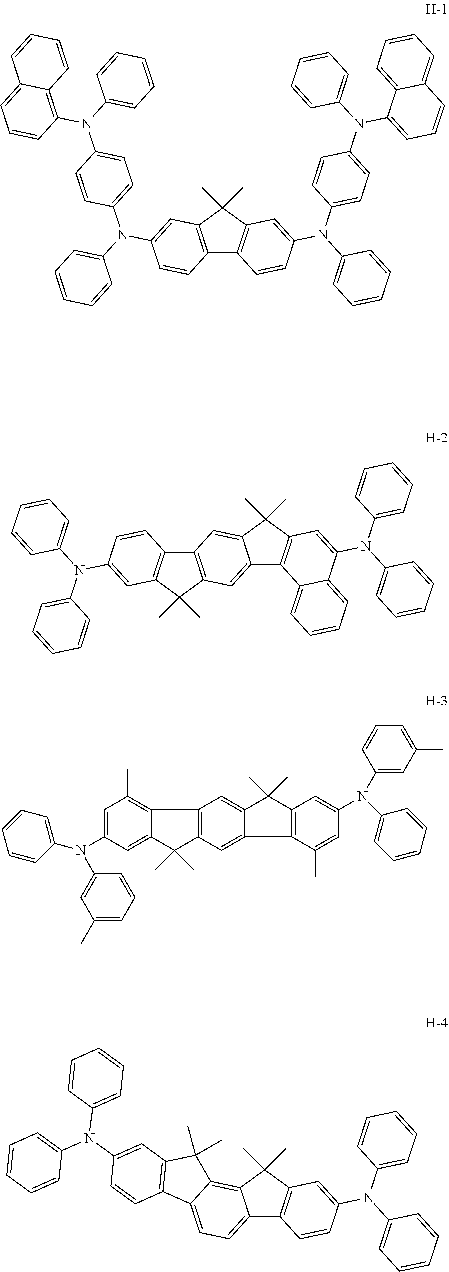 [SMILES: CC1(C)c2cc(N(c3ccccc3)c3ccc(N(c4ccccc4)c4cccc5ccccc45)cc3)ccc2-c2ccc(N(c3ccccc3)c3ccc(N(c4ccccc4)c4cccc5ccccc45)cc3)cc21 ; CC1(C)c2cc(N(c3ccccc3)c3ccccc3)ccc2-c2cc3c(cc21)-c1c(cc(N(c2ccccc2)c2ccccc2)c2ccccc12)C3(C)C ; Cc1cccc(N(c2ccccc2)c2cc(C)c3c(c2)C(C)(C)c2cc4c(cc2-3)C(C)(C)c2cc(N(c3ccccc3)c3cccc(C)c3)cc(C)c2-4)c1 ; CC1(C)c2cc(N(c3ccccc3)c3ccccc3)ccc2-c2ccc3c(c21)C(C)(C)c1cc(N(c2ccccc2)c2ccccc2)ccc1-3]

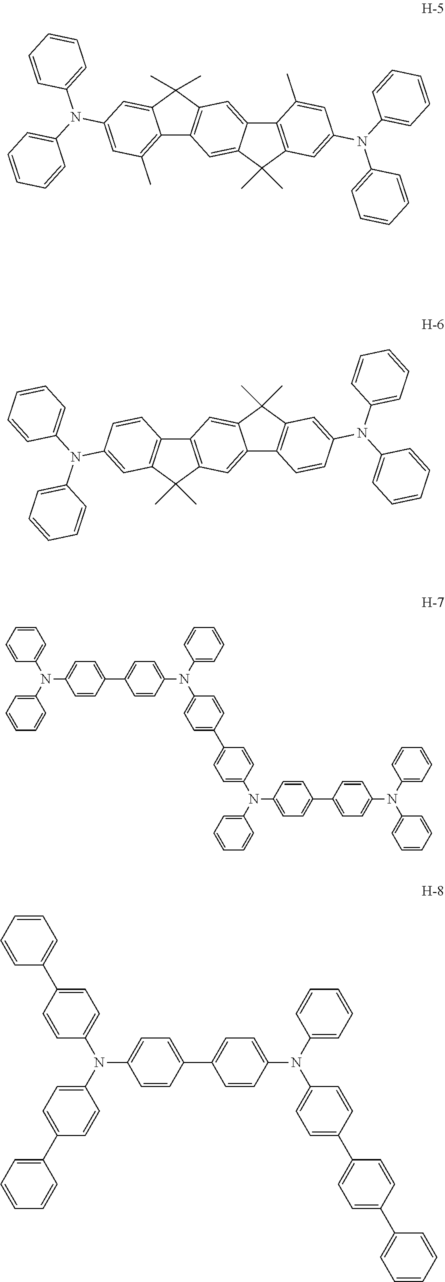 [SMILES: Cc1cc(N(c2ccccc2)c2ccccc2)cc2c1-c1cc3c(cc1C2(C)C)-c1c(C)cc(N(c2ccccc2)c2ccccc2)cc1C3(C)C ; CC1(C)c2cc(N(c3ccccc3)c3ccccc3)ccc2-c2cc3c(cc21)-c1ccc(N(c2ccccc2)c2ccccc2)cc1C3(C)C ; c1ccc(N(c2ccccc2)c2ccc(-c3ccc(N(c4ccccc4)c4ccc(-c5ccc(N(c6ccccc6)c6ccc(-c7ccc(N(c8ccccc8)c8ccccc8)cc7)cc6)cc5)cc4)cc3)cc2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1]

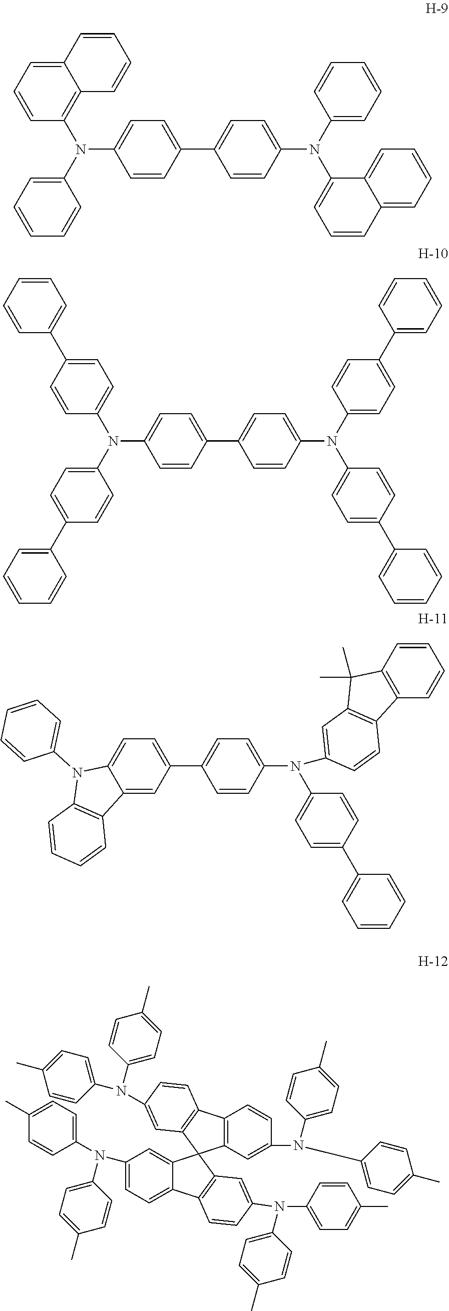

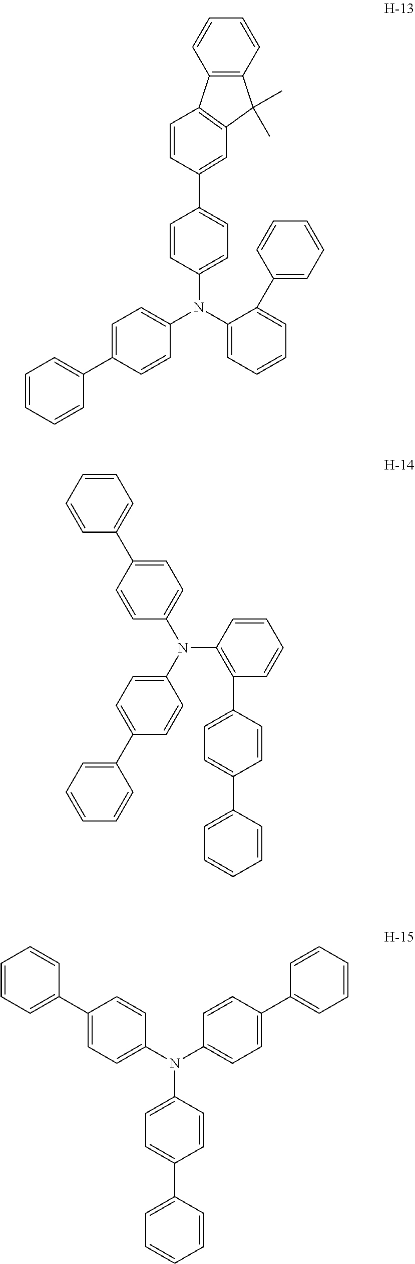 [SMILES: CC1(C)c2ccccc2-c2ccc(-c3ccc(N(c4ccc(-c5ccccc5)cc4)c4ccccc4-c4ccccc4)cc3)cc21 ; c1ccc(-c2ccc(-c3ccccc3N(c3ccc(-c4ccccc4)cc3)c3ccc(-c4ccccc4)cc3)cc2)cc1 ; c1ccc(-c2ccc(N(c3ccc(-c4ccccc4)cc3)c3ccc(-c4ccccc4)cc3)cc2)cc1]

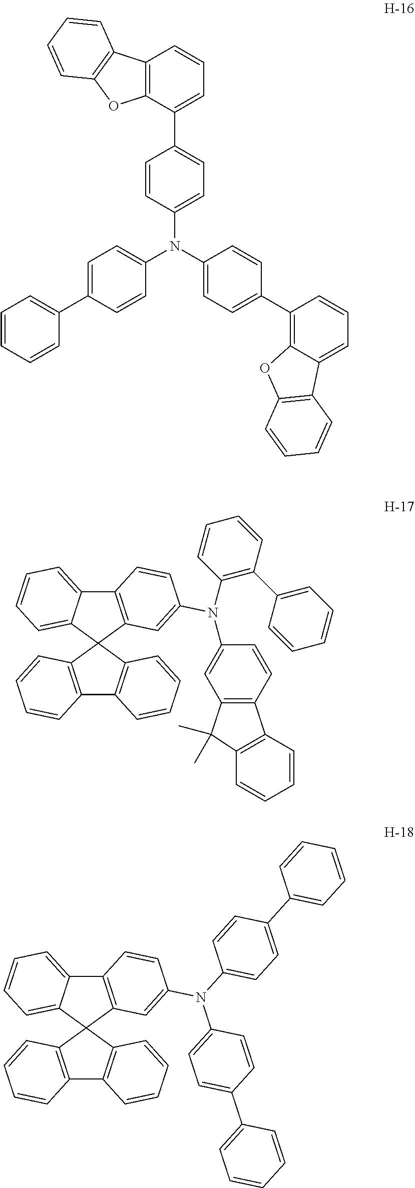 [SMILES: c1ccc(-c2ccc(N(c3ccc(-c4cccc5c4oc4ccccc45)cc3)c3ccc(-c4cccc5c4oc4ccccc45)cc3)cc2)cc1 ; CC1(C)c2ccccc2-c2ccc(N(c3ccc4c(c3)C3(c5ccccc5-c5ccccc53)c3ccccc3-4)c3ccccc3-c3ccccc3)cc21 ; c1ccc(-c2ccc(N(c3ccc(-c4ccccc4)cc3)c3ccc4c(c3)C3(c5ccccc5-c5ccccc53)c3ccccc3-4)cc2)cc1]

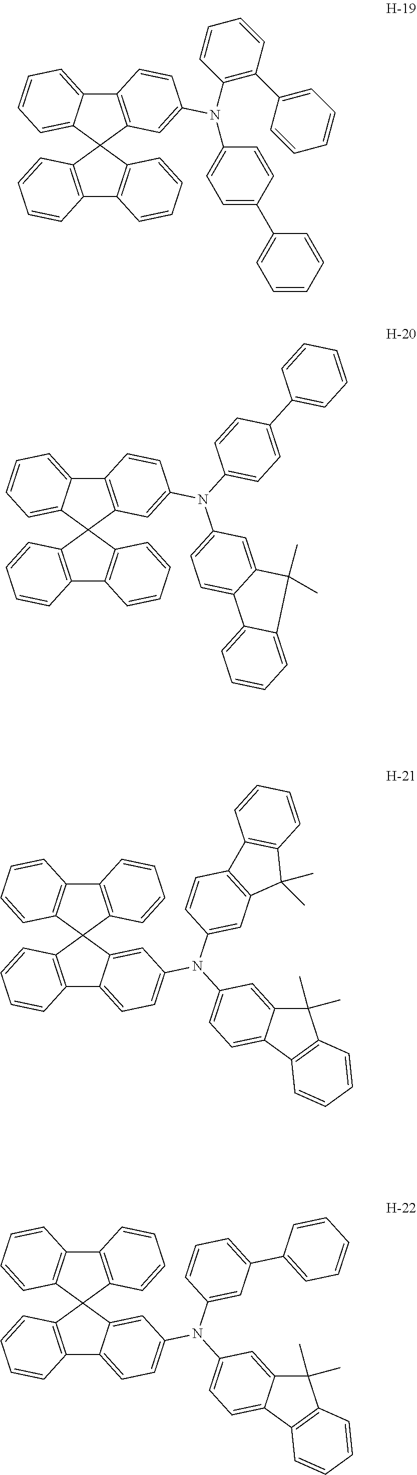 [SMILES: c1ccc(-c2ccc(N(c3ccc4c(c3)C3(c5ccccc5-c5ccccc53)c3ccccc3-4)c3ccccc3-c3ccccc3)cc2)cc1 ; CC1(C)c2ccccc2-c2ccc(N(c3ccc(-c4ccccc4)cc3)c3ccc4c(c3)C3(c5ccccc5-c5ccccc53)c3ccccc3-4)cc21 ; CC1(C)c2ccccc2-c2ccc(N(c3ccc4c(c3)C(C)(C)c3ccccc3-4)c3ccc4c(c3)C3(c5ccccc5-c5ccccc53)c3ccccc3-4)cc21 ; CC1(C)c2ccccc2-c2ccc(N(c3cccc(-c4ccccc4)c3)c3ccc4c(c3)C3(c5ccccc5-c5ccccc53)c3ccccc3-4)cc21]

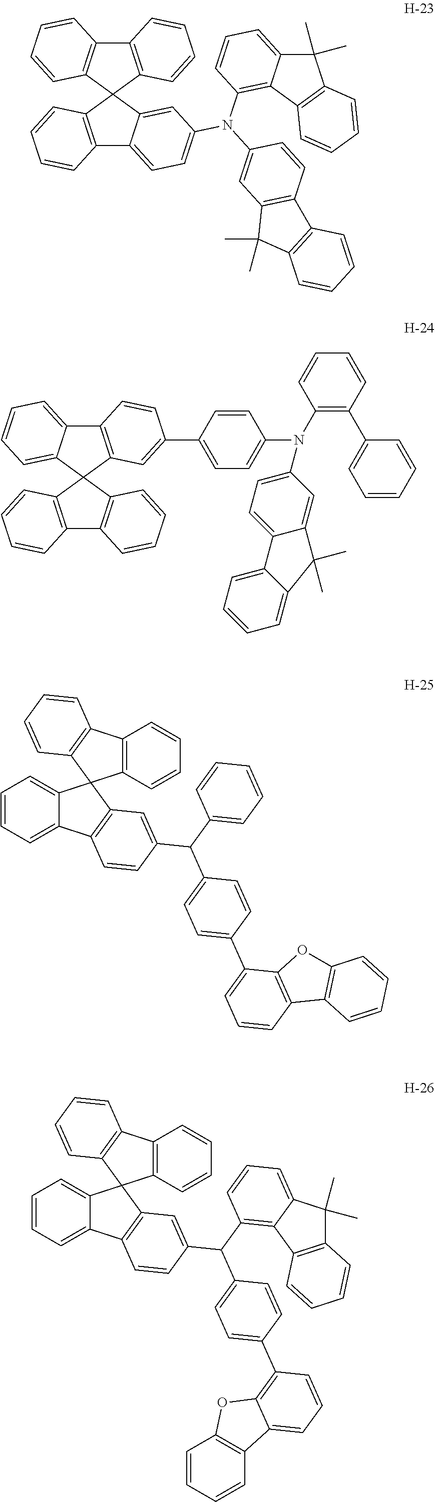 [SMILES: CC1(C)c2ccccc2-c2ccc(N(c3ccc4c(c3)C3(c5ccccc5-c5ccccc53)c3ccccc3-4)c3cccc4c3-c3ccccc3C4(C)C)cc21 ; CC1(C)c2ccccc2-c2ccc(N(c3ccc(-c4ccc5c(c4)C4(c6ccccc6-c6ccccc64)c4ccccc4-5)cc3)c3ccccc3-c3ccccc3)cc21 ; c1ccc(C(c2ccc(-c3cccc4c3oc3ccccc34)cc2)c2ccc3c(c2)C2(c4ccccc4-c4ccccc42)c2ccccc2-3)cc1 ; CC1(C)c2ccccc2-c2c(C(c3ccc(-c4cccc5c4oc4ccccc45)cc3)c3ccc4c(c3)C3(c5ccccc5-c5ccccc53)c3ccccc3-4)cccc21]

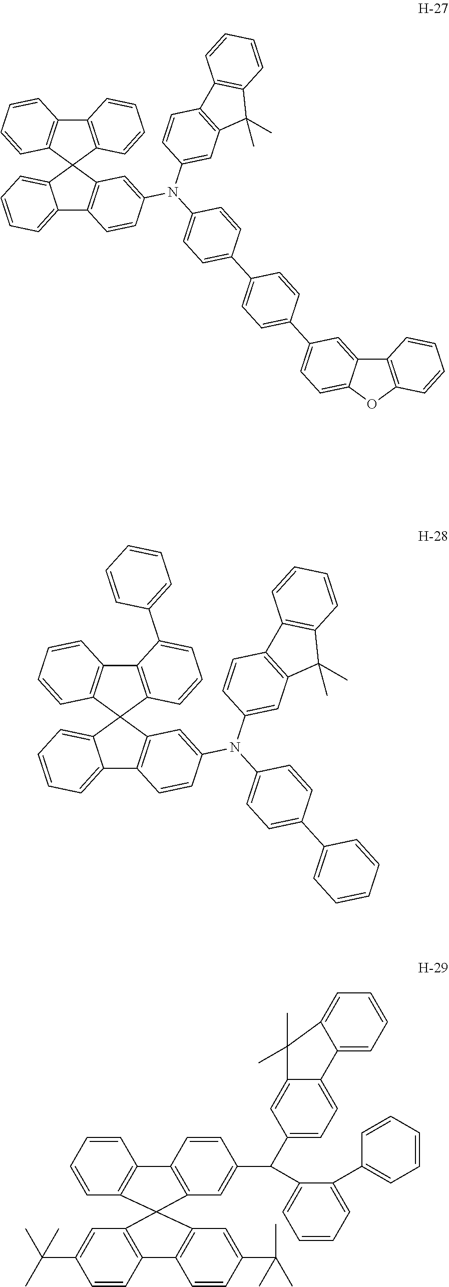

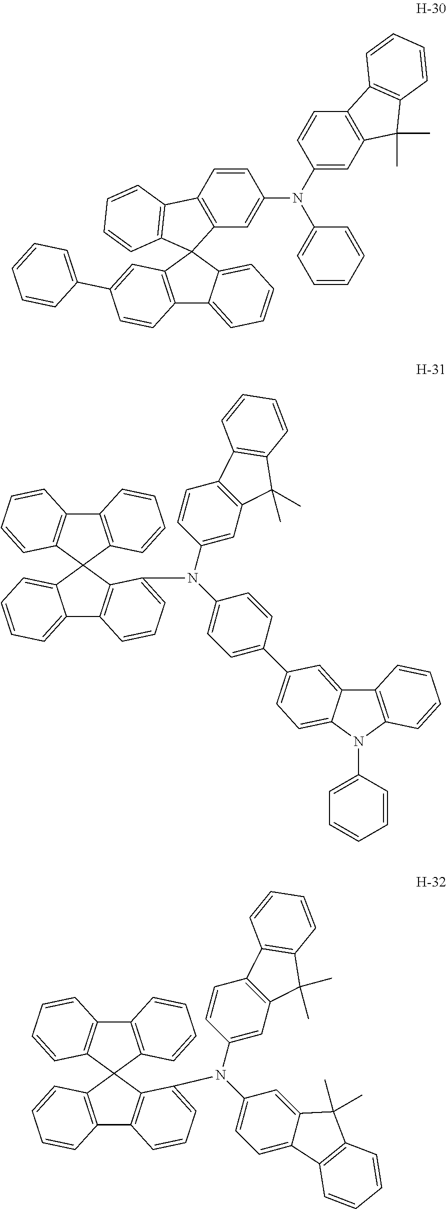 [SMILES: CC1(C)c2ccccc2-c2ccc(N(c3ccccc3)c3ccc4c(c3)C3(c5ccccc5-c5ccc(-c6ccccc6)cc53)c3ccccc3-4)cc21 ; CC1(C)c2ccccc2-c2ccc(N(c3ccc(-c4ccc5c(c4)c4ccccc4n5-c4ccccc4)cc3)c3cccc4c3C3(c5ccccc5-c5ccccc53)c3ccccc3-4)cc21 ; CC1(C)c2ccccc2-c2ccc(N(c3ccc4c(c3)C(C)(C)c3ccccc3-4)c3cccc4c3C3(c5ccccc5-c5ccccc53)c3ccccc3-4)cc21]

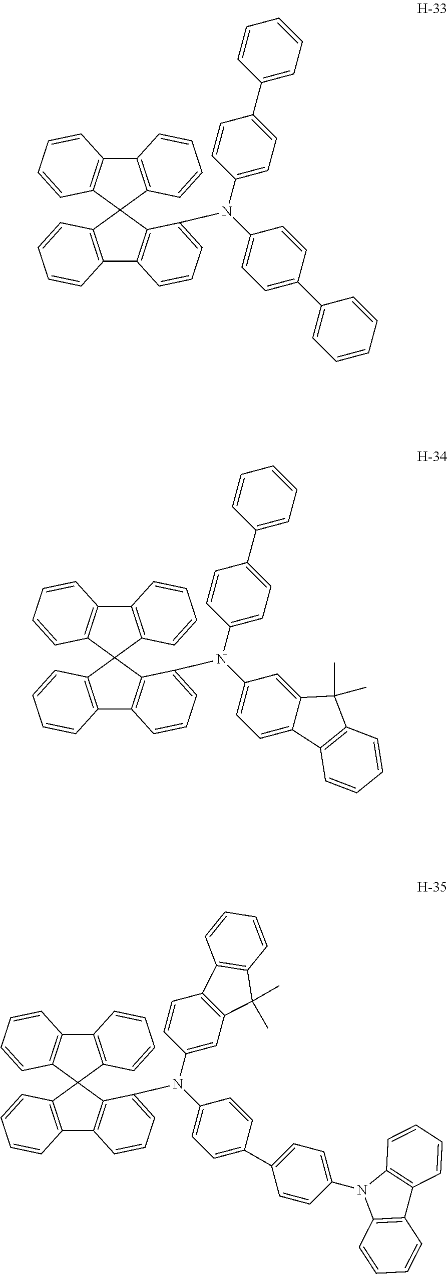 [SMILES: c1ccc(-c2ccc(N(c3ccc(-c4ccccc4)cc3)c3cccc4c3C3(c5ccccc5-c5ccccc53)c3ccccc3-4)cc2)cc1 ; CC1(C)c2ccccc2-c2ccc(N(c3ccc(-c4ccccc4)cc3)c3cccc4c3C3(c5ccccc5-c5ccccc53)c3ccccc3-4)cc21 ; CC1(C)c2ccccc2-c2ccc(N(c3ccc(-c4ccc(-n5c6ccccc6c6ccccc65)cc4)cc3)c3cccc4c3C3(c5ccccc5-c5ccccc53)c3ccccc3-4)cc21]

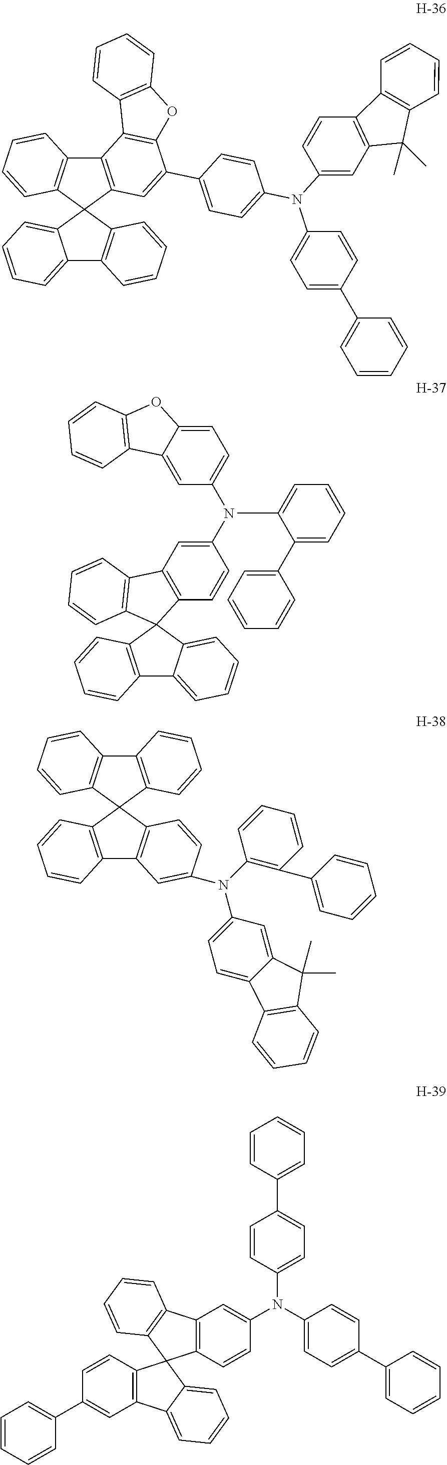

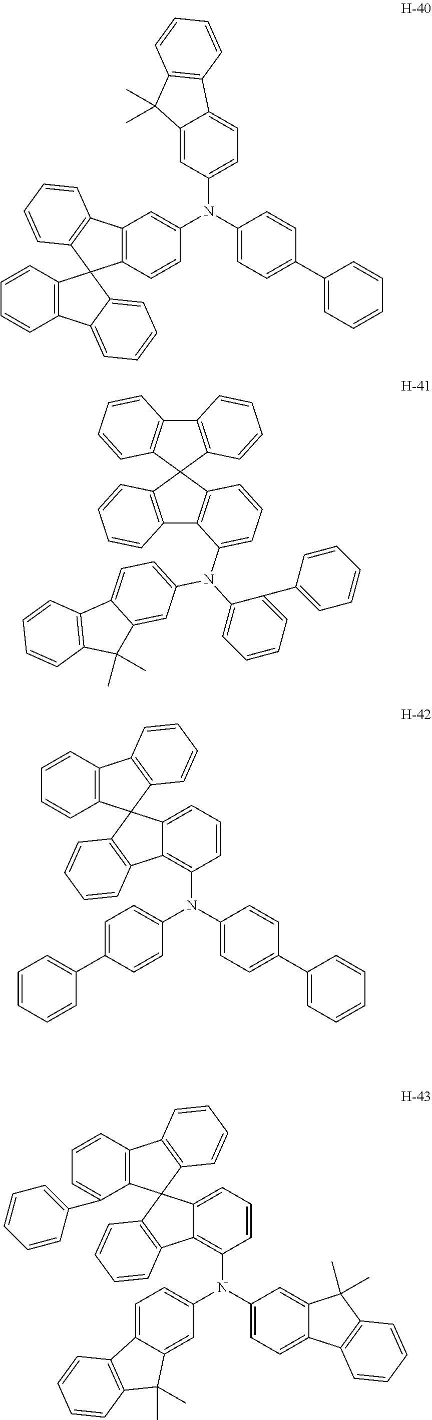

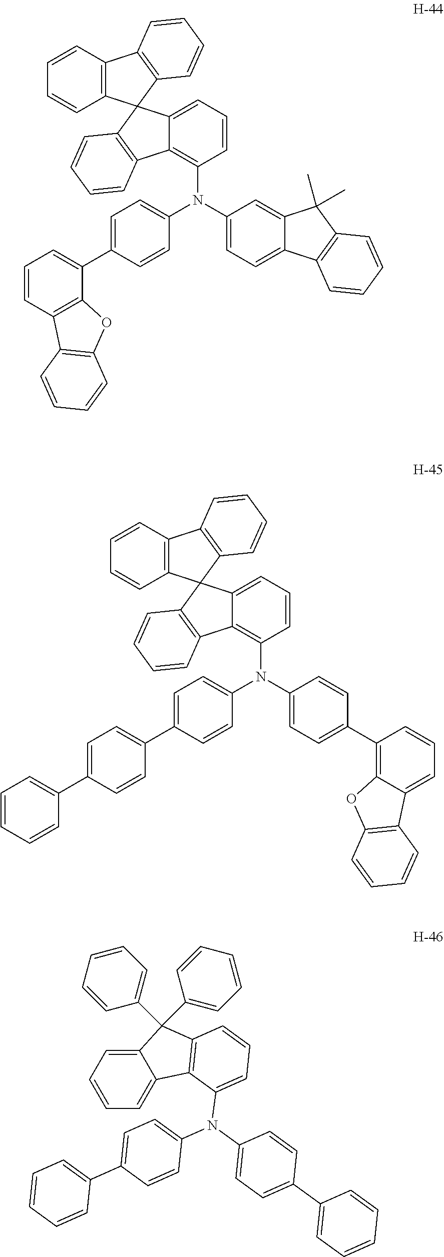 [SMILES: CC1(C)c2ccccc2-c2ccc(N(c3ccc(-c4cccc5c4oc4ccccc45)cc3)c3cccc4c3-c3ccccc3C43c4ccccc4-c4ccccc43)cc21 ; c1ccc(-c2ccc(-c3ccc(N(c4ccc(-c5cccc6c5oc5ccccc56)cc4)c4cccc5c4-c4ccccc4C54c5ccccc5-c5ccccc54)cc3)cc2)cc1 ; c1ccc(-c2ccc(N(c3ccc(-c4ccccc4)cc3)c3cccc4c3-c3ccccc3C4(c3ccccc3)c3ccccc3)cc2)cc1]

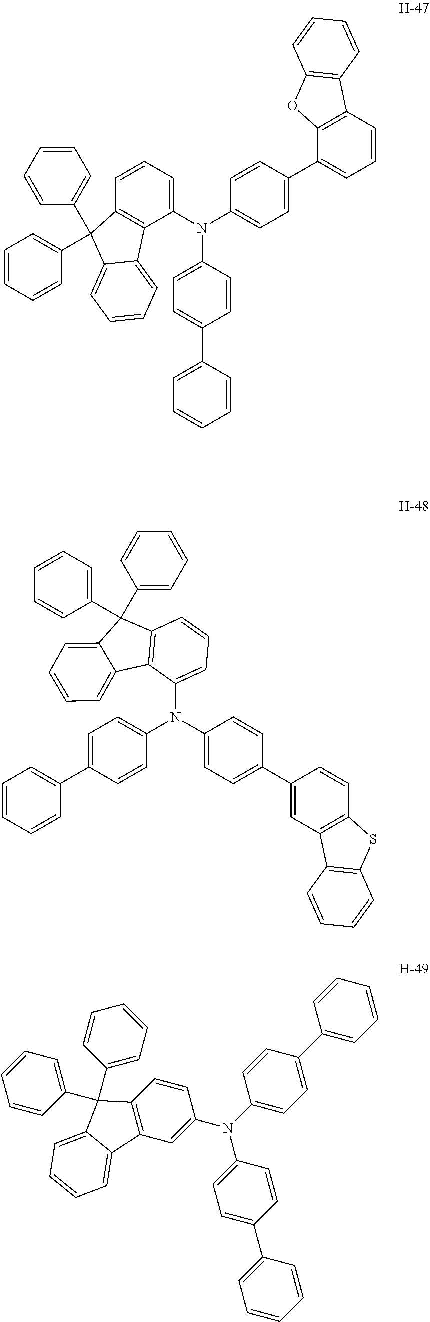 [SMILES: c1ccc(-c2ccc(N(c3ccc(-c4cccc5c4oc4ccccc45)cc3)c3cccc4c3-c3ccccc3C4(c3ccccc3)c3ccccc3)cc2)cc1 ; c1ccc(-c2ccc(N(c3ccc(-c4ccc5sc6ccccc6c5c4)cc3)c3cccc4c3-c3ccccc3C4(c3ccccc3)c3ccccc3)cc2)cc1 ; c1ccc(-c2ccc(N(c3ccc(-c4ccccc4)cc3)c3ccc4c(c3)-c3ccccc3C4(c3ccccc3)c3ccccc3)cc2)cc1]

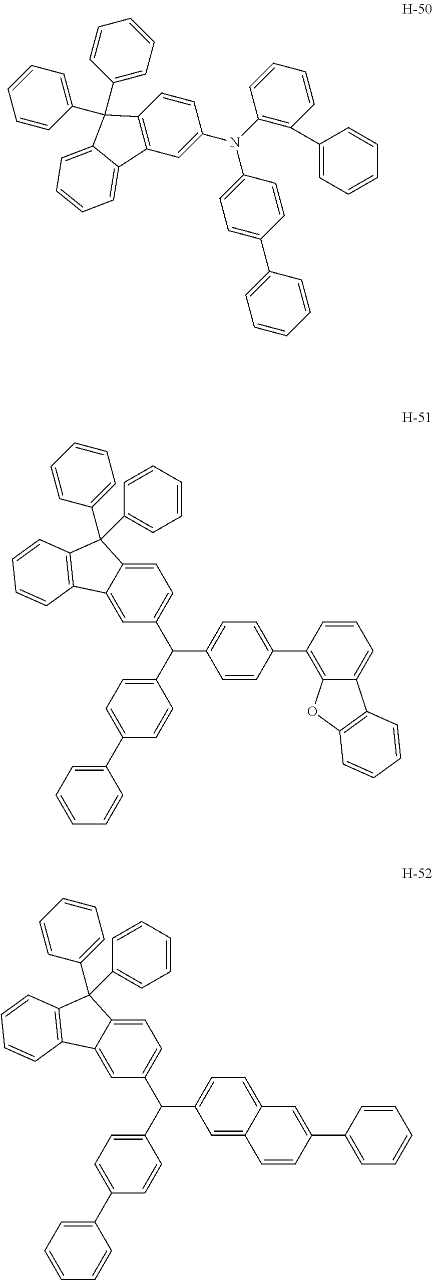

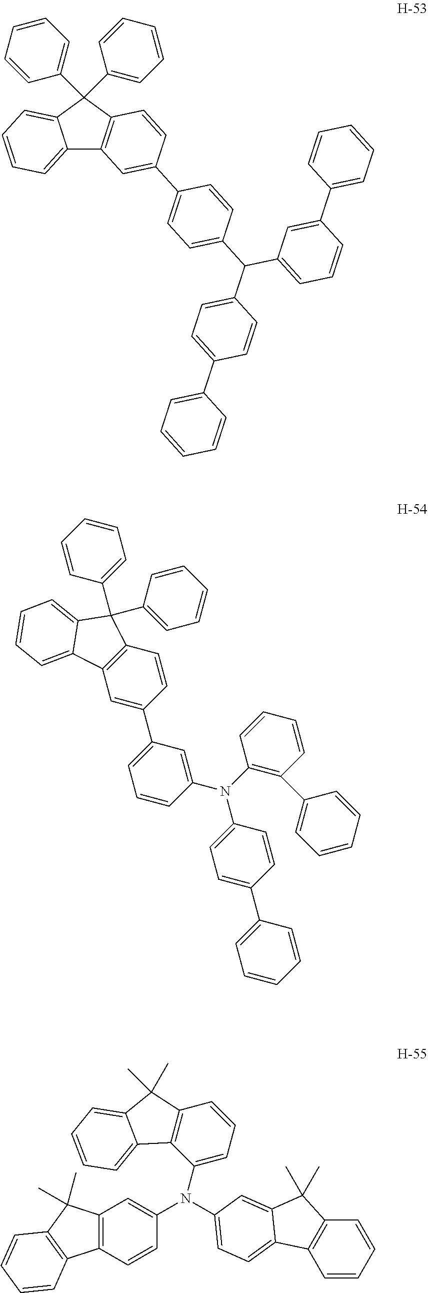

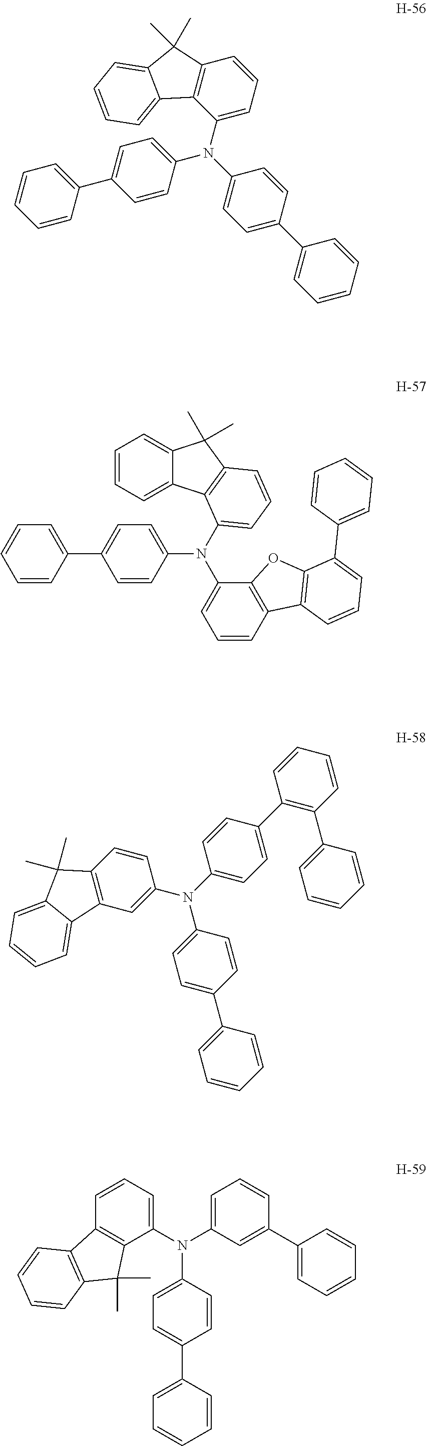 [SMILES: CC1(C)c2ccccc2-c2c(N(c3ccc(-c4ccccc4)cc3)c3ccc(-c4ccccc4)cc3)cccc21 ; CC1(C)c2ccccc2-c2c(N(c3ccc(-c4ccccc4)cc3)c3cccc4c3oc3c(-c5ccccc5)cccc34)cccc21 ; CC1(C)c2ccccc2-c2cc(N(c3ccc(-c4ccccc4)cc3)c3ccc(-c4ccccc4-c4ccccc4)cc3)ccc21 ; CC1(C)c2ccccc2-c2cccc(N(c3ccc(-c4ccccc4)cc3)c3cccc(-c4ccccc4)c3)c21]

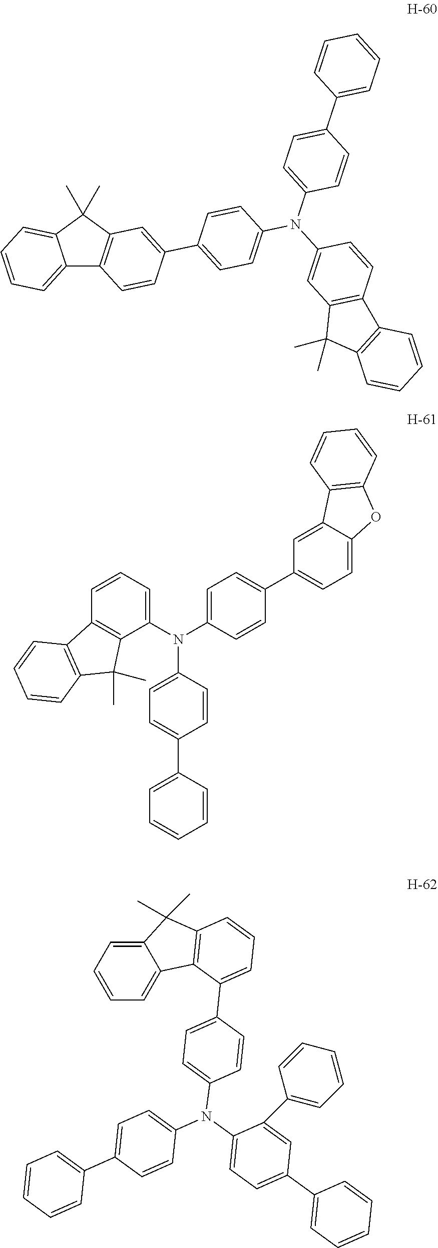

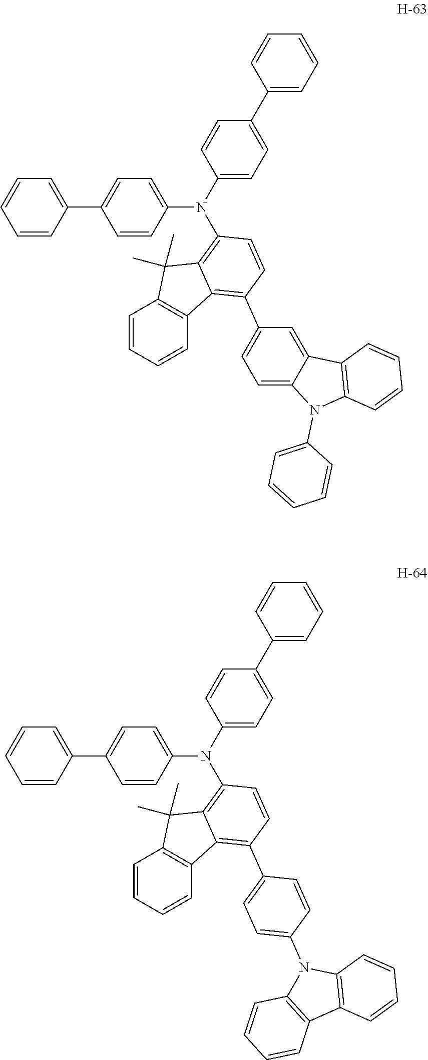 [SMILES: CC1(C)c2ccccc2-c2c(-c3ccc4c(c3)c3ccccc3n4-c3ccccc3)ccc(N(c3ccc(-c4ccccc4)cc3)c3ccc(-c4ccccc4)cc3)c21 ; CC1(C)c2ccccc2-c2c(-c3ccc(-n4c5ccccc5c5ccccc54)cc3)ccc(N(c3ccc(-c4ccccc4)cc3)c3ccc(-c4ccccc4)cc3)c21]

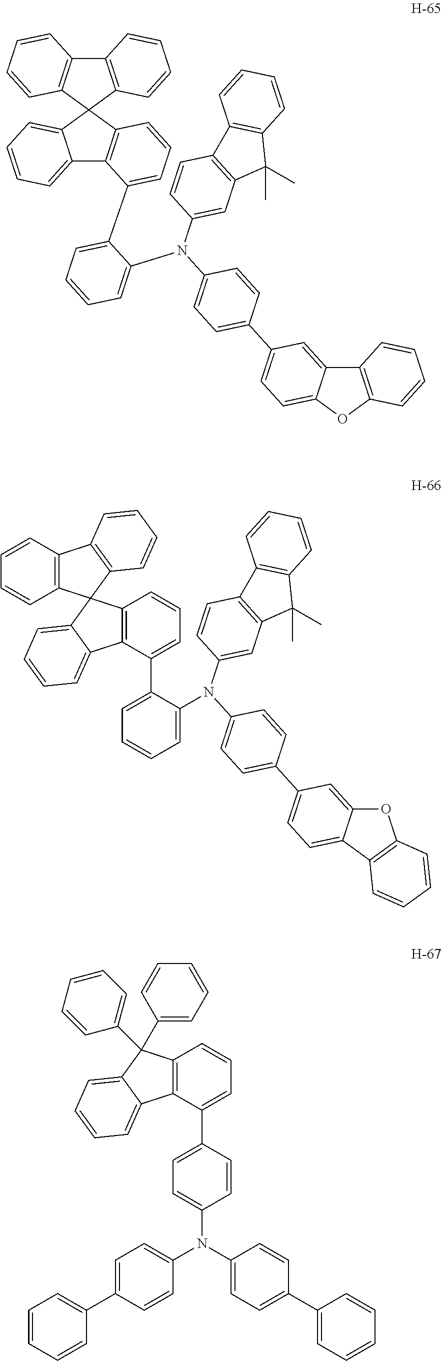

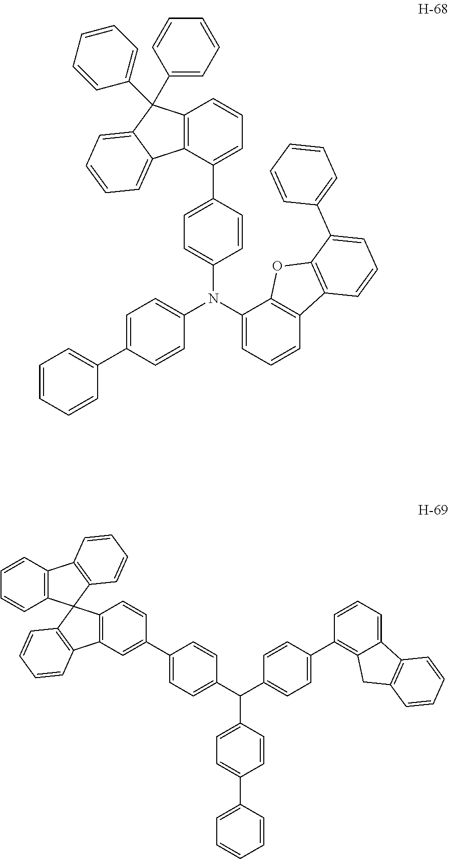

Methods of synthesis of compounds such as H-31, H-45 and H-69, for example, are disclosed in published specification WO2013/120577.

Preferred cathodes of the electronic device are metals having a low work function, metal alloys or multilayer structures composed of various metals, for example alkaline earth metals, alkali metals, main group metals or lanthanoids (e.g. Ca, Ba, Mg, Al, In, Mg, Yb, Sm, etc.). Additionally suitable are alloys composed of an alkali metal or alkaline earth metal and silver, for example an alloy composed of magnesium and silver. In the case of multilayer structures, in addition to the metals mentioned, it is also possible to use further metals having a relatively high work function, for example Ag or Al, in which case combinations of the metals such as Ca/Ag, Mg/Ag or Ba/Ag, for example, are generally used. It may also be preferable to introduce a thin interlayer of a material having a high dielectric constant between a metallic cathode and the organic semiconductor. Examples of useful materials for this purpose are alkali metal or alkaline earth metal fluorides, but also the corresponding oxides or carbonates (e.g. LiF, Li₂O, BaF₂, MgO, NaF, CsF, Cs₂CO₃, etc.). It is also possible to use lithium quinolinate (LiQ) for this purpose. The layer thickness of this layer is preferably between 0.5 and 5 nm.

Preferred anodes are materials having a high work function. Preferably, the anode has a work function of greater than 4.5 eV versus vacuum. Firstly, metals having a high redox potential are suitable for this purpose, for example Ag, Pt or Au. Secondly, metal/metal oxide electrodes (e.g. Al/Ni/NiOₓ, Al/PtOₓ) may also be preferred. For some applications, at least one of the electrodes has to be transparent or partly transparent in order to enable either the irradiation of the organic material (organic solar cell) or the emission of light (OLED, O-LASER). Preferred anode materials here are conductive mixed metal oxides. Particular preference is given to indium tin oxide (ITO) or indium zinc oxide (IZO). Preference is further given to conductive doped organic materials, especially conductive doped polymers. In addition, the anode may also consist of two or more layers, for example of an inner layer of ITO and an outer layer of a metal oxide, preferably tungsten oxide, molybdenum oxide or vanadium oxide.

The device is structured appropriately (according to the application), contact-connected and finally sealed, in order to rule out damaging effects of water and air.

In a preferred embodiment, the electronic device is characterized in that one or more layers are coated by a sublimation process. In this case, the materials are applied by vapor deposition in vacuum sublimation systems at an initial pressure of less than 10⁻⁵ mbar, preferably less than 10⁻⁶ mbar. In this case, however, it is also possible that the initial pressure is even lower, for example less than 10⁻⁷ mbar.

Preference is likewise given to an electronic device, characterized in that one or more layers are coated by the OVPD (organic vapor phase deposition) method or with the aid of a carrier gas sublimation. In this case, the materials are applied at a pressure between 10⁻⁵ mbar and 1 bar. A special case of this method is the OVJP (organic vapor jet printing) method, in which the materials are applied directly by a nozzle and thus structured (for example M. S. Arnold et al., Appl. Phys. Lett. 2008, 92, 053301).

Preference is additionally given to an electronic device, characterized in that one or more layers are produced from solution, for example by spin-coating, or by any printing method, for example screen printing, flexographic printing, nozzle printing or offset printing, but more preferably LITI (light-induced thermal imaging, thermal transfer printing) or inkjet printing. For this purpose, soluble compounds are needed. High solubility can be achieved by suitable substitution of the compounds.

It is further preferable that an electronic device of the invention is produced by applying one or more layers from solution and one or more layers by a sublimation method.

Electronic devices comprising one or more compounds as defined above are preferably used in displays, as light sources in lighting applications and as light sources in medical and/or cosmetic applications (e.g. light therapy).

### EXAMPLES

### A) Synthesis Examples

### Step a: Synthesis of Intermediates

### Example a: 10-(4-Bromophenyl)benzo[h]quinoline

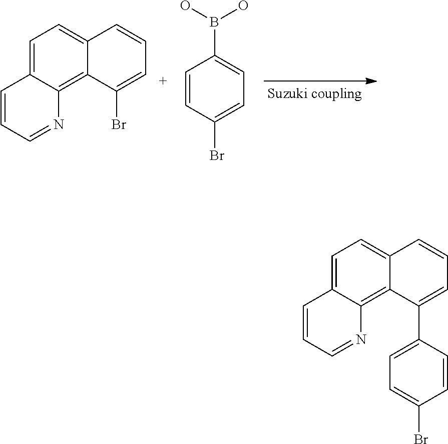

15.47 g (75 mmol) of 4-bromobenzeneboronic acid, 19.4 g of 10-bromobenzo[h]quinoline (75 mmol) and 110 ml of an aqueous 2M NaHCO₃ solution (163 mmol) are suspended in 500 ml of dimethoxyethane. 3.0 g (3.45 mmol) of tetrakis(triphenylphosphine)palladium(0) is added to this suspension, and the reaction mixture is heated under reflux for 22 h. After cooling, the organic phase is removed, filtered through silica gel, washed four times with 400 ml of water and then concentrated to dryness. After filtration of the crude product through silica gel with heptane/toluene (10:1), 39 g (71%) of 10-(4-bromophenyl)benzo[h]quinoline is obtained.

The following compounds are prepared in an analogous manner:

| | Reactant 1 | Reactant 2 | Product | Yield |
|---|---|---|---|---|
| 1a | 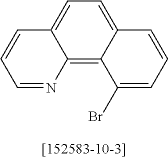 [SMILES: Brc1cccc2ccc3cccnc3c12] | 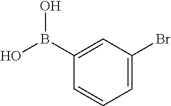 | 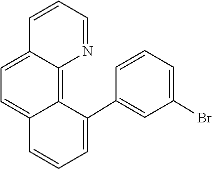 [SMILES: Brc1cccc(-c2cccc3ccc4cccnc4c23)c1] | 58% |
| 2a | 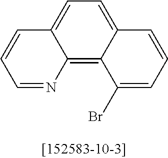 [SMILES: Brc1cccc2ccc3cccnc3c12] | 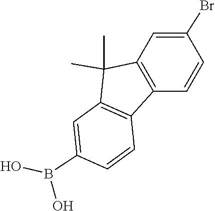 [SMILES: CC1(C)c2cc(Br)ccc2-c2ccc(B(O)O)cc21] | 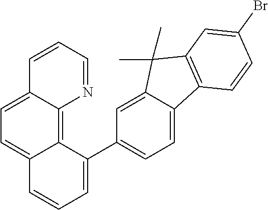 [SMILES: CC1(C)c2cc(Br)ccc2-c2ccc(-c3cccc4ccc5cccnc5c34)cc21] | 49% |
| 3a | 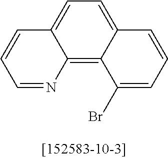 [SMILES: Brc1cccc2ccc3cccnc3c12] | 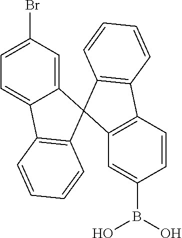 [SMILES: OB(O)c1ccc2c(c1)C1(c3ccccc3-c3ccc(Br)cc31)c1ccccc1-2] | 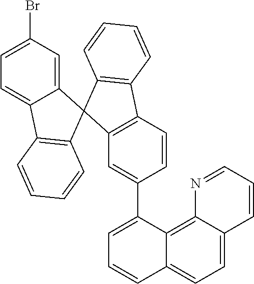 [SMILES: Brc1ccc2c(c1)C1(c3ccccc3-2)c2ccccc2-c2ccc(-c3cccc4ccc5cccnc5c34)cc21] | 51% |
| 4a | 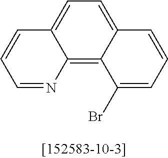 [SMILES: Brc1cccc2ccc3cccnc3c12] | 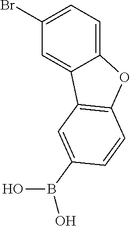 [SMILES: OB(O)c1ccc2oc3ccc(Br)cc3c2c1] | 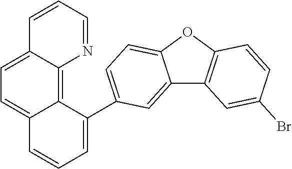 [SMILES: Brc1ccc2oc3ccc(-c4cccc5ccc6cccnc6c45)cc3c2c1] | 56% |
| 5a | 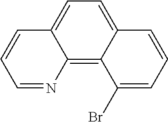 [SMILES: Brc1cccc2ccc3cccnc3c12] | 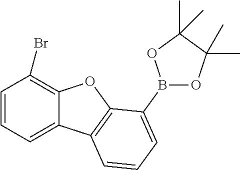 [SMILES: CC1(C)OB(c2cccc3c2oc2c(Br)cccc23)OC1(C)C] | 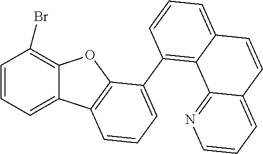 [SMILES: Brc1cccc2c1oc1c(-c3cccc4ccc5cccnc5c34)cccc12] | 43% |
| 6a | 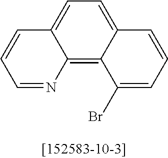 [SMILES: Brc1cccc2ccc3cccnc3c12] | 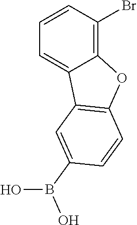 [SMILES: OB(O)c1ccc2oc3c(Br)cccc3c2c1] | 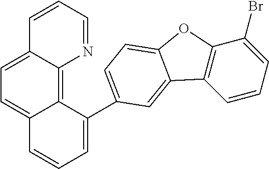 [SMILES: Brc1cccc2c1oc1ccc(-c3cccc4ccc5cccnc5c34)cc12] | 54% |
| 7a | 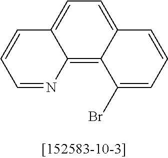 [SMILES: Brc1cccc2ccc3cccnc3c12] | 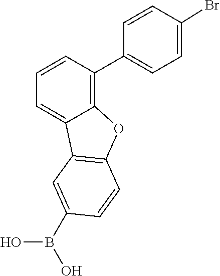 [SMILES: OB(O)c1ccc2oc3c(-c4ccc(Br)cc4)cccc3c2c1] | 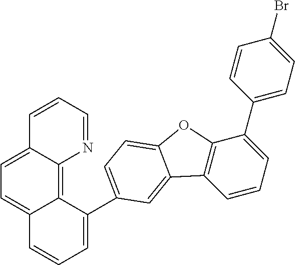 [SMILES: Brc1ccc(-c2cccc3c2oc2ccc(-c4cccc5ccc6cccnc6c45)cc23)cc1] | 49% |
| 8a | 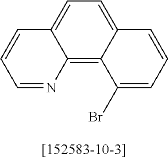 [SMILES: Brc1cccc2ccc3cccnc3c12] | 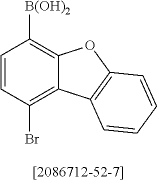 [SMILES: OB(O)c1ccc(Br)c2c1oc1ccccc12] | 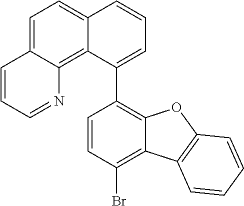 [SMILES: Brc1ccc(-c2cccc3ccc4cccnc4c23)c2oc3ccccc3c12] | 52% |
| 9a | 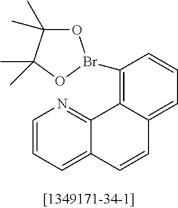 | 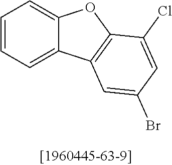 [SMILES: Clc1cc(Br)cc2c1oc1ccccc12] | 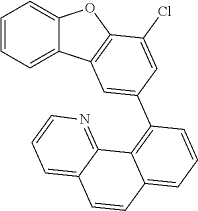 [SMILES: Clc1cc(-c2cccc3ccc4cccnc4c23)cc2c1oc1ccccc12] | 66% |
| 10a | 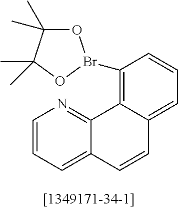 | 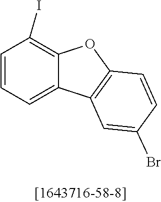 [SMILES: Brc1ccc2oc3c(I)cccc3c2c1] | 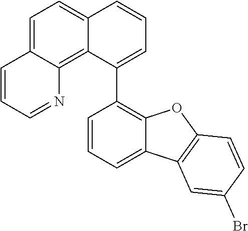 [SMILES: Brc1ccc2oc3c(-c4cccc5ccc6cccnc6c45)cccc3c2c1] | 71% |
| 11a | 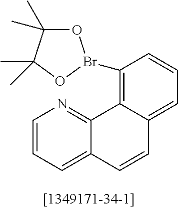 | 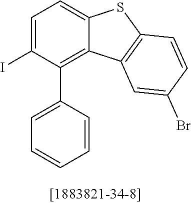 [SMILES: Brc1ccc2sc3ccc(I)c(-c4ccccc4)c3c2c1] | 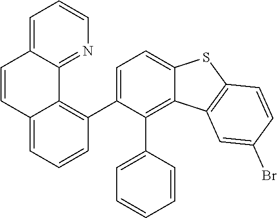 [SMILES: Brc1ccc2sc3ccc(-c4cccc5ccc6cccnc6c45)c(-c4ccccc4)c3c2c1] | 68% |
| 12a | 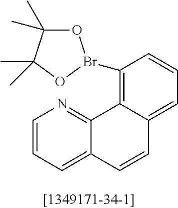 | 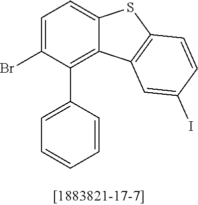 [SMILES: Brc1ccc2sc3ccc(I)cc3c2c1-c1ccccc1] | 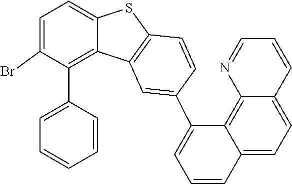 [SMILES: Brc1ccc2sc3ccc(-c4cccc5ccc6cccnc6c45)cc3c2c1-c1ccccc1] | 75% |
| 13a | 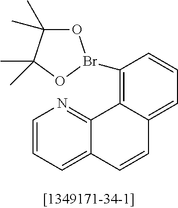 | 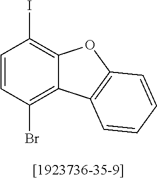 [SMILES: Brc1ccc(I)c2oc3ccccc3c12] | 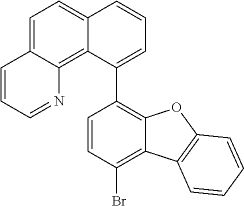 [SMILES: Brc1ccc(-c2cccc3ccc4cccnc4c23)c2oc3ccccc3c12] | 70% |

### Step b: Synthesis of Compounds of the Invention

### Example b: 10-(4-Dibenzothiophen-4-ylphenyl)benzo[h]quinoline

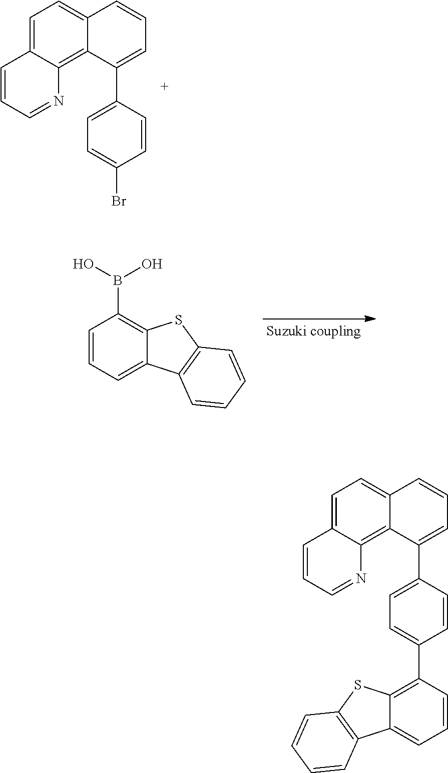 [SMILES: Brc1ccc(-c2cccc3ccc4cccnc4c23)cc1 ; OB(O)c1cccc2c1sc1ccccc12 ; c1cnc2c(c1)ccc1cccc(-c3ccc(-c4cccc5c4sc4ccccc45)cc3)c12]

The following compounds are prepared under conditions analogous to those in example 1:

| | Reactant 1 | Reactant 2 | Product | Yield |
|---|---|---|---|---|
| 1b | 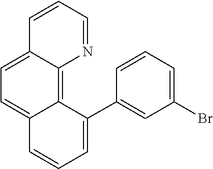 [SMILES: Brc1cccc(-c2cccc3ccc4cccnc4c23)c1] | 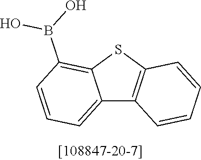 [SMILES: OB(O)c1cccc2c1sc1ccccc12] | 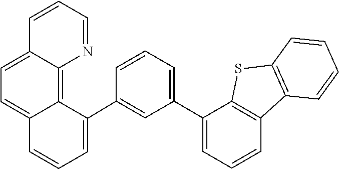 [SMILES: c1cc(-c2cccc3c2sc2ccccc23)cc(-c2cccc3ccc4cccnc4c23)c1] | 67% |
| 2b | 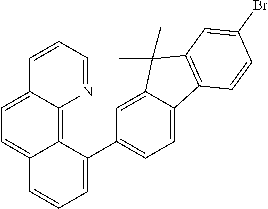 [SMILES: CC1(C)c2cc(Br)ccc2-c2ccc(-c3cccc4ccc5cccnc5c34)cc21] | 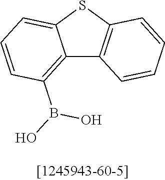 [SMILES: OB(O)c1cccc2sc3ccccc3c12] | 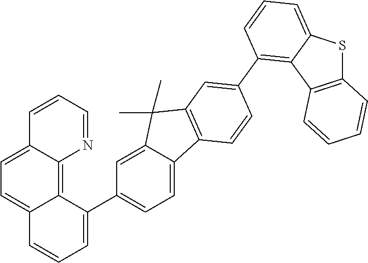 [SMILES: CC1(C)c2cc(-c3cccc4sc5ccccc5c34)ccc2-c2ccc(-c3cccc4ccc5cccnc5c34)cc21] | 68% |
| 3b | 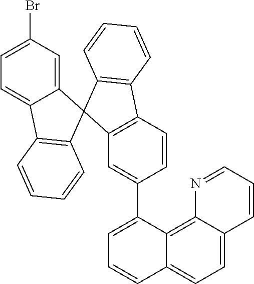 [SMILES: Brc1ccc2c(c1)C1(c3ccccc3-2)c2ccccc2-c2ccc(-c3cccc4ccc5cccnc5c34)cc21] | 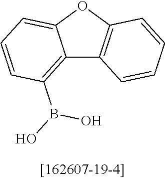 [SMILES: OB(O)c1cccc2oc3ccccc3c12] | 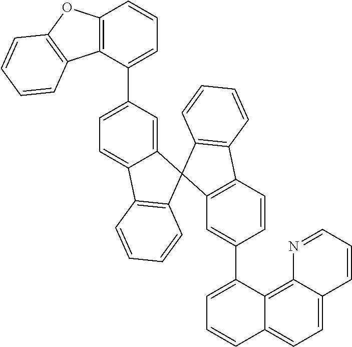 [SMILES: c1ccc2c(c1)-c1ccc(-c3cccc4oc5ccccc5c34)cc1C21c2ccccc2-c2ccc(-c3cccc4ccc5cccnc5c34)cc21] | 60% |
| 4b | 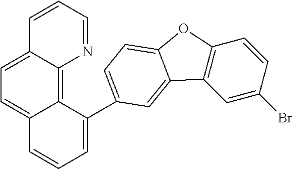 [SMILES: Brc1ccc2oc3ccc(-c4cccc5ccc6cccnc6c45)cc3c2c1] | 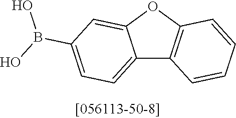 [SMILES: OB(O)c1ccc2c(c1)oc1ccccc12] | 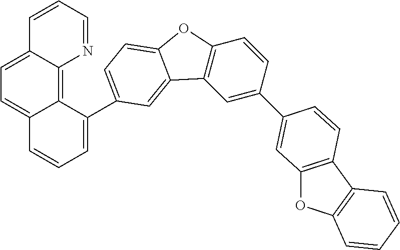 [SMILES: c1cnc2c(c1)ccc1cccc(-c3ccc4oc5ccc(-c6ccc7c(c6)oc6ccccc67)cc5c4c3)c12] | 72% |
| 5b | 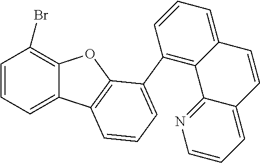 [SMILES: Brc1cccc2c1oc1c(-c3cccc4ccc5cccnc5c34)cccc12] | 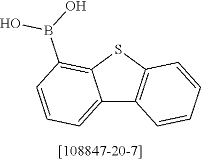 [SMILES: OB(O)c1cccc2c1sc1ccccc12] | 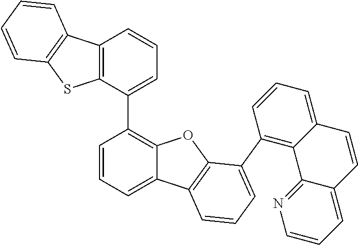 [SMILES: c1cnc2c(c1)ccc1cccc(-c3cccc4c3oc3c(-c5cccc6c5sc5ccccc56)cccc34)c12] | 65% |
| 6b | 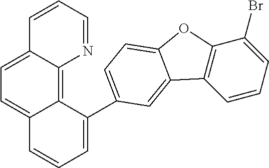 [SMILES: Brc1cccc2c1oc1ccc(-c3cccc4ccc5cccnc5c34)cc12] | 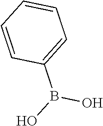 | 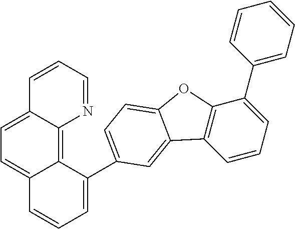 [SMILES: c1ccc(-c2cccc3c2oc2ccc(-c4cccc5ccc6cccnc6c45)cc23)cc1] | 74% |
| 7b | 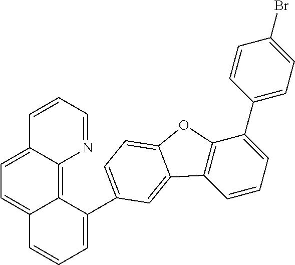 [SMILES: Brc1ccc(-c2cccc3c2oc2ccc(-c4cccc5ccc6cccnc6c45)cc23)cc1] | 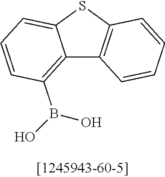 [SMILES: OB(O)c1cccc2sc3ccccc3c12] | 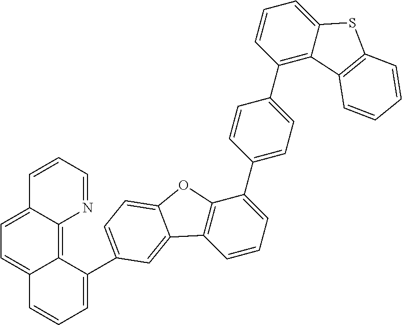 [SMILES: c1cnc2c(c1)ccc1cccc(-c3ccc4oc5c(-c6ccc(-c7cccc8sc9ccccc9c78)cc6)cccc5c4c3)c12] | 73% |
| 8b | 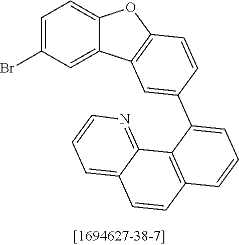 [SMILES: Brc1ccc2oc3ccc(-c4cccc5ccc6cccnc6c45)cc3c2c1] | 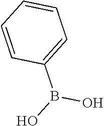 | 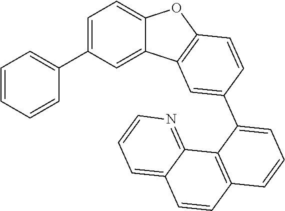 [SMILES: c1ccc(-c2ccc3oc4ccc(-c5cccc6ccc7cccnc7c56)cc4c3c2)cc1] | 80% |
| 9b | 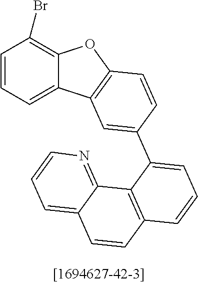 [SMILES: Brc1cccc2c1oc1ccc(-c3cccc4ccc5cccnc5c34)cc12] | 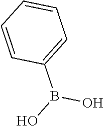 | 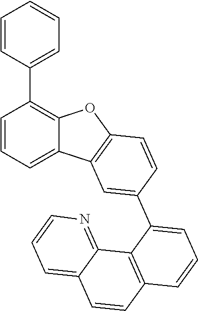 [SMILES: c1ccc(-c2cccc3c2oc2ccc(-c4cccc5ccc6cccnc6c45)cc23)cc1] | 82% |
| 10b | 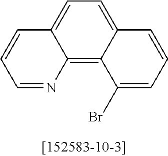 [SMILES: Brc1cccc2ccc3cccnc3c12] | 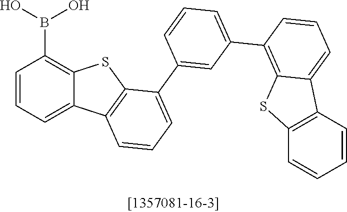 [SMILES: OB(O)c1cccc2c1sc1c(-c3cccc(-c4cccc5c4sc4ccccc45)c3)cccc12] | 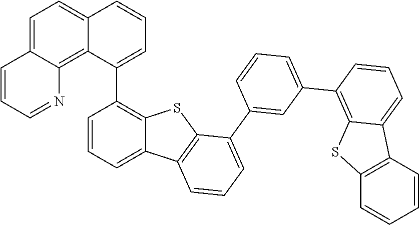 [SMILES: c1cc(-c2cccc3c2sc2ccccc23)cc(-c2cccc3c2sc2c(-c4cccc5ccc6cccnc6c45)cccc23)c1] | 73% |
| 11b | 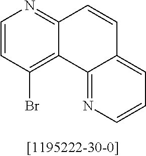 [SMILES: Brc1ccnc2ccc3cccnc3c12] | 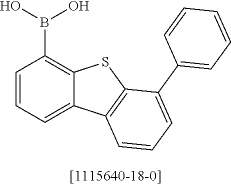 [SMILES: OB(O)c1cccc2c1sc1c(-c3ccccc3)cccc12] | 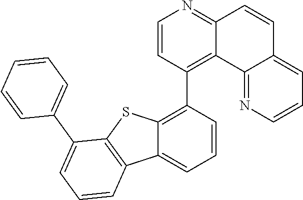 [SMILES: c1ccc(-c2cccc3c2sc2c(-c4ccnc5ccc6cccnc6c45)cccc23)cc1] | 70% |
| 12b | 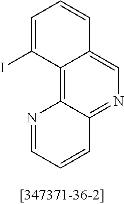 [SMILES: Ic1cccc2cnc3cccnc3c12] | 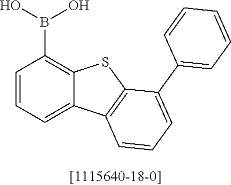 [SMILES: OB(O)c1cccc2c1sc1c(-c3ccccc3)cccc12] | 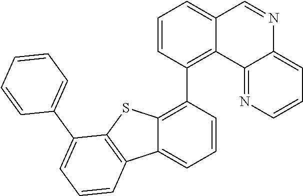 [SMILES: c1ccc(-c2cccc3c2sc2c(-c4cccc5cnc6cccnc6c45)cccc23)cc1] | 72% |
| 13b | 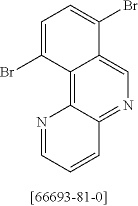 [SMILES: Brc1ccc(Br)c2c1cnc1cccnc12] | 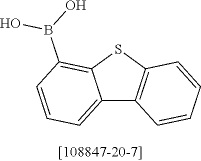 [SMILES: OB(O)c1cccc2c1sc1ccccc12] | 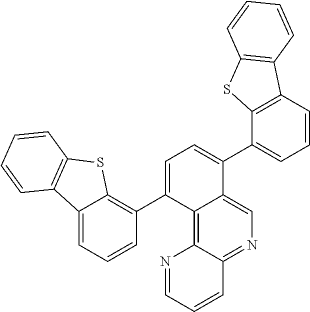 [SMILES: c1cnc2c(c1)ncc1c(-c3cccc4c3sc3ccccc34)ccc(-c3cccc4c3sc3ccccc34)c12] | 69% |
| 14b | 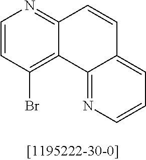 [SMILES: Brc1ccnc2ccc3cccnc3c12] | 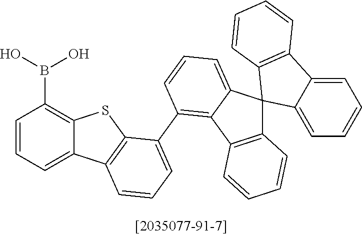 [SMILES: OB(O)c1cccc2c1sc1c(-c3cccc4c3-c3ccccc3C43c4ccccc4-c4ccccc43)cccc12] | 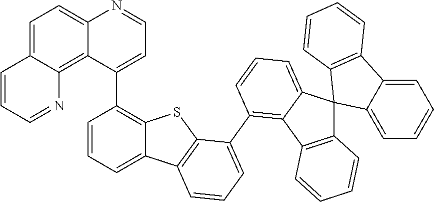 [SMILES: c1ccc2c(c1)-c1ccccc1C21c2ccccc2-c2c(-c3cccc4c3sc3c(-c5ccnc6ccc7cccnc7c56)cccc34)cccc21] | 66% |
| 15b | 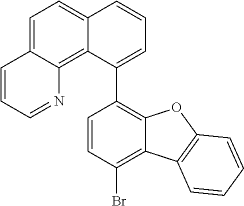 [SMILES: Brc1ccc(-c2cccc3ccc4cccnc4c23)c2oc3ccccc3c12] | 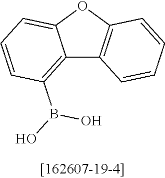 [SMILES: OB(O)c1cccc2oc3ccccc3c12] | 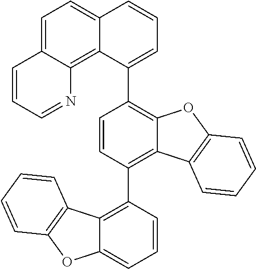 [SMILES: c1cnc2c(c1)ccc1cccc(-c3ccc(-c4cccc5oc6ccccc6c45)c4c3oc3ccccc34)c12] | 75% |
| 16b | 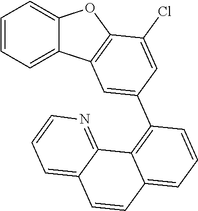 [SMILES: Clc1cc(-c2cccc3ccc4cccnc4c23)cc2c1oc1ccccc12] | 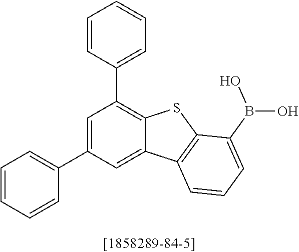 [SMILES: OB(O)c1cccc2c1sc1c(-c3ccccc3)cc(-c3ccccc3)cc12] | 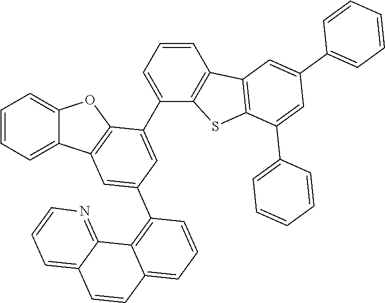 [SMILES: c1ccc(-c2cc(-c3ccccc3)c3sc4c(-c5cc(-c6cccc7ccc8cccnc8c67)cc6c5oc5ccccc56)cccc4c3c2)cc1] | 65% |
| 17b | 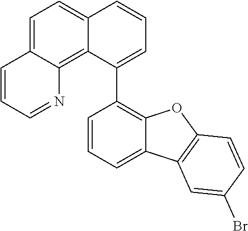 [SMILES: Brc1ccc2oc3c(-c4cccc5ccc6cccnc6c45)cccc3c2c1] | 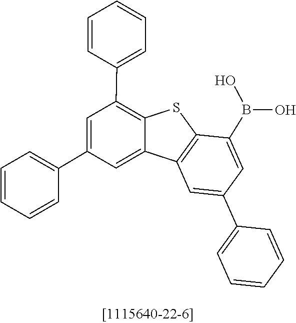 [SMILES: OB(O)c1cc(-c2ccccc2)cc2c1sc1c(-c3ccccc3)cc(-c3ccccc3)cc12] | 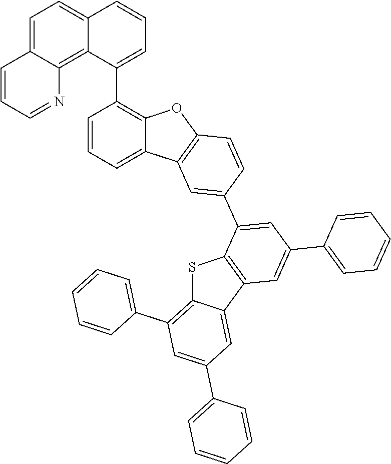 [SMILES: c1ccc(-c2cc(-c3ccccc3)c3sc4c(-c5ccc6oc7c(-c8cccc9ccc%10cccnc%10c89)cccc7c6c5)cc(-c5ccccc5)cc4c3c2)cc1] | 63% |
| 18b | 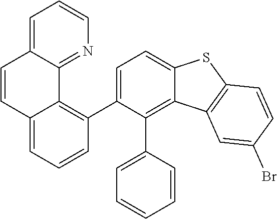 [SMILES: Brc1ccc2sc3ccc(-c4cccc5ccc6cccnc6c45)c(-c4ccccc4)c3c2c1] | 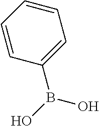 | 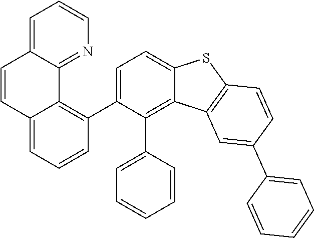 [SMILES: c1ccc(-c2ccc3sc4ccc(-c5cccc6ccc7cccnc7c56)c(-c5ccccc5)c4c3c2)cc1] | 81% |
| 19b | 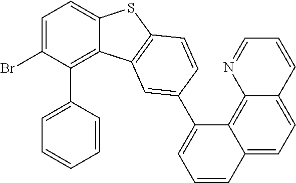 [SMILES: Brc1ccc2sc3ccc(-c4cccc5ccc6cccnc6c45)cc3c2c1-c1ccccc1] | 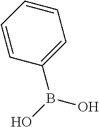 | 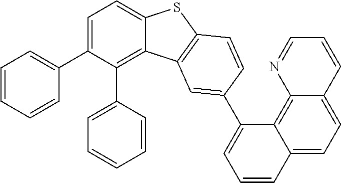 [SMILES: c1ccc(-c2ccc3sc4ccc(-c5cccc6ccc7cccnc7c56)cc4c3c2-c2ccccc2)cc1] | 84% |
| 20b | 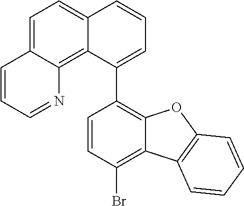 [SMILES: Brc1ccc(-c2cccc3ccc4cccnc4c23)c2oc3ccccc3c12] | 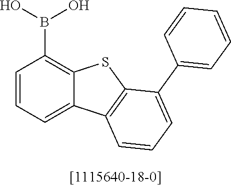 [SMILES: OB(O)c1cccc2c1sc1c(-c3ccccc3)cccc12] | 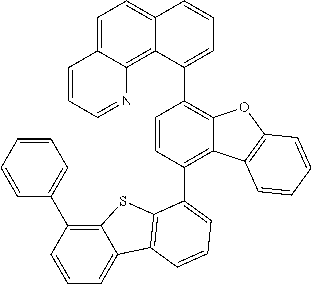 [SMILES: c1ccc(-c2cccc3c2sc2c(-c4ccc(-c5cccc6ccc7cccnc7c56)c5oc6ccccc6c45)cccc23)cc1] | 80% |
| 21b | 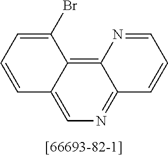 [SMILES: Brc1cccc2cnc3cccnc3c12] | 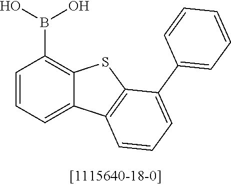 [SMILES: OB(O)c1cccc2c1sc1c(-c3ccccc3)cccc12] | 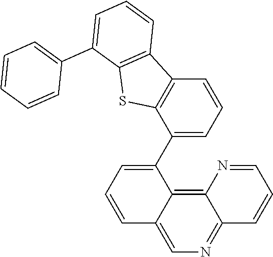 [SMILES: c1ccc(-c2cccc3c2sc2c(-c4cccc5cnc6cccnc6c45)cccc23)cc1] | 78% |
| 22b | 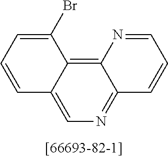 [SMILES: Brc1cccc2cnc3cccnc3c12] | 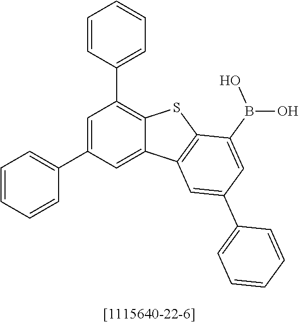 [SMILES: OB(O)c1cc(-c2ccccc2)cc2c1sc1c(-c3ccccc3)cc(-c3ccccc3)cc12] | 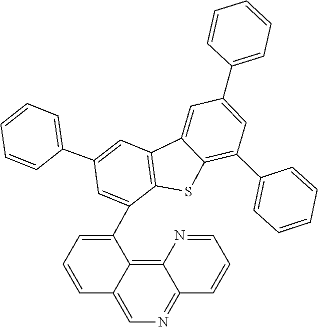 [SMILES: c1ccc(-c2cc(-c3ccccc3)c3sc4c(-c5cccc6cnc7cccnc7c56)cc(-c5ccccc5)cc4c3c2)cc1] | 71% |
| 23b | 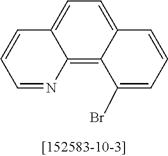 [SMILES: Brc1cccc2ccc3cccnc3c12] | 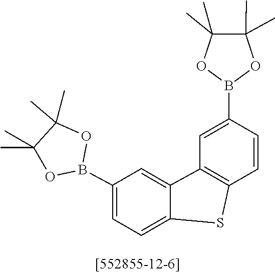 | 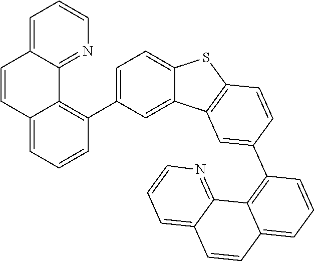 [SMILES: c1cnc2c(c1)ccc1cccc(-c3ccc4sc5ccc(-c6cccc7ccc8cccnc8c67)cc5c4c3)c12] | 82% |
| 24b | 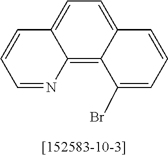 [SMILES: Brc1cccc2ccc3cccnc3c12] | 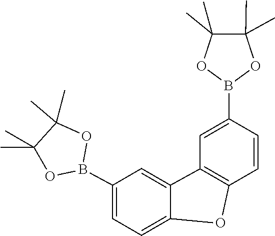 | 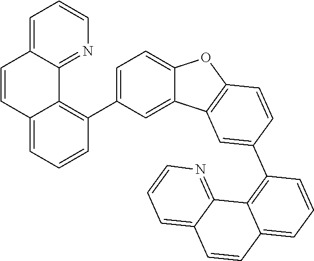 [SMILES: c1cnc2c(c1)ccc1cccc(-c3ccc4oc5ccc(-c6cccc7ccc8cccnc8c67)cc5c4c3)c12] | 80% |

### Step c: Synthesis of Compounds of the Invention

### Example c: 10-(4-Biphenyl-4-yldibenzofuran-1-yl)benzo[h]quinoline

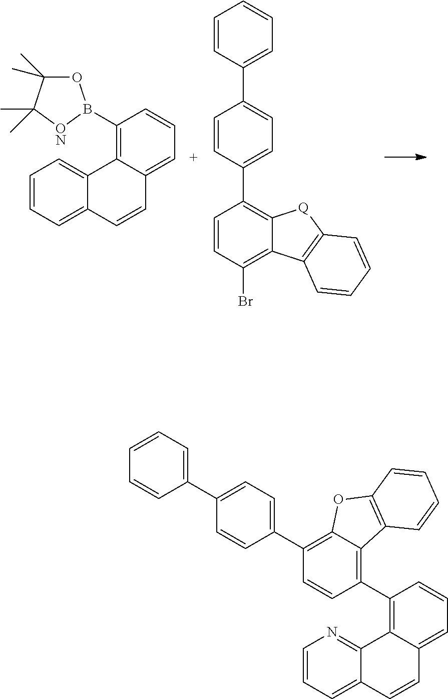

Under conditions analogous to those in example c, the following compounds are prepared proceeding from 10-(4,4,5,5-tetramethyl-[1,3,2]dioxaborolan-2-yl)benzo[h]quinoline:

| | Reactant 1 | Reactant 2 | Product | Yield |
|---|---|---|---|---|
| 1c | 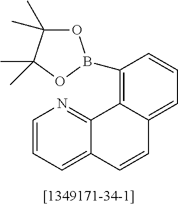 [SMILES: CC1(C)OB(c2cccc3ccc4cccnc4c23)OC1(C)C] | 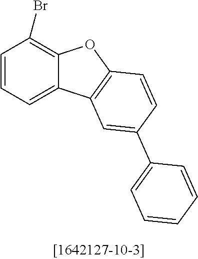 [SMILES: Brc1cccc2c1oc1ccc(-c3ccccc3)cc12] | 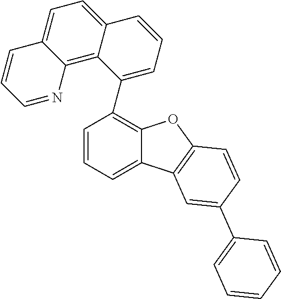 [SMILES: c1ccc(-c2ccc3oc4c(-c5cccc6ccc7cccnc7c56)cccc4c3c2)cc1] | 86% |
| 2c | 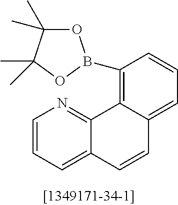 [SMILES: CC1(C)OB(c2cccc3ccc4cccnc4c23)OC1(C)C] | 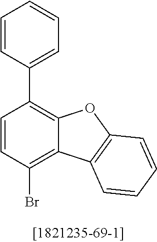 [SMILES: Brc1ccc(-c2ccccc2)c2oc3ccccc3c12] | 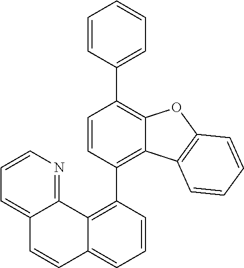 [SMILES: c1ccc(-c2ccc(-c3cccc4ccc5cccnc5c34)c3c2oc2ccccc23)cc1] | 81% |
| 3c | 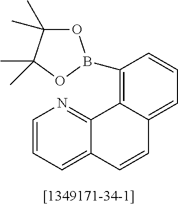 [SMILES: CC1(C)OB(c2cccc3ccc4cccnc4c23)OC1(C)C] | 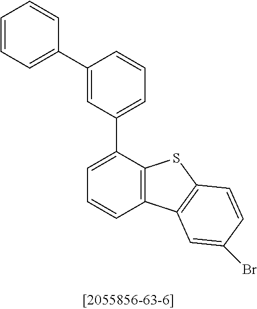 [SMILES: Brc1ccc2sc3c(-c4cccc(-c5ccccc5)c4)cccc3c2c1] | 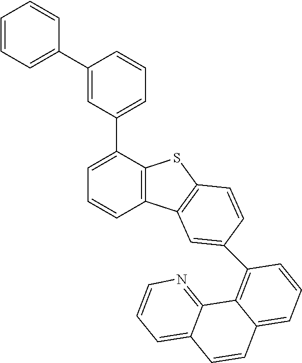 [SMILES: c1ccc(-c2cccc(-c3cccc4c3sc3ccc(-c5cccc6ccc7cccnc7c56)cc34)c2)cc1] | 80% |
| 4c | 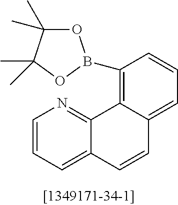 [SMILES: CC1(C)OB(c2cccc3ccc4cccnc4c23)OC1(C)C] | 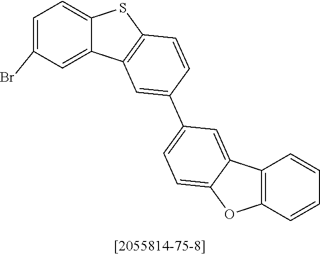 [SMILES: Brc1ccc2sc3ccc(-c4ccc5oc6ccccc6c5c4)cc3c2c1] | 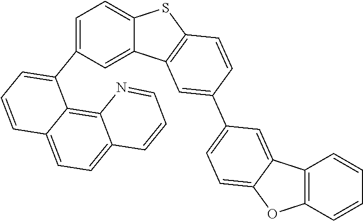 [SMILES: c1cnc2c(c1)ccc1cccc(-c3ccc4sc5ccc(-c6ccc7oc8ccccc8c7c6)cc5c4c3)c12] | 79% |
| 5c | 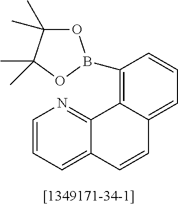 [SMILES: CC1(C)OB(c2cccc3ccc4cccnc4c23)OC1(C)C] | 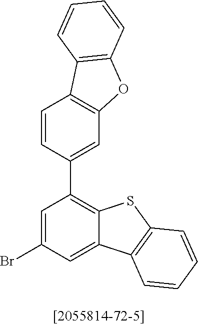 [SMILES: Brc1cc(-c2ccc3c(c2)oc2ccccc23)c2sc3ccccc3c2c1] | 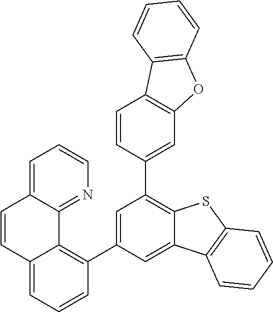 [SMILES: c1cnc2c(c1)ccc1cccc(-c3cc(-c4ccc5c(c4)oc4ccccc45)c4sc5ccccc5c4c3)c12] | 77% |
| 6c | 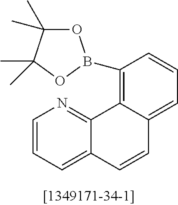 [SMILES: CC1(C)OB(c2cccc3ccc4cccnc4c23)OC1(C)C] | 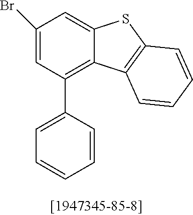 [SMILES: Brc1cc(-c2ccccc2)c2c(c1)sc1ccccc12] | 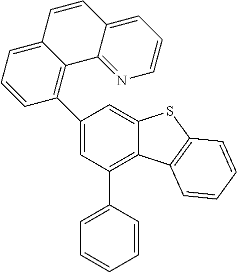 [SMILES: c1ccc(-c2cc(-c3cccc4ccc5cccnc5c34)cc3sc4ccccc4c23)cc1] | 82% |
| 7c | 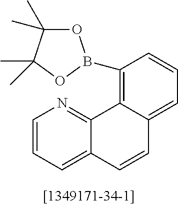 [SMILES: CC1(C)OB(c2cccc3ccc4cccnc4c23)OC1(C)C] | 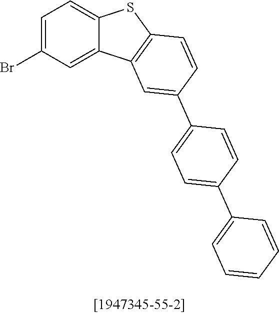 [SMILES: Brc1ccc2sc3ccc(-c4ccc(-c5ccccc5)cc4)cc3c2c1] | 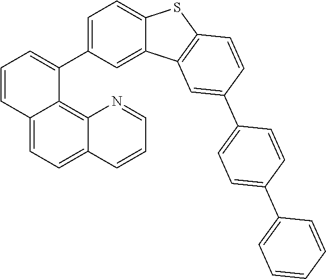 [SMILES: c1ccc(-c2ccc(-c3ccc4sc5ccc(-c6cccc7ccc8cccnc8c67)cc5c4c3)cc2)cc1] | 81% |
| 8c | 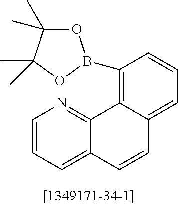 [SMILES: CC1(C)OB(c2cccc3ccc4cccnc4c23)OC1(C)C] | 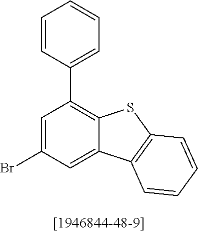 [SMILES: Brc1cc(-c2ccccc2)c2sc3ccccc3c2c1] | 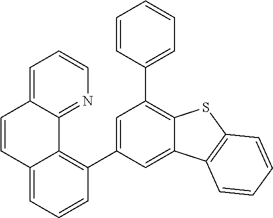 [SMILES: c1ccc(-c2cc(-c3cccc4ccc5cccnc5c34)cc3c2sc2ccccc23)cc1] | 60% |
| 9c | 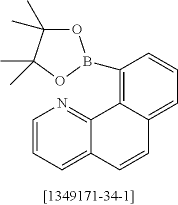 [SMILES: CC1(C)OB(c2cccc3ccc4cccnc4c23)OC1(C)C] | 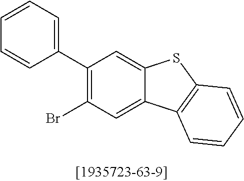 [SMILES: Brc1cc2c(cc1-c1ccccc1)sc1ccccc12] | 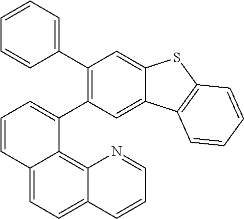 [SMILES: c1ccc(-c2cc3sc4ccccc4c3cc2-c2cccc3ccc4cccnc4c23)cc1] | 62% |
| 10c | 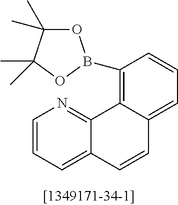 [SMILES: CC1(C)OB(c2cccc3ccc4cccnc4c23)OC1(C)C] | 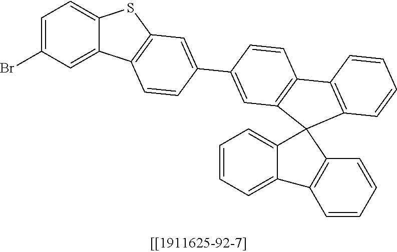 [SMILES: Brc1ccc2sc3cc(-c4ccc5c(c4)C4(c6ccccc6-c6ccccc64)c4ccccc4-5)ccc3c2c1] | 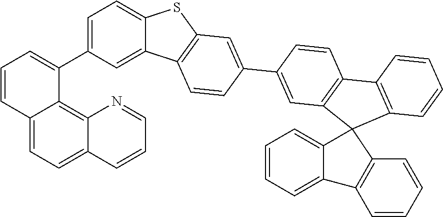 [SMILES: c1ccc2c(c1)-c1ccccc1C21c2ccccc2-c2ccc(-c3ccc4c(c3)sc3ccc(-c5cccc6ccc7cccnc7c56)cc34)cc21] | 66% |
| 11c | 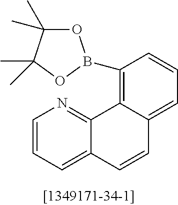 [SMILES: CC1(C)OB(c2cccc3ccc4cccnc4c23)OC1(C)C] | 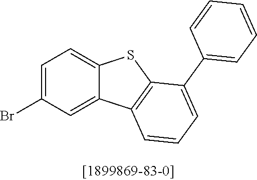 [SMILES: Brc1ccc2sc3c(-c4ccccc4)cccc3c2c1] | 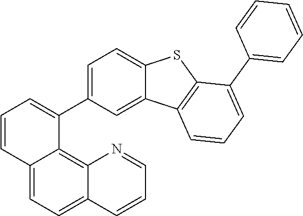 [SMILES: c1ccc(-c2cccc3c2sc2ccc(-c4cccc5ccc6cccnc6c45)cc23)cc1] | 78% |
| 12c | 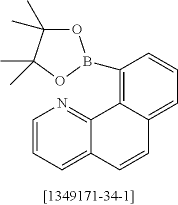 [SMILES: CC1(C)OB(c2cccc3ccc4cccnc4c23)OC1(C)C] | 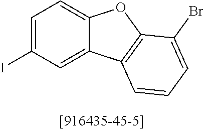 [SMILES: Brc1cccc2c1oc1ccc(I)cc12] | 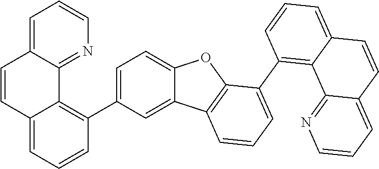 [SMILES: c1cnc2c(c1)ccc1cccc(-c3ccc4oc5c(-c6cccc7ccc8cccnc8c67)cccc5c4c3)c12] | 65% |
| 13c | 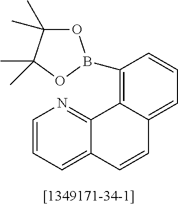 [SMILES: CC1(C)OB(c2cccc3ccc4cccnc4c23)OC1(C)C] | 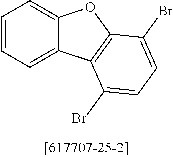 [SMILES: Brc1ccc(Br)c2c1oc1ccccc12] | 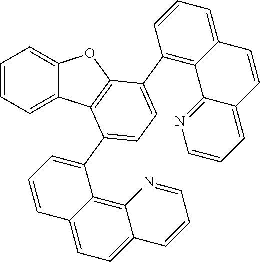 [SMILES: c1cnc2c(c1)ccc1cccc(-c3ccc(-c4cccc5ccc6cccnc6c45)c4c3oc3ccccc34)c12] | 69% |
| 14c | 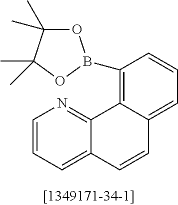 [SMILES: CC1(C)OB(c2cccc3ccc4cccnc4c23)OC1(C)C] | 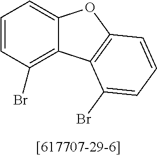 [SMILES: Brc1cccc2oc3cccc(Br)c3c12] | 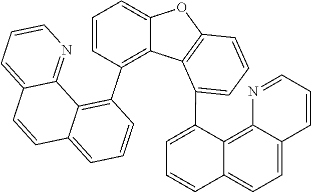 [SMILES: c1cnc2c(c1)ccc1cccc(-c3cccc4oc5cccc(-c6cccc7ccc8cccnc8c67)c5c34)c12] | 54% |
| 15c | 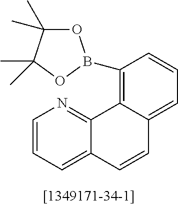 [SMILES: CC1(C)OB(c2cccc3ccc4cccnc4c23)OC1(C)C] | 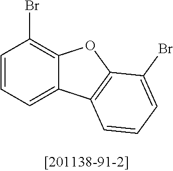 [SMILES: Brc1cccc2c1oc1c(Br)cccc12] | 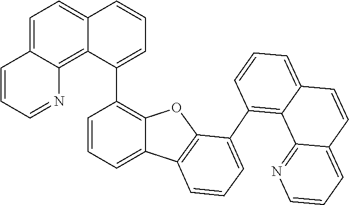 [SMILES: c1cnc2c(c1)ccc1cccc(-c3cccc4c3oc3c(-c5cccc6ccc7cccnc7c56)cccc34)c12] | 87% |
| 16c | 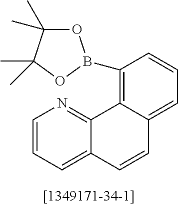 [SMILES: CC1(C)OB(c2cccc3ccc4cccnc4c23)OC1(C)C] | 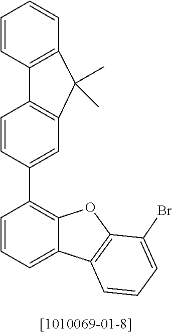 [SMILES: CC1(C)c2ccccc2-c2ccc(-c3cccc4c3oc3c(Br)cccc34)cc21] | 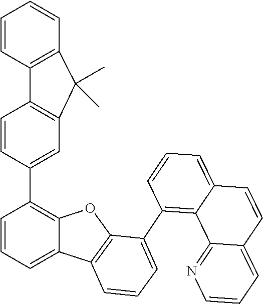 [SMILES: CC1(C)c2ccccc2-c2ccc(-c3cccc4c3oc3c(-c5cccc6ccc7cccnc7c56)cccc34)cc21] | 82% |
| 17c | 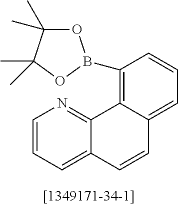 [SMILES: CC1(C)OB(c2cccc3ccc4cccnc4c23)OC1(C)C] | 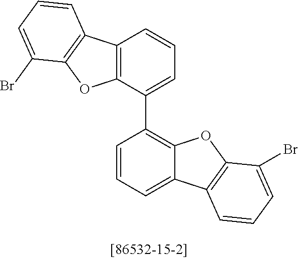 [SMILES: Brc1cccc2c1oc1c(-c3cccc4c3oc3c(Br)cccc34)cccc12] | 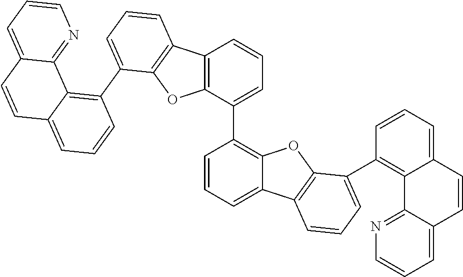 [SMILES: c1cnc2c(c1)ccc1cccc(-c3cccc4c3oc3c(-c5cccc6c5oc5c(-c7cccc8ccc9cccnc9c78)cccc56)cccc34)c12] | 75% |
| 18c | 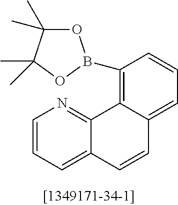 [SMILES: CC1(C)OB(c2cccc3ccc4cccnc4c23)OC1(C)C] | 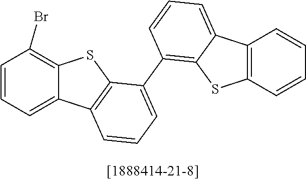 [SMILES: Brc1cccc2c1sc1c(-c3cccc4c3sc3ccccc34)cccc12] | 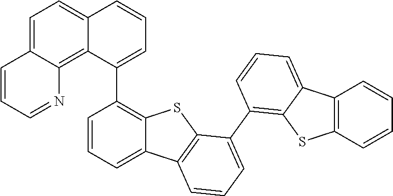 [SMILES: c1cnc2c(c1)ccc1cccc(-c3cccc4c3sc3c(-c5cccc6c5sc5ccccc56)cccc34)c12] | 80% |
| 19c | 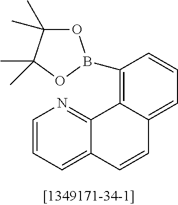 [SMILES: CC1(C)OB(c2cccc3ccc4cccnc4c23)OC1(C)C] | 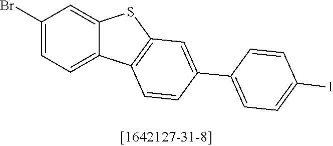 [SMILES: Brc1ccc2c(c1)sc1cc(-c3ccc(I)cc3)ccc12] | 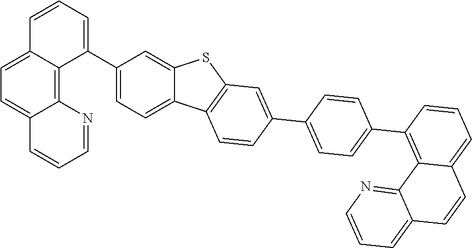 [SMILES: c1cnc2c(c1)ccc1cccc(-c3ccc(-c4ccc5c(c4)sc4cc(-c6cccc7ccc8cccnc8c67)ccc45)cc3)c12] | 78% |
| 20c | 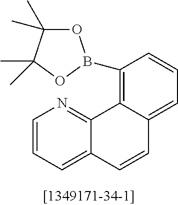 [SMILES: CC1(C)OB(c2cccc3ccc4cccnc4c23)OC1(C)C] | 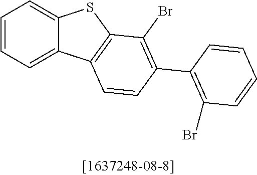 [SMILES: Brc1ccccc1-c1ccc2c(sc3ccccc32)c1Br] | 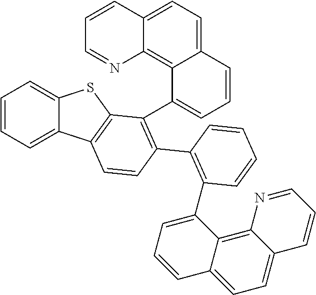 [SMILES: c1ccc(-c2cccc3ccc4cccnc4c23)c(-c2ccc3c(sc4ccccc43)c2-c2cccc3ccc4cccnc4c23)c1] | 45% |
| 21c | 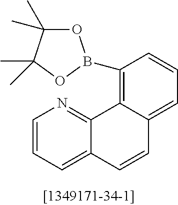 [SMILES: CC1(C)OB(c2cccc3ccc4cccnc4c23)OC1(C)C] | 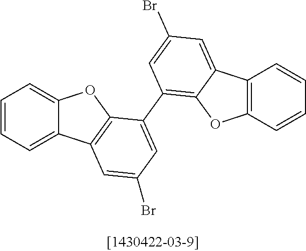 [SMILES: Brc1cc(-c2cc(Br)cc3c2oc2ccccc23)c2oc3ccccc3c2c1] | 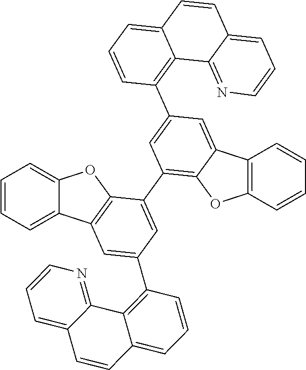 [SMILES: c1cnc2c(c1)ccc1cccc(-c3cc(-c4cc(-c5cccc6ccc7cccnc7c56)cc5c4oc4ccccc45)c4oc5ccccc5c4c3)c12] | 62% |
| 22c | 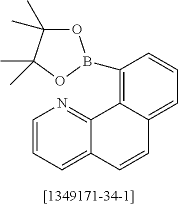 [SMILES: CC1(C)OB(c2cccc3ccc4cccnc4c23)OC1(C)C] | 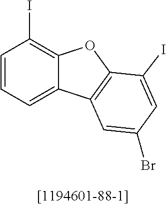 [SMILES: Brc1cc(I)c2oc3c(I)cccc3c2c1] | 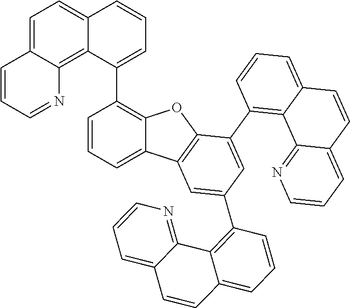 [SMILES: c1cnc2c(c1)ccc1cccc(-c3cc(-c4cccc5ccc6cccnc6c45)c4oc5c(-c6cccc7ccc8cccnc8c67)cccc5c4c3)c12] | 46% |
| 23c | 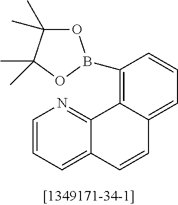 [SMILES: CC1(C)OB(c2cccc3ccc4cccnc4c23)OC1(C)C] | 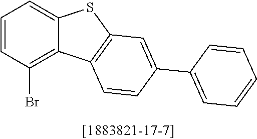 [SMILES: Brc1cccc2sc3cc(-c4ccccc4)ccc3c12] | 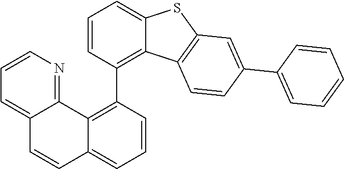 [SMILES: c1ccc(-c2ccc3c(c2)sc2cccc(-c4cccc5ccc6cccnc6c45)c23)cc1] | 82% |
| 24c | 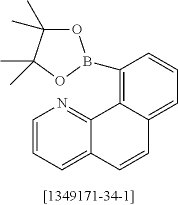 [SMILES: CC1(C)OB(c2cccc3ccc4cccnc4c23)OC1(C)C] | 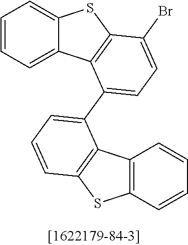 [SMILES: Brc1ccc(-c2cccc3sc4ccccc4c23)c2c1sc1ccccc12] | 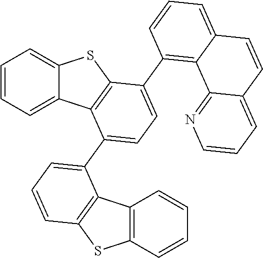 [SMILES: c1cnc2c(c1)ccc1cccc(-c3ccc(-c4cccc5sc6ccccc6c45)c4c3sc3ccccc34)c12] | 76% |
| 25c | 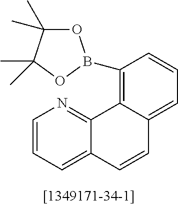 [SMILES: CC1(C)OB(c2cccc3ccc4cccnc4c23)OC1(C)C] | 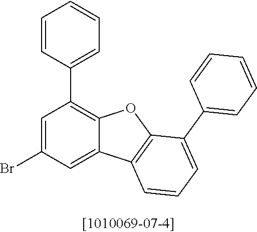 [SMILES: Brc1cc(-c2ccccc2)c2oc3c(-c4ccccc4)cccc3c2c1] | 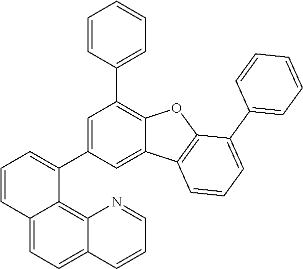 [SMILES: c1ccc(-c2cccc3c2oc2c(-c4ccccc4)cc(-c4cccc5ccc6cccnc6c45)cc23)cc1] | 73% |
| 26c | 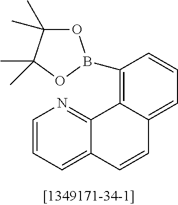 [SMILES: CC1(C)OB(c2cccc3ccc4cccnc4c23)OC1(C)C] | 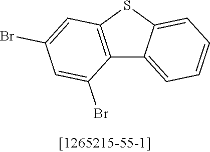 [SMILES: Brc1cc(Br)c2c(c1)sc1ccccc12] | 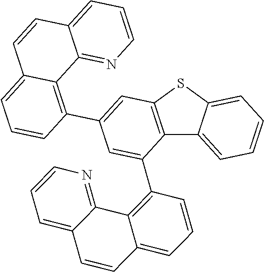 [SMILES: c1cnc2c(c1)ccc1cccc(-c3cc(-c4cccc5ccc6cccnc6c45)c4c(c3)sc3ccccc34)c12] | 60% |
| 27c | 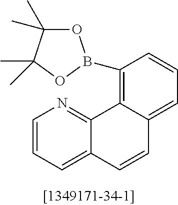 [SMILES: CC1(C)OB(c2cccc3ccc4cccnc4c23)OC1(C)C] | 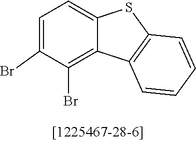 [SMILES: Brc1ccc2sc3ccccc3c2c1Br] | 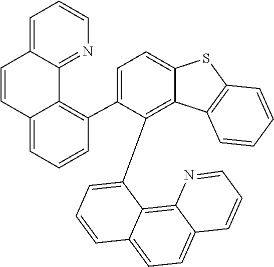 [SMILES: c1cnc2c(c1)ccc1cccc(-c3ccc4sc5ccccc5c4c3-c3cccc4ccc5cccnc5c34)c12] | 64% |
| 28c | 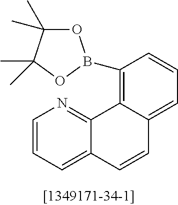 [SMILES: CC1(C)OB(c2cccc3ccc4cccnc4c23)OC1(C)C] | 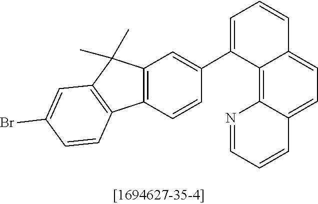 [SMILES: CC1(C)c2cc(Br)ccc2-c2ccc(-c3cccc4ccc5cccnc5c34)cc21] | 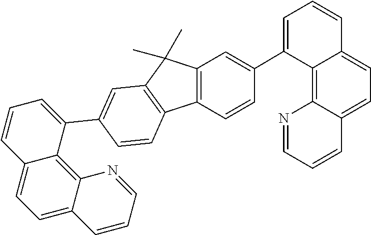 [SMILES: CC1(C)c2cc(-c3cccc4ccc5cccnc5c34)ccc2-c2ccc(-c3cccc4ccc5cccnc5c34)cc21] | 83% |
| 29c | 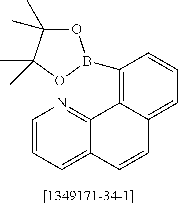 [SMILES: CC1(C)OB(c2cccc3ccc4cccnc4c23)OC1(C)C] | 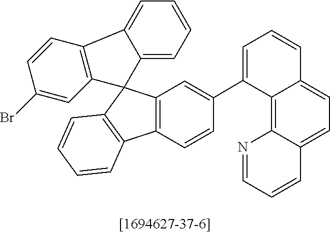 [SMILES: Brc1ccc2c(c1)C1(c3ccccc3-2)c2ccccc2-c2ccc(-c3cccc4ccc5cccnc5c34)cc21] | 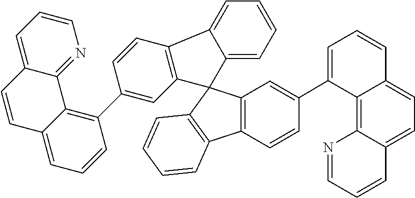 [SMILES: c1ccc2c(c1)-c1ccc(-c3cccc4ccc5cccnc5c34)cc1C21c2ccccc2-c2ccc(-c3cccc4ccc5cccnc5c34)cc21] | 80% |
| 30c | 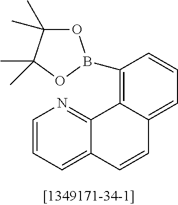 [SMILES: CC1(C)OB(c2cccc3ccc4cccnc4c23)OC1(C)C] | 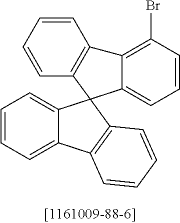 [SMILES: Brc1cccc2c1-c1ccccc1C21c2ccccc2-c2ccccc21] | 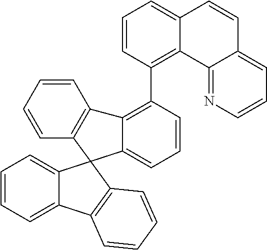 [SMILES: c1ccc2c(c1)-c1ccccc1C21c2ccccc2-c2c(-c3cccc4ccc5cccnc5c34)cccc21] | 86% |
| 31c | 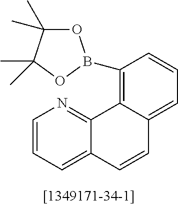 [SMILES: CC1(C)OB(c2cccc3ccc4cccnc4c23)OC1(C)C] | 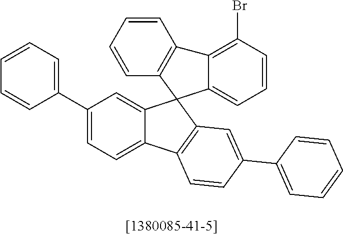 [SMILES: Brc1cccc2c1-c1ccccc1C21c2cc(-c3ccccc3)ccc2-c2ccc(-c3ccccc3)cc21] | 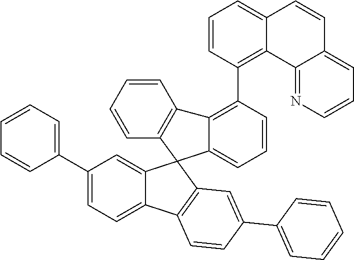 [SMILES: c1ccc(-c2ccc3c(c2)C2(c4cc(-c5ccccc5)ccc4-3)c3ccccc3-c3c(-c4cccc5ccc6cccnc6c45)cccc32)cc1] | 87% |
| 32c | 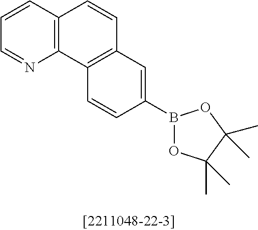 [SMILES: CC1(C)OB(c2ccc3c(ccc4cccnc43)c2)OC1(C)C] | 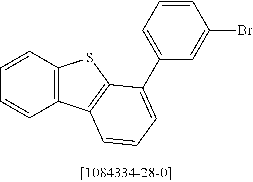 [SMILES: Brc1cccc(-c2cccc3c2sc2ccccc23)c1] | 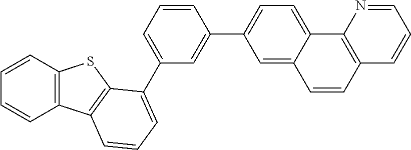 [SMILES: c1cc(-c2ccc3c(ccc4cccnc43)c2)cc(-c2cccc3c2sc2ccccc23)c1] | 80% |
| 33c | 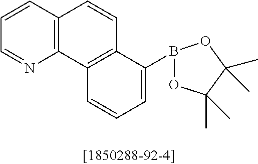 [SMILES: CC1(C)OB(c2cccc3c2ccc2cccnc23)OC1(C)C] | 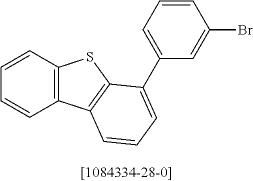 [SMILES: Brc1cccc(-c2cccc3c2sc2ccccc23)c1] | 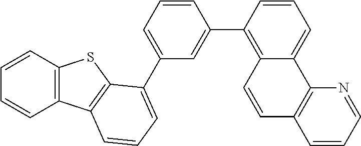 [SMILES: c1cc(-c2cccc3c2ccc2cccnc23)cc(-c2cccc3c2sc2ccccc23)c1] | 79% |
| 34c | 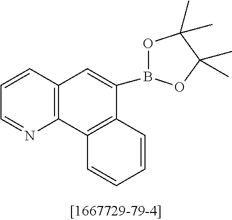 [SMILES: CC1(C)OB(c2cc3cccnc3c3ccccc23)OC1(C)C] | 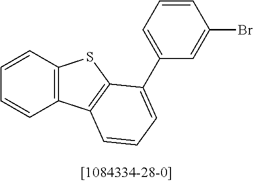 [SMILES: Brc1cccc(-c2cccc3c2sc2ccccc23)c1] | 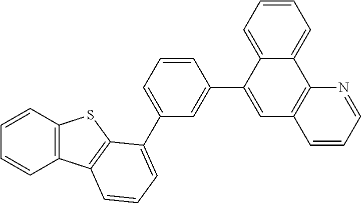 [SMILES: c1cc(-c2cc3cccnc3c3ccccc23)cc(-c2cccc3c2sc2ccccc23)c1] | 83% |
| 35c | 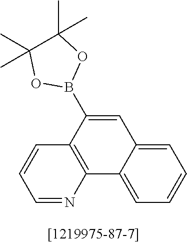 [SMILES: CC1(C)OB(c2cc3ccccc3c3ncccc23)OC1(C)C] | 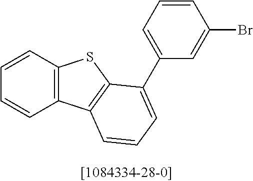 [SMILES: Brc1cccc(-c2cccc3c2sc2ccccc23)c1] | 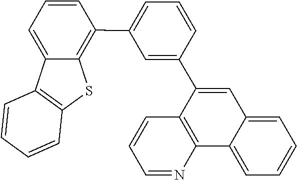 [SMILES: c1cc(-c2cc3ccccc3c3ncccc23)cc(-c2cccc3c2sc2ccccc23)c1] | 67% |
| 36c | 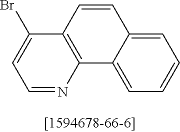 [SMILES: Brc1ccnc2c1ccc1ccccc12] | 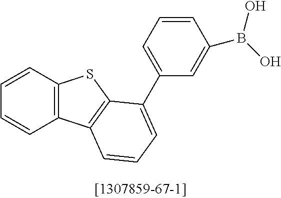 [SMILES: OB(O)c1cccc(-c2cccc3c2sc2ccccc23)c1] | 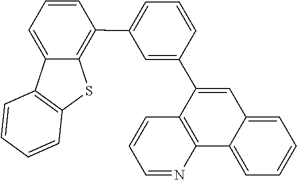 [SMILES: c1cc(-c2cc3ccccc3c3ncccc23)cc(-c2cccc3c2sc2ccccc23)c1] | 75% |
| 37c | 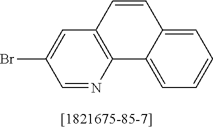 [SMILES: Brc1cnc2c(ccc3ccccc32)c1] | 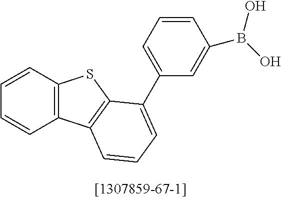 [SMILES: OB(O)c1cccc(-c2cccc3c2sc2ccccc23)c1] | 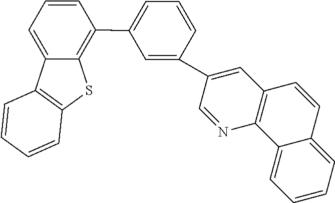 [SMILES: c1cc(-c2cnc3c(ccc4ccccc43)c2)cc(-c2cccc3c2sc2ccccc23)c1] | 79% |
| 38c | 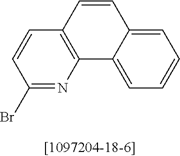 [SMILES: Brc1ccc2ccc3ccccc3c2n1] | 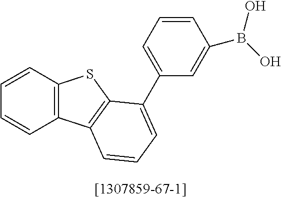 [SMILES: OB(O)c1cccc(-c2cccc3c2sc2ccccc23)c1] | 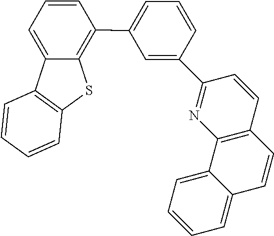 [SMILES: c1cc(-c2ccc3ccc4ccccc4c3n2)cc(-c2cccc3c2sc2ccccc23)c1] | 80% |
| 39c | 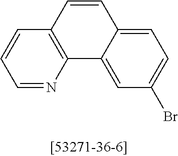 [SMILES: Brc1ccc2ccc3cccnc3c2c1] | 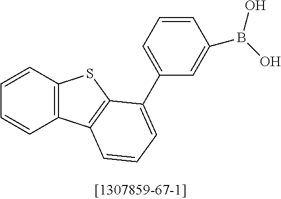 [SMILES: OB(O)c1cccc(-c2cccc3c2sc2ccccc23)c1] | 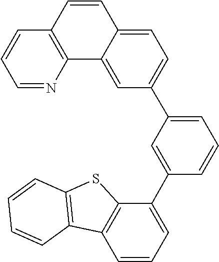 [SMILES: c1cc(-c2ccc3ccc4cccnc4c3c2)cc(-c2cccc3c2sc2ccccc23)c1] | 83% |
| 40c | 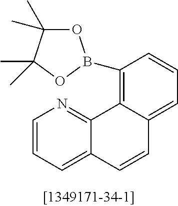 [SMILES: CC1(C)OB(c2cccc3ccc4cccnc4c23)OC1(C)C] | 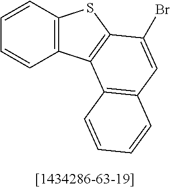 [SMILES: Brc1cc2ccccc2c2c1sc1ccccc12] | 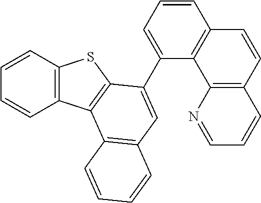 [SMILES: c1ccc2c(c1)cc(-c1cccc3ccc4cccnc4c13)c1sc3ccccc3c12] | 82% |

### B) Production and Characterization of OLEDs

Glass plaques coated with structured ITO (indium tin oxide) of thickness 50 nm are treated prior to coating with an oxygen plasma, followed by an argon plasma. These plasma-treated glass plaques form the substrates to which the OLEDs are applied.

The OLEDs have the following layer structure: substrate/hole injection layer (HIL)/hole transport layer (HTL)/electron blocker layer (EBL)/emission layer (EML)/hole blocker layer (HBL)/electron transport layer (ETL)/electron injection layer (EIL) and finally a cathode. The cathode is formed by an aluminum layer of thickness 100 nm. The exact structure of the OLEDs can be found in table 1. The materials required for production of the OLEDs are shown in table 2. The data of the OLEDs are listed in table 3.

All materials are applied by thermal vapor deposition in a vacuum chamber. In this case, the emission layer always consists of at least one matrix material (host material) and an emitting dopant (emitter) which is added to the matrix material(s) in a particular proportion by volume by coevaporation. In the case of mixed layers, data in such a form as IC1:1b:TEG1 (40%:40%:10%) mean that the material IC1 is present in the layer in a proportion by volume of 40%, 1b in a proportion by volume of 40%, and TEG1 in a proportion by volume of 10%.

The OLEDs are characterized in a standard manner. For this purpose, the electroluminescence spectra, the operating voltage, the current efficiency (CE, measured in cd/A) and the external quantum efficiency (EQE, measured in %) are determined as a function of luminance, calculated from current-voltage-luminance characteristics assuming Lambertian emission characteristics. The electroluminescence spectra are determined at a luminance of 1000 cd/m², and the CIE 1931 x and y color coordinates are calculated therefrom. The parameter U1000 in table 3 refers to the voltage which is required for a luminance of 1000 cd/m². CE1000 and EQE1000 respectively denote the current efficiency and external quantum efficiency that are attained at 1000 cd/m².

The materials of the invention can be used in green-phosphorescing emission layers of OLEDs, as shown by the following examples:

TABLE 1
Structure of the OLEDs

| | HIL | HTL | EBL | EML | HBL | ETL | EIL |
|---|---|---|---|---|---|---|---|
| Ex. | thickness | thickness | thickness | thickness | thickness | thickness | thickness |
| E1 | HATCN | SpMA1 | SpMA2 | IC1:1b:TEG1 | ST2 | ST2:LiQ (50%:50%) | LiQ 1 nm |
| | 5 nm | 230 nm | 20 nm | (58%:30%:12%) 40 nm | 5 nm | 30 nm | |
| E2 | HATCN | SpMA1 | SpMA2 | IC1:7b:TEG1 | ST2 | ST2:LiQ (50%:50%) | LiQ 1 nm |
| | 5 nm | 230 nm | 20 nm | (58%:30%:12%) 40 nm | 5 nm | 30 nm | |
| E3 | HATCN | SpMA1 | SpMA2 | IC1:11b:TEG1 | ST2 | ST2:LiQ (50%:50%) | LiQ 1 nm |
| | 5 nm | 230 nm | 20 nm | (58%:30%:12%) 40 nm | 5 nm | 30 nm | |
| E4 | HATCN | SpMA1 | SpMA2 | IC1:30c:TEG1 | ST2 | ST2:LiQ (50%:50%) | LiQ 1 nm |
| | 5 nm | 230 nm | 20 nm | (58%:30%:12%) 40 nm | 5 nm | 30 nm | |
| E5 | HATCN | SpMA1 | SpMA2 | IC1:32c:TEG1 | ST2 | ST2:LiQ (50%:50%) | LiQ 1 nm |
| | 5 nm | 230 nm | 20 nm | (58%:30%:12%) 40 nm | 5 nm | 30 nm | |
| E6 | HATCN | SpMA1 | SpMA2 | IC1:34c:TEG1 | ST2 | ST2:LiQ (50%:50%) | LiQ 1 nm |
| | 5 nm | 230 nm | 20 nm | (58%:30%:12%) 40 nm | 5 nm | 30 nm | |

TABLE 2
Materials used

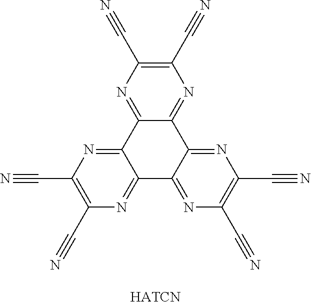

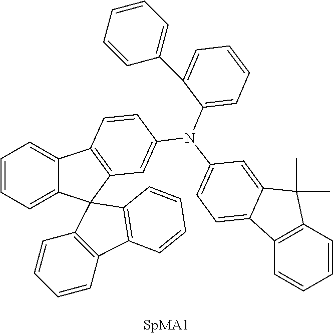 [SMILES: CC1(C)c2ccccc2-c2ccc(N(c3ccc4c(c3)C3(c5ccccc5-c5ccccc53)c3ccccc3-4)c3ccccc3-c3ccccc3)cc21]

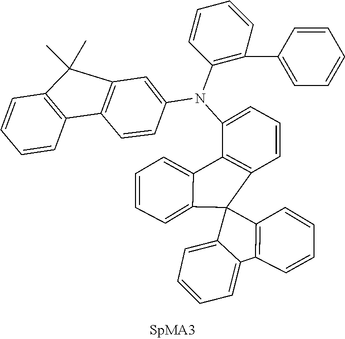 [SMILES: CC1(C)c2ccccc2-c2ccc(N(c3ccccc3-c3ccccc3)c3cccc4c3-c3ccccc3C43c4ccccc4-c4ccccc43)cc21]

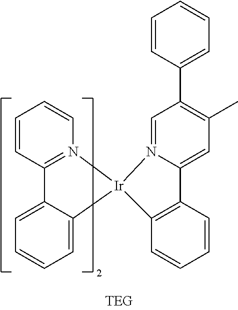

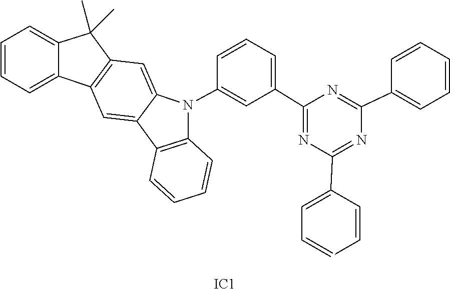 [SMILES: C=C/C=C(\C=C/C)c1nc(-c2ccccc2)nc(-c2cccc(-n3c4ccccc4c4cc5c(cc43)C(C)(C)c3ccccc3-5)c2)n1 ; "IC1"]

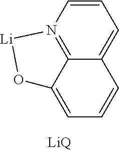

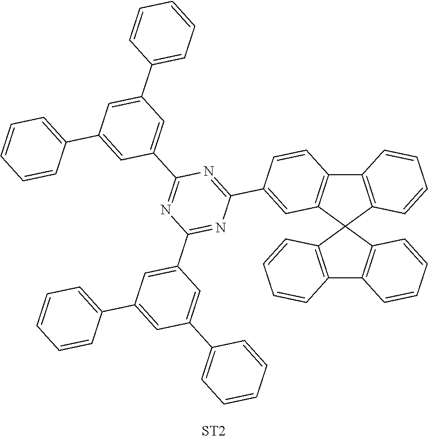 [SMILES: c1ccc(-c2cc(-c3ccccc3)cc(-c3nc(-c4cc(-c5ccccc5)cc(-c5ccccc5)c4)nc(-c4ccc5c(c4)C4(c6ccccc6-c6ccccc64)c4ccccc4-5)n3)c2)cc1 ; "ST2"]

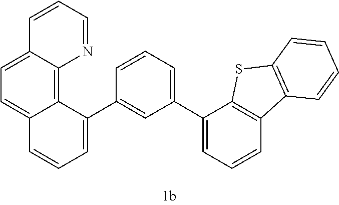 [SMILES: c1cc(-c2cccc3c2sc2ccccc23)cc(-c2cccc3ccc4cccnc4c23)c1]

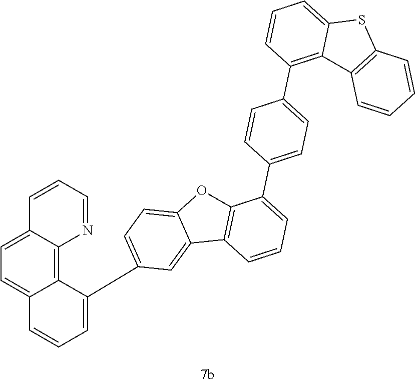 [SMILES: c1cnc2c(c1)ccc1cccc(-c3ccc4oc5c(-c6ccc(-c7cccc8sc9ccccc9c78)cc6)cccc5c4c3)c12]

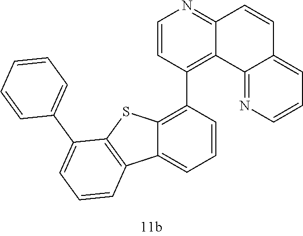 [SMILES: c1ccc(-c2cccc3c2sc2c(-c4ccnc5ccc6cccnc6c45)cccc23)cc1]

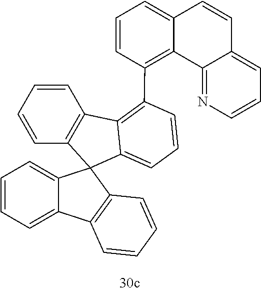 [SMILES: c1ccc2c(c1)-c1ccccc1C21c2ccccc2-c2c(-c3cccc4ccc5cccnc5c34)cccc21]

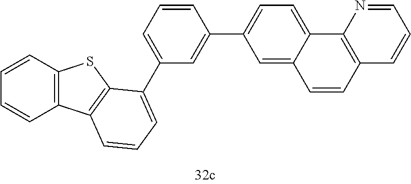 [SMILES: c1cc(-c2ccc3c(ccc4cccnc43)c2)cc(-c2cccc3c2sc2ccccc23)c1]

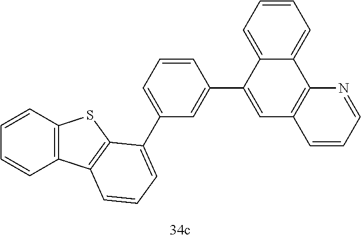 [SMILES: c1cc(-c2cc3cccnc3c3ccccc23)cc(-c2cccc3c2sc2ccccc23)c1]

TABLE 3
Data of the OLEDs

| | U1000 | SE1000 | EQE 1000 | CIE x/y at |
|---|---|---|---|---|
| Ex. | (V) | (cd/A) | (%) | 1000 cd/m² |
| E1 | 3.4 | 62 | 17 | 0.32/0.62 |
| E2 | 3.7 | 62 | 17 | 0.32/0.62 |
| E3 | 3.7 | 56 | 16 | 0.32/0.62 |
| E4 | 3.6 | 57 | 16 | 0.32/0.62 |
| E5 | 3.6 | 59 | 17 | 0.32/0.62 |
| E6 | 3.7 | 55 | 16 | 0.32/0.62 |

The data obtained show that the compounds of the application can be used in OLEDs. More particularly, the OLED use examples adduced above show high efficiency and low operating voltage.

It is also possible to use compounds 2b-6b, 8b-10b, 12b-24b, 1c-29c, 31c, 33c and 35c-40c, the synthesis of which is detailed above in part A, to obtain OLEDs having good efficiency and low operating voltage.

1.-22. (canceled)

23. A compound of formula (I) or (II) 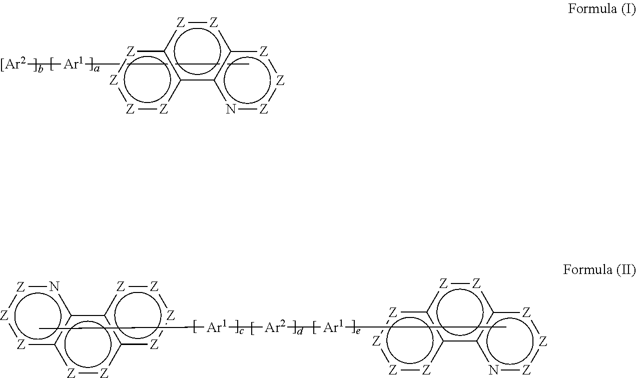
where the variables that occur are as follows:
Z, when no Ar¹ unit binds thereto, is the same or different at each instance and is selected from CR¹ and N; and Z, when an Ar¹ unit binds thereto, is C;
Ar¹ is the same or different at each instance and is selected from a single bond, aromatic ring system which has 6 to 40 aromatic ring atoms and may be substituted by one or more R² radicals, dibenzothiophene which may be substituted by one or more R² radicals, and dibenzofuran which may be substituted by one or more R¹ radicals;
Ar² is the same or different at each instance and is a group of formula (Ar²) 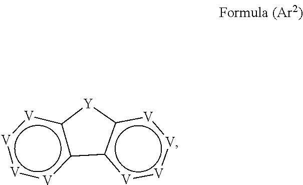
Y is the same or different at each instance and is selected from O, S, C(R³)₂, Si(R³)₂, 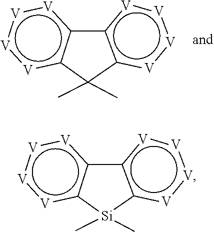
where, in the formulae 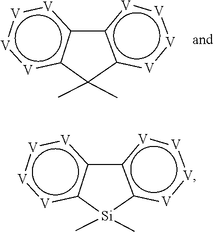
the free bonds are the bonds proceeding from the Y group to the rest of the group of the formula (Ar²);
V is the same or different at each instance and is CR³ or N if the bond to the rest of the formula is not at the position in question, and V is C if the bond to the rest of the formula is at the position in question;
where one or more pairs V-V may each be replaced by a unit selected from the following units: 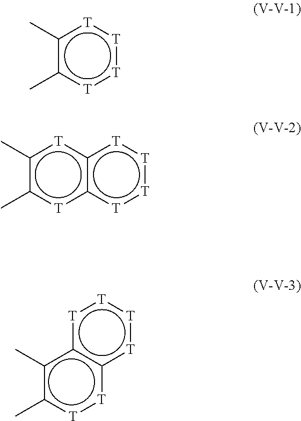
where the free bonds indicate the bonds to the rest of the formula, and where T is the same or different at each instance and is CR³ or N if the bond to the rest of the formula is not at the position in question, and where T is C if the bond to the rest of the formula is at the position in question;
R¹, R² are the same or different at each instance and are selected from H, D, F, C(═O)R⁴, CN, Si(R⁴)₃, P(═O)(R⁴)₂, OR⁴, S(═O)R⁴, S(═O)₂R⁴, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R¹ or R² radicals may be joined to one another and may form a ring; where the alkyl, alkoxy, alkenyl and alkynyl groups mentioned and the aromatic ring systems and heteroaromatic ring systems mentioned may each be substituted by one or more R⁴ radicals; and where one or more CH₂ groups in the alkyl, alkoxy, alkenyl and alkynyl groups mentioned may be replaced by —R⁴C═CR⁴—, —C≡C—, Si(R⁴)₂, C═O, C═NR⁴, —C(═O)O—, —C(═O)NR⁴—, P(═O)(R⁴), —O—, —S—, SO or SO₂;
R³ is the same or different at each instance and is selected from H, D, F, C(═O)R⁴, CN, Si(R⁴)₃, P(═O)(R⁴)₂, OR⁴, S(═O)R⁴, S(═O)₂R⁴, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, heteroaromatic N-free ring systems having 5 to 40 aromatic ring atoms; and electron-deficient heteroaryl groups having 6 to 40 aromatic ring atoms, where two or more R³ radicals may be joined to one another and may form a ring; where the alkyl, alkoxy, alkenyl and alkynyl groups mentioned and the aromatic ring systems and heteroaromatic N-free ring systems and electron-deficient heteroaryl groups mentioned may each be substituted by one or more R⁴ radicals; and where one or more CH₂ groups in the alkyl, alkoxy, alkenyl and alkynyl groups mentioned may be replaced by —R⁴C═CR⁴—, —C≡C—, Si(R⁴)₂, C═O, C═NR⁴, —C(═O)O—, —C(═O)NR⁴—, P(═O)(R⁴), —O—, —S—, SO or SO₂;
R⁴ is the same or different at each instance and is selected from H, D, F, C(═O)R⁵, CN, Si(R⁵)₃, P(═O)(R⁵)₂, OR⁵, S(═O)R⁵, S(═O)₂R⁵, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R⁴ radicals may be joined to one another and may form a ring; where the alkyl, alkoxy, alkenyl and alkynyl groups mentioned and the aromatic ring systems and heteroaromatic ring systems mentioned may each be substituted by one or more R⁵ radicals; and where one or more CH₂ groups in the alkyl, alkoxy, alkenyl and alkynyl groups mentioned may be replaced by —R⁵C═CR⁵—, —C≡C—, Si(R⁵)₂, C═O, C═NR⁵, —C(═O)O—, —C(═O)NR⁵—, P(═O)(R⁵), —O—, —S—, SO or SO₂;
R⁵ is the same or different at each instance and is selected from H, D, F, CN, alkyl or alkoxy groups having 1 to 20 carbon atoms, alkenyl or alkynyl groups having 2 to 20 carbon atoms, aromatic ring systems having 6 to 40 aromatic ring atoms and heteroaromatic ring systems having 5 to 40 aromatic ring atoms; where the alkyl, alkoxy, alkenyl and alkynyl groups, aromatic ring systems and heteroaromatic ring systems mentioned may be substituted by one or more radicals selected from F and CN;
a, b, c, d, e are the same or different and are selected from 1, 2, 3 and 4.

24. The compound as claimed in 23, wherein, for every six-membered ring in one unit 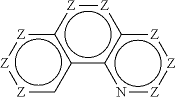
in the formulae (I) and (II), not more than one Z group is N.

25. The compound as claimed in 23, wherein Ar¹ is the same or different at each instance and is selected from a single bond and the formulae: 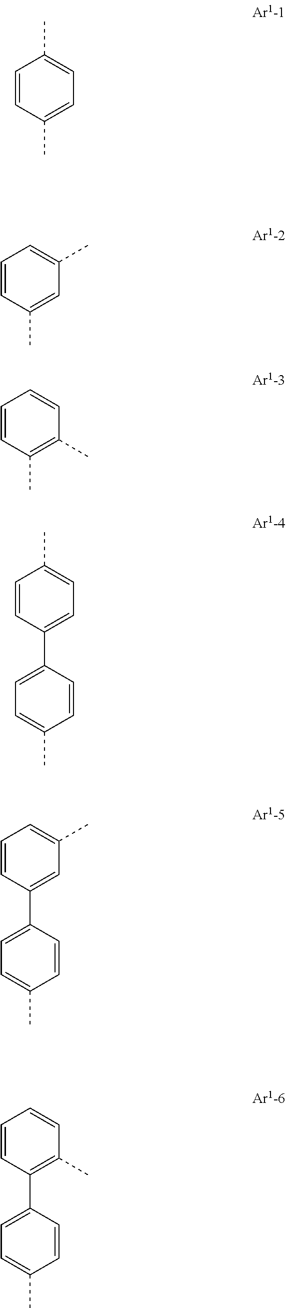 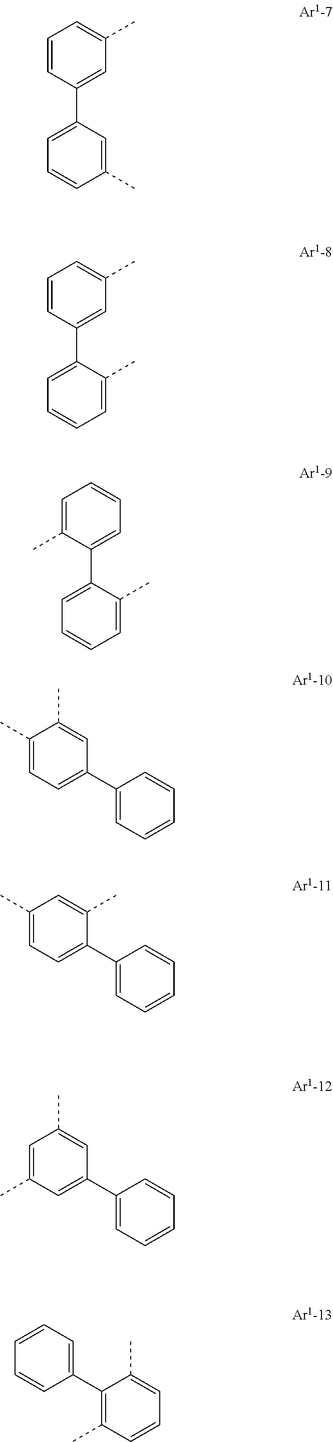 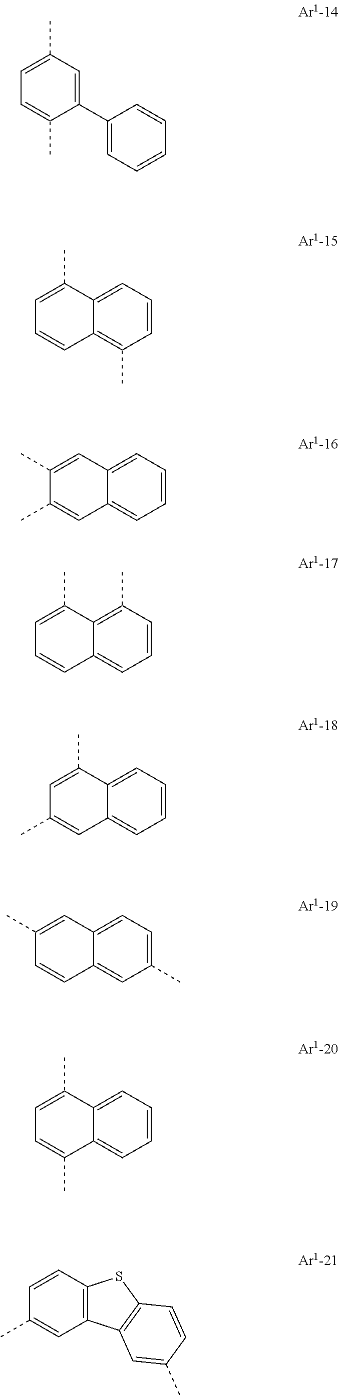 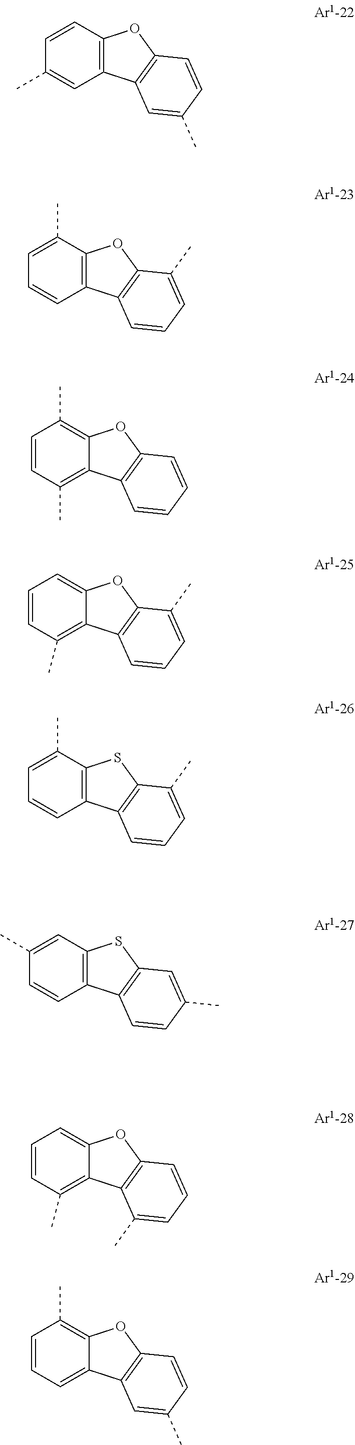 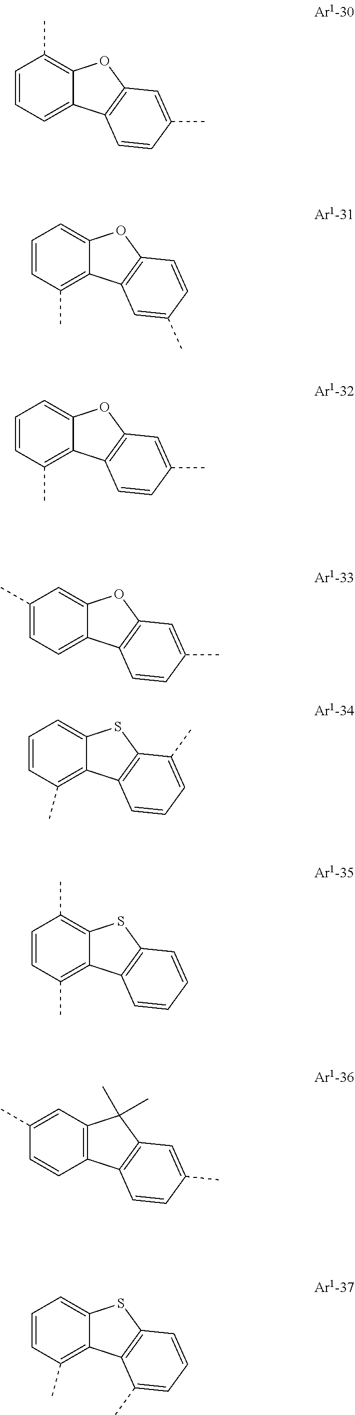 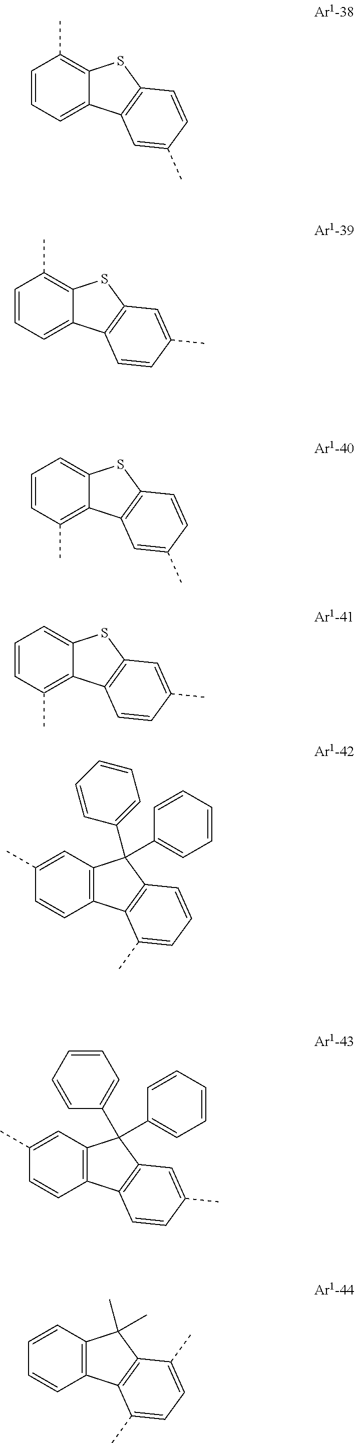 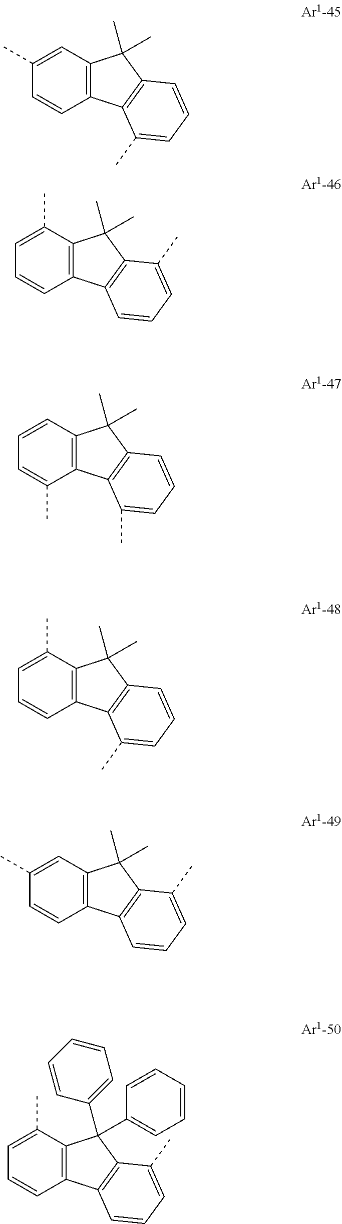 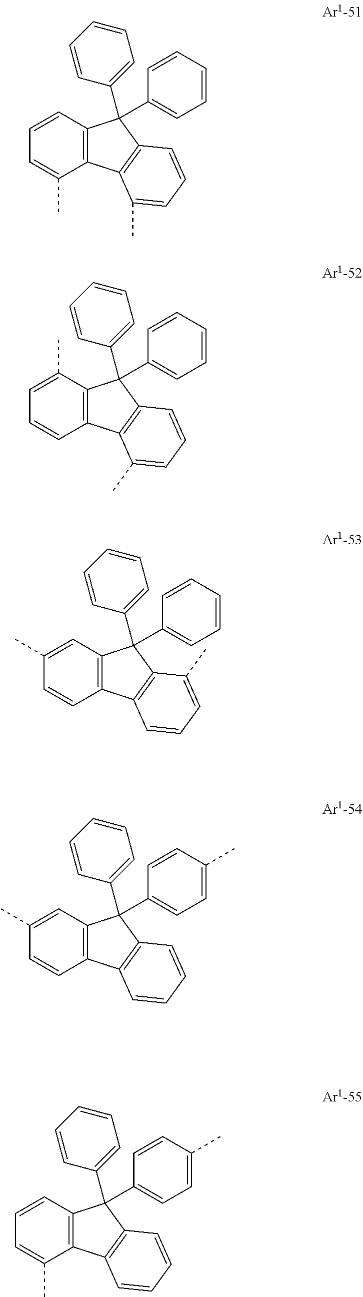 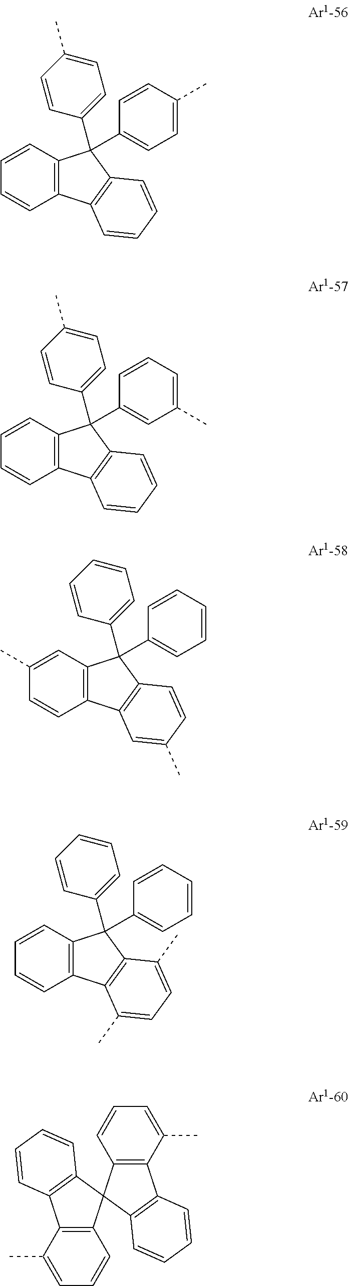 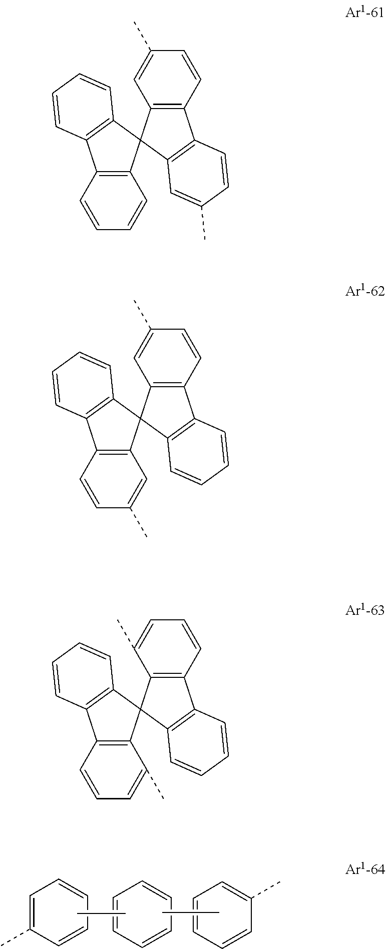
where the formulae may each be substituted by one or more R² radicals.

26. The compound as claimed in 23, wherein Y is the same or different at each instance and is selected from O and S.

27. The compound as claimed in 23, wherein Ar² conforms to a formula selected from the following formulae: 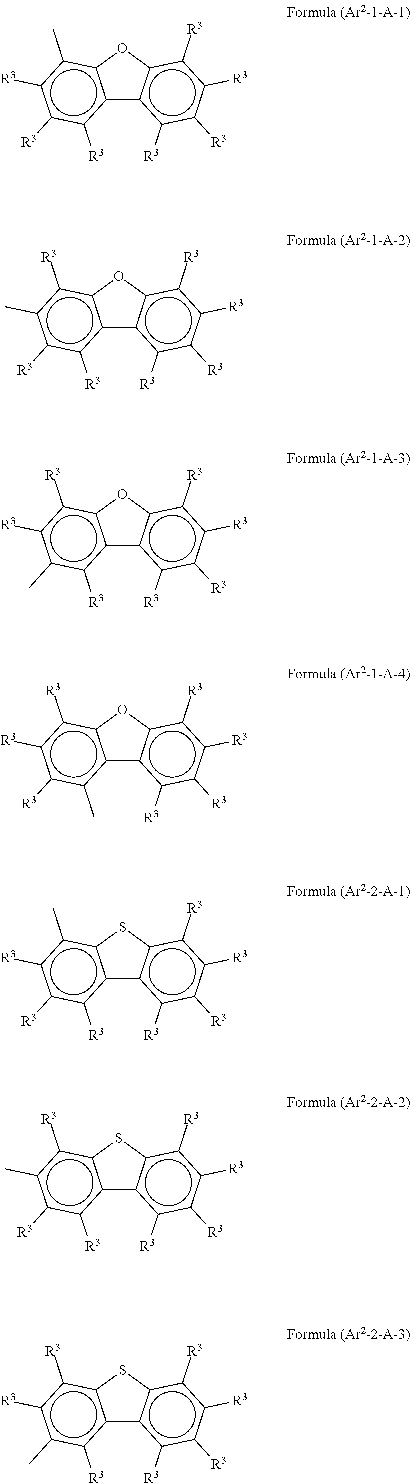 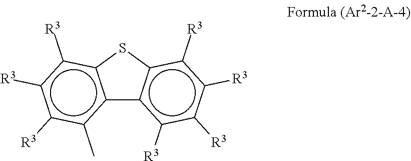
where the free bond denotes the bond to the rest of the formula.

28. The compound as claimed in 23, wherein R¹ is the same or different at each instance and is selected from H, D, F, CN, aromatic ring systems which have 6 to 40 aromatic ring atoms and may each be substituted by one or more R⁴ radicals, and heteroaromatic ring systems which have 5 to 40 aromatic ring atoms and may each be substituted by one or more R⁴ radicals.

29. The compound as claimed in 23, wherein R² is the same or different at each instance and is selected from H, D, F, CN, aromatic ring systems which have 6 to 40 aromatic ring atoms and may each be substituted by one or more R⁴ radicals, and heteroaromatic ring systems which have 5 to 40 aromatic ring atoms and may each be substituted by one or more R⁴ radicals.

30. The compound as claimed in 23, wherein R³ is the same or different at each instance and is selected from H, D, F, CN, aromatic ring systems which have 6 to 40 aromatic ring atoms and may each be substituted by one or more R⁴ radicals, and electron-deficient heteroaryl groups which have 6 to 40 aromatic ring atoms and may each be substituted by one or more R⁴ radicals.

31. The compound as claimed in 23, wherein R⁴ is the same or different at each instance and is selected from H, D, F, CN, Si(R⁵)₃, straight-chain alkyl or alkoxy groups having 1 to 20 carbon atoms, branched or cyclic alkyl or alkoxy groups having 3 to 20 carbon atoms, aromatic ring systems having 6 to 40 aromatic ring atoms and heteroaromatic ring systems having 5 to 40 aromatic ring atoms; where the alkyl and alkoxy groups mentioned, the aromatic ring systems mentioned and the heteroaromatic ring systems mentioned may each be substituted by one or more R⁵ radicals; and where one or more CH₂ groups in the alkyl or alkoxy groups mentioned may be replaced by —C≡C—, —R⁵C═CR⁵—, Si(R⁵)₂, C═O, C═NR⁵, —O—, —S—, —C(═O)O— or —C(═O)NR⁵—.

32. The compound as claimed in 23, wherein a=1 and b=1; or characterized in that a=1 and b=2, where the two Ar² groups chosen are the same or different.

33. The compound as claimed in 23, wherein c=1, e=1 and d=1; or characterized in that c=1, e=1 and d=2, where the two Ar² groups chosen are the same or different.

34. The compound as claimed in 23, wherein compound conforms to one of the formulae (I-A-1), (I-A-2), (I-A-3), (II-A-1) and (II-A-2) 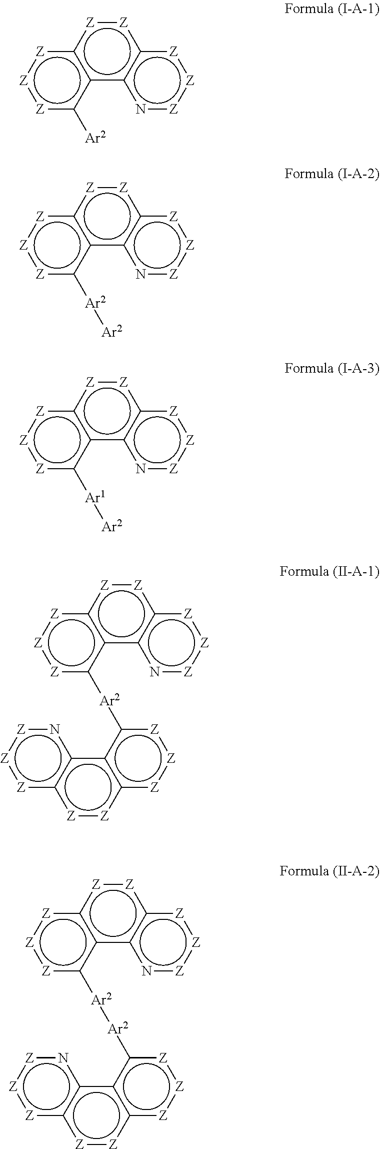
where the groups that occur are as defined in claim 23, except that Ar¹ is not a single bond.

35. The compound as claimed in 25, wherein the compound conforms to one of the formulae (I-A-1), (I-A-2) and (I-A-3) 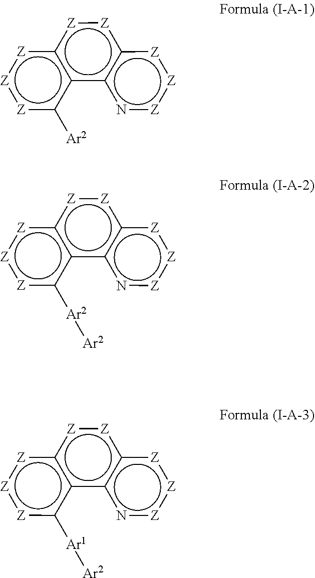
where Z is CR³,
Ar¹ is as defined in claim 25,
Ar² is conforms to a formula selected from the following formulae: 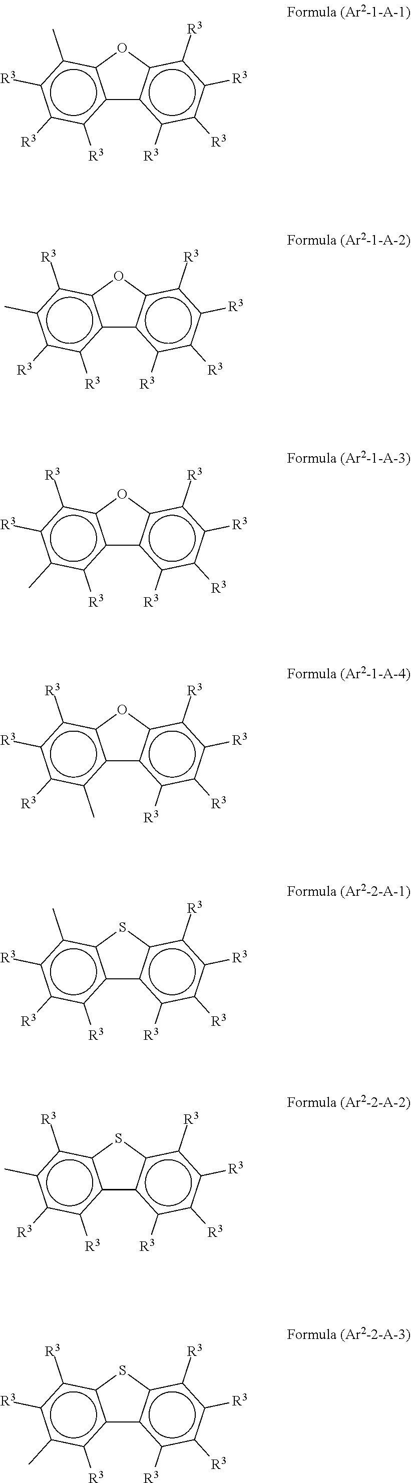 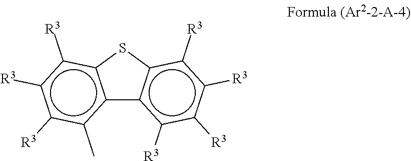
where the free bond denotes the bond to the rest of the formula.
R¹ is the same or different at each instance and is selected from H, D, F, CN, aromatic ring systems which have 6 to 40 aromatic ring atoms and may each be substituted by one or more R⁴ radicals, and heteroaromatic ring systems which have 5 to 40 aromatic ring atoms and may each be substituted by one or more R⁴ radicals,
R² is the same or different at each instance and is selected from H, D, F, CN, aromatic ring systems which have 6 to 40 aromatic ring atoms and may each be substituted by one or more R⁴ radicals, and heteroaromatic ring systems which have 5 to 40 aromatic ring atoms and may each be substituted by one or more R⁴ radicals,
R³ is the same or different at each instance and is selected from H, D, F, CN, aromatic ring systems which have 6 to 40 aromatic ring atoms and may each be substituted by one or more R⁴ radicals, and electron-deficient heteroaryl groups which have 6 to 40 aromatic ring atoms and may each be substituted by one or more R⁴ radicals, and
R⁴ is the same or different at each instance and is selected from H, D, F, CN, Si(R⁵)₃, straight-chain alkyl or alkoxy groups having 1 to 20 carbon atoms, branched or cyclic alkyl or alkoxy groups having 3 to 20 carbon atoms, aromatic ring systems having 6 to 40 aromatic ring atoms and heteroaromatic ring systems having 5 to 40 aromatic ring atoms; where the alkyl and alkoxy groups mentioned, the aromatic ring systems mentioned and the heteroaromatic ring systems mentioned may each be substituted by one or more R⁵ radicals; and where one or more CH₂ groups in the alkyl or alkoxy groups mentioned may be replaced by —C≡C—, —R⁵C═CR⁵—, Si(R⁵)₂, C═O, C═NR⁵, O, S, C(═O)O— or —C(═O)NR⁵—.

36. A process for preparing the compound as claimed in 23, which comprises converting a compound of the formula (Z) 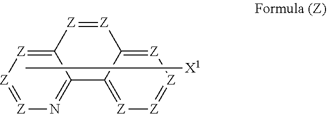
where X¹ is selected from B(OR⁴)₂, Cl, Br and I in a Suzuki reaction.

37. An oligomer, polymer or dendrimer comprising one or more compounds of formula (I) or (II) as claimed in claim 23, wherein the bond(s) to the polymer, oligomer or dendrimer may be localized at any desired positions substituted by R¹, R² or R³ in formula (I) or (II).

38. A formulation comprising the at least one compound as claimed in claim 23 and at least one solvent.

39. A formulation comprising the polymer, oligomer or dendrimer as claimed in claim 37 and at least one solvent.

40. An electronic device comprising at least one compound as claimed in claim 23.

41. An organic electroluminescent device which comprises at least one compound as claimed in claim 23, is present as matrix material in an emitting layer together with at least one phosphorescent emitting compound and at least one further matrix material.

42. The device as claimed in claim 41, wherein the further matrix material is selected from bipolar matrix materials.

43. The electronic device as claimed in claim 41, wherein the further matrix material is selected from compounds of the formulae (BP-1) and (BP-2) 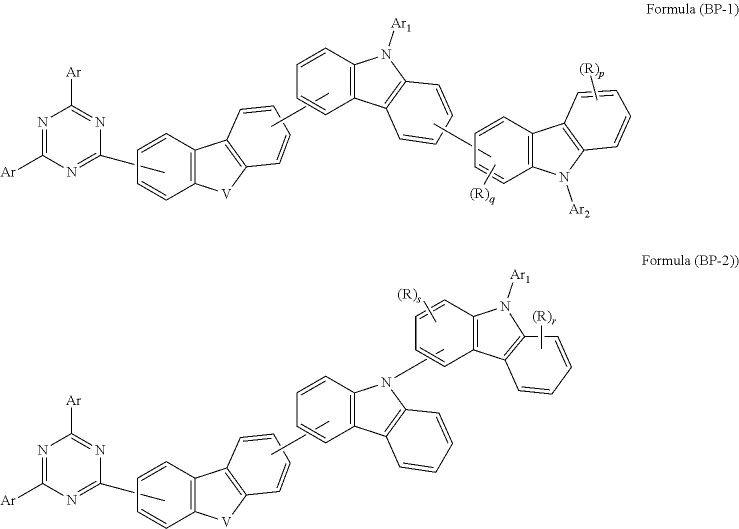
where the variables that occur are as follows:
V is O or S;
Ar, Ar₁, Ar₂ at each instance are each independently an aryl or heteroaryl group which has 5 to 40 ring atoms and may be substituted by one or more R₃ radicals or an aromatic or heteroaromatic ring system which has 6 to 40 ring atoms and may be substituted by one or more R₃ radicals;
p, q are each independently 0, 1, 2, 3 or 4;
s, r are each independently 0, 1, 2, 3 or 4;
R is the same or different at each instance and is selected from the group consisting of D, F, Cl, Br, I, CN, NO₂, N(Ar)₂, N(R₂)₂, C(═O)Ar, C(═O)R₂, P(═O)(Ar)₂, P(Ar)₂, B(Ar)₂, Si(Ar)₃, Si(R₂)₃, a straight-chain alkyl, alkoxy or thioalkyl group having 1 to 20 carbon atoms or a branched or cyclic alkyl, alkoxy or thioalkyl group having 3 to 20 carbon atoms or an alkenyl group having 2 to 20 carbon atoms, each of which may be substituted by one or more R² radicals, where one or more nonadjacent CH₂ groups may be replaced by R₂C═CR₂, Si(R₂)₂, C═O, C═S, C═NR₂, P(═O)(R₂), SO, SO₂, NR₂, O, S or CONR₂ and where one or more hydrogen atoms may be replaced by D, F, Cl, Br, I, CN or NO₂, an aromatic or heteroaromatic ring system which has 5 to 40 ring atoms and may be substituted in each case by one or more R₂ radicals, an aryloxy or heteroaryloxy group which has 5 to 40 ring atoms and may be substituted by one or more R₂ radicals, or an aralkyl or heteroaralkyl group which has 5 to 40 ring atoms and may be substituted by one or more R₂ radicals; at the same time, it is possible for a maximum of one substituent R together with Ar₁ to form a monocyclic or polycyclic, aliphatic, aromatic or heteroaromatic ring system which may be substituted by one or more R₂ radicals;
R2 is the same or different at each instance and is selected from the group consisting of H, D, F, Cl, Br, I, CN, NO₂, N(Ar)₂, NH₂, N(R₃)₂, C(═O)Ar, C(═O)H, C(═O)R₃, P(═O)(Ar)₂, a straight-chain alkyl, alkoxy or thioalkyl group having 1 to 40 carbon atoms or a branched or cyclic alkyl, alkoxy or thioalkyl group having 3 to 40 carbon atoms or an alkenyl or alkynyl group having 2 to 40 carbon atoms, each of which may be substituted by one or more R₃ radicals, where one or more nonadjacent CH₂ groups may be replaced by HC═CH, R₃C═CR₃, C≡C, Si(R₃)₂, Ge(R₃)₂, Sn(R₃)₂, C═O, C═S, C═Se, C═NR₃, P(═O)(R₃), SO, SO₂, NH, NR₃, O, S, CONH or CONR₃ and where one or more hydrogen atoms may be replaced by D, F, Cl, Br, I, CN or NO₂, an aromatic or heteroaromatic ring system which has 5 to 60 ring atoms and may be substituted in each case by one or more R₃ radicals, an aryloxy or heteroaryloxy group which has 5 to 60 ring atoms and may be substituted by one or more R₃ radicals, or a combination of these systems, where it is optionally possible for two or more adjacent substituents R₂ to form a monocyclic or polycyclic, aliphatic, aromatic or heteroaromatic ring system which may be substituted by one or more R₃ radicals;
R₃ is the same or different at each instance and is selected from the group consisting of H, D, F, CN, an aliphatic hydrocarbyl radical having 1 to 20 carbon atoms, or an aromatic or heteroaromatic ring system having 5 to 30 ring atoms in which one or more hydrogen atoms may be replaced by D, F, Cl, Br, I or CN and which may be substituted by one or more alkyl groups each having 1 to 4 carbon atoms; at the same time, it is possible for two or more adjacent substituents R₃ together to form a mono- or polycyclic, aliphatic ring system.

44. A mixture comprising at least one compound as claimed in claim 23 and at least one further compound selected from bipolar matrix materials.
